(19) United States
(12) Patent Application Publication
(10) Pub. No.: US 2024/0219403 A1
(43) Pub. Date: Jul. 4, 2024

(54) METHODS FOR INHIBITING KRAS ONCOPROTEIN THROUGH ENHANCED GTPASE ACTIVITY

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventor: Piro LITO, New York, NY (US)

(21) Appl. No.: 18/554,176

(22) PCT Filed: Apr. 8, 2022
(86) PCT No.: PCT/US22/24052

§ 371 Date: Oct. 5, 2023

Related U.S. Application Data
(60) Provisional application No. 63/172,946, filed on Apr. 9, 2021.

(51) Int. Cl. G01N 33/68; A61K 45/06; C12Q 1/6886

(52) U.S. Cl. CPC G01N 33/6893; A61K 45/06; C12Q 1/6886; G01N 33/6854; C12Q 2600/158; G01N 2800/52

(57) ABSTRACT

The present disclosure relates to methods for determining whether a cancer patient harboring a constitutively active KRAS mutation will be responsive to treatment with a KRAS^{G12C} inhibitor that selectively targets the inactive state of KRAS. These methods are based on assaying regulators of G-protein signaling (RGS) expression in the cancer patient.

### CROSS-REFERENCE TO RELATED APPLICATIONS

This application is a U.S. national stage application of International Application PCT/US2022/024052, filed Apr. 8, 2022, which claims the benefit of and priority to U.S. Provisional Patent Application No. 63/172,946, filed Apr. 9, 2021, the entire contents of which are incorporated herein by reference.

### STATEMENT OF GOVERNMENT SUPPORT

This invention was made with government support under grant number CA008748 awarded by the National Cancer Institute. The government has certain rights in the invention.

### TECHNICAL FIELD

The present technology provides methods for determining whether a cancer patient harboring a constitutively active KRAS mutation will be responsive to treatment with a KRAS^{G12C} inhibitor that selectively targets the inactive state of KRAS. These methods are based on assaying regulators of G-protein signaling (RGS) expression in the cancer patient.

### SEQUENCE LISTING

The instant application contains a Sequence Listing which has been submitted electronically in ASCII format and is hereby incorporated by reference in its entirety. Said ASCII copy, created on May 18, 2022, is named 115872-2527_SL.txt and is 92,397 bytes in size.

### BACKGROUND

The following description of the background of the present technology is provided simply as an aid in understanding the present technology and is not admitted to describe or constitute prior art to the present technology.

KRAS hydrolyzes guanosine triphosphate (GTP) to guanosine diphosphate (GDP) and controls diverse cellular functions by cycling between an active (GTP-bound) and an inactive (GDP-bound) conformation (1-4). Its weak intrinsic GTPase activity is enhanced by GTPase-activating proteins (GAPs) and the exchange of GDP for GTP is enhanced by guanine-nucleotide exchange factors (GEFs) (5-7). KRAS mutations are among the most common activating alterations found in cancer (4, 8). Most cancer-associated hotspot mutations (˜80%) result in single amino acid substitutions of glycine 12 (e.g. G12C/D/V/ etc.), which prevent the catalytic arginine residue in GAPs from enhancing GTP-hydrolysis (3, 9, 10). Insensitivity to GAPs is thought to render KRAS oncoproteins constitutively active and independent of upstream input for activation.

Emerging evidence suggests that some KRAS oncoproteins signal in a semiautonomous manner and require nucleotide exchange for activation (reviewed in 11). Perhaps the strongest evidence is provided by KRAS^{G12C} inhibitors (12-18), which selectively target the inactive state of the oncoprotein to prevent its activation by nucleotide exchange. It was previously shown that these drugs induce responses in ˜32% of lung cancer patients (19) and that inhibition requires an intact GTPase activity by KRAS^{G12C} (13, 18). If KRAS^{G12C} is insensitive to GAPs, it is unknown how KRAS^{G12C} hydrolyzes sufficient GTP to be susceptible to inactive state-selective inhibitors (FIG. 1A).

### SUMMARY OF THE PRESENT TECHNOLOGY

In one aspect, the present disclosure provides a method for selecting a cancer patient harboring a constitutively active KRAS mutation for treatment with a KRAS^{G12C} inhibitor that selectively targets the inactive state of KRAS comprising (a) detecting mRNA or polypeptide expression levels and/or activity of one or more regulators of G-protein signaling in a biological sample obtained from the cancer patient; and administering to the cancer patient an effective amount of the KRAS^{G12C} inhibitor when expression levels and/or activity of the one or more regulators of G-protein signaling are comparable to a control sample obtained from a healthy subject or a predetermined threshold. In another aspect, the present disclosure provides a method for treating a cancer patient harboring a constitutively active KRAS mutation comprising administering to the cancer patient an effective amount of a KRAS^{G12C} inhibitor that selectively targets the inactive state of KRAS, wherein mRNA or polypeptide expression and/or activity levels of one or more regulators of G-protein signaling in a biological sample obtained from the cancer patient are comparable to a control sample obtained from a healthy subject or a predetermined threshold. Examples of KRAS^{G12C} inhibitors that selectively target the inactive state of KRAS comprises one or more of MRTX1257, MRTX849, AMG510, ARS-1620, ARS-3248, LY3499446, LY3537982, GDC-6036, D-1553, JDQ443, BI 1823911, CodeBreak 100, ARS-853, WW peptide, DC-032-759, PTD-RBD-VIF, AU-8653, or ADT-007.

In one aspect, the present disclosure provides a method for selecting a cancer patient harboring a constitutively active KRAS mutation for treatment with a downstream inhibitor of RAS signaling pathway comprising (a) detecting mRNA or polypeptide expression levels and/or activity of one or more regulators of G-protein signaling in a biological sample obtained from the cancer patient; and (b) administering to the cancer patient an effective amount of the downstream inhibitor of RAS signaling pathway when expression levels and/or activity of the one or more regulators of G-protein signaling are reduced relative to a control sample obtained from a healthy subject or a predetermined threshold. Also disclosed herein are methods for treating a cancer patient harboring a constitutively active KRAS mutation comprising administering to the cancer patient an effective amount of a downstream inhibitor of RAS signaling pathway, wherein mRNA or polypeptide expression and/or activity levels of one or more regulators of G-protein signaling in a biological sample obtained from the cancer patient are reduced relative to a control sample obtained from a healthy subject or a predetermined threshold. Additionally or alternatively, in some embodiments, the downstream inhibitor of RAS signaling pathway comprises one or more of BRAF inhibitors, MEK/ERK inhibitors, AURK inhibitors, and PI3K/Akt inhibitors.

Examples of BRAF inhibitors include, but are not limited to, GDC-0879, SB590885, Encorafenib, RAF265, TAK-632, PLX4720, CEP-32496, AZ628, Sorafenib Tosylate, Sorafenib, Vemurafenib (Zelboraf) and Dabrafenib (GSK2118436).

Examples of MEK/ERK inhibitors include, but are not limited to, MLN2480, Cobimetinib (GDC-0973), MEK 162, RO5126766, GDC-0623, VTX11e, Selumetinib (AZD6244), PD0325901, Trametinib (GSK1120212), U0126-EtOH, PD184352 (CI-1040), Refametinib, PD98059, BIX02189, Binimetinib, Pimasertib (AS-703026), SL327, BIX02188, AZD8330, TAK-733, PD318088, SCH772984, and FR 180204.

Examples of PI3K/Akt inhibitors include, but are not limited to, alpelisib, AMG319, apitolisib, AZD8186, BKM120, BGT226, bimiralisib, buparlisib, CH5132799, copanlisib, CUDC-907, dactolisisb, duvelisib, GDC-0941, GDC-0084, gedatolisib, GSK2292767, GSK2636771, idelalisib, IPI-549, leniolisib, LY294002, LY3023414, nemiralisib, omipalisib, PF-04691502, pictilisib, pilaralisib, PX866, RV-1729, SAR260301, SAR245408, serabelisib, SF1126, sonolisib, taselisib, umbralisib, voxtalisib, VS-5584, wortmannin, WX-037, ZSTK474, MK-2206, A-674563, A-443654, acetoxy-tirucallic acid, 3α- and 3β-acetoxy-tirucallic acids, afuresertib (GSK2110183), 4-amino-pyrido[2,3-d]pyrimidine derivative API-1, 3-aminopyrrolidine, anilinotriazole derivatives, ARQ751, ARQ 092, AT7867, AT13148, 7-azaindole, AZD5363, (−)-balanol derivatives, BAY 1125976, Boc-Phe-vinyl ketone, CCT128930, 3-chloroacetylindole, diethyl 6-methoxy-5,7-dihydroindolo [2,3-b]carbazole-2,10-dicarboxylate, diindolylmethane, 2,3-diphenylquinoxaline derivatives, DM-PIT-1, edelfosine, erucylphosphocholine, erufosine, frenolicin B, GSK-2141795, GSK690693, H-8, H-89, 4-hydroxynonenal, ilmofosine, imidazo-1,2-pyridine derivatives, indole-3-carbinol, ipatasertib, kalafungin, lactoquinomycin, medermycin, 3-methyl-xanthine, miltefosine, 1,6-naphthyridinone derivatives, NL-71-101, N-[(1-methyl-1H-pyrazol-4-yl)carbonyl]-N′-(3-bromophenyl)-thiourea, OSU-A9, perifosine, 3-oxo-tirucallic acid, PH-316, 3-phenyl-3H-imidazo[4,5-b]pyridine derivatives, 6-phenylpurine derivatives, PHT-427, PIT-1, PIT-2, 2-pyrimidyl-5-amidothiophene derivative, pyrrolo[2,3-d]pyrimidine derivatives, quinoline-4-carboxamide, 2-[4-(cyclohexa-1,3-dien-1-yl)-1H-pyrazol-3-yl]phenol, spiroindoline derivatives, triazolo[3,4-f][1,6]naphthyridin-3(2H)-one derivative, triciribine, triciribine mono-phosphate active analogue, and uprosertib.

Examples of AURK inhibitors include, but are not limited to, alisertib, tozasertib, SP-96, AT9283, danusertib (PHA-739358), AMG900, cenisertib, SNS-314, barasertib, hesperadin, AZD1152, GSK1070916, CYC116, BI 811283, AZD2811, PHA680632, reversine, CCT129202, CCT137690, quercetin, VX-680, PF-03814735, XL228, ENMD-2076, BI-847325, Ilorasertib/ABT-348, MK-5108/VX-689, and Chiauranib/CS-2164.

In any and all embodiments of the methods disclosed herein, the constitutively active KRAS mutation comprises a substitution at codon 12, 13 or 61 of KRAS. In certain embodiments, the constitutively active KRAS mutation is G12C, G12V, G12D, G12A, G12R, G12S, G12F, G12L, G13C, G13D, G13R, G13A, G13S, G13V, G13E, Q61H, Q61K, Q61L, Q61R, Q61P, or Q61E.

Additionally or alternatively, in some embodiments of the methods disclosed herein, the cancer patient suffers from a cancer selected from among pancreatic cancer, colon cancer, lung cancer, small intestine cancer, urinary tract cancer, endometrial cancer, cervical cancer, bladder cancer, liver cancer, myeloid leukemia breast cancer and biliary tract cancer. In some embodiments, the lung cancer is non-small-cell lung cancer (NSCLC) or small-cell lung cancer (SCLC).

In any and all embodiments of the methods disclosed herein, the one or more regulators of G-protein signaling comprises RGS3 and/or RGS4. In certain embodiments, RGS3 comprises p75 and/or p25 RGS3 isoforms. Additionally or alternatively, in some embodiments, the methods of the present technology comprise assaying mRNA or polypeptide expression levels encoded by a nucleic acid sequence selected from any of SEQ ID NOs: 3-20.

Additionally or alternatively, in some embodiments of the methods disclosed herein, mRNA expression levels are detected via next-generation sequencing, RNA-seq, real-time quantitative PCR (qPCR), digital PCR (dPCR), Reverse transcriptase-PCR (RT-PCR), Northern blotting, microarray, dot or slot blots, in situ hybridization, or fluorescent in situ hybridization (FISH). Additionally or alternatively, in certain embodiments of the methods disclosed herein, polypeptide expression levels are detected via Western blotting, enzyme-linked immunosorbent assays (ELISA), dot blotting, immunohistochemistry, immunofluorescence, immunoprecipitation, immunoelectrophoresis, or mass-spectrometry.

In any of the preceding embodiments of the methods disclosed herein, the biological sample obtained from the cancer patient comprises biopsied tumor tissue, whole blood, plasma, or serum.

In any and all embodiments of the methods disclosed herein, the KRAS^{G12C} inhibitor or the downstream inhibitor of RAS signaling pathway is administered orally, intranasally, systemically, intravenously, intramuscularly, intraperitoneally, intradermally, intraocularly, iontophoretically, transmucosally, subcutaneously, rectally, intrathecally, intratumorally or topically.

### BRIEF DESCRIPTION OF THE DRAWINGS

FIGS. 1A-1G demonstrate that cancer cell extracts enhance the GTPase activity of KRAS^{G12C}. FIG. 1A shows a simplified schematic of the KRAS^{G12C} nucleotide cycle. FIGS. 1B-1C: KRAS^{G12C}-mutant cell lines were serum-deprived for 16 h (in order to suppress nucleotide exchange) and then treated with the indicated inhibitors. The level of active KRAS (GTP-bound) was determined by RAS-binding domain (RBD)-pulldown and quantified by densitometry. The potency of drug binding to ^{G12C} (kinac/KI) and the half-life for cellular inhibition (mean from the indicated number of cell lines) are shown in (FIG. 1C). FIG. 1D: KRAS^{G12C} was loaded with GTP[γ³²P] and its intrinsic GTPase activity was assayed over time, ensuring sufficient time for the reaction to reach completion (mean±s.e.m, n=2). Also shown is the effect of MRTX1257 treatment on the ratio of active to total cellular KRAS. FIG. 1E: GTP[γ³²P] hydrolysis by KRAS^{G12C} was assayed in the absence or presence of whole-cell extracts (WCE) from H358 lung cancer cells (mean±s.e.m, n=3, two-tailed p value). WCE were either added directly or after fractionation with a 3 kDa molecular cut-off column. FIG. 1F: KRAS splice variants 4A or 4B, either WT or G12C mutant, were loaded with GTP[γ³²P] and assayed for GTP-hydrolysis over time in the absence or presence of cellular extracts. FIG. 1G: Thin layer chromatography (TLC) was used to separate α³²P-labeled nucleotides that were eluted from KRAS^{G12C} incubated with the NF1 GAP-related domain (GRD) or H358 cellular extracts. Shown are the fold increase in the absolute GDP[α³²P] signal and the percent α³²P-labeled nucleotide relative to the total signal per sample. FC: fold change. A representative of at least two experimental repeats is shown in FIGS. 1F-1G.

FIGS. 2A-2D show identification of a protein that enhances the GTPase activity of KRAS^{G12C}. FIG. 2A: The purification schema included size-exclusion chromatography (SEC, steps 1 and 2), desalting (step 3), anion-exchange chromatography (AEC, step 4) and mass spectrometry (MS, step 5). FIG. 2B: H358 cell extracts (2 g) were subjected to SEC and eluted fractions (1 mL each) were incubated with GTP[γ³²P]-loaded KRAS^{G12C} for 1 h and subjected to the γ-phosphate assay. The % GTP[γ³²P] signal in each fraction from step 2 is shown. See FIG. 7A for step 1 analysis. FIG. 2C: Active fractions from step 2 (12-17 mL) were pooled, desalted and subjected to AEC using a linear salt-gradient. The effect of each fraction on GTPase activity is shown. See also FIG. 7B. FIG. 2D: The elution of RGS3 p75 and p25 variants in fractions from peak 1 (P1) and the effect on KRAS^{G12C} GTPase activity.

FIGS. 3A-3H show the mechanistic basis for RGS3-assisted GTP-hydrolysis by KRAS^{G12C}. FIGS. 3A-3C: GTP[γ³²P]-loaded KRAS variants were incubated with buffer, NF1 GAP-related domain (GRD) or RGS3 (p75) for the indicated times, followed by determination of GTPase activity by using the γ-phosphate assay (FIG. 3A) or the α-phosphate assay (FIGS. 3B-3C). FIG. 3D: As in FIG. 3A, except that the hydrolysis transition-state mutation A59G was engineered alongside G12C. FIG. 3E: Molecular model of the interaction of KRAS-GMPPNP with the GAP-domain of RGS3 (RGSD). The catalytic arginine residue of RASA1 (R) is superimposed as a reference point (not part of the modeling). FIGS. 3F-3G: KRAS^{G12C} was loaded with GTP[γ³²P] (FIG. 3F) or GTP[α³²P] (FIG. 3G) and then incubated with WT or asparagine-to-histidine (NH) mutant RGS3 followed by determination of GTPase activity using the γ-phosphate (FIG. 3F) or the α-phosphate (FIG. 3G) assay. A representative of at least two experimental repeats is shown in FIGS. 3A-3G. FIG. 3H: Schematic of the mechanism that enables KRAS mutant-inclusive GAP activity. Cancer-associated hotspot mutations at the G12 residue (in the P-loop) prevent the catalytic arginine of canonical RAS-GAPs (e.g., NF1, RASA1 etc.) from stabilizing the transition state of the hydrolysis reaction. RGS proteins compensate for this deficiency to enhance the GTPase activity of mutant KRAS, a process dependent on an arginine residue in the GAP domain of RGS.

FIGS. 4A-4H demonstrate that RGS3 diminishes KRAS^{G12C} activation in cancer cells. FIG. 4A: H358 cells expressing control or RGS3-specific sgRNAs were immunoblotted to determine RGS3 expression or subjected to RBD-pulldown to determine the effect on active (GTP-bound) KRAS. A representative of at least three experimental repeats is shown. FIG. 4B: Control or RGS3^{-/-} clones were implanted in athymic mice to determine the effect on tumor growth (n=10 for control and n=5 for each sgRGS3 clone). Mean±s.e.m and two-tailed p values are shown. FIGS. 4C-4D: Mice bearing control or RGS3^{-/-} xenografts were treated as shown to determine the effect on tumor growth (n=10 for the control and n=5 for each sgRGS3 clone). Two-tailed p values (FIG. 4C) and endpoint tumor volumes (FIG. 4D) are shown. FIG. 4E: Purified KRAS^{G12C} was loaded with GTP[γ³²P] and assayed for GTP-hydrolysis in the absence or presence of extracts (0.4 μg/μL) from control and RGS3^{-/-} cells as well as extracts from RGS3^{-/-} cells engineered to re-express WT or N460H mutant RGS3. FIG. 4F: Purified KRASWT or KRAS^{G12C} proteins were loaded with GTP[γ³²P] and assayed for hydrolysis in the absence (intrinsic) or in the presence of extracts (WCE) from control or RGS3^{-/-} cells (0, 0.1, 0.2 μg/μL). FIG. 4G: Whole cell extracts from the indicated cell lines were evaluated by RBD-pulldown to determine the level of active KRAS. FIG. 4H: The cells were cultured as 3D tumor spheroids in collagen I-containing medium for 17 days.

FIGS. 5A-5E demonstrates that cellular KRAS^{G12C} inhibition is faster than its intrinsic hydrolysis rate. FIG. 5A: WCE from cancer cell lines treated with the indicated G12Ci (500 nM) for 2 h were subjected to RBD-pull down to determine the level of active KRAS. FIG. 5B: KRAS^{G12C}-mutant cells were treated in complete medium containing a MRTX1257 (500 nM). FIG. 5C: Schematic of the γ-phosphate assay used to measure the GTPase activity of KRAS. Bottom: purified KRAS^{G12C} was loaded with GTP[γ³²P] and assayed to determine its intrinsic GTPase activity over time. Rep: replicate. FIG. 5D: Intrinsic hydrolysis half-lives (t_{1/2}) and rate constants (k) from the literature. FIG. 5E: KRAS pulldowns were quantified by densitometry and analyzed to determine the half-life for cellular inhibition by MRTX1257 (mean±s.e.m, n=4). The dotted lines represent simulated data using mean rate constants from the literature.

FIGS. 6A-6G demonstrates KRAS^{G12C}-directed GTPase-enhancing activity in cell extracts. FIGS. 6A-6B: GTP[γ³²P] hydrolysis was assayed in the absence or presence of WCE from the indicated cancer models. WCE were either added directly or after fractionation with a 3 kDa molecular cut-off column. 4A and 4B denote KRAS splice variants. A representative of at least two experimental repeats is shown. FIGS. 6C-6D: The indicated His-tagged KRAS variants were purified, loaded with GTP[γ³²P], and subjected to GTPase assays in the absence or presence of cellular extracts. The reactions were subjected to SDS-PAGE followed by Coomasie staining (CBB) or immunoblotting with a His-specific antibody. M: molecular weight marker. FIGS. 6E-6F: Purified KRASWT or KRAS^{G12C} proteins were loaded with GTP[γ³²P] and assayed for hydrolysis over time in the presence of cellular extracts. FIG. 6G: Schematic of the α-phosphate assay used to measure the GTPase activity. TLC: thin layer chromatography.

FIGS. 7A-7F shows the identification of RGS3 during chromatographic analysis. FIG. 7A: H358 cell extracts (2 g) were subjected to SEC (step 1). Eluted fractions (1 mL each) were incubated with GTP[γ³²P]-loaded KRAS^{G12C} for 1 h and subjected to the γ-phosphate hydrolysis assay. The optical density (OD, 280 nm) and the % GTP[γ³²P] signal in each fraction are shown. FIG. 7B: Active fractions from SEC step 2 (12-17 mL, see FIG. 2A) were pooled, desalted (step 3) and subjected to AEC using a linear salt-gradient (step 4). The conductivity and optical density are shown. See FIG. 2C for the effect of each fraction on GTPase activity. FIGS. 7C-7E: The mass spectrometry sequence coverage of the RGS3 GAP domain (RGSD, (FIG. 7C)) as well as the score corresponding to several identified peptides (FIG. 7D) and a representative MS spectrum (FIG. 7E) are shown. FIG. 7C discloses SEQ ID NO: 21. FIG. 7D discloses SEQ ID NOS 22, 23, 23, 24, 25, 25, and 26, respectively, in order of appearance. Residue numbering in FIG. 7C is from P49796-1 (Uniprot). FIG. 7F: Phylogenetic tree of 35 human RGS domain-containing proteins, based on their primary protein sequence similarity. Red text denotes RGS family members, black text denotes other proteins containing an RGS GAP domain.

FIGS. 8A-8C demonstrate that RGS3 interacts with KRAS^{G12C} in a nucleotide-dependent manner. FIG. 8A: Co-immunoprecipitation (IP) of endogenous KRAS and RGS3 from KRAS^{G12C}-mutant cell lines treated as shown. BRAF is included as an internal control. The G12Ci does not interact with wild-type KRAS, NRAS or HRAS and only displaces effectors from KRAS^{G12C}. Thus, the decrease in RGS3 co-IP with G12Ci-treatment strongly suggests an interaction with KRAS^{G12C}. I: IgG antibody agarose-conjugate, K: KRAS antibody agarose-conjugate. FIG. 8B: HEK293 cells expressing the indicated GST-KRAS variants and FLAG-RGS3 were subjected to a GST-pulldown. FIG. 8C: His-tagged purified KRAS^{G12C} was loaded with non-hydrolyzable GTPYS or GDP. Top: The proteins were incubated with purified GST-RGS domain (RGSD) and subjected to GST-pulldown to determine the presence of an interaction. Bottom: A pulldown with the RAS-binding domain (RBD) or RAF1, which is known to selectively interact with GTP-bound RAS, was used as a control. A representative of at least two experimental repeats are shown in FIGS. 8A-8C.

FIGS. 9A-9F demonstrate that RGS3 enhances GTP hydrolysis by KRAS^{G12C}. FIG. 9A: Schematic of the continuous γ-PO₄ release assay used to measure the GTPase activity of KRAS. MESG: 2-amino-6-mercapto-7-methylpurine riboside, PNP: purine nucleoside phosphorylase, MMP: ribose 1-phosphate and 2-amino-6-mercapto-7-methylpurine. FIGS. 9B-9C: GTP-loaded KRAS variants were desalted and incubated at room temperature with either buffer or the indicated RGS proteins in the presence of MESG and PNP. Except as indicated in FIG. 9C, KRAS and RGS3 were incubated at a 1:1 ratio (50 μM each). The indicated GAP was added just before 0 min. FIG. 9D: The half-life and rate constant (k) for the RGS-assisted KRAS^{G12C} GTP-hydrolysis were determined by fitting a one-phase association curve to the data (0-60 min). FIGS. 9E-9F: GTP-loaded KRAS^{G12C} was reacted with RGS3, RASA1 or NF1 at the indicated molar ratios. NF1 mutated at the catalytic R-finger was assayed. The kinetic profile is shown in FIG. 9D and the reaction half-life in FIG. 9E. A representative of two experimental repeats is shown in FIGS. 9B-9C and 9E.

FIGS. 10A-10C demonstrate that RGS3 enhances GTP-hydrolysis by several G12/G13 KRAS mutants. FIG. 10A: The indicated G12 mutant KRAS oncoproteins were loaded with GTP[γ³²P] and assayed for GTP hydrolysis in the presence of NF1 (GRD) or RGS3 (p75). FIG. 10B: The purity of the protein preparation was determined by Coomasie staining. FIG. 10C: The indicated GTP-loaded KRAS variants were subjected to hydrolysis assay either alone or in the presence of RGS3 (25 μM each).

FIGS. 11A-11C demonstrate that RGS3 deletion results in enhanced KRAS activation and proliferation. FIG. 11A: WCE from H358 cells expressing control of RGS3-specific sgRNAs were immunoblotted to determine RGS3 expression or subjected to RBD-pulldown to determine the effect on active (GTP-bound) KRAS (mean±s.e.m, n=7, two tailed p value). FIG. 11B: Control or RGS3-null cells were transfected with a non-targeting (NT) or a KRAS^{G12C}-specific siRNA. The effect on cell proliferation (% confluence) was determined by continuous monitoring using Incucyte. FIG. 11C: H358 cells expressing G12C- or G12C/A59G-mutant KRAS were transfected with control or RGS3-specific siRNAs to determine the effect on proliferation over time.

FIGS. 12A-12G demonstrate RGS3 expression inversely correlates with mutant KRAS transcriptional output in lung cancer patients. FIGS. 12A-12B: RNAseq from the lung adenocarcinoma TCGA dataset was used to determine differentially expressed genes between KRAS mutant and wild-type cancers. The KRAS activation score was derived from the mean normalized expression of distinct up- (FIG. 12A) or down- (FIG. 12B) regulated genes and scaled across samples. Median, upper/lower quantiles and outliers as well as two-tailed p values are shown. Dn: down. FIG. 12C: Validation of the KRAS score in KRAS^{G12C}-mutant (H358) cells treated with a G12Ci over time. FIG. 12D: Contingency table of lung cancer patients used to correlate RGS3 expression with mutant KRAS-driven transcriptional output. FIG. 12E: KRAS activation score in lung cancers with low, mid and high RGS3 expression (categorized). ANOVA p values are shown. FIGS. 12F-12G: The correlation coefficient between normalized RGS3 expression (continuous) and up-regulated (FIG. 12F) or down-regulated (FIG. 12G) KRAS activation scores in lung cancers with wild-type or mutant KRAS.

FIGS. 13A-13D demonstrate that RGS3 is required for maximal inactive state-selective inhibition. FIG. 13A: Control or RGS3^{-/-} cells were treated with MRTX849 (500 nM) to determine the effect on KRAS activation. FIGS. 13B-13C: The indicated cells were cultured as 3D tumor spheroids in collagen I-containing medium for 14 days, either in the absence or presence of G12Ci (500 nM). Shown are representative images (FIG. 13B) and spheroid volumes (FIG. 13C, mean±s.e.m). FIG. 13D: As in FIG. 4(C), but AUCs (instead of endpoint volumes) were used to contrast tumor growth inhibition. Two tailed p values are shown.

FIGS. 14A-14E demonstrate that RGS3 expression correlates with G12Ci sensitivity in patient-derived xenografts. FIG. 14A: Mice bearing KRAS^{G12C}-mutant lung cancer PDX models were treated with vehicle or MRTX1257 (50 mg/kg). The best treatment response in each model is shown (n=4 per group). FIG. 14B: Representative vehicle-treated tumors from (FIG. 14A) (2 tumors per PDX) were subjected to RNA sequencing to determine the expression of RGS3 and its correlation with response. FIG. 14C: Control or RGS3-null H358 xenograft tumors were subjected to immunohistochemistry with IgG or RGS3-specific antibodies. FIG. 14D: Representative images of vehicle treated PDX tumors subjected to IHC with an RGS3-specific antibody. FIG. 14E: IHC images were quantified using QuPath and the H-score was correlated with pattern of G12Ci-response. ANOVA and group-specific comparison p values are shown in FIG. 14B and FIG. 14E.

### DETAILED DESCRIPTION

It is to be appreciated that certain aspects, modes, embodiments, variations and features of the present methods are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In practicing the present methods, many conventional techniques in molecular biology, protein biochemistry, cell biology, microbiology and recombinant DNA are used. See, e.g., Sambrook and Russell eds. (2001) Molecular Cloning: A Laboratory Manual, 3rd edition; the series Ausubel et al., eds. (2007) Current Protocols in Molecular Biology; the series Methods in Enzymology (Academic Press, Inc., N.Y.); MacPherson et al., (1991) PCR 1: A Practical Approach (IRL Press at Oxford University Press); MacPherson et al., (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al., eds (1996) Weir's Handbook of Experimental Immunology.

Direct targeting of KRAS by small molecule inhibitors has been previously found to be challenging. Ostrem J M et al., Nat Rev Drug Discov. 2016; 15(11): 771-85; Liu P et al., Acta Pharm Sin B. 2019; 9(5):871-9; Ryan M B, Nat Rev Clin Oncol. 2018; 15(11):709-20; Cox A D et al., Nat Rev Drug Discov. 2014; 13(11):828-51. Indeed, the historical model suggests that mutant RAS is completely unresponsive to cellular GAPs. In contrast, the present application demonstrates that KRAS^{G12C} is sensitive to cellular GAPs; albeit still much less sensitive than KRAS^{WT}. Without wishing to be bound by theory, it is believed that the difference in sensitivity lies on the mechanism responsible for the enhanced GTPase activity by cellular extracts. For KRAS^{WT} the activity is mediated by canonical RAS-GAPs, such as NF1 and RASA1, in an arginine-dependent manner. This process is very fast, reaching steady state in a matter of seconds. As demonstrated herein, for KRAS^{G12C} the activity is mediated by atypical RAS-GAPs, a process that is faster than intrinsic hydrolysis but ˜1-2 orders of magnitude slower than the activity of canonical GAPs towards KRAS^{WT} Taken together, the methods of the present technology are useful for determining whether a cancer patient harboring a constitutively active KRAS mutation will be responsive to treatment with a KRAS^{G12C} inhibitor that selectively targets the inactive state of KRAS by assaying RGS expression levels in the cancer patient.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

The term “adapter” refers to a short, chemically synthesized, nucleic acid sequence which can be used to ligate to the end of a nucleic acid sequence in order to facilitate attachment to another molecule. The adapter can be single-stranded or double-stranded. An adapter can incorporate a short (typically less than 50 base pairs) sequence useful for PCR amplification or sequencing.

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

As used herein, an “alteration” of a gene or gene product (e.g., a marker gene or gene product) refers to the presence of a mutation or mutations within the gene or gene product, e.g., a mutation, which affects the quantity or activity of the gene or gene product, as compared to the normal or wild-type gene. The genetic alteration can result in changes in the quantity, structure, and/or activity of the gene or gene product in a cancer tissue or cancer cell, as compared to its quantity, structure, and/or activity, in a normal or healthy tissue or cell (e.g., a control). For example, an alteration which is associated with cancer, or predictive of responsiveness to KRAS inhibitors, can have an altered nucleotide sequence (e.g., a mutation), amino acid sequence, chromosomal translocation, intra-chromosomal inversion, copy number, expression level, protein level, protein activity, in a cancer tissue or cancer cell, as compared to a normal, healthy tissue or cell. Exemplary mutations include, but are not limited to, point mutations (e.g., silent, missense, or nonsense), deletions, insertions, inversions, linking mutations, duplications, translocations, inter- and intra-chromosomal rearrangements. Mutations can be present in the coding or non-coding region of the gene.

As used herein, the terms “amplify” or “amplification” with respect to nucleic acid sequences, refer to methods that increase the representation of a population of nucleic acid sequences in a sample. Nucleic acid amplification methods are well known to the skilled artisan and include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), recombinase-polymerase amplification (RPA)(TwistDx, Cambridge, UK), transcription mediated amplification, signal mediated amplification of RNA technology, loop-mediated isothermal amplification of DNA, helicase-dependent amplification, single primer isothermal amplification, and self-sustained sequence replication (3SR), including multiplex versions or combinations thereof. Copies of a particular nucleic acid sequence generated in vitro in an amplification reaction are called “amplicons” or “amplification products.”

The terms “cancer” or “tumor” are used interchangeably and refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell. As used herein, the term “cancer” includes premalignant, as well as malignant cancers.

The terms “complementary” or “complementarity” as used herein with reference to polynucleotides (i.e., a sequence of nucleotides such as an oligonucleotide or a target nucleic acid) refer to the base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3′” is complementary to the sequence “3′-T-C-A-5.” Certain bases not commonly found in naturally-occurring nucleic acids may be included in the nucleic acids described herein. These include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be an RNA sequence complementary to the DNA sequence or its complement sequence, and can also be a cDNA.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

A “control nucleic acid sample” or “reference nucleic acid sample” as used herein, refers to nucleic acid molecules from a control or reference sample. In certain embodiments, the reference or control nucleic acid sample is a wild type or a non-mutated DNA or RNA sequence. In certain embodiments, the reference nucleic acid sample is purified or isolated (e.g., it is removed from its natural state). In other embodiments, the reference nucleic acid sample is from a non-tumor sample, e.g., a blood control, a normal adjacent tumor (NAT), or any other non-cancerous sample from the same or a different subject.

“Detecting” as used herein refers to determining the presence of a mutation or alteration in a nucleic acid of interest in a sample. Detection does not require the method to provide 100% sensitivity. Analysis of nucleic acid markers can be performed using techniques known in the art including, but not limited to, sequence analysis, and electrophoretic analysis. Non-limiting examples of sequence analysis include Maxam-Gilbert sequencing, Sanger sequencing, capillary array DNA sequencing, thermal cycle sequencing (Sears et al., Biotechniques, 13:626-633 (1992)), solid-phase sequencing (Zimmerman et al., Methods Mol. Cell Biol, 3:39-42 (1992)), sequencing with mass spectrometry such as matrix-assisted laser desorption/ionization time-of-flight mass spectrometry (MALDI-TOF/MS; Fu et al., Nat. Biotechnol, 16:381-384 (1998)), and sequencing by hybridization. Chee et al., Science, 274:610-614 (1996); Drmanac et al., Science, 260:1649-1652 (1993); Drmanac et al., Nat. Biotechnol, 16:54-58 (1998). Non-limiting examples of electrophoretic analysis include slab gel electrophoresis such as agarose or polyacrylamide gel electrophoresis, capillary electrophoresis, and denaturing gradient gel electrophoresis. Additionally, next generation sequencing methods can be performed using commercially available kits and instruments from companies such as the Life Technologies/Ion Torrent PGM or Proton, the Illumina HiSEQ or MiSEQ, and the Roche/454 next generation sequencing system.

“Detectable label” as used herein refers to a molecule or a compound or a group of molecules or a group of compounds used to identify a nucleic acid or protein of interest. In some embodiments, the detectable label may be detected directly. In other embodiments, the detectable label may be a part of a binding pair, which can then be subsequently detected. Signals from the detectable label may be detected by various means and will depend on the nature of the detectable label. Detectable labels may be isotopes, fluorescent moieties, colored substances, and the like. Examples of means to detect detectable labels include but are not limited to spectroscopic, photochemical, biochemical, immunochemical, electromagnetic, radiochemical, or chemical means, such as fluorescence, chemifluorescence, or chemiluminescence, or any other appropriate means.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

“Gene” as used herein refers to a DNA sequence that comprises regulatory and coding sequences necessary for the production of an RNA, which may have a non-coding function (e.g., a ribosomal or transfer RNA) or which may include a polypeptide or a polypeptide precursor. The RNA or polypeptide may be encoded by a full length coding sequence or by any portion of the coding sequence so long as the desired activity or function is retained. Although a sequence of the nucleic acids may be shown in the form of DNA, a person of ordinary skill in the art recognizes that the corresponding RNA sequence will have a similar sequence with the thymine being replaced by uracil, i.e., “T” is replaced with “U.”

The term “hybridize” as used herein refers to a process where two substantially complementary nucleic acid strands (at least about 65% complementary over a stretch of at least 14 to 25 nucleotides, at least about 75%, or at least about 90% complementary) anneal to each other under appropriately stringent conditions to form a duplex or heteroduplex through formation of hydrogen bonds between complementary base pairs. Hybridizations are typically and preferably conducted with probe-length nucleic acid molecules, preferably 15-100 nucleotides in length, more preferably 18-50 nucleotides in length. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Hybridization and the strength of hybridization (i.e., the strength of the association between the nucleic acids) is influenced by such factors as the degree of complementarity between the nucleic acids, stringency of the conditions involved, and the thermal melting point (Tₘ) of the formed hybrid. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology, John Wiley & Sons, Secaucus, N.J. In some embodiments, specific hybridization occurs under stringent hybridization conditions. An oligonucleotide or polynucleotide (e.g., a probe or a primer) that is specific for a target nucleic acid will “hybridize” to the target nucleic acid under suitable conditions.

As used herein, the term “library” refers to a collection of nucleic acid sequences, e.g., a collection of nucleic acids derived from whole genomic, subgenomic fragments, cDNA, cDNA fragments, RNA, RNA fragments, or a combination thereof. In one embodiment, a portion or all of the library nucleic acid sequences comprises an adapter sequence. The adapter sequence can be located at one or both ends. The adapter sequence can be useful, e.g., for a sequencing method (e.g., an NGS method), for amplification, for reverse transcription, or for cloning into a vector.

The library can comprise a collection of nucleic acid sequences, e.g., a target nucleic acid sequence (e.g., a tumor nucleic acid sequence), a reference nucleic acid sequence, or a combination thereof. In some embodiments, the nucleic acid sequences of the library can be derived from a single subject. In other embodiments, a library can comprise nucleic acid sequences from more than one subject (e.g., 2, 3, 4, 5, 6, 7, 8, 9, 10, 20, 30 or more subjects). In some embodiments, two or more libraries from different subjects can be combined to form a library having nucleic acid sequences from more than one subject.

A “library nucleic acid sequence” refers to a nucleic acid molecule, e.g., a DNA, RNA, or a combination thereof, that is a member of a library. Typically, a library nucleic acid sequence is a DNA molecule, e.g., genomic DNA or cDNA. In some embodiments, a library nucleic acid sequence is fragmented, e.g., sheared or enzymatically prepared, genomic DNA. In certain embodiments, the library nucleic acid sequences comprise sequence from a subject and sequence not derived from the subject, e.g., adapter sequence, a primer sequence, or other sequences that allow for identification, e.g., “barcode” sequences.

The term “multiplex PCR” as used herein refers to amplification of two or more PCR products or amplicons which are each primed using a distinct primer pair.

“Next-generation sequencing or NGS” as used herein, refers to any sequencing method that determines the nucleotide sequence of either individual nucleic acid molecules (e.g., in single molecule sequencing) or clonally expanded proxies for individual nucleic acid molecules in a high throughput parallel fashion (e.g., greater than 10³, 10⁴, 10⁵ or more molecules are sequenced simultaneously). In one embodiment, the relative abundance of the nucleic acid species in the library can be estimated by counting the relative number of occurrences of their cognate sequences in the data generated by the sequencing experiment. Next generation sequencing methods are known in the art, and are described, e.g., in Metzker, M. Nature Biotechnology Reviews 11:31-46 (2010).

As used herein, “oligonucleotide” refers to a molecule that has a sequence of nucleic acid bases on a backbone comprised mainly of identical monomer units at defined intervals. The bases are arranged on the backbone in such a way that they can bind with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group at the 2′ position. Oligonucleotides may also include derivatives, in which the hydrogen of the hydroxyl group is replaced with organic groups, e.g., an allyl group. Oligonucleotides of the method which function as primers or probes are generally at least about 10-15 nucleotides long and more preferably at least about 15 to 25 nucleotides long, although shorter or longer oligonucleotides may be used in the method. The exact size will depend on many factors, which in turn depend on the ultimate function or use of the oligonucleotide. The oligonucleotide may be generated in any manner, including, for example, chemical synthesis, DNA replication, restriction endonuclease digestion of plasmids or phage DNA, reverse transcription, PCR, or a combination thereof. The oligonucleotide may be modified e.g., by addition of a methyl group, a biotin or digoxigenin moiety, a fluorescent tag or by using radioactive nucleotides.

As used herein, the term “primer” refers to an oligonucleotide, which is capable of acting as a point of initiation of nucleic acid sequence synthesis when placed under conditions in which synthesis of a primer extension product which is complementary to a target nucleic acid strand is induced, i.e., in the presence of different nucleotide triphosphates and a polymerase in an appropriate buffer (“buffer” includes pH, ionic strength, cofactors etc.) and at a suitable temperature. One or more of the nucleotides of the primer can be modified for instance by addition of a methyl group, a biotin or digoxigenin moiety, a fluorescent tag or by using radioactive nucleotides. A primer sequence need not reflect the exact sequence of the template. For example, a non-complementary nucleotide fragment may be attached to the 5′ end of the primer, with the remainder of the primer sequence being substantially complementary to the strand. The term primer as used herein includes all forms of primers that may be synthesized including peptide nucleic acid primers, locked nucleic acid primers, phosphorothioate modified primers, labeled primers, and the like. The term “forward primer” as used herein means a primer that anneals to the anti-sense strand of dsDNA. A “reverse primer” anneals to the sense-strand of dsDNA.

As used herein, “primer pair” refers to a forward and reverse primer pair (i.e., a left and right primer pair) that can be used together to amplify a given region of a nucleic acid of interest.

“Probe” as used herein refers to nucleic acid that interacts with a target nucleic acid via hybridization. A probe may be fully complementary to a target nucleic acid sequence or partially complementary. The level of complementarity will depend on many factors based, in general, on the function of the probe. A probe or probes can be used, for example to detect the presence or absence of a mutation in a nucleic acid sequence by virtue of the sequence characteristics of the target. Probes can be labeled or unlabeled, or modified in any of a number of ways well known in the art. A probe may specifically hybridize to a target nucleic acid. Probes may be DNA, RNA or a RNA/DNA hybrid. Probes may be oligonucleotides, artificial chromosomes, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may comprise modified nucleobases, modified sugar moieties, and modified internucleotide linkages. A probe may be used to detect the presence or absence of a target nucleic acid. Probes are typically at least about 10, 15, 20, 25, 30, 35, 40, 50, 60, 75, 100 nucleotides or more in length.

As used herein, a “sample” refers to a substance that is being assayed for the presence of a mutation in a nucleic acid of interest. Processing methods to release or otherwise make available a nucleic acid for detection are well known in the art and may include steps of nucleic acid manipulation. A biological sample may be a body fluid or a tissue sample. In some cases, a biological sample may consist of or comprise blood, plasma, sera, urine, feces, epidermal sample, vaginal sample, skin sample, cheek swab, sperm, amniotic fluid, cultured cells, bone marrow sample, tumor biopsies, aspirate and/or chorionic villi, cultured cells, and the like. Fresh, fixed or frozen tissues may also be used. In one embodiment, the sample is preserved as a frozen sample or as formaldehyde- or paraformaldehyde-fixed paraffin-embedded (FFPE) tissue preparation. For example, the sample can be embedded in a matrix, e.g., an FFPE block or a frozen sample. Whole blood samples of about 0.5 to 5 ml collected with EDTA, ACD or heparin as anti-coagulant are suitable.

The term “sensitivity,” as used herein in reference to the methods of the present technology, is a measure of the ability of a method to detect a preselected sequence variant in a heterogeneous population of sequences. A method has a sensitivity of S % for variants of F % if, given a sample in which the preselected sequence variant is present as at least F % of the sequences in the sample, the method can detect the preselected sequence at a preselected confidence of C %, S % of the time. By way of example, a method has a sensitivity of 90% for variants of 5% if, given a sample in which the preselected variant sequence is present as at least 5% of the sequences in the sample, the method can detect the preselected sequence at a preselected confidence of 99%, 9 out of 10 times (F=5%; C=99%; S=90%).

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

The term “specific” as used herein in reference to an oligonucleotide primer means that the nucleotide sequence of the primer has at least 12 bases of sequence identity with a portion of the nucleic acid to be amplified when the oligonucleotide and the nucleic acid are aligned. An oligonucleotide primer that is specific for a nucleic acid is one that, under the stringent hybridization or washing conditions, is capable of hybridizing to the target of interest and not substantially hybridizing to nucleic acids which are not of interest. Higher levels of sequence identity are preferred and include at least 75%, at least 80%, at least 85%, at least 90%, at least 95% and more preferably at least 98% sequence identity.

“Specificity,” as used herein, is a measure of the ability of a method to distinguish a truly occurring preselected sequence variant from sequencing artifacts or other closely related sequences. It is the ability to avoid false positive detections. False positive detections can arise from errors introduced into the sequence of interest during sample preparation, sequencing error, or inadvertent sequencing of closely related sequences like pseudo-genes or members of a gene family. A method has a specificity of X % if, when applied to a sample set of N_{Total} sequences, in which X_{True} sequences are truly variant and X_{Not true} are not truly variant, the method selects at least X % of the not truly variant as not variant. E.g., a method has a specificity of 90% if, when applied to a sample set of 1,000 sequences, in which 500 sequences are truly variant and 500 are not truly variant, the method selects 90% of the 500 not truly variant sequences as not variant. Exemplary specificities include 90, 95, 98, and 99%.

The term “stringent hybridization conditions” as used herein refers to hybridization conditions at least as stringent as the following: hybridization in 50% formamide, 5×SSC, 50 mM NaH₂PO₄, pH 6.8, 0.5% SDS, 0.1 mg/mL sonicated salmon sperm DNA, and 5× Denhart's solution at 42º C. overnight; washing with 2×SSC, 0.1% SDS at 45° C.; and washing with 0.2×SSC, 0.1% SDS at 45° C. In another example, stringent hybridization conditions should not allow for hybridization of two nucleic acids which differ over a stretch of 20 contiguous nucleotides by more than two bases.

As used herein, the terms “subject”, “patient”, or “individual” can be an individual organism, a vertebrate, a mammal, or a human. In some embodiments, the subject, patient or individual is a human.

As used herein, the terms “target sequence” and “target nucleic acid sequence” refer to a specific nucleic acid sequence to be detected and/or quantified in the sample to be analyzed.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

### KRAS

KRAS (Kirsten rat sarcoma 2 viral oncogene homolog) gene is a proto-oncogene that encodes a small GTPase transductor protein called KRAS. KRAS belongs to a group of small guanosine triphosphate (GTP) binding proteins, known as RAS superfamily or RAS-like GTPases. Members of RAS superfamily are divided into families and subfamilies based on their structure, sequence and function. The five main families are RAS, RHO, RAN, RAB and ARF GTPases. The RAS family itself is further divided into 6 subfamilies (RAS, RAL, RAP, RHEB, RAD and RIT) and each subfamily shares the common core G domain, which provides essential GTPase and nucleotide exchange activity. RAS is the most frequently studied proteins in the RAS subfamily. In humans, three RAS genes encode highly homologous RAS proteins, HRAS, NRAS and KRAS. KRAS protein exists as two splice variants, KRAS4A and KRAS4B, in which KRAS4B is the dominant form in human cells.

KRAS protein contains four domains. The first domain at the N-terminus is identical in the three RAS forms, and the second domain exhibits relatively lower sequence identity. Both regions are important for the signaling function of the KRAS protein and jointly form the G-domain. The G-domain of the KRAS protein includes the GTP-binding pocket, a region within which is essential for the interactions between the putative downstream effectors and GTPase-activating proteins (GAPs). KRAS protein also contains a hypervariable region at the C-terminus, which guides posttranslational modifications and determines plasma membrane anchoring. This region plays an important role in the regulation of the biological activity of RAS protein.

KRAS protein switches between an inactive to an active form via binding to GTP and guanosine diphosphate (GDP), respectively. Under physiological conditions, the transition between these two states is regulated by guanine nucleotide exchange factors (GEFs), such as Son of Sevenless (SOS), or GAPs via different mechanisms that involve catalyzing the exchange of GDP for GTP, potentiating intrinsic GTPase activity or accelerating RAS-mediated GTP hydrolysis. Under physiological conditions, KRAS is predominantly GDP-bound. Upon stimulation like growth factors, nucleotide binding of RAS-GEFs is disabled and releases the nucleotide. Upon binding to GTP, KRAS undergoes conformational changes known to result in two major consequences: 1) affecting KRAS interactions with GAPs, which amplify the GTPase activity of the RAS protein around 100,000-fold; 2) affecting the interactions with GEFs and promoting the release of GTP.

Regardless of the tremendous attempts in the past decades that covered the multiple aspects of KRAS activation, KRAS mutant remains being considered as undruggable.

Methods for Detecting Polynucleotides Associated with Responsiveness to KRAS^{G12C} Inhibitors

Polynucleotides associated with responsiveness to direct KRAS^{G12C} inhibitors may be detected by a variety of methods known in the art. Non-limiting examples of detection methods are described below. The detection assays in the methods of the present technology may include purified or isolated DNA (genomic or cDNA), RNA or protein or the detection step may be performed directly from a biological sample without the need for further DNA, RNA or protein purification/isolation.

### Nucleic Acid Amplification and or Detection

Polynucleotides associated with responsiveness to direct KRAS^{G12C} inhibitors can be detected by the use of nucleic acid amplification techniques that are well known in the art. The starting material may be genomic DNA, cDNA, RNA or mRNA. Nucleic acid amplification can be linear or exponential. Specific variants or mutations may be detected by the use of amplification methods with the aid of oligonucleotide primers or probes designed to interact with or hybridize to a particular target sequence in a specific manner, thus amplifying only the target variant.

Non-limiting examples of nucleic acid amplification techniques include polymerase chain reaction (PCR), real-time quantitative PCR (qPCR), digital PCR (dPCR), reverse transcriptase polymerase chain reaction (RT-PCR), nested PCR, ligase chain reaction (see Abravaya, K. et al., Nucleic Acids Res. (1995), 23:675-682), branched DNA signal amplification (see Urdea, M. S. et al., AIDS (1993), 7(suppl 2):S11-S14), amplifiable RNA reporters, Q-beta replication, transcription-based amplification, boomerang DNA amplification, strand displacement activation, cycling probe technology, isothermal nucleic acid sequence based amplification (NASBA) (see Kievits, T. et al., J Virological Methods (1991), 35:273-286), Invader Technology, next-generation sequencing technology or other sequence replication assays or signal amplification assays.

Primers: Oligonucleotide primers for use in amplification methods can be designed according to general guidance well known in the art as described herein, as well as with specific requirements as described herein for each step of the particular methods described. In some embodiments, oligonucleotide primers for cDNA synthesis and PCR are 10 to 100 nucleotides in length, preferably between about 15 and about 60 nucleotides in length, more preferably 25 and about 50 nucleotides in length, and most preferably between about 25 and about 40 nucleotides in length.

Tₘ of a polynucleotide affects its hybridization to another polynucleotide (e.g., the annealing of an oligonucleotide primer to a template polynucleotide). In certain embodiments of the disclosed methods, the oligonucleotide primer used in various steps selectively hybridizes to a target template or polynucleotides derived from the target template (i.e., first and second strand cDNAs and amplified products). Typically, selective hybridization occurs when two polynucleotide sequences are substantially complementary (at least about 65% complementary over a stretch of at least 14 to 25 nucleotides, preferably at least about 75%, more preferably at least about 90% complementary). See Kanehisa, M., Polynucleotides Res. (1984), 12:203, incorporated herein by reference. As a result, it is expected that a certain degree of mismatch at the priming site is tolerated. Such mismatch may be small, such as a mono-, di- or tri-nucleotide. In certain embodiments, 100% complementarity exists.

Probes: Probes are capable of hybridizing to at least a portion of the nucleic acid of interest or a reference nucleic acid (i.e., wild-type sequence). Probes may be an oligonucleotide, artificial chromosome, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may be used for detecting and/or capturing/purifying a nucleic acid of interest.

Typically, probes can be about 10 nucleotides, about 20 nucleotides, about 25 nucleotides, about 30 nucleotides, about 35 nucleotides, about 40 nucleotides, about 50 nucleotides, about 60 nucleotides, about 75 nucleotides, or about 100 nucleotides long. However, longer probes are possible. Longer probes can be about 200 nucleotides, about 300 nucleotides, about 400 nucleotides, about 500 nucleotides, about 750 nucleotides, about 1,000 nucleotides, about 1,500 nucleotides, about 2,000 nucleotides, about 2,500 nucleotides, about 3,000 nucleotides, about 3,500 nucleotides, about 4,000 nucleotides, about 5,000 nucleotides, about 7,500 nucleotides, or about 10,000 nucleotides long.

Probes may also include a detectable label or a plurality of detectable labels. The detectable label associated with the probe can generate a detectable signal directly. Additionally, the detectable label associated with the probe can be detected indirectly using a reagent, wherein the reagent includes a detectable label, and binds to the label associated with the probe.

In some embodiments, detectably labeled probes can be used in hybridization assays including, but not limited to Northern blots, Southern blots, microarray, dot or slot blots, and in situ hybridization assays such as fluorescent in situ hybridization (FISH) to detect a target nucleic acid sequence within a biological sample. Certain embodiments may employ hybridization methods for measuring expression of a polynucleotide gene product, such as mRNA. Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y., 1989); Berger and Kimmel Methods in Enzymology, Vol. 152, Guide to Molecular Cloning Techniques (Academic Press, Inc., San Diego, Calif, 1987); Young and Davis, PNAS. 80: 1194 (1983).

Detectably labeled probes can also be used to monitor the amplification of a target nucleic acid sequence. In some embodiments, detectably labeled probes present in an amplification reaction are suitable for monitoring the amount of amplicon(s) produced as a function of time. Examples of such probes include, but are not limited to, the 5′-exonuclease assay (TAQMAN® probes described herein (see also U.S. Pat. No. 5,538,848) various stem-loop molecular beacons (see for example, U.S. Pat. Nos. 6,103,476 and 5,925,517 and Tyagi and Kramer, 1996, Nature Biotechnology 14:303-308), stemless or linear beacons (see, e.g., WO 99/21881), PNA Molecular Beacons™ (see, e.g., U.S. Pat. Nos. 6,355,421 and 6,593,091), linear PNA beacons (see, for example, Kubista et al., 2001, SPIE 4264:53-58), non-FRET probes (see, for example, U.S. Pat. No. 6,150,097), Sunrise®/Amplifluor™ probes (U.S. Pat. No. 6,548,250), stem-loop and duplex Scorpion probes (Solinas et al., 2001, Nucleic Acids Research 29:E96 and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB Eclipse™ probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471; Whitcombe et al., 1999, Nature Biotechnology. 17:804-807; Isacsson et al., 2000, Molecular Cell Probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem. Res. Toxicol. 15:118-126; and Yu et al., 2001, J. Am. Chem. Soc 14:11155-11161.

In some embodiments, the detectable label is a fluorophore. Suitable fluorescent moieties include but are not limited to the following fluorophores working individually or in combination: 4-acetamido-4′-isothiocyanatostilbene-2,2′disulfonic acid; acridine and derivatives: acridine, acridine isothiocyanate; Alexa Fluors: Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes); 5-(2-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Black Hole Quencher™ (BHQ™) dyes (biosearch Technologies); BODIPY dyes: BODIPY® R-6G, BOPIPY® 530/550, BODIPY® FL; Brilliant Yellow; coumarin and derivatives: coumarin, 7-amino-4-methylcoumarin (AMC, Coumarin 120), 7-amino-4-trifluoromethylcouluarin (Coumarin 151); Cy2®, Cy3®, Cy3.5®, Cy5®, Cy5.5®; cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); Eclipse™ (Epoch Biosciences Inc.); eosin and derivatives: eosin, eosin isothiocyanate; erythrosin and derivatives: erythrosin B, erythrosin isothiocyanate; ethidium; fluorescein and derivatives: 5-carboxyfluorescein (FAM), 5-(4,6-dichlorotriazin-2-yl)amino fluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescem (TET); fiuorescamine; IR144; IR1446; lanthamide phosphors; Malachite Green isothiocyanate; 4-methylumbelliferone; ortho cresolphthalein; nitrotyrosine; pararosaniline; Phenol Red; B-phycoerythrin, R-phycoerythrin; allophycocyanin; o-phthaldialdehyde; Oregon Green®; propidium iodide; pyrene and derivatives: pyrene, pyrene butyrate, succinimidyl 1-pyrene butyrate; QSY® 7; QSY® 9; QSY® 21; QSY® 35 (Molecular Probes); Reactive Red 4 (Cibacron® Brilliant Red 3B-A); rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, riboflavin, rosolic acid, sulforhodamine B, sulforhodamine 101, sulfonyl chloride derivative of sulforhodamine 101 (Texas Red); terbium chelate derivatives; N,N,N′,N′-tetramethyl-6-carboxyrhodamine (TAMRA); tetramethyl rhodamine; tetramethyl rhodamine isothiocyanate (TRITC); and VIC®. Detector probes can also comprise sulfonate derivatives of fluorescenin dyes with S03 instead of the carboxylate group, phosphoramidite forms of fluorescein, phosphoramidite forms of CY 5 (commercially available for example from Amersham).

Detectably labeled probes can also include quenchers, including without limitation black hole quenchers (Biosearch), Iowa Black (IDT), QSY quencher (Molecular Probes), and Dabsyl and Dabcel sulfonate/carboxylate Quenchers (Epoch).

Detectably labeled probes can also include two probes, wherein for example a fluorophore is on one probe, and a quencher is on the other probe, wherein hybridization of the two probes together on a target quenches the signal, or wherein hybridization on the target alters the signal signature via a change in fluorescence.

In some embodiments, interchelating labels such as ethidium bromide, SYBR® Green I (Molecular Probes), and PicoGreen® (Molecular Probes) are used, thereby allowing visualization in real-time, or at the end point, of an amplification product in the absence of a detector probe. In some embodiments, real-time visualization may involve the use of both an intercalating detector probe and a sequence-based detector probe. In some embodiments, the detector probe is at least partially quenched when not hybridized to a complementary sequence in the amplification reaction, and is at least partially unquenched when hybridized to a complementary sequence in the amplification reaction.

In some embodiments, the amount of probe that gives a fluorescent signal in response to an excited light typically relates to the amount of nucleic acid produced in the amplification reaction. Thus, in some embodiments, the amount of fluorescent signal is related to the amount of product created in the amplification reaction. In such embodiments, one can therefore measure the amount of amplification product by measuring the intensity of the fluorescent signal from the fluorescent indicator.

Primers or probes may be designed to selectively hybridize to any portion of a nucleic acid sequence encoding a polypeptide selected from among RGS3, or RGS4. Exemplary nucleic acid sequences of the human orthologs of these genes are provided below:

NM_001276260.2 Homo sapiens regulator of G protein signaling 3
(RGS3), transcript variant 7, mRNA
(SEQ ID NO: 3)
ACTTCCCTGCGCCTCTGGCTGCCTCAATGTCACTGGGGATTAGTGTGCATTTGCGGCTTTTGGCGGACAC
TCCCCTCCTGGCTGCAGCCTCACCCTCTGGAGCCCAGGGATGGGCTTGGGCCTGGGAGCCCCCTGAACCA
GGGAGCAGCCCTGACCGTCCGTTTCTCTATTTTCCCTGACGTCAGAAGGCAGAGTGCTTATTCACTTTGG
AAGCGCACTCGCAGGAGCAGAAGAAGAGAGTGTGCTGGTGCCTGTCGGAGAACATCGCCAAGCAGCAACA
GCTGGCAGCATCACCCCCGGACAGCAAGATGTTTGAGACGGAGGCAGATGAGAAGAGGGAGATGGCCTTG
GAGGAAGGGAAGGGGCCTGGTGCCGAGGATTCCCCACCCAGCAAGGAGCCCTCTCCTGGCCAGGAGCTTC
CTCCAGGACAAGACCTTCCACCCAACAAGGACTCCCCTTCTGGGCAGGAACCCGCTCCCAGCCAAGAACC
ACTGTCCAGCAAAGACTCAGCTACCTCTGAAGGATCCCCTCCAGGCCCAGATGCTCCGCCCAGCAAGGAT
GTGCCACCATGCCAGGAACCCCCTCCAGCCCAAGACCTCTCACCCTGCCAGGACCTACCTGCTGGTCAAG
AACCCCTGCCTCACCAGGACCCTCTACTCACCAAAGACCTCCCTGCCATCCAGGAATCCCCCACCCGGGA
CCTTCCACCCTGTCAAGATCTGCCTCCTAGCCAGGTCTCCCTGCCAGCCAAGGCCCTTACTGAGGACACC
ATGAGCTCCGGGGACCTACTAGCAGCTACTGGGGACCCACCTGCGGCCCCCAGGCCAGCCTTCGTGATCC
CTGAGGTCCGGCTGGATAGCACCTACAGCCAGAAGGCAGGGGCAGAGCAGGGCTGCTCGGGAGATGAGGA
GGATGCAGAAGAGGCCGAGGAGGTGGAGGAGGGGGAGGAAGGGGAGGAGGACGAGGATGAGGACACCAGC
GATGACAACTACGGAGAGCGCAGTGAGGCCAAGCGCAGCAGCATGATCGAGACGGGCCAGGGGGCTGAGG
GTGGCCTCTCACTGCGTGTGCAGAACTCGCTGCGGCGCCGGACGCACAGCGAGGGCAGCCTGCTGCAGGA
GCCCCGAGGGCCCTGCTTTGCCTCCGACACCACCTTGCACTGCTCAGACGGTGAGGGCGCCGCCTCCACC
TGGGGCATGCCTTCGCCCAGCACCCTCAAGAAAGAGCTGGGCCGCAATGGTGGCTCCATGCACCACCTTT
CCCTCTTCTTCACAGGACACAGGAAGATGAGCGGGGCTGACACCGTTGGGGATGATGACGAAGCCTCCCG
GAAGAGAAAGAGCAAAAACCTAGCCAAGGACATGAAGAACAAGCTGGGGATCTTCAGACGGCGGAATGAG
TCCCCTGGAGCCCCTCCCGCGGGCAAGGCAGACAAAATGATGAAGTCATTCAAGCCCACCTCAGAGGAAG
CCCTCAAGTGGGGCGAGTCCTTGGAGAAGCTGCTGGTTCACAAATACGGGTTAGCAGTGTTCCAAGCCTT
CCTTCGCACTGAGTTCAGTGAGGAGAATCTGGAGTTCTGGTTGGCTTGTGAGGACTTCAAGAAGGTCAAG
TCACAGTCCAAGATGGCATCCAAGGCCAAGAAGATCTTTGCTGAATACATCGCGATCCAGGCATGCAAGG
AGGTCAACCTGGACTCCTACACGCGGGAGCACACCAAGGACAACCTGCAGAGCGTCACGCGGGGCTGCTT
CGACCTGGCACAGAAGCGCATCTTCGGGCTCATGGAAAAGGACTCGTACCCTCGCTTTCTCCGTTCTGAC
CTCTACCTGGACCTTATTAACCAGAAGAAGATGAGTCCCCCGCTTTAGGGGCCACTGGAGTCGAGCTCAG
CGTTCACACCAGGCGGGCTGGGTCCCCTGCCCACCTGCCTCCCTGCCCCCTGTGACGGAGGGGGCAAGCA
AGCCCCCAGAGGCTGTGTCTCTGGACAGACGGATAGACATACGGAAGCGAGGCCTGGACCAAGAGAGGCC
CAGGCTACTGGAGGAGTAGAAGGATGGGCCCCGTGGGGTCCCCACTGCCCCGGTACGAGGGGGCCCAAGA
CCCTGGCAGGTCAGGGGCCCTGGCCAAGCCAGATCTGGAGCTGCTGCTCCCTGCTGCGGAGACCGCGGAG
GCTTCGCGTTGACCAAGTTCCTTAAAGAACTGGCTGATGGGGCAGGAGGTCCAGGCCTGGGCTCTCGGGC
CCTCCTAGAGGGCCATTGGAGCTTGCAGCTCAGACCCCCACTTTGAGTTTTATTTATTTAAATAGTAGTT
GGATGCTTGGCACGTCGTCCTGTAATAGGAAACCCTTGCCTCATCAGTTTTCCTGATTTACAAGTGCAAT
ATTTTAGCCAATGCCTTGGGAGAAGCTGCCATGCAAAGGTGGACACCATTCTCCAGCTTCAGGGGATATG
CTCGTCCCGGGCACCGGTGGCAGGCAGCTGGCCTTCTGGACTAAGGCAGCCTGGGGGGACACTGCAGTCT
GGCTACACACAGAGATCTGGCACCCCCTGGGTGGAGTGTCCCTCGGGGGCTTTGGGAAAGCATGGCACCC
TCAGACCACACAGTAGCCAAGTTCTGGAGCAAATAAAAGGCCTGTGTTATTTCTTGTTCTTGA
>NM_001276261.2 Homo sapiens regulator of G protein signaling 3
(RGS3), transcript variant 8, mRNA
(SEQ ID NO: 4)
AGTGCGGGCTCCCGCTGCTGGGGGAGAGCTGGGTTTTCATGGGGCGGCAGCCGAGGCAGGACCCGCAGCC
ATGAACCGCTTCAATGGGCTCTGCAAGGTGTGCTCGGAGCGCCGCTACCGCCAGATCACCATCCCGAGGG
GAAAGGACGGCTTTGGCTTCACCATCTGCTGCGACTCTCCAGTTCGAGTCCAGGCCGTGGATTCCGGGGG
TCCGGCGGAACGGGCAGGGCTGCAGCAGCTGGACACGGTGCTGCAGCTGAATGAGAGGCCTGTGGAGCAC
TGGAAATGTGTGGAGCTGGCCCACGAGATCCGGAGCTGCCCCAGTGAGATCATCCTACTCGTGTGGCGCA
TGGTCCCCCAGGTCAAGCCAGGACCAGATGGCGGGGTCCTGCGGCGGGCCTCCTGCAAGTCGACACATGA
CCTCCAGTCACCCCCCAACAAACGGGAGAAGAACTGCACCCATGGGGTCCAGGCACGGCCTGAGCAGCGC
CACAGCTGCCACCTGGTATGTGACAGCTCTGATGGGCTGCTGCTCGGCGGCTGGGAGCGCTACACCGAGG
TGGCCAAGCGCGGGGGCCAGCACACCCTGCCTGCACTGTCCCGTGCCACTGCCCCCACCGACCCCAACTA
CATCATCCTGGCCCCGCTGAATCCTGGGAGCCAGCTGCTCCGGCCTGTGTACCAGGAGGATACCATCCCC
GAAGAATCAGGGAGTCCCAGTAAAGGGAAGTCCTACACAGGCCTGGGGAAGAAGTCCCGGCTGATGAAGA
CAGTGCAGACCATGAAGGGCCACGGGAACTACCAAAACTGCCCGGTTGTGAGGCCGCATGCCACGCACTC
AAGCTATGGCACCTACGTCACCCTGGCCCCCAAAGTCCTGGTGTTCCCTGTCTTTGTTCAGCCTCTAGAT
CTCTGTAATCCTGCCCGGACCCTCCTGCTGTCAGAGGAGCTGCTGCTGTATGAAGGGAGGAACAAGGCTG
CCGAGGTGACACTGTTTGCCTATTCGGACCTGCTGCTCTTCACCAAGGAGGACGAGCCTGGCCGCTGCGA
CGTCCTGAGGAACCCCCTCTACCTCCAGAGTGTGAAGCTGCAGGAAGGTTCTTCAGAAGACCTGAAATTC
TGCGTGCTCTATCTAGCAGAGAAGGCAGAGTGCTTATTCACTTTGGAAGCGCACTCGCAGGAGCAGAAGA
AGAGAGTGTGCTGGTGCCTGTCGGAGAACATCGCCAAGCAGCAACAGCTGGCAGCATCACCCCCGGACAG
CAAGATGAGCGGGGCTGACACCGTTGGGGATGATGACGAAGCCTCCCGGAAGAGAAAGAGCAAAAACCTA
GCCAAGGACATGAAGAACAAGCTGGGGATCTTCAGACGGCGGAATGAGTCCCCTGGAGCCCCTCCCGCGG
GCAAGGCAGACAAAATGATGAAGTCATTCAAGCCCACCTCAGAGGAAGCCCTCAAGTGGGGCGAGTCCTT
GGAGAAGCTGCTGGTTCACAAATACGGGTTAGCAGTGTTCCAAGCCTTCCTTCGCACTGAGTTCAGTGAG
GAGAATCTGGAGTTCTGGTTGGCTTGTGAGGACTTCAAGAAGGTCAAGTCACAGTCCAAGATGGCATCCA
AGGCCAAGAAGATCTTTGCTGAATACATCGCGATCCAGGCATGCAAGGAGGTCAACCTGGACTCCTACAC
GCGGGAGCACACCAAGGACAACCTGCAGAGCGTCACGCGGGGCTGCTTCGACCTGGCACAGAAGCGCATC
TTCGGGCTCATGGAAAAGGACTCGTACCCTCGCTTTCTCCGTTCTGACCTCTACCTGGACCTTATTAACC
AGAAGAAGATGAGTCCCCCGCTTTAGGGGCCACTGGAGTCGAGCTCAGCGTTCACACCAGGCGGGCTGGG
TCCCCTGCCCACCTGCCTCCCTGCCCCCTGTGACGGAGGGGGCAAGCAAGCCCCCAGAGGCTGTGTCTCT
GGACAGACGGATAGACATACGGAAGCGAGGCCTGGACCAAGAGAGGCCCAGGCTACTGGAGGAGTAGAAG
GATGGGCCCCGTGGGGTCCCCACTGCCCCGGTACGAGGGGGCCCAAGACCCTGGCAGGTCAGGGGCCCTG
GCCAAGCCAGATCTGGAGCTGCTGCTCCCTGCTGCGGAGACCGCGGAGGCTTCGCGTTGACCAAGTTCCT
TAAAGAACTGGCTGATGGGGCAGGAGGTCCAGGCCTGGGCTCTCGGGCCCTCCTAGAGGGCCATTGGAGC
TTGCAGCTCAGACCCCCACTTTGAGTTTTATTTATTTAAATAGTAGTTGGATGCTTGGCACGTCGTCCTG
TAATAGGAAACCCTTGCCTCATCAGTTTTCCTGATTTACAAGTGCAATATTTTAGCCAATGCCTTGGGAG
AAGCTGCCATGCAAAGGTGGACACCATTCTCCAGCTTCAGGGGATATGCTCGTCCCGGGCACCGGTGGCA
GGCAGCTGGCCTTCTGGACTAAGGCAGCCTGGGGGGACACTGCAGTCTGGCTACACACAGAGATCTGGCA
CCCCCTGGGTGGAGTGTCCCTCGGGGGCTTTGGGAAAGCATGGCACCCTCAGACCACACAGTAGCCAAGT
TCTGGAGCAAATAAAAGGCCTGTGTTATTTCTTGTTCTTGA
NM_001276262.2 Homo sapiens regulator of G protein signaling 3
(RGS3), transcript variant 9, mRNA
(SEQ ID NO: 5)
AGTCATCAGGCCAGGATTCCAGAGAGCGTGTGTGGCTGCAGCCTGCACCGTTGCTGCCCGCTGCCCAGGA
CGCGGGGTGGGGGACAGGAGCCAGAGTGGTGCCTCCTACAGACCAATCTGCGGCCCCAAGGTGGGGGGCC
CTACAGAGATGCTCCGAGAGCCAAGGACATGAAGAACAAGCTGGGGATCTTCAGACGGCGGAATGAGTCC
CCTGGAGCCCCTCCCGCGGGCAAGGCAGACAAAATGATGAAGTCATTCAAGCCCACCTCAGAGGAAGCCC
TCAAGTGGGGCGAGTCCTTGGAGAAGCTGCTGGTTCACAAATACGGGTTAGCAGTGTTCCAAGCCTTCCT
TCGCACTGAGTTCAGTGAGGAGAATCTGGAGTTCTGGTTGGCTTGTGAGGACTTCAAGAAGGTCAAGTCA
CAGTCCAAGATGGCATCCAAGGCCAAGAAGATCTTTGCTGAATACATCGCGATCCAGGCATGCAAGGAGG
TCAACCTGGACTCCTACACGCGGGAGCACACCAAGGACAACCTGCAGAGCGTCACGCGGGGCTGCTTCGA
CCTGGCACAGAAGCGCATCTTCGGGCTCATGGAAAAGGACTCGTACCCTCGCTTTCTCCGTTCTGACCTC
TACCTGGACCTTATTAACCAGAAGAAGATGAGTCCCCCGCTTTAGGGGCCACTGGAGTCGAGCTCAGCGT
TCACACCAGGCGGGCTGGGTCCCCTGCCCACCTGCCTCCCTGCCCCCTGTGACGGAGGGGGCAAGCAAGC
CCCCAGAGGCTGTGTCTCTGGACAGACGGATAGACATACGGAAGCGAGGCCTGGACCAAGAGAGGCCCAG
GCTACTGGAGGAGTAGAAGGATGGGCCCCGTGGGGTCCCCACTGCCCCGGTACGAGGGGGCCCAAGACCC
TGGCAGGTCAGGGGCCCTGGCCAAGCCAGATCTGGAGCTGCTGCTCCCTGCTGCGGAGACCGCGGAGGCT
TCGCGTTGACCAAGTTCCTTAAAGAACTGGCTGATGGGGCAGGAGGTCCAGGCCTGGGCTCTCGGGCCCT
CCTAGAGGGCCATTGGAGCTTGCAGCTCAGACCCCCACTTTGAGTTTTATTTATTTAAATAGTAGTTGGA
TGCTTGGCACGTCGTCCTGTAATAGGAAACCCTTGCCTCATCAGTTTTCCTGATTTACAAGTGCAATATT
TTAGCCAATGCCTTGGGAGAAGCTGCCATGCAAAGGTGGACACCATTCTCCAGCTTCAGGGGATATGCTC
GTCCCGGGCACCGGTGGCAGGCAGCTGGCCTTCTGGACTAAGGCAGCCTGGGGGGACACTGCAGTCTGGC
TACACACAGAGATCTGGCACCCCCTGGGTGGAGTGTCCCTCGGGGGCTTTGGGAAAGCATGGCACCCTCA
GACCACACAGTAGCCAAGTTCTGGAGCAAATAAAAGGCCTGTGTTATTTCTTGTTCTTGA
NM_001282922.2 Homo sapiens regulator of G protein signaling 3
(RGS3), transcript variant 2, mRNA
(SEQ ID NO: 6)
ACTCTGTGCCACTCCGTGCCAGGCCCTGAGGGCACCCGGTTGCTGCTTCCTTCCGTCTTTCCCCAAGGAC
TATCAGAGATGCCAGCGTGACCCCTGACACGTGTGTGCAGCAGCCTGCAGCTGCCCCAAGCCATGGCTGA
ACACTGACTCCCAGCTGTGGGGCTTCACCATTACAGACTCCCCAGGGCTTCAAAGACTTCTCAGCTTCGA
GCATGGCTTTTGGCTGTCAGGGCAGCTGTACAATAGTGGATGTTTGAGACGGAGGCAGATGAGAAGAGGG
AGATGGCCTTGGAGGAAGGGAAGGGGCCTGGTGCCGAGGATTCCCCACCCAGCAAGGAGCCCTCTCCTGG
CCAGGAGCTTCCTCCAGGACAAGACCTTCCACCCAACAAGGACTCCCCTTCTGGGCAGGAACCCGCTCCC
AGCCAAGAACCACTGTCCAGCAAAGACTCAGCTACCTCTGAAGGATCCCCTCCAGGCCCAGATGCTCCGC
CCAGCAAGGATGTGCCACCATGCCAGGAACCCCCTCCAGCCCAAGACCTCTCACCCTGCCAGGACCTACC
TGCTGGTCAAGAACCCCTGCCTCACCAGGACCCTCTACTCACCAAAGACCTCCCTGCCATCCAGGAATCC
CCCACCCGGGACCTTCCACCCTGTCAAGATCTGCCTCCTAGCCAGGTCTCCCTGCCAGCCAAGGCCCTTA
CTGAGGACACCATGAGCTCCGGGGACCTACTAGCAGCTACTGGGGACCCACCTGCGGCCCCCAGGCCAGC
CTTCGTGATCCCTGAGGTCCGGCTGGATAGCACCTACAGCCAGAAGGCAGGGGCAGAGCAGGGCTGCTCG
GGAGATGAGGAGGATGCAGAAGAGGCCGAGGAGGTGGAGGAGGGGGAGGAAGGGGAGGAGGACGAGGATG
AGGACACCAGCGATGACAACTACGGAGAGCGCAGTGAGGCCAAGCGCAGCAGCATGATCGAGACGGGCCA
GGGGGCTGAGGGTGGCCTCTCACTGCGTGTGCAGAACTCGCTGCGGCGCCGGACGCACAGCGAGGGCAGC
CTGCTGCAGGAGCCCCGAGGGCCCTGCTTTGCCTCCGACACCACCTTGCACTGCTCAGACGGTGAGGGCG
CCGCCTCCACCTGGGGCATGCCTTCGCCCAGCACCCTCAAGAAAGAGCTGGGCCGCAATGGTGGCTCCAT
GCACCACCTTTCCCTCTTCTTCACAGGACACAGGAAGATGAGCGGGGCTGACACCGTTGGGGATGATGAC
GAAGCCTCCCGGAAGAGAAAGAGCAAAAACCTAGCCAAGGACATGAAGAACAAGCTGGGGATCTTCAGAC
GGCGGAATGAGTCCCCTGGAGCCCCTCCCGCGGGCAAGGCAGACAAAATGATGAAGTCATTCAAGCCCAC
CTCAGAGGAAGCCCTCAAGTGGGGCGAGTCCTTGGAGAAGCTGCTGGTTCACAAATACGGGTTAGCAGTG
TTCCAAGCCTTCCTTCGCACTGAGTTCAGTGAGGAGAATCTGGAGTTCTGGTTGGCTTGTGAGGACTTCA
AGAAGGTCAAGTCACAGTCCAAGATGGCATCCAAGGCCAAGAAGATCTTTGCTGAATACATCGCGATCCA
GGCATGCAAGGAGGTCAACCTGGACTCCTACACGCGGGAGCACACCAAGGACAACCTGCAGAGCGTCACG
CGGGGCTGCTTCGACCTGGCACAGAAGCGCATCTTCGGGCTCATGGAAAAGGACTCGTACCCTCGCTTTC
TCCGTTCTGACCTCTACCTGGACCTTATTAACCAGAAGAAGATGAGTCCCCCGCTTTAGGGGCCACTGGA
GTCGAGCTCAGCGTTCACACCAGGCGGGCTGGGTCCCCTGCCCACCTGCCTCCCTGCCCCCTGTGACGGA
GGGGGCAAGCAAGCCCCCAGAGGCTGTGTCTCTGGACAGACGGATAGACATACGGAAGCGAGGCCTGGAC
CAAGAGAGGCCCAGGCTACTGGAGGAGTAGAAGGATGGGCCCCGTGGGGTCCCCACTGCCCCGGTACGAG
GGGGCCCAAGACCCTGGCAGGTCAGGGGCCCTGGCCAAGCCAGATCTGGAGCTGCTGCTCCCTGCTGCGG
AGACCGCGGAGGCTTCGCGTTGACCAAGTTCCTTAAAGAACTGGCTGATGGGGCAGGAGGTCCAGGCCTG
GGCTCTCGGGCCCTCCTAGAGGGCCATTGGAGCTTGCAGCTCAGACCCCCACTTTGAGTTTTATTTATTT
AAATAGTAGTTGGATGCTTGGCACGTCGTCCTGTAATAGGAAACCCTTGCCTCATCAGTTTTCCTGATTT
ACAAGTGCAATATTTTAGCCAATGCCTTGGGAGAAGCTGCCATGCAAAGGTGGACACCATTCTCCAGCTT
CAGGGGATATGCTCGTCCCGGGCACCGGTGGCAGGCAGCTGGCCTTCTGGACTAAGGCAGCCTGGGGGGA
CACTGCAGTCTGGCTACACACAGAGATCTGGCACCCCCTGGGTGGAGTGTCCCTCGGGGGCTTTGGGAAA
GCATGGCACCCTCAGACCACACAGTAGCCAAGTTCTGGAGCAAATAAAAGGCCTGTGTTATTTCTTGTTC
TTGA
NM_001282923.2 Homo sapiens regulator of G protein signaling 3
(RGS3), transcript variant 10, mRNA
(SEQ ID NO: 7)
GGAGACTCCGGTTACTGGGGAGCAACACAGCCGCCTCGGGTTGCAGACGCTCCTGTCCGGGTCGCAGTGG
GACGCCATGGAGCGCTCCCTGCACCGCGTCTCCCTCGGGAGCCGGCGTGCCCACCCGGACTTGTCCTTCT
ACCTCACCACCTTTGGTCAGCTGAGGCTGTCCATTGATGCCCAGGACCGGGTTCTGCTGCTTCACAGTCT
CTTTATTCGTGTGGATGGATATGTCTATGTGTGTCTCTCTTTCTCGCTGTGTGTGTGTGTATGTTTCCAT
TCATCCACCCCAATGTCTGAATTCTCTTTTAGTTATAGAAGGTAAAGGCCTGATCAGCAAACAGCCTGGC
ACCTGTGATCCGTATGTGAAGATTTCTTTGATCCCTGAAGATAGTAGACTACGCCACCAGAAGACGCAGA
CCGTTCCAGACTGCAGAGACCCGGCTTTCCACGAGCACTTCTTCTTTCCTGTCCAAGAGGAGGATGATCA
GAAGCGTCTCTTGGTTACTGTGTGGAACAGGGCCAGCCAGTCCAGACAGAGTGGACTCATTGGCTGCATG
AGCTTTGGGGTGAAGTCTCTCCTGACTCCAGACAAGGAGATCAGTGGTTGGTACTACCTCCTAGGGGAGC
ACCTGGGCCGGACCAAGCACTTGAAGGTGGCCAGGCGGCGACTGCGGCCGCTGAGAGACCCGCTGCTGAG
AATGCCAGGAGGTGGGGACACTGAGAATGGGAAGAAACTAAAGATCACCATCCCGAGGGGAAAGGACGGC
TTTGGCTTCACCATCTGCTGCGACTCTCCAGTTCGAGTCCAGGCCGTGGATTCCGGGGGTCCGGCGGAAC
GGGCAGGGCTGCAGCAGCTGGACACGGTGCTGCAGCTGAATGAGAGGCCTGTGGAGCACTGGAAATGTGT
GGAGCTGGCCCACGAGATCCGGAGCTGCCCCAGTGAGATCATCCTACTCGTGTGGCGCATGGTCCCCCAG
GTCAAGCCAGGACCAGATGGCGGGGTCCTGCGGCGGGCCTCCTGCAAGTCGACACATGACCTCCAGTCAC
CCCCCAACAAACGGGAGAAGAACTGCACCCATGGGGTCCAGGCACGGCCTGAGCAGCGCCACAGCTGCCA
CCTGGTATGTGACAGCTCTGATGGGCTGCTGCTCGGCGGCTGGGAGCGCTACACCGAGGTGGCCAAGCGC
GGGGGCCAGCACACCCTGCCTGCACTGTCCCGTGCCACTGCCCCCACCGACCCCAACTACATCATCCTGG
CCCCGCTGAATCCTGGGAGCCAGCTGCTCCGGCCTGTGTACCAGGAGGATACCATCCCCGAAGAATCAGG
GAGTCCCAGTAAAGGGAAGTCCTACACAGGCCTGGGGAAGAAGTCCCGGCTGATGAAGACAGTGCAGACC
ATGAAGGGCCACGGGAACTACCAAAACTGCCCGGTTGTGAGGCCGCATGCCACGCACTCAAGCTATGGCA
CCTACGTCACCCTGGCCCCCAAAGTCCTGGTGTTCCCTGTCTTTGTTCAGCCTCTAGATCTCTGTAATCC
TGCCCGGACCCTCCTGCTGTCAGAGGAGCTGCTGCTGTATGAAGGGAGGAACAAGGCTGCCGAGGTGACA
CTGTTTGCCTATTCGGACCTGCTGCTCTTCACCAAGGAGGACGAGCCTGGCCGCTGCGACGTCCTGAGGA
ACCCCCTCTACCTCCAGAGTGTGAAGCTGCAGGAAGGTTCTTCAGAAGACCTGAAATTCTGCGTGCTCTA
TCTAGCAGAGAAGGCAGAGTGCTTATTCACTTTGGAAGCGCACTCGCAGGAGCAGAAGAAGAGAGTGTGC
TGGTGCCTGTCGGAGAACATCGCCAAGCAGCAACAGCTGGCAGCATCACCCCCGGACAGCAAGATGTTTG
AGACGGAGGCAGATGAGAAGAGGGAGATGGCCTTGGAGGAAGGGAAGGGGCCTGGTGCCGAGGATTCCCC
ACCCAGCAAGGAGCCCTCTCCTGGCCAGGAGCTTCCTCCAGGACAAGACCTTCCACCCAACAAGGACTCC
CCTTCTGGGCAGGAACCCGCTCCCAGCCAAGAACCACTGTCCAGCAAAGACTCAGCTACCTCTGAAGGAT
CCCCTCCAGGCCCAGATGCTCCGCCCAGCAAGGATGTGCCACCATGCCAGGAACCCCCTCCAGCCCAAGA
CCTCTCACCCTGCCAGGACCTACCTGCTGGTCAAGAACCCCTGCCTCACCAGGACCCTCTACTCACCAAA
GACCTCCCTGCCATCCAGGAATCCCCCACCCGGGACCTTCCACCCTGTCAAGATCTGCCTCCTAGCCAGG
TCTCCCTGCCAGCCAAGGCCCTTACTGAGGACACCATGAGCTCCGGGGACCTACTAGCAGCTACTGGGGA
CCCACCTGCGGCCCCCAGGCCAGCCTTCGTGATCCCTGAGGTCCGGCTGGATAGCACCTACAGCCAGAAG
GCAGGGGCAGAGCAGGGCTGCTCGGGAGATGAGGAGGATGCAGAAGAGGCCGAGGAGGTGGAGGAGGGGG
AGGAAGGGGAGGAGGACGAGGATGAGGACACCAGCGATGACAACTACGGAGAGCGCAGTGAGGCCAAGCG
CAGCAGCATGATCGAGACGGGCCAGGGGGCTGAGGGTGGCCTCTCACTGCGTGTGCAGAACTCGCTGCGG
CGCCGGACGCACAGCGAGGGCAGCCTGCTGCAGGAGCCCCGAGGGCCCTGCTTTGCCTCCGACACCACCT
TGCACTGCTCAGACGGTGAGGGCGCCGCCTCCACCTGGGGCATGCCTTCGCCCAGCACCCTCAAGAAAGA
GCTGGGCCGCAATGGTGGCTCCATGCACCACCTTTCCCTCTTCTTCACAGGACACAGGAAGATGAGCGGG
GCTGACACCGTTGGGGATGATGACGAAGCCTCCCGGAAGAGAAAGAGCAAAAACCTAGCCAAGGACATGA
AGAACAAGCTGGGGATCTTCAGACGGCGGAATGAGTCCCCTGGAGCCCCTCCCGCGGGCAAGGCAGACAA
AATGATGAAGTCATTCAAGCCCACCTCAGAGGAAGCCCTCAAGTGGGGCGAGTCCTTGGAGAAGCTGCTG
GTTCACAAATACGGGTTAGCAGTGTTCCAAGCCTTCCTTCGCACTGAGTTCAGTGAGGAGAATCTGGAGT
TCTGGTTGGCTTGTGAGGACTTCAAGAAGGTCAAGTCACAGTCCAAGATGGCATCCAAGGCCAAGAAGAT
CTTTGCTGAATACATCGCGATCCAGGCATGCAAGGAGGTCAACCTGGACTCCTACACGCGGGAGCACACC
AAGGACAACCTGCAGAGCGTCACGCGGGGCTGCTTCGACCTGGCACAGAAGCGCATCTTCGGGCTCATGG
AAAAGGACTCGTACCCTCGCTTTCTCCGTTCTGACCTCTACCTGGACCTTATTAACCAGAAGAAGATGAG
TCCCCCGCTTTAGGGGCCACTGGAGTCGAGCTCAGCGTTCACACCAGGCGGGCTGGGTCCCCTGCCCACC
TGCCTCCCTGCCCCCTGTGACGGAGGGGGCAAGCAAGCCCCCAGAGGCTGTGTCTCTGGACAGACGGATA
GACATACGGAAGCGAGGCCTGGACCAAGAGAGGCCCAGGCTACTGGAGGAGTAGAAGGATGGGCCCCGTG
GGGTCCCCACTGCCCCGGTACGAGGGGGCCCAAGACCCTGGCAGGTCAGGGGCCCTGGCCAAGCCAGATC
TGGAGCTGCTGCTCCCTGCTGCGGAGACCGCGGAGGCTTCGCGTTGACCAAGTTCCTTAAAGAACTGGCT
GATGGGGCAGGAGGTCCAGGCCTGGGCTCTCGGGCCCTCCTAGAGGGCCATTGGAGCTTGCAGCTCAGAC
CCCCACTTTGAGTTTTATTTATTTAAATAGTAGTTGGATGCTTGGCACGTCGTCCTGTAATAGGAAACCC
TTGCCTCATCAGTTTTCCTGATTTACAAGTGCAATATTTTAGCCAATGCCTTGGGAGAAGCTGCCATGCA
AAGGTGGACACCATTCTCCAGCTTCAGGGGATATGCTCGTCCCGGGCACCGGTGGCAGGCAGCTGGCCTT
CTGGACTAAGGCAGCCTGGGGGGACACTGCAGTCTGGCTACACACAGAGATCTGGCACCCCCTGGGTGGA
GTGTCCCTCGGGGGCTTTGGGAAAGCATGGCACCCTCAGACCACACAGTAGCCAAGTTCTGGAGCAAATA
AAAGGCCTGTGTTATTTCTTGTTCTTGA
NM_001322214.3 Homo sapiens regulator of G protein signaling 3
(RGS3), transcript variant 11, mRNA
(SEQ ID NO: 8)
AGTGCGGGCTCCCGCTGCTGGGGGAGAGCTGGGTTTTCATGGGGCGGCAGCCGAGGCAGGACCCGCAGCC
ATGAACCGCTTCAATGGGCTCTGCAAGGTGTGCTCGGAGCGCCGCTACCGCCAGATCACCATCCCGAGGG
GAAAGGACGGCTTTGGCTTCACCATCTGCTGCGACTCTCCAGTTCGAGTCCAGGCCGTGGATTCCGGGGG
TCCGGCGGAACGGGCAGGGCTGCAGCAGCTGGACACGGTGCTGCAGCTGAATGAGAGGCCTGTGGAGCAC
TGGAAATGTGTGGAGCTGGCCCACGAGATCCGGAGCTGCCCCAGTGAGATCATCCTACTCGTGTGGCGCA
TGGTCCCCCAGGTCAAGCCAGGACCAGATGGCGGGGTCCTGCGGCGGGCCTCCTGCAAGTCGACACATGA
CCTCCAGTCACCCCCCAACAAACGGGAGAAGAACTGCACCCATGGGGTCCAGGCACGGCCTGAGCAGCGC
CACAGCTGCCACCTGGTATGTGACAGCTCTGATGGGCTGCTGCTCGGCGGCTGGGAGCGCTACACCGAGG
TGGCCAAGCGCGGGGGCCAGCACACCCTGCCTGCACTGTCCCGTGCCACTGCCCCCACCGACCCCAACTA
CATCATCCTGGCCCCGCTGAATCCTGGGAGCCAGCTGCTCCGGCCTGTGTACCAGGAGGATACCATCCCC
GAAGAATCAGGGAGTCCCAGTAAAGGGAAGTCCTACACAGGCCTGGGGAAGAAGTCCCGGCTGATGAAGA
CAGTGCAGACCATGAAGGGCCACGGGAACTACCAAAACTGCCCGGTTGTGAGGCCGCATGCCACGCACTC
AAGCTATGGCACCTACGTCACCCTGGCCCCCAAAGTCCTGGTGTTCCCTGTCTTTGTTCAGCCTCTAGAT
CTCTGTAATCCTGCCCGGACCCTCCTGCTGTCAGAGGAGCTGCTGCTGTATGAAGGGAGGAACAAGGCTG
CCGAGGTGACACTGTTTGCCTATTCGGACCTGCTGCTCTTCACCAAGGAGGACGAGCCTGGCCGCTGCGA
CGTCCTGAGGAACCCCCTCTACCTCCAGAGTGTGAAGCTGCAGGAAGGTTCTTCAGAAGACCTGAAATTC
TGCGTGCTCTATCTAGCAGAGAAGGCAGAGTGCTTATTCACTTTGGAAGCGCACTCGCAGGAGCAGAAGA
AGAGAGTGTGCTGGTGCCTGTCGGAGAACATCGCCAAGCAGCAACAGCTGGCAGCATCACCCCCGGACAG
CAAGAAACTCCACCCTTTCGGCTCTCTCCAGCAGGAGATGGGGCCGGTCAACTCAACCAATGCCACCCAG
GATAGAAGCTTTACCTCACCAGGACAGACTCTGATTGGCTGAGCAAACCCAAGGGCGGGACTCTGCTTCT
GGCAACTTAACCCTTTCTTGGGCATCCCCTACCACACAGTAACCTCACCTCAACTGGACCTGAAACAGAG
GACCAAGATGGTGGGTCTAGGGTCATCTGAAGGGAGTGGTCCCAAGGGTGCATGTGGATGTCAACAGAAG
GACCACAATGGTGTGAGAGGCCTTGTGGGCATGCAGAGACTGGCAGCAGAAAGTCCCAAGAGGAGAGGAC
TCAGAGGCTTCTTCCTTCCAAGGAGGAGGAGGAGGAGGACATTCCACATCTGGGCTTCCAGTGGTGGCTC
CCGGACCTGCCTTGGGGGCAGCAGGAGAGGTTGGGAGAGGTCACCCTGATGAAGAACAATGCCATGATCT
GCTGCTGCCAGAGGACATCTCCGCCCATCCACTCATTTTCCACCTGTACACATTGGCTCATTCATTCGAT
GGATATTTCGGGTGCATCTACTTTATGCCAGGCTCTATGCTAGGAGCTGGGGATACTGTGGAGAAAAAGT
CGGATATGGTCTCGCTCTCATCAAGATTTCAGTCTAGCTGGGAGTGAGCGTTAAATAAGTCAATACCAAC
ACACACACGTGCACACACGTGATGGCAAGCTGTGATCATCTATGAAAGAAAAAACACAGATTTCTTTAAG
AGGGAATTGTACTTAGAGTGCTGTGTGTGGGGGTGGGGATGGGGAAGGACATAACTTTTAAACCGAGACT
TGTGGGCTGGAGAGGAAGGAGCCAGTCCTGGACTTGGGGAAGAGTGTTCGCGGTGGATGGAATAGCACGT
GGAGAGTCCCTGAGATGGGAAGGAGCTCAGTGGGCTGGAGGAATGGCGAGCAGGCCAGTGTGGCTGGAGC
TTAGTGAGGTGCAAGACACAGTGGGTGAGGTAGTTGATGTCTTGTTGATTTCATTCTAGATACACTGGAG
AGCCATTGAAGGCTTTTGAGGAAGGGAGTTGCAAGTACATTTTTACAAGATCCGTTTACACATAGGAGGG
GCCCAAGTGGGTGTGAGATCAGTTAGGGGGCTCGTTCAGTTCTCTAGGTGAGGACTGTCCTGGTGGCTTG
TCCTGGATGGTGGCTGGGGAAGTGGATGCCTGAATGCCAGGCTCTGGAGAAGAGATGACTCCCTGCCCCA
TCCCCCTGCCTACCCTCCTCCTCAATGGCCCCTCTCTCAGTGGATGTGAGTGAGGATTAGGACCTTCCCC
ACAGAGCCACCTTTTGGCCAGGCCTCGTGGGGGATAGTGGGGGCAGTGCTGAGTTCTAGGAGTGTTCCTG
GAAGTTGGGGGCAGTGAGTGCTCAGCCTGGCCCAGGTGGGAGAGTTCACTGCTGCCCAGGCCTTAGCAGG
GGCTGCTGTGCTGTCTTTCTGTCCCATGCTGAGCATTGTGATAGGAAGCTGCCACACAGTGCCCTGGAAA
CTGACTTCGCATGCTTTCCATGGGGCTTCCGAGGGCCAGGGACTGGGCTGGGGGTGGCCTAGCAGCTCTA
GTGGAGACTGTGCTGCCGCAGGGCTGAAGGAAGACCAACCAAGGGGACAGCCGTTTGAGCCCAGCCCACT
CACTGCCTGCTGCACCTCCAGCTGGAGCCCAGGGTTGGGCAGCTCTCATACTCTTATCTAACTGCCAGAC
CTAGAGGGAGGGAAGCCCAAGGCTGCAGGTCTGAGGGACACGGCTCATGGGCAAAAGGGTATCAAGAGGA
TTAGAGTCACTTGCATAGGAAAAGAGGGTCAGAGAGGCTGAGGGCTGGCTGAGGCCAGAGAGGTTCCCTG
ACCTTGGACATTCTTTGTGTTGTGTAAGACCTCGGCATCTTTAGAGAAAGGTTCTTGTGCCCCTGTGAGC
TTGCTCAGCTGCTATGGCCACTTGAACCCCAAAAATGGTGGCCTAGAAGAGAATACACCCCTTACTTCCC
ACTTATCCTCCTCTCAACCTAGGAAAGATCAGACATATTGACTCAACAGTCTAACAGGGGATGGGCTGGA
TCTGCCCGTGACCAGACCAATGCCCCCCACCCAGCAAGGGAGTAAACGCTGCCTGAAGTGGGACCAGTGA
TACACAGCATCTCTGTCTGCCAGGGCTCAGCCCGCGGGGGGGATTTGTGAGAGGCTGAGCAGCTGGGTCT
GCACAGACAGCAAGCTGCACTTTGATTTCATTTGATTCCCTTACAAAATGACATTAAGGGGAATGTGAGG
CACAGGAGACTCGCCTGTCTGGGAAGGCAGCAAGCTCATCTCTCCTGGACCTGGTGACCTACCTCTGTCT
ACCAGACAGGTGCAGAGGACAGGAGCTCTGGCCACAAAGAAATGGACAGTCTGAATGCAGCACACAGCCC
TGGGGACAGGACTTTTCACCCAGCCTGTCCTGACAAGCTGCTGAGTCGAGCAGATGCTCACATCAGGGTC
CCAGGTCCGGCTGTTAGGGAAGTGGACATCAGTTCCTGGTGGGCCCTGGGCAGTGACTGTGGTGAGCCAC
AGCACCATTGCTGCTCTGCCTCTCAAATCGATCGAACAATTAATTTGGCTCCACAGGCTCGAGAAGATGA
AACATGACCTGATGGCCACTGCTGTCCAGCATCCCAGTCCATCCTAGCTGTCCAGGATGATGAACAAGCT
ATGGAAATTGCACTTGAGAGCTATAATCACGGGAAGCATTGCCGAGCCCCTCCCTGCTACATAAATCATG
CTAAGAAGCACACAATGGCCTCTTTCCTGATGGGCTAATTGTCTTGGTTTGGTGTCTCAGGCCCAGGAGC
TGGGAAGATGGGAGCACAACCATTTATTGTTGGAAAGACCATCTGTGCAACTTGAATACAAAGTGATTCT
TTTAGTTCCCCACACACCCCCGACCCTCTGATCCCATTTTCATGTTTTTTTTTTTTTTTTCTTTTTATGT
CTCAGGCTCTGGTTCTTTTAAAACTCTTCATCTATCCTGTAAGCCATCAGGCCTGGATCTCTGCTGGCCT
GGGTCTTTTGGCTATTTGGGGGTTGGGACATTGCAGACCTTTTGGAAATAATGACATTTATTTATATATG
TCTTATATTTTTATTCTTTATTTTTCCCCATAAATTCTATTACCAAAGTCCTCATTTGTCACACATGGCA
CCAAGGCACCAAGAGGGTTTTTGCAAAGTGAAAATGCAGGCAATTTTGCAACGTGGAAGGGCTCCCCACA
GCGAAAGTCCCACAGTGAGAGCCTAAACTTTGGAGATGGCAACTGGGGTCCCCAGGCATGATCTCCCCCC
AGCCTCCCAGCAGCCTTGTTTTATGGATGAAAGACACCAAGGCTCTGAGAAGTTAAGTGTTTTATCCTAG
GTCACACAGCTGGTAAGGGCCAAGCTTGCCCTCAAGCCAAGGCCTTTGAACCTAATCTACCACTCATTTC
TGATAAAAAGGTTTGTCTTTTAGGGATAGAAGTAAGAGAAATCAATAATCTCCAGGAAGAGGCAAGAGAG
GGATATCCGGCATGTCAGAAGCTGTGGCCCATCAAGCTGTAGAGGGAATGCTTTTAGGATAAATAAAATG
AGATGCCTTATTTTCCACCCAGTGGAGAATAAACTTCCAGAATGCATTTTCCTGAGGGTGGCACAGGCTG
AAAAGATCAATAGCTTCCCACAAGATTTGAAAAAAGCCTAAAATGAGAGATCAGTTTGAGCTCCTTAAGA
CAGGTTCTCAGGGAGCTCTGGAATGGTTAGGTTACACCCCTTCCCCTTTTGAATTTGGCATCAAAAGGAT
CAGCAGACAGAGTCATGGAGTGCTTGGCAGTGATGGAATATTAGCCAAAGGCAGAGAGGTATAGCTTCAG
GCTCAGGCTGGCTTGTGCCCAGCGTAAGTACTGGAAGGAGGCCTTTGACCTGAGACCAGCTGAGGGCACA
GAGAGGGCCTGCCCATGTGGAACCTGCCAGGTTTTGTGCGCAGATTTTGCTAATTGACTGATTTAGTATC
TCAGCCCTGAATAAAGGGGAATCAAATGGATCTGATTTCAGCCATCTCCCCTAAGAGATGCAGACTCTTC
TCCCTAAAAACTGTTTCCAGGCAGCCACCAGCTCTCCTCCCCATTCCTTGCTGCTGTCCTATCAGTGGAC
AGACTGCCTTCTTCGCTGTCTTGCTCTTGGTCTCAGCCATCTTGCTATTGTTTTTTTTAGCCCCTGAAGA
AGACAGTGCAACCTTACCCTCTATCCTGTCTTCGCCTTCTCTTCCTCGAAGACTTTCTCTGAATAGGGCC
ATCCATCCTCCAAACTGTTCCCAGATCCTTGGTTTTAAAGGTAGAATGATAACATAAGGTAACCTGGTGG
GCACTTACAAGATGAGCCAGGCACAGGGTCAAGCTTTTTACCTACCCTGTCTCATCTAATCCTCATGCAG
ACTTACAAGGTGTGTTACCCCTGTTATACATTTACTCTCATTATGCCCAATTTAGATATGAAGACGCTGA
GTCTCAGAGAAGTTAAGAAACGTGCCCAAGGTCACACAGCTAGTGGGTAGCTAGGATTGAAACTTGGACA
GCCCAGCTCCAGGGTACCTGCTTTTGATTATTTTGCTATATGGCTTTCCTGTAACTGCAGACCATCCAAG
CTGGAAGGGCCAGGGAGACTGGACTGTCACACTCCTGCTTAAAGACCATTGTGGGAGGCCACTGTCTTCA
GAGTAAAGCTTGGCTTTCCAGGCTCTCCTTGGTAGCACTACACTCCTGCTTTGCACCTGGAGCCCGGCCA
CACCATAAATTTTCCCCTTCTCTAAACTCACTGTGCACATTCACAGTTAGGGTGTTTGATCTGCCTGACG
TTGACTGCCCTCCTTGGCTGTCCCTGCTGGATGCAATCCTCAACCTTCAAGGCTTAGTGGCTCCTCTGAG
AACACCTTTCTCAGGCATGCATCAGGAGTCCTGACTTCCCCCTTCGTGGTTCCCAGAGTACTTTTTCCCC
CTTAGCACAATTCATCCTTAGCATCTGGATGACTGTGAGCTTCTTCGGAGCAAGTAGATGTTATATTTTA
TTTGTCTTTGATTCTATTGTTGTCCGGTCCGTAGTAGGTGCTCAGTAGAGGTTGTAGGATTGAATTAGGC
TGTCCAGTTCAACTCTCCTATATTCTAGAATTTAGACAAGGAAACAGGTGCAAAAAGAGAAGTGACCTGG
CCAGGGTCATGGGAAAAGTTAGTGTCAGAGCCAGGACTAGAGCTCAGGTCTCCTGATTCTTTGGATTCAG
AGCTTTTTCTGTTGCACCTCAGCTTTGTGGAGACCAGAGCATGGATCTGCACTTGGTTTATGAAGGTCTG
ACCAGGTTCACTTCAGGGTCATGGATACCACTGTGTTCCCATGCTGACACTGGGGAATTCCCAGAGGGAA
AGGAAGCCCAGCTGGAGCTGCTGCTGTAGGTTGCGGGGCTCACCTCTCACCGAGCCCCTTGCTTACAGAG
AAAACCAGCGAGTGATTCTGAAAATCTTGCCTCGAGTCCTCATTTTGCTGGTATTCACCGTGTGACCTTG
AGCAAATTGCTTCACATCTCTGATCTTCTGTTCCTTTACCAAGAAAAATGAGGGAGTTGAATTCAGATGG
TCTCCAGGAGTCATCTGATTCCAGAATATCAGGATATAACAATAAAGATGAGGATAAAGTAGCTACTCCC
CTCAGAAAAGGTGGAGATCCAGATCTAGGGGGAGAATTGGCTAGGTTCTTCATCACAGTCACTGTCTTGA
GACACATAGGTGGTCCATGGAAACATTTCCATGAGGGATTTCAGAGGGGAGAATGGGCAGACTGCAAAGC
CTCGGTACTCTAATACCAGGCAAAAAGAAGAAAGGGAACAGGCATTTTAGTTTTCACATTGAAACAGAGT
GTATGTGTATGGCCCTATGCAAATGTTGTTCCTGGTGAATATTCATCAATGTACTCATCACACATAGGTA
TATTTAGACAGAAAGGGGTCCTCATCACAGACACCCAAATTATGAGCACATTACTGCCTCCTCTCCCCTA
GAGGTGCTTCTTCCAGCCTCCCCCATTGGAGGCCAGCACAAGACGGGCTCTCCAACCCAATATCTTCCCC
TTCCCTCCTTCCTTTCTGCTCAGGTGGCTGAGTATACACATTGAACTCTTTTGTTTCAACTTCACGTGTA
TATAGGTTGGTGCAAAAGTAACTGTGGTGTTTTCCATTAAAAGCAATGTCAAGAACCGCAATTACTTTGG
CACCAACCTAATATGTATGCAAGTACATTCTTTAACCTTTCCAACTCCCAGTCCAAGCCATCAGGCTAAT
TTCCGCGTGGAATTTGTGGTCACTGGTTTGCAGTCCTTTCATTTCTGGTGTGGTCAGAAGTCTCCTTCAG
ACTCTTCCCGATAAGTTGCCATTGGCTTTTTCATCACCTTTAGTACCTACCCACTTTTGCTGGGATAACA
CTACCCAAGATAAATTCAAAAAAATTGTCCTAAGTGTTAGTGTGTTTCTGTTTTGGTTGAAGTCTATTAT
TGCTATATGGTTTTCTACTGTTTCTACTTGATGGAAGACTATTAAGGGTTAAGTAGGCTGGCCTAGGAAG
CTGACCACCATTTATAACATACTTTTGAGTGGGAAAATGCATTCAGAGGCCTGTAATCAATACTTAGGTG
GACTTTGGGAATATAGTATTTATTTTATGAGTGACTAGAATATTGGTAGAACCATAGAAACTTTGAATGT
GGAGCTGGAAAGCCTCCTCCTTCCCCCACTGAGATCAATTGATTAACAAGCTAGGTTGTAGAAACCAAGT
GAAAGAGGCAAAAACTTGGCCAAGGGCATGTAGTCGGGGACTTCTGAAGATTCTGCTGGGCTGGGGGCAG
GAAAGGGGTCACAGACGGTCTTAGGGTTGTCTCACTCAGCAACTCACTGTGTTGGCCTTGGGAAATGTAA
GTCACTGTGTTGGGCTGTAATGGAAATATTGAGAAAGCAGCTCAGCTAGTGGCGTGGGGAGAGGTTGTCT
AGTCCTCTGGCTTATCTGTAAATTCCTGGCATGTGCTTGTTGGAGCAGAATGAATGGGGCTGCTGTGTTT
TACCTGCAGCTAGGAGCATGGCTGGAGTGGTGGAGACACTCCCTGTGGTCTGTTCTAAAAATAGCAGTGG
GAACAGAGCTGAGGGGAAGAGGAGGGGGCTCCTTCGGGAGCTGGGTGGGGAGGCCTCACCCCCTTCCTCT
TCCTGCCAGGCCCGATGTGAGGAAGTCCCATGGAGTCACATAATTCCATCTGGGAGAGTCCTGGAGCCAT
CAGCCCTCACACCCCCTCCTCATACAGGCGAGGAGGCCCTGGAGGCCCGGAGAGCAGAAAGCACTGGCTG
GTGTCAAGCAAGCCCAGAGAGAAGGGCCCAGTTGGCAGGCTGTTTTTCCCTGGCTGTTTCAGCACAGTGG
CTGCAGGCCTTGTGCTGAGGTTTGCTGTCACTGCCTTTGGGCGCACAGAGACTGGGAACTCCTGGGACCG
GATGTGGTCAGGCTTCCTGAGAGTCGCCTGGCAGGCGAGGAGTCCCCAGCTGGCCCACCCTGAGCCCTGA
CCTCAGCCAGCCGGCTGTCCAGTGCCACACCTCCTCTTGCTGGTGTCCCCTCCCTTGCTGACTTGTCCTC
CTTTCTTTGGACTGTCTGCAGGGAGGGAGGACAAGGGCCAGCCTAGATCTGGCCTGCAGGACAGAGGCCT
CAGTGGCTCAGGACCTTTCCCTGCCCCTCCCCAGGAACAAGCAGAGGCAGCTGAGGTAGTAGCAGCCTCC
TGCAGGTTTAGAGACAGACAGGCCAGGGTTCAAATCCTAGCTCTTCCTCTCACTAGCCATGGGATTACTG
ATGGGTCACAGAGCATCTGTAAAATGAGACAAGAACGCTGATTTCACTGGGGTCTTGTGAAGATCAAAGG
GGATCTGTTGTAATGCCTGGCATGCAGTAGGTACTTAATAGTAGCTCTTGTTGCAAAACCAAGCAGTGAA
CATGTATCTGACTTTCTATAACCCCCTTCCTTGGGGCCTCCACTCATGAGGCATGGGAAGGTGTTTTTTT
TCTTTGTTTGTTTGTTTGTTTGAATGCAGTTGACTTTTTTTTTGTTGTATTGTTGCAAAAGTAATCCATG
TGATTGATGTTTATTATGAAGAAAACATTTAATACAGATGAACCAGGAGAAGAAAATGAAGTTCATCCTT
AATCCCAGCACCCAGAGATAAACCACTGTTAACATTTTGGAATGTGTCCTGTCAGGCATTCGTTTATACA
CACATATATCTTATTTGCAAATTGGGGTTACTGGTGCAATTTTTCTAACCTGCTTTTTCTCCCCCATTTA
ACAGCGTAGTGTATCCATGTTTCCATTTCAATAAATATTCTTCTACAACATCA
NM_001322215.2 Homo sapiens regulator of G protein signaling 3
(RGS3), transcript variant 12, mRNA
(SEQ ID NO: 9)
AGTGCCAGGGGGTGCCGTTGTGAGGGATGGACGCCTCTCCCCCGGAGCAGCACTTTGGGGCCAGCTTGGG
CCCTGGGGATGAGCCACAGGGGACCCACAGCCTATGCGTGTGAGCATGTAACCCGGGAGCCAGCTGGGCC
CCTCGCGGGGTGGGCAGAAGGACGGGCTGGCCCAGGACCCTCTTCTTTGAGACATCCCGGACTCCATCTC
GGATGAAAGTGCTTTGAGAAACCACAGAGTTTTCTGTTTAATGGAAGAAAGAAATCCAGCCTCTCTCCAG
AGTGGCGGTGGCCGGCTAGACAGGATGTTTGAGACGGAGGCAGATGAGAAGAGGGAGATGGCCTTGGAGG
AAGGGAAGGGGCCTGGTGCCGAGGATTCCCCACCCAGCAAGGAGCCCTCTCCTGGCCAGGAGCTTCCTCC
AGGACAAGACCTTCCACCCAACAAGGACTCCCCTTCTGGGCAGGAACCCGCTCCCAGCCAAGAACCACTG
TCCAGCAAAGACTCAGCTACCTCTGAAGGATCCCCTCCAGGCCCAGATGCTCCGCCCAGCAAGGATGTGC
CACCATGCCAGGAACCCCCTCCAGCCCAAGACCTCTCACCCTGCCAGGACCTACCTGCTGGTCAAGAACC
CCTGCCTCACCAGGACCCTCTACTCACCAAAGACCTCCCTGCCATCCAGGAATCCCCCACCCGGGACCTT
CCACCCTGTCAAGATCTGCCTCCTAGCCAGGTCTCCCTGCCAGCCAAGGCCCTTACTGAGGACACCATGA
GCTCCGGGGACCTACTAGCAGCTACTGGGGACCCACCTGCGGCCCCCAGGCCAGCCTTCGTGATCCCTGA
GGTCCGGCTGGATAGCACCTACAGCCAGAAGGCAGGGGCAGAGCAGGGCTGCTCGGGAGATGAGGAGGAT
GCAGAAGAGGCCGAGGAGGTGGAGGAGGGGGAGGAAGGGGAGGAGGACGAGGATGAGGACACCAGCGATG
ACAACTACGGAGAGCGCAGTGAGGCCAAGCGCAGCAGCATGATCGAGACGGGCCAGGGGGCTGAGGGTGG
CCTCTCACTGCGTGTGCAGAACTCGCTGCGGCGCCGGACGCACAGCGAGGGCAGCCTGCTGCAGGAGCCC
CGAGGGCCCTGCTTTGCCTCCGACACCACCTTGCACTGCTCAGACGGTGAGGGCGCCGCCTCCACCTGGG
GCATGCCTTCGCCCAGCACCCTCAAGAAAGAGCTGGGCCGCAATGGTGGCTCCATGCACCACCTTTCCCT
CTTCTTCACAGGACACAGGAAGATGAGCGGGGCTGACACCGTTGGGGATGATGACGAAGCCTCCCGGAAG
AGAAAGAGCAAAAACCTAGCCAAGGACATGAAGAACAAGCTGGGGATCTTCAGACGGCGGAATGAGTCCC
CTGGAGCCCCTCCCGCGGGCAAGGCAGACAAAATGATGAAGTCATTCAAGCCCACCTCAGAGGAAGCCCT
CAAGTGGGGCGAGTCCTTGGAGAAGCTGCTGGTTCACAAATACGGGTTAGCAGTGTTCCAAGCCTTCCTT
CGCACTGAGTTCAGTGAGGAGAATCTGGAGTTCTGGTTGGCTTGTGAGGACTTCAAGAAGGTCAAGTCAC
AGTCCAAGATGGCATCCAAGGCCAAGAAGATCTTTGCTGAATACATCGCGATCCAGGCATGCAAGGAGGT
CAACCTGGACTCCTACACGCGGGAGCACACCAAGGACAACCTGCAGAGCGTCACGCGGGGCTGCTTCGAC
CTGGCACAGAAGCGCATCTTCGGGCTCATGGAAAAGGACTCGTACCCTCGCTTTCTCCGTTCTGACCTCT
ACCTGGACCTTATTAACCAGAAGAAGATGAGTCCCCCGCTTTAGGGGCCACTGGAGTCGAGCTCAGCGTT
CACACCAGGCGGGCTGGGTCCCCTGCCCACCTGCCTCCCTGCCCCCTGTGACGGAGGGGGCAAGCAAGCC
CCCAGAGGCTGTGTCTCTGGACAGACGGATAGACATACGGAAGCGAGGCCTGGACCAAGAGAGGCCCAGG
CTACTGGAGGAGTAGAAGGATGGGCCCCGTGGGGTCCCCACTGCCCCGGTACGAGGGGGCCCAAGACCCT
GGCAGGTCAGGGGCCCTGGCCAAGCCAGATCTGGAGCTGCTGCTCCCTGCTGCGGAGACCGCGGAGGCTT
CGCGTTGACCAAGTTCCTTAAAGAACTGGCTGATGGGGCAGGAGGTCCAGGCCTGGGCTCTCGGGCCCTC
CTAGAGGGCCATTGGAGCTTGCAGCTCAGACCCCCACTTTGAGTTTTATTTATTTAAATAGTAGTTGGAT
GCTTGGCACGTCGTCCTGTAATAGGAAACCCTTGCCTCATCAGTTTTCCTGATTTACAAGTGCAATATTT
TAGCCAATGCCTTGGGAGAAGCTGCCATGCAAAGGTGGACACCATTCTCCAGCTTCAGGGGATATGCTCG
TCCCGGGCACCGGTGGCAGGCAGCTGGCCTTCTGGACTAAGGCAGCCTGGGGGGACACTGCAGTCTGGCT
ACACACAGAGATCTGGCACCCCCTGGGTGGAGTGTCCCTCGGGGGCTTTGGGAAAGCATGGCACCCTCAG
ACCACACAGTAGCCAAGTTCTGGAGCAAATAAAAGGCCTGTGTTATTTCTTGTTCTTGA
NM_001351526.2 Homo sapiens regulator of G protein signaling 3
(RGS3), transcript variant 13, mRNA
(SEQ ID NO: 10)
AGTGCCAGGGGGTGCCGTTGTGAGGGATGGACGCCTCTCCCCCGGAGCAGCACTTTGGGGCCAGCTTGGG
CCCTGGGGATGAGCCACAGGGGACCCACAGCCTATGCGTGTGAGCATGTAACCCGGGAGCCAGCTGGGCC
CCTCGCGGGGTGGGCAGAAGGACGGGCTGGCCCAGGACCCTCTTCTTTGAGACATCCCGGACTCCATCTC
GGATGAAAGTGCTTTGAGAAACCACAGAGTTTTCTGTTTAATGGAAGAAAGAAATCCAGCCTCTCTCCAG
AGTGGCGGTGGCCGGCTAGACAGGGTATCGCTCCCCTGGGGAACTTGGGTAAGTCCATAAATAGCTCTGC
TGACACAAAGGAGCTCTGTCTGGGGAAGGCAGCCGCTTGACACGGCCGCCAGGAGCTGCCACAGCCACGG
CCGCCCGCCTGCGGGAGGAGCCGGGTGGAAACCTGGGCGGGCGAGCTGGCAGGAGCGGCAGCCAGGAGTC
GCCTGGGTCCCTGCTGGAGTATGCTGGATGCCAGCGTGACCCCTGACACGTGTGTGCAGCAGCCTGCAGC
TGCCCCAAGCCATGGCTGAACACTGACTCCCAGCTGTGGGGCTTCACCATTACAGACTCCCCAGGGCTTC
AAAGACTTCTCAGCTTCGAGCATGGCTTTTGGCTGTCAGGGCAGCTGTACAATAGTGGATGTTTGAGACG
GAGGCAGATGAGAAGAGGGAGATGGCCTTGGAGGAAGGGAAGGGGCCTGGTGCCGAGGATTCCCCACCCA
GCAAGGAGCCCTCTCCTGGCCAGGAGCTTCCTCCAGGACAAGACCTTCCACCCAACAAGGACTCCCCTTC
TGGGCAGGAACCCGCTCCCAGCCAAGAACCACTGTCCAGCAAAGACTCAGCTACCTCTGAAGGATCCCCT
CCAGGCCCAGATGCTCCGCCCAGCAAGGATGTGCCACCATGCCAGGAACCCCCTCCAGCCCAAGACCTCT
CACCCTGCCAGGACCTACCTGCTGGTCAAGAACCCCTGCCTCACCAGGACCCTCTACTCACCAAAGACCT
CCCTGCCATCCAGGAATCCCCCACCCGGGACCTTCCACCCTGTCAAGATCTGCCTCCTAGCCAGGTCTCC
CTGCCAGCCAAGGCCCTTACTGAGGACACCATGAGCTCCGGGGACCTACTAGCAGCTACTGGGGACCCAC
CTGCGGCCCCCAGGCCAGCCTTCGTGATCCCTGAGGTCCGGCTGGATAGCACCTACAGCCAGAAGGCAGG
GGCAGAGCAGGGCTGCTCGGGAGATGAGGAGGATGCAGAAGAGGCCGAGGAGGTGGAGGAGGGGGAGGAA
GGGGAGGAGGACGAGGATGAGGACACCAGCGATGACAACTACGGAGAGCGCAGTGAGGCCAAGCGCAGCA
GCATGATCGAGACGGGCCAGGGGGCTGAGGGTGGCCTCTCACTGCGTGTGCAGAACTCGCTGCGGCGCCG
GACGCACAGCGAGGGCAGCCTGCTGCAGGAGCCCCGAGGGCCCTGCTTTGCCTCCGACACCACCTTGCAC
TGCTCAGACGGTGAGGGCGCCGCCTCCACCTGGGGCATGCCTTCGCCCAGCACCCTCAAGAAAGAGCTGG
GCCGCAATGGTGGCTCCATGCACCACCTTTCCCTCTTCTTCACAGGACACAGGAAGATGAGCGGGGCTGA
CACCGTTGGGGATGATGACGAAGCCTCCCGGAAGAGAAAGAGCAAAAACCTAGCCAAGGACATGAAGAAC
AAGCTGGGGATCTTCAGACGGCGGAATGAGTCCCCTGGAGCCCCTCCCGCGGGCAAGGCAGACAAAATGA
TGAAGTCATTCAAGCCCACCTCAGAGGAAGCCCTCAAGTGGGGCGAGTCCTTGGAGAAGCTGCTGGTTCA
CAAATACGGGTTAGCAGTGTTCCAAGCCTTCCTTCGCACTGAGTTCAGTGAGGAGAATCTGGAGTTCTGG
TTGGCTTGTGAGGACTTCAAGAAGGTCAAGTCACAGTCCAAGATGGCATCCAAGGCCAAGAAGATCTTTG
CTGAATACATCGCGATCCAGGCATGCAAGGAGGTCAACCTGGACTCCTACACGCGGGAGCACACCAAGGA
CAACCTGCAGAGCGTCACGCGGGGCTGCTTCGACCTGGCACAGAAGCGCATCTTCGGGCTCATGGAAAAG
GACTCGTACCCTCGCTTTCTCCGTTCTGACCTCTACCTGGACCTTATTAACCAGAAGAAGATGAGTCCCC
CGCTTTAGGGGCCACTGGAGTCGAGCTCAGCGTTCACACCAGGCGGGCTGGGTCCCCTGCCCACCTGCCT
CCCTGCCCCCTGTGACGGAGGGGGCAAGCAAGCCCCCAGAGGCTGTGTCTCTGGACAGACGGATAGACAT
ACGGAAGCGAGGCCTGGACCAAGAGAGGCCCAGGCTACTGGAGGAGTAGAAGGATGGGCCCCGTGGGGTC
CCCACTGCCCCGGTACGAGGGGGCCCAAGACCCTGGCAGGTCAGGGGCCCTGGCCAAGCCAGATCTGGAG
CTGCTGCTCCCTGCTGCGGAGACCGCGGAGGCTTCGCGTTGACCAAGTTCCTTAAAGAACTGGCTGATGG
GGCAGGAGGTCCAGGCCTGGGCTCTCGGGCCCTCCTAGAGGGCCATTGGAGCTTGCAGCTCAGACCCCCA
CTTTGAGTTTTATTTATTTAAATAGTAGTTGGATGCTTGGCACGTCGTCCTGTAATAGGAAACCCTTGCC
TCATCAGTTTTCCTGATTTACAAGTGCAATATTTTAGCCAATGCCTTGGGAGAAGCTGCCATGCAAAGGT
GGACACCATTCTCCAGCTTCAGGGGATATGCTCGTCCCGGGCACCGGTGGCAGGCAGCTGGCCTTCTGGA
CTAAGGCAGCCTGGGGGGACACTGCAGTCTGGCTACACACAGAGATCTGGCACCCCCTGGGTGGAGTGTC
CCTCGGGGGCTTTGGGAAAGCATGGCACCCTCAGACCACACAGTAGCCAAGTTCTGGAGCAAATAAAAGG
CCTGTGTTATTTCTTGTTCTTGA
NM_001394167.1 Homo sapiens regulator of G protein signaling 3
(RGS3), transcript variant 14, mRNA
(SEQ ID NO: 11)
GGAGACTCCGGTTACTGGGGAGCAACACAGCCGCCTCGGGTTGCAGACGCTCCTGTCCGGGTCGCAGTGG
GACGCCATGGAGCGCTCCCTGCACCGCGTCTCCCTCGGGAGCCGGCGTGCCCACCCGGACTTGTCCTTCT
ACCTCACCACCTTTGGTCAGCTGAGGCTGTCCATTGATGCCCAGGACCGGGTTCTGCTGCTTCACATTAT
AGAAGGTAAAGGCCTGATCAGCAAACAGCCTGGCACCTGTGATCCGTATGTGAAGATTTCTTTGATCCCT
GAAGATAGTAGACTACGCCACCAGAAGACGCAGACCGTTCCAGACTGCAGAGACCCGGCTTTCCACGAGC
ACTTCTTCTTTCCTGTCCAAGAGGAGGATGATCAGAAGCGTCTCTTGGTTACTGTGTGGAACAGGGCCAG
CCAGTCCAGACAGAGTGGACTCATTGGCTGCATGAGCTTTGGGGTGAAGTCTCTCCTGACTCCAGACAAG
GAGATCAGTGGTTGGTACTACCTCCTAGGGGAGCACCTGGGCCGGACCAAGCACTTGAAGGTGGCCAGGC
GGCGACTGCGGCCGCTGAGAGACCCGCTGCTGAGAATGCCAGGAGGTGGGGACACTGAGAATGGGAAGAA
ACTAAAGATCACCATCCCGAGGGGAAAGGACGGCTTTGGCTTCACCATCTGCTGCGACTCTCCAGTTCGA
GTCCAGGCCGTGGATTCCGGGGGTCCGGCGGAACGGGCAGGGCTGCAGCAGCTGGACACGGTGCTGCAGC
TGAATGAGAGGCCTGTGGAGCACTGGAAATGTGTGGAGCTGGCCCACGAGATCCGGAGCTGCCCCAGTGA
GATCATCCTACTCGTGTGGCGCATGGTCCCCCAGGTCAAGCCAGGACCAGATGGCGGGGTCCTGCGGCGG
GCCTCCTGCAAGTCGACACATGACCTCCAGTCACCCCCCAACAAACGGGAGAAGAACTGCACCCATGGGG
TCCAGGCACGGCCTGAGCAGCGCCACAGCTGCCACCTGGTATGTGACAGCTCTGATGGGCTGCTGCTCGG
CGGCTGGGAGCGCTACACCGAGGTGGCCAAGCGCGGGGGCCAGCACACCCTGCCTGCACTGTCCCGTGCC
ACTGCCCCCACCGACCCCAACTACATCATCCTGGCCCCGCTGAATCCTGGGAGCCAGCTGCTCCGGCCTG
TGTACCAGGAGGATACCATCCCCGAAGAATCAGGGAGTCCCAGTAAAGGGAAGTCCTACACAGGCCTGGG
GAAGAAGTCCCGGCTGATGAAGACAGTGCAGACCATGAAGGGCCACGGGAACTACCAAAACTGCCCGGTT
GTGAGGCCGCATGCCACGCACTCAAGCTATGGCACCTACGTCACCCTGGCCCCCAAAGTCCTGGTGTTCC
CTGTCTTTGTTCAGCCTCTAGATCTCTGTAATCCTGCCCGGACCCTCCTGCTGTCAGAGGAGCTGCTGCT
GTATGAAGGGAGGAACAAGGCTGCCGAGGTGACACTGTTTGCCTATTCGGACCTGCTGCTCTTCACCAAG
GAGGACGAGCCTGGCCGCTGCGACGTCCTGAGGAACCCCCTCTACCTCCAGAGTGTGAAGCTGCAGGAAG
GTTCTTCAGAAGACCTGAAATTCTGCGTGCTCTATCTAGCAGAGAAGGCAGAGTGCTTATTCACTTTGGA
AGCGCACTCGCAGGAGCAGAAGAAGAGAGTGTGCTGGTGCCTGTCGGAGAACATCGCCAAGCAGCAACAG
CTGGCAGCATCACCCCCGGACAGCAAGATGTTTGAGACGGAGGCAGATGAGAAGAGGGAGATGGCCTTGG
AGGAAGGGAAGGGGCCTGGTGCCGAGGATTCCCCACCCAGCAAGGAGCCCTCTCCTGGCCAGGAGCTTCC
TCCAGGACAAGACCTTCCACCCAACAAGGACTCCCCTTCTGGGCAGGAACCCGCTCCCAGCCAAGAACCA
CTGTCCAGCAAAGACTCAGCTACCTCTGAAGGATCCCCTCCAGGCCCAGATGCTCCGCCCAGCAAGGATG
TGCCACCATGCCAGGAACCCCCTCCAGCCCAAGACCTCTCACCCTGCCAGGACCTACCTGCTGGTCAAGA
ACCCCTGCCTCACCAGGACCCTCTACTCACCAAAGACCTCCCTGCCATCCAGGAATCCCCCACCCGGGAC
CTTCCACCCTGTCAAGATCTGCCTCCTAGCCAGGTCTCCCTGCCAGCCAAGGCCCTTACTGAGGACACCA
TGAGCTCCGGGGACCTACTAGCAGCTACTGGGGACCCACCTGCGGCCCCCAGGCCAGCCTTCGTGATCCC
TGAGGTCCGGCTGGATAGCACCTACAGCCAGAAGGCAGGGGCAGAGCAGGGCTGCTCGGGAGATGAGGAG
GATGCAGAAGAGGCCGAGGAGGTGGAGGAGGGGGAGGAAGGGGAGGAGGACGAGGATGAGGACACCAGCG
ATGACAACTACGGAGAGCGCAGTGAGGCCAAGCGCAGCAGCATGATCGAGACGGGCCAGGGGGCTGAGGG
TGGCCTCTCACTGCGTGTGCAGAACTCGCTGCGGCGCCGGACGCACAGCGAGGGCAGCCTGCTGCAGGAG
CCCCGAGGGCCCTGCTTTGCCTCCGACACCACCTTGCACTGCTCAGACGGTGAGGGCGCCGCCTCCACCT
GGGGCATGCCTTCGCCCAGCACCCTCAAGAAAGAGCTGGGCCGCAATGGTGGCTCCATGCACCACCTTTC
CCTCTTCTTCACAGGACACAGGAAGATGAGCGGGGCTGACACCGTTGGGGATGATGACGAAGCCTCCCGG
AAGAGAAAGAGCAAAAACCTAGCCAAGGACATGAAGAACAAGCTGGGGATCTTCAGACGGCGGAATGAGT
CCCCTGGAGCCCCTCCCGCGGGCAAGGCAGACAAAATGATGAAGTCATTCAAGCCCACCTCAGAGGAAGC
CCTCAAGTGGGGCGAGTCCTTGGAGAAGCTGCTGGTTCACAAATACGGGTTAGCAGTGTTCCAAGCCTTC
CTTCGCACTGAGTTCAGTGAGGAGAATCTGGAGTTCTGGTTGGCTTGTGAGGACTTCAAGAAGGTCAAGT
CACAGTCCAAGATGGCATCCAAGGCCAAGAAGATCTTTGCTGAATACATCGCGATCCAGGCATGCAAGGA
GGTCAACCTGGACTCCTACACGCGGGAGCACACCAAGGACAACCTGCAGAGCGTCACGCGGGGCTGCTTC
GACCTGGCACAGAAGCGCATCTTCGGGCTCATGGAAAAGGACTCGTACCCTCGCTTTCTCCGTTCTGACC
TCTACCTGGACCTTATTAACCAGAAGAAGATGAGTCCCCCGCTTTAGGGGCCACTGGAGTCGAGCTCAGC
GTTCACACCAGGCGGGCTGGGTCCCCTGCCCACCTGCCTCCCTGCCCCCTGTGACGGAGGGGGCAAGCAA
GCCCCCAGAGGCTGTGTCTCTGGACAGACGGATAGACATACGGAAGCGAGGCCTGGACCAAGAGAGGCCC
AGGCTACTGGAGGAGTAGAAGGATGGGCCCCGTGGGGTCCCCACTGCCCCGGTACGAGGGGGCCCAAGAC
CCTGGCAGGTCAGGGGCCCTGGCCAAGCCAGATCTGGAGCTGCTGCTCCCTGCTGCGGAGACCGCGGAGG
CTTCGCGTTGACCAAGTTCCTTAAAGAACTGGCTGATGGGGCAGGAGGTCCAGGCCTGGGCTCTCGGGCC
CTCCTAGAGGGCCATTGGAGCTTGCAGCTCAGACCCCCACTTTGAGTTTTATTTATTTAAATAGTAGTTG
GATGCTTGGCACGTCGTCCTGTAATAGGAAACCCTTGCCTCATCAGTTTTCCTGATTTACAAGTGCAATA
TTTTAGCCAATGCCTTGGGAGAAGCTGCCATGCAAAGGTGGACACCATTCTCCAGCTTCAGGGGATATGC
TCGTCCCGGGCACCGGTGGCAGGCAGCTGGCCTTCTGGACTAAGGCAGCCTGGGGGGACACTGCAGTCTG
GCTACACACAGAGATCTGGCACCCCCTGGGTGGAGTGTCCCTCGGGGGCTTTGGGAAAGCATGGCACCCT
CAGACCACACAGTAGCCAAGTTCTGGAGCAAATAAAAGGCCTGTGTTATTTCTTGTTCTTGA
NM_017790.6 Homo sapiens regulator of G protein signaling 3
(RGS3), transcript variant 3, mRNA
(SEQ ID NO: 12)
GGAGACTCCGGTTACTGGGGAGCAACACAGCCGCCTCGGGTTGCAGACGCTCCTGTCCGGGTCGCAGTGG
GACGCCATGGAGCGCTCCCTGCACCGCGTCTCCCTCGGGAGCCGGCGTGCCCACCCGGACTTGTCCTTCT
ACCTCACCACCTTTGGTCAGCTGAGGCTGTCCATTGATGCCCAGGACCGGGTTCTGCTGCTTCACATTAT
AGAAGGTAAAGGCCTGATCAGCAAACAGCCTGGCACCTGTGATCCGTATGTGAAGATTTCTTTGATCCCT
GAAGATAGTAGACTACGCCACCAGAAGACGCAGACCGTTCCAGACTGCAGAGACCCGGCTTTCCACGAGC
ACTTCTTCTTTCCTGTCCAAGAGGAGGATGATCAGAAGCGTCTCTTGGTTACTGTGTGGAACAGGGCCAG
CCAGTCCAGACAGAGTGGACTCATTGGCTGCATGAGCTTTGGGGTGAAGTCTCTCCTGACTCCAGACAAG
GAGATCAGTGGTTGGTACTACCTCCTAGGGGAGCACCTGGGCCGGACCAAGCACTTGAAGGTGGCCAGGC
GGCGACTGCGGCCGCTGAGAGACCCGCTGCTGAGAATGCCAGGAGGTGGGGACACTGAGAATGGGAAGAA
ACTAAAGATCACCATCCCGAGGGGAAAGGACGGCTTTGGCTTCACCATCTGCTGCGACTCTCCAGTTCGA
GTCCAGGCCGTGGATTCCGGGGGTCCGGCGGAACGGGCAGGGCTGCAGCAGCTGGACACGGTGCTGCAGC
TGAATGAGAGGCCTGTGGAGCACTGGAAATGTGTGGAGCTGGCCCACGAGATCCGGAGCTGCCCCAGTGA
GATCATCCTACTCGTGTGGCGCATGGTCCCCCAGGTCAAGCCAGGACCAGATGGCGGGGTCCTGCGGCGG
GCCTCCTGCAAGTCGACACATGACCTCCAGTCACCCCCCAACAAACGGGAGAAGAACTGCACCCATGGGG
TCCAGGCACGGCCTGAGCAGCGCCACAGCTGCCACCTGGTATGTGACAGCTCTGATGGGCTGCTGCTCGG
CGGCTGGGAGCGCTACACCGAGGTGGCCAAGCGCGGGGGCCAGCACACCCTGCCTGCACTGTCCCGTGCC
ACTGCCCCCACCGACCCCAACTACATCATCCTGGCCCCGCTGAATCCTGGGAGCCAGCTGCTCCGGCCTG
TGTACCAGGAGGATACCATCCCCGAAGAATCAGGGAGTCCCAGTAAAGGGAAGTCCTACACAGGCCTGGG
GAAGAAGTCCCGGCTGATGAAGACAGTGCAGACCATGAAGGGCCACGGGAACTACCAAAACTGCCCGGTT
GTGAGGCCGCATGCCACGCACTCAAGCTATGGCACCTACGTCACCCTGGCCCCCAAAGTCCTGGTGTTCC
CTGTCTTTGTTCAGCCTCTAGATCTCTGTAATCCTGCCCGGACCCTCCTGCTGTCAGAGGAGCTGCTGCT
GTATGAAGGGAGGAACAAGGCTGCCGAGGTGACACTGTTTGCCTATTCGGACCTGCTGCTCTTCACCAAG
GAGGACGAGCCTGGCCGCTGCGACGTCCTGAGGAACCCCCTCTACCTCCAGAGTGTGAAGCTGCAGGAAG
GTTCTTCAGAAGACCTGAAATTCTGCGTGCTCTATCTAGCAGAGAAGGCAGAGTGCTTATTCACTTTGGA
AGCGCACTCGCAGGAGCAGAAGAAGAGAGTGTGCTGGTGCCTGTCGGAGAACATCGCCAAGCAGCAACAG
CTGGCAGCATCACCCCCGGACAGCAAGAAACTCCACCCTTTCGGCTCTCTCCAGCAGGAGATGGGGCCGG
TCAACTCAACCAATGCCACCCAGGATAGAAGCTTTACCTCACCAGGACAGACTCTGATTGGCTGAGCAAA
CCCAAGGGCGGGACTCTGCTTCTGGCAACTTAACCCTTTCTTGGGCATCCCCTACCACACAGTAACCTCA
CCTCAACTGGACCTGAAACAGAGGACCAAGATGGTGGGTCTAGGGTCATCTGAAGGGAGTGGTCCCAAGG
GTGCATGTGGATGTCAACAGAAGGACCACAATGGTGTGAGAGGCCTTGTGGGCATGCAGAGACTGGCAGC
AGAAAGTCCCAAGAGGAGAGGACTCAGAGGCTTCTTCCTTCCAAGGAGGAGGAGGAGGAGGACATTCCAC
ATCTGGGCTTCCAGTGGTGGCTCCCGGACCTGCCTTGGGGGCAGCAGGAGAGGTTGGGAGAGGTCACCCT
GATGAAGAACAATGCCATGATCTGCTGCTGCCAGAGGACATCTCCGCCCATCCACTCATTTTCCACCTGT
ACACATTGGCTCATTCATTCGATGGATATTTCGGGTGCATCTACTTTATGCCAGGCTCTATGCTAGGAGC
TGGGGATACTGTGGAGAAAAAGTCGGATATGGTCTCGCTCTCATCAAGATTTCAGTCTAGCTGGGAGTGA
GCGTTAAATAAGTCAATACCAACACACACACGTGCACACACGTGATGGCAAGCTGTGATCATCTATGAAA
GAAAAAACACAGATTTCTTTAAGAGGGAATTGTACTTAGAGTGCTGTGTGTGGGGGTGGGGATGGGGAAG
GACATAACTTTTAAACCGAGACTTGTGGGCTGGAGAGGAAGGAGCCAGTCCTGGACTTGGGGAAGAGTGT
TCGCGGTGGATGGAATAGCACGTGGAGAGTCCCTGAGATGGGAAGGAGCTCAGTGGGCTGGAGGAATGGC
GAGCAGGCCAGTGTGGCTGGAGCTTAGTGAGGTGCAAGACACAGTGGGTGAGGTAGTTGATGTCTTGTTG
ATTTCATTCTAGATACACTGGAGAGCCATTGAAGGCTTTTGAGGAAGGGAGTTGCAAGTACATTTTTACA
AGATCCGTTTACACATAGGAGGGGCCCAAGTGGGTGTGAGATCAGTTAGGGGGCTCGTTCAGTTCTCTAG
GTGAGGACTGTCCTGGTGGCTTGTCCTGGATGGTGGCTGGGGAAGTGGATGCCTGAATGCCAGGCTCTGG
AGAAGAGATGACTCCCTGCCCCATCCCCCTGCCTACCCTCCTCCTCAATGGCCCCTCTCTCAGTGGATGT
GAGTGAGGATTAGGACCTTCCCCACAGAGCCACCTTTTGGCCAGGCCTCGTGGGGGATAGTGGGGGCAGT
GCTGAGTTCTAGGAGTGTTCCTGGAAGTTGGGGGCAGTGAGTGCTCAGCCTGGCCCAGGTGGGAGAGTTC
ACTGCTGCCCAGGCCTTAGCAGGGGCTGCTGTGCTGTCTTTCTGTCCCATGCTGAGCATTGTGATAGGAA
GCTGCCACACAGTGCCCTGGAAACTGACTTCGCATGCTTTCCATGGGGCTTCCGAGGGCCAGGGACTGGG
CTGGGGGTGGCCTAGCAGCTCTAGTGGAGACTGTGCTGCCGCAGGGCTGAAGGAAGACCAACCAAGGGGA
CAGCCGTTTGAGCCCAGCCCACTCACTGCCTGCTGCACCTCCAGCTGGAGCCCAGGGTTGGGCAGCTCTC
ATACTCTTATCTAACTGCCAGACCTAGAGGGAGGGAAGCCCAAGGCTGCAGGTCTGAGGGACACGGCTCA
TGGGCAAAAGGGTATCAAGAGGATTAGAGTCACTTGCATAGGAAAAGAGGGTCAGAGAGGCTGAGGGCTG
GCTGAGGCCAGAGAGGTTCCCTGACCTTGGACATTCTTTGTGTTGTGTAAGACCTCGGCATCTTTAGAGA
AAGGTTCTTGTGCCCCTGTGAGCTTGCTCAGCTGCTATGGCCACTTGAACCCCAAAAATGGTGGCCTAGA
AGAGAATACACCCCTTACTTCCCACTTATCCTCCTCTCAACCTAGGAAAGATCAGACATATTGACTCAAC
AGTCTAACAGGGGATGGGCTGGATCTGCCCGTGACCAGACCAATGCCCCCCACCCAGCAAGGGAGTAAAC
GCTGCCTGAAGTGGGACCAGTGATACACAGCATCTCTGTCTGCCAGGGCTCAGCCCGCGGGGGGGATTTG
TGAGAGGCTGAGCAGCTGGGTCTGCACAGACAGCAAGCTGCACTTTGATTTCATTTGATTCCCTTACAAA
ATGACATTAAGGGGAATGTGAGGCACAGGAGACTCGCCTGTCTGGGAAGGCAGCAAGCTCATCTCTCCTG
GACCTGGTGACCTACCTCTGTCTACCAGACAGGTGCAGAGGACAGGAGCTCTGGCCACAAAGAAATGGAC
AGTCTGAATGCAGCACACAGCCCTGGGGACAGGACTTTTCACCCAGCCTGTCCTGACAAGCTGCTGAGTC
GAGCAGATGCTCACATCAGGGTCCCAGGTCCGGCTGTTAGGGAAGTGGACATCAGTTCCTGGTGGGCCCT
GGGCAGTGACTGTGGTGAGCCACAGCACCATTGCTGCTCTGCCTCTCAAATCGATCGAACAATTAATTTG
GCTCCACAGGCTCGAGAAGATGAAACATGACCTGATGGCCACTGCTGTCCAGCATCCCAGTCCATCCTAG
CTGTCCAGGATGATGAACAAGCTATGGAAATTGCACTTGAGAGCTATAATCACGGGAAGCATTGCCGAGC
CCCTCCCTGCTACATAAATCATGCTAAGAAGCACACAATGGCCTCTTTCCTGATGGGCTAATTGTCTTGG
TTTGGTGTCTCAGGCCCAGGAGCTGGGAAGATGGGAGCACAACCATTTATTGTTGGAAAGACCATCTGTG
CAACTTGAATACAAAGTGATTCTTTTAGTTCCCCACACACCCCCGACCCTCTGATCCCATTTTCATGTTT
TTTTTTTTTTTTTCTTTTTATGTCTCAGGCTCTGGTTCTTTTAAAACTCTTCATCTATCCTGTAAGCCAT
CAGGCCTGGATCTCTGCTGGCCTGGGTCTTTTGGCTATTTGGGGGTTGGGACATTGCAGACCTTTTGGAA
ATAATGACATTTATTTATATATGTCTTATATTTTTATTCTTTATTTTTCCCCATAAATTCTATTACCAAA
GTCCTCATTTGTCACACATGGCACCAAGGCACCAAGAGGGTTTTTGCAAAGTGAAAATGCAGGCAATTTT
GCAACGTGGAAGGGCTCCCCACAGCGAAAGTCCCACAGTGAGAGCCTAAACTTTGGAGATGGCAACTGGG
GTCCCCAGGCATGATCTCCCCCCAGCCTCCCAGCAGCCTTGTTTTATGGATGAAAGACACCAAGGCTCTG
AGAAGTTAAGTGTTTTATCCTAGGTCACACAGCTGGTAAGGGCCAAGCTTGCCCTCAAGCCAAGGCCTTT
GAACCTAATCTACCACTCATTTCTGATAAAAAGGTTTGTCTTTTAGGGATAGAAGTAAGAGAAATCAATA
ATCTCCAGGAAGAGGCAAGAGAGGGATATCCGGCATGTCAGAAGCTGTGGCCCATCAAGCTGTAGAGGGA
ATGCTTTTAGGATAAATAAAATGAGATGCCTTATTTTCCACCCAGTGGAGAATAAACTTCCAGAATGCAT
TTTCCTGAGGGTGGCACAGGCTGAAAAGATCAATAGCTTCCCACAAGATTTGAAAAAAGCCTAAAATGAG
AGATCAGTTTGAGCTCCTTAAGACAGGTTCTCAGGGAGCTCTGGAATGGTTAGGTTACACCCCTTCCCCT
TTTGAATTTGGCATCAAAAGGATCAGCAGACAGAGTCATGGAGTGCTTGGCAGTGATGGAATATTAGCCA
AAGGCAGAGAGGTATAGCTTCAGGCTCAGGCTGGCTTGTGCCCAGCGTAAGTACTGGAAGGAGGCCTTTG
ACCTGAGACCAGCTGAGGGCACAGAGAGGGCCTGCCCATGTGGAACCTGCCAGGTTTTGTGCGCAGATTT
TGCTAATTGACTGATTTAGTATCTCAGCCCTGAATAAAGGGGAATCAAATGGATCTGATTTCAGCCATCT
CCCCTAAGAGATGCAGACTCTTCTCCCTAAAAACTGTTTCCAGGCAGCCACCAGCTCTCCTCCCCATTCC
TTGCTGCTGTCCTATCAGTGGACAGACTGCCTTCTTCGCTGTCTTGCTCTTGGTCTCAGCCATCTTGCTA
TTGTTTTTTTTAGCCCCTGAAGAAGACAGTGCAACCTTACCCTCTATCCTGTCTTCGCCTTCTCTTCCTC
GAAGACTTTCTCTGAATAGGGCCATCCATCCTCCAAACTGTTCCCAGATCCTTGGTTTTAAAGGTAGAAT
GATAACATAAGGTAACCTGGTGGGCACTTACAAGATGAGCCAGGCACAGGGTCAAGCTTTTTACCTACCC
TGTCTCATCTAATCCTCATGCAGACTTACAAGGTGTGTTACCCCTGTTATACATTTACTCTCATTATGCC
CAATTTAGATATGAAGACGCTGAGTCTCAGAGAAGTTAAGAAACGTGCCCAAGGTCACACAGCTAGTGGG
TAGCTAGGATTGAAACTTGGACAGCCCAGCTCCAGGGTACCTGCTTTTGATTATTTTGCTATATGGCTTT
CCTGTAACTGCAGACCATCCAAGCTGGAAGGGCCAGGGAGACTGGACTGTCACACTCCTGCTTAAAGACC
ATTGTGGGAGGCCACTGTCTTCAGAGTAAAGCTTGGCTTTCCAGGCTCTCCTTGGTAGCACTACACTCCT
GCTTTGCACCTGGAGCCCGGCCACACCATAAATTTTCCCCTTCTCTAAACTCACTGTGCACATTCACAGT
TAGGGTGTTTGATCTGCCTGACGTTGACTGCCCTCCTTGGCTGTCCCTGCTGGATGCAATCCTCAACCTT
CAAGGCTTAGTGGCTCCTCTGAGAACACCTTTCTCAGGCATGCATCAGGAGTCCTGACTTCCCCCTTCGT
GGTTCCCAGAGTACTTTTTCCCCCTTAGCACAATTCATCCTTAGCATCTGGATGACTGTGAGCTTCTTCG
GAGCAAGTAGATGTTATATTTTATTTGTCTTTGATTCTATTGTTGTCCGGTCCGTAGTAGGTGCTCAGTA
GAGGTTGTAGGATTGAATTAGGCTGTCCAGTTCAACTCTCCTATATTCTAGAATTTAGACAAGGAAACAG
GTGCAAAAAGAGAAGTGACCTGGCCAGGGTCATGGGAAAAGTTAGTGTCAGAGCCAGGACTAGAGCTCAG
GTCTCCTGATTCTTTGGATTCAGAGCTTTTTCTGTTGCACCTCAGCTTTGTGGAGACCAGAGCATGGATC
TGCACTTGGTTTATGAAGGTCTGACCAGGTTCACTTCAGGGTCATGGATACCACTGTGTTCCCATGCTGA
CACTGGGGAATTCCCAGAGGGAAAGGAAGCCCAGCTGGAGCTGCTGCTGTAGGTTGCGGGGCTCACCTCT
CACCGAGCCCCTTGCTTACAGAGAAAACCAGCGAGTGATTCTGAAAATCTTGCCTCGAGTCCTCATTTTG
CTGGTATTCACCGTGTGACCTTGAGCAAATTGCTTCACATCTCTGATCTTCTGTTCCTTTACCAAGAAAA
ATGAGGGAGTTGAATTCAGATGGTCTCCAGGAGTCATCTGATTCCAGAATATCAGGATATAACAATAAAG
ATGAGGATAAAGTAGCTACTCCCCTCAGAAAAGGTGGAGATCCAGATCTAGGGGGAGAATTGGCTAGGTT
CTTCATCACAGTCACTGTCTTGAGACACATAGGTGGTCCATGGAAACATTTCCATGAGGGATTTCAGAGG
GGAGAATGGGCAGACTGCAAAGCCTCGGTACTCTAATACCAGGCAAAAAGAAGAAAGGGAACAGGCATTT
TAGTTTTCACATTGAAACAGAGTGTATGTGTATGGCCCTATGCAAATGTTGTTCCTGGTGAATATTCATC
AATGTACTCATCACACATAGGTATATTTAGACAGAAAGGGGTCCTCATCACAGACACCCAAATTATGAGC
ACATTACTGCCTCCTCTCCCCTAGAGGTGCTTCTTCCAGCCTCCCCCATTGGAGGCCAGCACAAGACGGG
CTCTCCAACCCAATATCTTCCCCTTCCCTCCTTCCTTTCTGCTCAGGTGGCTGAGTATACACATTGAACT
CTTTTGTTTCAACTTCACGTGTATATAGGTTGGTGCAAAAGTAACTGTGGTGTTTTCCATTAAAAGCAAT
GTCAAGAACCGCAATTACTTTGGCACCAACCTAATATGTATGCAAGTACATTCTTTAACCTTTCCAACTC
CCAGTCCAAGCCATCAGGCTAATTTCCGCGTGGAATTTGTGGTCACTGGTTTGCAGTCCTTTCATTTCTG
GTGTGGTCAGAAGTCTCCTTCAGACTCTTCCCGATAAGTTGCCATTGGCTTTTTCATCACCTTTAGTACC
TACCCACTTTTGCTGGGATAACACTACCCAAGATAAATTCAAAAAAATTGTCCTAAGTGTTAGTGTGTTT
CTGTTTTGGTTGAAGTCTATTATTGCTATATGGTTTTCTACTGTTTCTACTTGATGGAAGACTATTAAGG
GTTAAGTAGGCTGGCCTAGGAAGCTGACCACCATTTATAACATACTTTTGAGTGGGAAAATGCATTCAGA
GGCCTGTAATCAATACTTAGGTGGACTTTGGGAATATAGTATTTATTTTATGAGTGACTAGAATATTGGT
AGAACCATAGAAACTTTGAATGTGGAGCTGGAAAGCCTCCTCCTTCCCCCACTGAGATCAATTGATTAAC
AAGCTAGGTTGTAGAAACCAAGTGAAAGAGGCAAAAACTTGGCCAAGGGCATGTAGTCGGGGACTTCTGA
AGATTCTGCTGGGCTGGGGGCAGGAAAGGGGTCACAGACGGTCTTAGGGTTGTCTCACTCAGCAACTCAC
TGTGTTGGCCTTGGGAAATGTAAGTCACTGTGTTGGGCTGTAATGGAAATATTGAGAAAGCAGCTCAGCT
AGTGGCGTGGGGAGAGGTTGTCTAGTCCTCTGGCTTATCTGTAAATTCCTGGCATGTGCTTGTTGGAGCA
GAATGAATGGGGCTGCTGTGTTTTACCTGCAGCTAGGAGCATGGCTGGAGTGGTGGAGACACTCCCTGTG
GTCTGTTCTAAAAATAGCAGTGGGAACAGAGCTGAGGGGAAGAGGAGGGGGCTCCTTCGGGAGCTGGGTG
GGGAGGCCTCACCCCCTTCCTCTTCCTGCCAGGCCCGATGTGAGGAAGTCCCATGGAGTCACATAATTCC
ATCTGGGAGAGTCCTGGAGCCATCAGCCCTCACACCCCCTCCTCATACAGGCGAGGAGGCCCTGGAGGCC
CGGAGAGCAGAAAGCACTGGCTGGTGTCAAGCAAGCCCAGAGAGAAGGGCCCAGTTGGCAGGCTGTTTTT
CCCTGGCTGTTTCAGCACAGTGGCTGCAGGCCTTGTGCTGAGGTTTGCTGTCACTGCCTTTGGGCGCACA
GAGACTGGGAACTCCTGGGACCGGATGTGGTCAGGCTTCCTGAGAGTCGCCTGGCAGGCGAGGAGTCCCC
AGCTGGCCCACCCTGAGCCCTGACCTCAGCCAGCCGGCTGTCCAGTGCCACACCTCCTCTTGCTGGTGTC
CCCTCCCTTGCTGACTTGTCCTCCTTTCTTTGGACTGTCTGCAGGGAGGGAGGACAAGGGCCAGCCTAGA
TCTGGCCTGCAGGACAGAGGCCTCAGTGGCTCAGGACCTTTCCCTGCCCCTCCCCAGGAACAAGCAGAGG
CAGCTGAGGTAGTAGCAGCCTCCTGCAGGTTTAGAGACAGACAGGCCAGGGTTCAAATCCTAGCTCTTCC
TCTCACTAGCCATGGGATTACTGATGGGTCACAGAGCATCTGTAAAATGAGACAAGAACGCTGATTTCAC
TGGGGTCTTGTGAAGATCAAAGGGGATCTGTTGTAATGCCTGGCATGCAGTAGGTACTTAATAGTAGCTC
TTGTTGCAAAACCAAGCAGTGAACATGTATCTGACTTTCTATAACCCCCTTCCTTGGGGCCTCCACTCAT
GAGGCATGGGAAGGTGTTTTTTTTCTTTGTTTGTTTGTTTGTTTGAATGCAGTTGACTTTTTTTTTGTTG
TATTGTTGCAAAAGTAATCCATGTGATTGATGTTTATTATGAAGAAAACATTTAATACAGATGAACCAGG
AGAAGAAAATGAAGTTCATCCTTAATCCCAGCACCCAGAGATAAACCACTGTTAACATTTTGGAATGTGT
CCTGTCAGGCATTCGTTTATACACACATATATCTTATTTGCAAATTGGGGTTACTGGTGCAATTTTTCTA
ACCTGCTTTTTCTCCCCCATTTAACAGCGTAGTGTATCCATGTTTCCATTTCAATAAATATTCTTCTACA
ACATCA
NM_130795.4 Homo sapiens regulator of G protein signaling 3
(RGS3), transcript variant 1, mRNA
(SEQ ID NO: 13)
AGTGCGGGCTCCCGCTGCTGGGGGAGAGCTGGGTTTTCATGGGGCGGCAGCCGAGGCAGGACCCGCAGCC
ATGAACCGCTTCAATGGGCTCTGCAAGGTGTGCTCGGAGCGCCGCTACCGCCAGATCACCATCCCGAGGG
GAAAGGACGGCTTTGGCTTCACCATCTGCTGCGACTCTCCAGTTCGAGTCCAGGCCGTGGATTCCGGGGG
TCCGGCGGAACGGGCAGGGCTGCAGCAGCTGGACACGGTGCTGCAGCTGAATGAGAGGCCTGTGGAGCAC
TGGAAATGTGTGGAGCTGGCCCACGAGATCCGGAGCTGCCCCAGTGAGATCATCCTACTCGTGTGGCGCA
TGGTCCCCCAGGTCAAGCCAGGACCAGATGGCGGGGTCCTGCGGCGGGCCTCCTGCAAGTCGACACATGA
CCTCCAGTCACCCCCCAACAAACGGGAGAAGAACTGCACCCATGGGGTCCAGGCACGGCCTGAGCAGCGC
CACAGCTGCCACCTGGTATGTGACAGCTCTGATGGGCTGCTGCTCGGCGGCTGGGAGCGCTACACCGAGG
TGGCCAAGCGCGGGGGCCAGCACACCCTGCCTGCACTGTCCCGTGCCACTGCCCCCACCGACCCCAACTA
CATCATCCTGGCCCCGCTGAATCCTGGGAGCCAGCTGCTCCGGCCTGTGTACCAGGAGGATACCATCCCC
GAAGAATCAGGGAGTCCCAGTAAAGGGAAGTCCTACACAGGCCTGGGGAAGAAGTCCCGGCTGATGAAGA
CAGTGCAGACCATGAAGGGCCACGGGAACTACCAAAACTGCCCGGTTGTGAGGCCGCATGCCACGCACTC
AAGCTATGGCACCTACGTCACCCTGGCCCCCAAAGTCCTGGTGTTCCCTGTCTTTGTTCAGCCTCTAGAT
CTCTGTAATCCTGCCCGGACCCTCCTGCTGTCAGAGGAGCTGCTGCTGTATGAAGGGAGGAACAAGGCTG
CCGAGGTGACACTGTTTGCCTATTCGGACCTGCTGCTCTTCACCAAGGAGGACGAGCCTGGCCGCTGCGA
CGTCCTGAGGAACCCCCTCTACCTCCAGAGTGTGAAGCTGCAGGAAGGTTCTTCAGAAGACCTGAAATTC
TGCGTGCTCTATCTAGCAGAGAAGGCAGAGTGCTTATTCACTTTGGAAGCGCACTCGCAGGAGCAGAAGA
AGAGAGTGTGCTGGTGCCTGTCGGAGAACATCGCCAAGCAGCAACAGCTGGCAGCATCACCCCCGGACAG
CAAGATGTTTGAGACGGAGGCAGATGAGAAGAGGGAGATGGCCTTGGAGGAAGGGAAGGGGCCTGGTGCC
GAGGATTCCCCACCCAGCAAGGAGCCCTCTCCTGGCCAGGAGCTTCCTCCAGGACAAGACCTTCCACCCA
ACAAGGACTCCCCTTCTGGGCAGGAACCCGCTCCCAGCCAAGAACCACTGTCCAGCAAAGACTCAGCTAC
CTCTGAAGGATCCCCTCCAGGCCCAGATGCTCCGCCCAGCAAGGATGTGCCACCATGCCAGGAACCCCCT
CCAGCCCAAGACCTCTCACCCTGCCAGGACCTACCTGCTGGTCAAGAACCCCTGCCTCACCAGGACCCTC
TACTCACCAAAGACCTCCCTGCCATCCAGGAATCCCCCACCCGGGACCTTCCACCCTGTCAAGATCTGCC
TCCTAGCCAGGTCTCCCTGCCAGCCAAGGCCCTTACTGAGGACACCATGAGCTCCGGGGACCTACTAGCA
GCTACTGGGGACCCACCTGCGGCCCCCAGGCCAGCCTTCGTGATCCCTGAGGTCCGGCTGGATAGCACCT
ACAGCCAGAAGGCAGGGGCAGAGCAGGGCTGCTCGGGAGATGAGGAGGATGCAGAAGAGGCCGAGGAGGT
GGAGGAGGGGGAGGAAGGGGAGGAGGACGAGGATGAGGACACCAGCGATGACAACTACGGAGAGCGCAGT
GAGGCCAAGCGCAGCAGCATGATCGAGACGGGCCAGGGGGCTGAGGGTGGCCTCTCACTGCGTGTGCAGA
ACTCGCTGCGGCGCCGGACGCACAGCGAGGGCAGCCTGCTGCAGGAGCCCCGAGGGCCCTGCTTTGCCTC
CGACACCACCTTGCACTGCTCAGACGGTGAGGGCGCCGCCTCCACCTGGGGCATGCCTTCGCCCAGCACC
CTCAAGAAAGAGCTGGGCCGCAATGGTGGCTCCATGCACCACCTTTCCCTCTTCTTCACAGGACACAGGA
AGATGAGCGGGGCTGACACCGTTGGGGATGATGACGAAGCCTCCCGGAAGAGAAAGAGCAAAAACCTAGC
CAAGGACATGAAGAACAAGCTGGGGATCTTCAGACGGCGGAATGAGTCCCCTGGAGCCCCTCCCGCGGGC
AAGGCAGACAAAATGATGAAGTCATTCAAGCCCACCTCAGAGGAAGCCCTCAAGTGGGGCGAGTCCTTGG
AGAAGCTGCTGGTTCACAAATACGGGTTAGCAGTGTTCCAAGCCTTCCTTCGCACTGAGTTCAGTGAGGA
GAATCTGGAGTTCTGGTTGGCTTGTGAGGACTTCAAGAAGGTCAAGTCACAGTCCAAGATGGCATCCAAG
GCCAAGAAGATCTTTGCTGAATACATCGCGATCCAGGCATGCAAGGAGGTCAACCTGGACTCCTACACGC
GGGAGCACACCAAGGACAACCTGCAGAGCGTCACGCGGGGCTGCTTCGACCTGGCACAGAAGCGCATCTT
CGGGCTCATGGAAAAGGACTCGTACCCTCGCTTTCTCCGTTCTGACCTCTACCTGGACCTTATTAACCAG
AAGAAGATGAGTCCCCCGCTTTAGGGGCCACTGGAGTCGAGCTCAGCGTTCACACCAGGCGGGCTGGGTC
CCCTGCCCACCTGCCTCCCTGCCCCCTGTGACGGAGGGGGCAAGCAAGCCCCCAGAGGCTGTGTCTCTGG
ACAGACGGATAGACATACGGAAGCGAGGCCTGGACCAAGAGAGGCCCAGGCTACTGGAGGAGTAGAAGGA
TGGGCCCCGTGGGGTCCCCACTGCCCCGGTACGAGGGGGCCCAAGACCCTGGCAGGTCAGGGGCCCTGGC
CAAGCCAGATCTGGAGCTGCTGCTCCCTGCTGCGGAGACCGCGGAGGCTTCGCGTTGACCAAGTTCCTTA
AAGAACTGGCTGATGGGGCAGGAGGTCCAGGCCTGGGCTCTCGGGCCCTCCTAGAGGGCCATTGGAGCTT
GCAGCTCAGACCCCCACTTTGAGTTTTATTTATTTAAATAGTAGTTGGATGCTTGGCACGTCGTCCTGTA
ATAGGAAACCCTTGCCTCATCAGTTTTCCTGATTTACAAGTGCAATATTTTAGCCAATGCCTTGGGAGAA
GCTGCCATGCAAAGGTGGACACCATTCTCCAGCTTCAGGGGATATGCTCGTCCCGGGCACCGGTGGCAGG
CAGCTGGCCTTCTGGACTAAGGCAGCCTGGGGGGACACTGCAGTCTGGCTACACACAGAGATCTGGCACC
CCCTGGGTGGAGTGTCCCTCGGGGGCTTTGGGAAAGCATGGCACCCTCAGACCACACAGTAGCCAAGTTC
TGGAGCAAATAAAAGGCCTGTGTTATTTCTTGTTCTTGA
NM_134427.3 Homo sapiens regulator of G protein signaling 3
(RGS3), transcript variant 4, mRNA
(SEQ ID NO: 14)
AGTGCCAGGGGGTGCCGTTGTGAGGGATGGACGCCTCTCCCCCGGAGCAGCACTTTGGGGCCAGCTTGGG
CCCTGGGGATGAGCCACAGGGGACCCACAGCCTATGCGTGTGAGCATGTAACCCGGGAGCCAGCTGGGCC
CCTCGCGGGGTGGGCAGAAGGACGGGCTGGCCCAGGACCCTCTTCTTTGAGACATCCCGGACTCCATCTC
GGATGAAAGTGCTTTGAGAAACCACAGAGTTTTCTGTTTAATGGAAGAAAGAAATCCAGCCTCTCTCCAG
AGTGGCGGTGGCCGGCTAGACAGGAGCCAAGGACATGAAGAACAAGCTGGGGATCTTCAGACGGCGGAAT
GAGTCCCCTGGAGCCCCTCCCGCGGGCAAGGCAGACAAAATGATGAAGTCATTCAAGCCCACCTCAGAGG
AAGCCCTCAAGTGGGGCGAGTCCTTGGAGAAGCTGCTGGTTCACAAATACGGGTTAGCAGTGTTCCAAGC
CTTCCTTCGCACTGAGTTCAGTGAGGAGAATCTGGAGTTCTGGTTGGCTTGTGAGGACTTCAAGAAGGTC
AAGTCACAGTCCAAGATGGCATCCAAGGCCAAGAAGATCTTTGCTGAATACATCGCGATCCAGGCATGCA
AGGAGGTCAACCTGGACTCCTACACGCGGGAGCACACCAAGGACAACCTGCAGAGCGTCACGCGGGGCTG
CTTCGACCTGGCACAGAAGCGCATCTTCGGGCTCATGGAAAAGGACTCGTACCCTCGCTTTCTCCGTTCT
GACCTCTACCTGGACCTTATTAACCAGAAGAAGATGAGTCCCCCGCTTTAGGGGCCACTGGAGTCGAGCT
CAGCGTTCACACCAGGCGGGCTGGGTCCCCTGCCCACCTGCCTCCCTGCCCCCTGTGACGGAGGGGGCAA
GCAAGCCCCCAGAGGCTGTGTCTCTGGACAGACGGATAGACATACGGAAGCGAGGCCTGGACCAAGAGAG
GCCCAGGCTACTGGAGGAGTAGAAGGATGGGCCCCGTGGGGTCCCCACTGCCCCGGTACGAGGGGGCCCA
AGACCCTGGCAGGTCAGGGGCCCTGGCCAAGCCAGATCTGGAGCTGCTGCTCCCTGCTGCGGAGACCGCG
GAGGCTTCGCGTTGACCAAGTTCCTTAAAGAACTGGCTGATGGGGCAGGAGGTCCAGGCCTGGGCTCTCG
GGCCCTCCTAGAGGGCCATTGGAGCTTGCAGCTCAGACCCCCACTTTGAGTTTTATTTATTTAAATAGTA
GTTGGATGCTTGGCACGTCGTCCTGTAATAGGAAACCCTTGCCTCATCAGTTTTCCTGATTTACAAGTGC
AATATTTTAGCCAATGCCTTGGGAGAAGCTGCCATGCAAAGGTGGACACCATTCTCCAGCTTCAGGGGAT
ATGCTCGTCCCGGGCACCGGTGGCAGGCAGCTGGCCTTCTGGACTAAGGCAGCCTGGGGGGACACTGCAG
TCTGGCTACACACAGAGATCTGGCACCCCCTGGGTGGAGTGTCCCTCGGGGGCTTTGGGAAAGCATGGCA
CCCTCAGACCACACAGTAGCCAAGTTCTGGAGCAAATAAAAGGCCTGTGTTATTTCTTGTTCTTGA
NM_144488.8 Homo sapiens regulator of G protein signaling 3
(RGS3), transcript variant 6, mRNA
(SEQ ID NO: 15)
GAACTTTGGCTCAGCCTGTCTTCATCCACTGTATCCTGACACCTCTCCTACTCCTTCTCTTTAATGTACA
GCTGAGACTGGGATACCCTTAGACTGAAAGGAAGGACACGGCTATGTAGAAAAGAATTGACTGAAGGGGA
GGAGTACTCGTGAACCCCTTGCTTCAGCCTCCCAATGTTCTGAGAGAGGAGGAGCCAGGCGCAGTGGCTC
ATGCCTGTAATCCCAGCACTTTGGGAGGTTGAGATGGGCAGATCGCAAGGTCAGGAGATCGAGACCATCC
TGGCTAACAGGTCACATTCAGACAGCACACCTTTGCCCAATTTTCTTTCTGGATCTCACCGTCCTGAGTG
TTGTACCTGCAGGTTGCTCACAGCCTCTGGAGCCCAAGATAGTCTCCCCTTTGGGAGGAGGCTCTACAGT
GGTCCCTGGCGAAGTTGTGAAGAGGTCTGCCACGTCTCTGTGCTCAGTGTCCTCTCTACATCCTGTGGCT
TGAGCCTGAGCTTGCCCATATTCCCTGGCTGGATGGAGTGGCTAAGCCCTGATATCGCTCTGCCCAGAAG
AGATGAGTGGACTCAAACTTCTCCAGCCAGGAAGAGGATCACGCATGCCAAAGTCCAGGGTGCAGGTCAG
CTGAGGCTGTCCATTGATGCCCAGGACCGGGTTCTGCTGCTTCACATTATAGAAGGTAAAGGCCTGATCA
GCAAACAGCCTGGCACCTGTGATCCGTATGTGAAGATTTCTTTGATCCCTGAAGATAGTAGACTACGCCA
CCAGAAGACGCAGACCGTTCCAGACTGCAGAGACCCGGCTTTCCACGAGCACTTCTTCTTTCCTGTCCAA
GAGGAGGATGATCAGAAGCGTCTCTTGGTTACTGTGTGGAACAGGGCCAGCCAGTCCAGACAGAGTGGAC
TCATTGGCTGCATGAGCTTTGGGGTGAAGTCTCTCCTGACTCCAGACAAGGAGATCAGTGGTTGGTACTA
CCTCCTAGGGGAGCACCTGGGCCGGACCAAGCACTTGAAGGTGGCCAGGCGGCGACTGCGGCCGCTGAGA
GACCCGCTGCTGAGAATGCCAGGAGGTGGGGACACTGAGAATGGGAAGAAACTAAAGATCACCATCCCGA
GGGGAAAGGACGGCTTTGGCTTCACCATCTGCTGCGACTCTCCAGTTCGAGTCCAGGCCGTGGATTCCGG
GGGTCCGGCGGAACGGGCAGGGCTGCAGCAGCTGGACACGGTGCTGCAGCTGAATGAGAGGCCTGTGGAG
CACTGGAAATGTGTGGAGCTGGCCCACGAGATCCGGAGCTGCCCCAGTGAGATCATCCTACTCGTGTGGC
GCATGGTCCCCCAGGTCAAGCCAGGACCAGATGGCGGGGTCCTGCGGCGGGCCTCCTGCAAGTCGACACA
TGACCTCCAGTCACCCCCCAACAAACGGGAGAAGAACTGCACCCATGGGGTCCAGGCACGGCCTGAGCAG
CGCCACAGCTGCCACCTGGTATGTGACAGCTCTGATGGGCTGCTGCTCGGCGGCTGGGAGCGCTACACCG
AGGTGGCCAAGCGCGGGGGCCAGCACACCCTGCCTGCACTGTCCCGTGCCACTGCCCCCACCGACCCCAA
CTACATCATCCTGGCCCCGCTGAATCCTGGGAGCCAGCTGCTCCGGCCTGTGTACCAGGAGGATACCATC
CCCGAAGAATCAGGGAGTCCCAGTAAAGGGAAGTCCTACACAGGCCTGGGGAAGAAGTCCCGGCTGATGA
AGACAGTGCAGACCATGAAGGGCCACGGGAACTACCAAAACTGCCCGGTTGTGAGGCCGCATGCCACGCA
CTCAAGCTATGGCACCTACGTCACCCTGGCCCCCAAAGTCCTGGTGTTCCCTGTCTTTGTTCAGCCTCTA
GATCTCTGTAATCCTGCCCGGACCCTCCTGCTGTCAGAGGAGCTGCTGCTGTATGAAGGGAGGAACAAGG
CTGCCGAGGTGACACTGTTTGCCTATTCGGACCTGCTGCTCTTCACCAAGGAGGACGAGCCTGGCCGCTG
CGACGTCCTGAGGAACCCCCTCTACCTCCAGAGTGTGAAGCTGCAGGAAGGTTCTTCAGAAGACCTGAAA
TTCTGCGTGCTCTATCTAGCAGAGAAGGCAGAGTGCTTATTCACTTTGGAAGCGCACTCGCAGGAGCAGA
AGAAGAGAGTGTGCTGGTGCCTGTCGGAGAACATCGCCAAGCAGCAACAGCTGGCAGCATCACCCCCGGA
CAGCAAGATGTTTGAGACGGAGGCAGATGAGAAGAGGGAGATGGCCTTGGAGGAAGGGAAGGGGCCTGGT
GCCGAGGATTCCCCACCCAGCAAGGAGCCCTCTCCTGGCCAGGAGCTTCCTCCAGGACAAGACCTTCCAC
CCAACAAGGACTCCCCTTCTGGGCAGGAACCCGCTCCCAGCCAAGAACCACTGTCCAGCAAAGACTCAGC
TACCTCTGAAGGATCCCCTCCAGGCCCAGATGCTCCGCCCAGCAAGGATGTGCCACCATGCCAGGAACCC
CCTCCAGCCCAAGACCTCTCACCCTGCCAGGACCTACCTGCTGGTCAAGAACCCCTGCCTCACCAGGACC
CTCTACTCACCAAAGACCTCCCTGCCATCCAGGAATCCCCCACCCGGGACCTTCCACCCTGTCAAGATCT
GCCTCCTAGCCAGGTCTCCCTGCCAGCCAAGGCCCTTACTGAGGACACCATGAGCTCCGGGGACCTACTA
GCAGCTACTGGGGACCCACCTGCGGCCCCCAGGCCAGCCTTCGTGATCCCTGAGGTCCGGCTGGATAGCA
CCTACAGCCAGAAGGCAGGGGCAGAGCAGGGCTGCTCGGGAGATGAGGAGGATGCAGAAGAGGCCGAGGA
GGTGGAGGAGGGGGAGGAAGGGGAGGAGGACGAGGATGAGGACACCAGCGATGACAACTACGGAGAGCGC
AGTGAGGCCAAGCGCAGCAGCATGATCGAGACGGGCCAGGGGGCTGAGGGTGGCCTCTCACTGCGTGTGC
AGAACTCGCTGCGGCGCCGGACGCACAGCGAGGGCAGCCTGCTGCAGGAGCCCCGAGGGCCCTGCTTTGC
CTCCGACACCACCTTGCACTGCTCAGACGGTGAGGGCGCCGCCTCCACCTGGGGCATGCCTTCGCCCAGC
ACCCTCAAGAAAGAGCTGGGCCGCAATGGTGGCTCCATGCACCACCTTTCCCTCTTCTTCACAGGACACA
GGAAGATGAGCGGGGCTGACACCGTTGGGGATGATGACGAAGCCTCCCGGAAGAGAAAGAGCAAAAACCT
AGCCAAGGACATGAAGAACAAGCTGGGGATCTTCAGACGGCGGAATGAGTCCCCTGGAGCCCCTCCCGCG
GGCAAGGCAGACAAAATGATGAAGTCATTCAAGCCCACCTCAGAGGAAGCCCTCAAGTGGGGCGAGTCCT
TGGAGAAGCTGCTGGTTCACAAATACGGGTTAGCAGTGTTCCAAGCCTTCCTTCGCACTGAGTTCAGTGA
GGAGAATCTGGAGTTCTGGTTGGCTTGTGAGGACTTCAAGAAGGTCAAGTCACAGTCCAAGATGGCATCC
AAGGCCAAGAAGATCTTTGCTGAATACATCGCGATCCAGGCATGCAAGGAGGTCAACCTGGACTCCTACA
CGCGGGAGCACACCAAGGACAACCTGCAGAGCGTCACGCGGGGCTGCTTCGACCTGGCACAGAAGCGCAT
CTTCGGGCTCATGGAAAAGGACTCGTACCCTCGCTTTCTCCGTTCTGACCTCTACCTGGACCTTATTAAC
CAGAAGAAGATGAGTCCCCCGCTTTAGGGGCCACTGGAGTCGAGCTCAGCGTTCACACCAGGCGGGCTGG
GTCCCCTGCCCACCTGCCTCCCTGCCCCCTGTGACGGAGGGGGCAAGCAAGCCCCCAGAGGCTGTGTCTC
TGGACAGACGGATAGACATACGGAAGCGAGGCCTGGACCAAGAGAGGCCCAGGCTACTGGAGGAGTAGAA
GGATGGGCCCCGTGGGGTCCCCACTGCCCCGGTACGAGGGGGCCCAAGACCCTGGCAGGTCAGGGGCCCT
GGCCAAGCCAGATCTGGAGCTGCTGCTCCCTGCTGCGGAGACCGCGGAGGCTTCGCGTTGACCAAGTTCC
TTAAAGAACTGGCTGATGGGGCAGGAGGTCCAGGCCTGGGCTCTCGGGCCCTCCTAGAGGGCCATTGGAG
CTTGCAGCTCAGACCCCCACTTTGAGTTTTATTTATTTAAATAGTAGTTGGATGCTTGGCACGTCGTCCT
GTAATAGGAAACCCTTGCCTCATCAGTTTTCCTGATTTACAAGTGCAATATTTTAGCCAATGCCTTGGGA
GAAGCTGCCATGCAAAGGTGGACACCATTCTCCAGCTTCAGGGGATATGCTCGTCCCGGGCACCGGTGGC
AGGCAGCTGGCCTTCTGGACTAAGGCAGCCTGGGGGGACACTGCAGTCTGGCTACACACAGAGATCTGGC
ACCCCCTGGGTGGAGTGTCCCTCGGGGGCTTTGGGAAAGCATGGCACCCTCAGACCACACAGTAGCCAAG
TTCTGGAGCAAATAAAAGGCCTGTGTTATTTCTTGTTCTTGA
NM_144489.4 Homo sapiens regulator of G protein signaling 3
(RGS3), transcript variant 5, mRNA
(SEQ ID NO: 16)
AGTCATCAGGCCAGGATTCCAGAGAGCGTGTGTGGCTGCAGCCTGCACCGTTGCTGCCCGCTGCCCAGGA
CGCGGGGTGGGGGACAGGAGCCAGAGTGGTGCCTCCTACAGACCAATCTGCGGCCCCAAGGTGGGGGGCC
CTACAGAGATGCTCCGAGGCATGTACCTCACTCGCAACGGGAACCTGCAGAGGCGACACACGATGAAGGA
AGCCAAGGACATGAAGAACAAGCTGGGGATCTTCAGACGGCGGAATGAGTCCCCTGGAGCCCCTCCCGCG
GGCAAGGCAGACAAAATGATGAAGTCATTCAAGCCCACCTCAGAGGAAGCCCTCAAGTGGGGCGAGTCCT
TGGAGAAGCTGCTGGTTCACAAATACGGGTTAGCAGTGTTCCAAGCCTTCCTTCGCACTGAGTTCAGTGA
GGAGAATCTGGAGTTCTGGTTGGCTTGTGAGGACTTCAAGAAGGTCAAGTCACAGTCCAAGATGGCATCC
AAGGCCAAGAAGATCTTTGCTGAATACATCGCGATCCAGGCATGCAAGGAGGTCAACCTGGACTCCTACA
CGCGGGAGCACACCAAGGACAACCTGCAGAGCGTCACGCGGGGCTGCTTCGACCTGGCACAGAAGCGCAT
CTTCGGGCTCATGGAAAAGGACTCGTACCCTCGCTTTCTCCGTTCTGACCTCTACCTGGACCTTATTAAC
CAGAAGAAGATGAGTCCCCCGCTTTAGGGGCCACTGGAGTCGAGCTCAGCGTTCACACCAGGCGGGCTGG
GTCCCCTGCCCACCTGCCTCCCTGCCCCCTGTGACGGAGGGGGCAAGCAAGCCCCCAGAGGCTGTGTCTC
TGGACAGACGGATAGACATACGGAAGCGAGGCCTGGACCAAGAGAGGCCCAGGCTACTGGAGGAGTAGAA
GGATGGGCCCCGTGGGGTCCCCACTGCCCCGGTACGAGGGGGCCCAAGACCCTGGCAGGTCAGGGGCCCT
GGCCAAGCCAGATCTGGAGCTGCTGCTCCCTGCTGCGGAGACCGCGGAGGCTTCGCGTTGACCAAGTTCC
TTAAAGAACTGGCTGATGGGGCAGGAGGTCCAGGCCTGGGCTCTCGGGCCCTCCTAGAGGGCCATTGGAG
CTTGCAGCTCAGACCCCCACTTTGAGTTTTATTTATTTAAATAGTAGTTGGATGCTTGGCACGTCGTCCT
GTAATAGGAAACCCTTGCCTCATCAGTTTTCCTGATTTACAAGTGCAATATTTTAGCCAATGCCTTGGGA
GAAGCTGCCATGCAAAGGTGGACACCATTCTCCAGCTTCAGGGGATATGCTCGTCCCGGGCACCGGTGGC
AGGCAGCTGGCCTTCTGGACTAAGGCAGCCTGGGGGGACACTGCAGTCTGGCTACACACAGAGATCTGGC
ACCCCCTGGGTGGAGTGTCCCTCGGGGGCTTTGGGAAAGCATGGCACCCTCAGACCACACAGTAGCCAAG
TTCTGGAGCAAATAAAAGGCCTGTGTTATTTCTTGTTCTTGA
NM_001102445. 3 Homo sapiens regulator of G protein signaling 4
(RGS4), transcript variant 1, mRNA
(SEQ ID NO: 17)
ACTGCGTGGAGACGATGATCCTGCCAGCTCCCTTTTGGAAATCGTGAGGATCAGATCTTGGACCATGTAT
AATATGATGCTTCTAATCCAAAAGAGGAAAGGCATTGGGAGTCAGCTCCTAAGGGCTGGAGAGGCAGAGG
GAGACAGAGGAGCTGGTACTGCAGAGCGGTCGTCTGATTGGCTGGACGGTCGTAGCTGGGCTATAAAAGA
GACCCCTACAGGCTTAGCAGGAAGACGCTCAGAGGATTCTGACAATATCTTTACCGGAGAAGAGGCAAAG
TACGCTCAAAGCCGAAGCCACAGCTCCTCCTGCCGCATTTCTTTCCTGCTTGCGAATTCCAAGCTGTTAA
ATAAGATGTGCAAAGGGCTTGCAGGTCTGCCGGCTTCTTGCTTGAGGAGTGCAAAAGATATGAAACATCG
GCTAGGTTTCCTGCTGCAAAAATCTGATTCCTGTGAACACAATTCTTCCCACAACAAGAAGGACAAAGTG
GTTATTTGCCAGAGAGTGAGCCAAGAGGAAGTCAAGAAATGGGCTGAATCACTGGAAAACCTGATTAGTC
ATGAATGTGGGCTGGCAGCTTTCAAAGCTTTCTTGAAGTCTGAATATAGTGAGGAGAATATTGACTTCTG
GATCAGCTGTGAAGAGTACAAGAAAATCAAATCACCATCTAAACTAAGTCCCAAGGCCAAAAAGATCTAT
AATGAATTCATCTCAGTCCAGGCAACCAAAGAGGTGAACCTGGATTCTTGCACCAGGGAAGAGACAAGCC
GGAACATGCTAGAGCCTACAATAACCTGCTTTGATGAGGCCCAGAAGAAGATTTTCAACCTGATGGAGAA
GGATTCCTACCGCCGCTTCCTCAAGTCTCGATTCTATCTTGATTTGGTCAACCCGTCCAGCTGTGGGGCA
GAAAAGCAGAAAGGAGCCAAGAGTTCAGCAGACTGTGCTTCCCTGGTCCCTCAGTGTGCCTAATTCTCAC
CTGAAGGCAGAGGGATGAAATGCCAAGACTCTATGCTCTGGAAAACCTGAGGCCAAATATTGATCTGTAT
TAAGCTCCAGTGCTTTATCCACATTGTAGCCTAATATTCATGCTGCCTGCCATGTGTGAGTCACTTCTAC
GCATAAACTAGATATAGCTTTTGGTGTTTGAGTGTTCATCAGGGTGGGACCCCATTCCAGTCCAATTTTC
CTAAGTTTCTTTGAGGGTTCCATGGGAGCAAATATCTAAATAATGGCCTGGTAGGTCTGGATTTTCAAAG
ATTGTTGGCAGTTTCCTCCTCCCAACAGTTTTACCTCGGGATGGTTGGTTAGTGCATGTCACATGACATC
CACATGCACATGTATTCTGTTGGCCAGCACGTTCTCCAGACTCTAGATGTTTAGATGAGGTTGAGCTATG
ATATGTGCTTGTGTGTATGTCTATGTGTATATATTATATATACATTAGACACACATATACATTATTTCTG
TATATAGATGTCTGTGTATACATATGTATGTGTGAGTGTATGTATACACACACACACACACACACACACA
CACTTTTGCAAGAGTGATGGGAAAGACCCTAGGTGCTCATAACTAGAGTATGTGTATGTACTTACATGGG
TGTTTTGATCTCTGTTCTTTCATACTACATTTGAACAGGGCAAAATGAACTAACTGCCATGTAGGCTAAG
AAAGAAATGCTAACCTGTGGAAAGTTGGTTTTGTAAAATTCCATGGATCTTGCTGGAGAAGCATCCAAGG
AACTTCATGCTTGATTTGACCACTGACAGCCTCCACCTTGAGCACTATTCTAAGGAGCAAATACCTTAGC
TCCCTTGAGCTGGTTTTCTCTGATGGCACTTTTGAGCTCCTAAGCTGCCAGCCTTCCCTTCTTTTCCTGG
GTGCTCAGGGCATGCTTATTAGCAGCTGGGTTGGTATGGAGTTGGCAGACAGGATGTTCAACTTAATGAA
GAAATACAGCTAAGGCCTTGCCAGCAACACCTGCCGTAAGTTACTGGCTGAGTGAGGGCATAGAAGTTAA
AGGTTACTGTTTTTATCCTCTATCCTTTTTTCCTTTCCTGATCAAGGTGCTCTTCTCATTTTTTCCTGAG
AACCTTAGCCATCAGATGAGGCTCCTTAGTTTATTGTGGTTGGTTGTTTTTTCTTTATAATGGCTCTGGG
CTATATGCCTATATTTATAAACCAGCAGCAGGGGAAAGATTATATTTTATAAGAGGGAACAAATTTTCAC
AATTTGAAAAGCCCACATAAGTTTTCTCTTTTAAGGTAGAATCTTGTTAATTTCATTCCAAACATCGGGG
CTAACAGAGACTGGAGGCATTTCTTTTTAGGCTCTGAGACTAAATGAGAGGAAAAGAAAAGAAAAAAAAA
ATGATTGTCTAACCAATTGTGAGAATTACTGTTTGAAACTTTTCAAGGCACATTGAAATACTTGAAAACT
TCTCATTTATGTTATTTATGATGTTATTTTGTACGTGTTATTATTATTATATTGTTTTATAAATGGAGGT
ACAGGATATCACCTGAATTATTAATGAATGCCCAGGAAGTAATTTTCTTCTCATTCTTCTAAAACTACTG
CCTTTCAAAGTGCACACACACGCGTCCACATACACTGCATTCGTTGCTCCAGTATAAATTACATGCATGA
GCACCTTTCTGGCTTTTAAGCCAATATAATGGGCTGCAAAATGAAGACACCAGAGTGTATGCATACAAAT
CTCACTGTATTAAAGATGCAGGTTTTCTAATTGTACCCTTCTTGTCTCTCTGGCAATCTTGCCCTTAATA
TCCCTGGAGTTCCTCATCAGTGTCATTTTCTGTTATACACAGTTCCACAATTTTGTCTCTAGTTGACTTC
AAATGTGTAACTTTATTGGTCTTGCCCTATTATAATTGTCATGACTTTCAGATTGTATCTGAACTCACAG
ACTGCTGTCTTACTAATAGGTCTGGAAGGTCACGCTGAATGAGAAGTAAATTATTTTATGTAATACATTT
TTGAGTGTGTTTTTCAGTTGTATTTCCCTGTTATTTCATCACTATTTCCAATGGTGAGCTTGCCTGCTCA
TGCTCCCTGGACAGAATACTCCTTCCTTTTGCATGCCTGTTTCTATCATGTGCTTGATAGGCCTCAAAGC
TAATGCTTCCAGTGAAACACACGCATCTTAATAATAAGGGTAAATAAACGCTCCATATGAAACTA
NM_001113380.1 Homo sapiens regulator of G protein signaling 4
(RGS4), transcript variant 3, mRNA
(SEQ ID NO: 18)
ACTTTCCCGAGGTGCTTCTACAGTTCCCTCTGCCAGCAGGGGAACAGATGGAAATAGCAATCACCTGCCA
GAAGGTGGCGTGCAGCAAGGATGTGCATCTTTTGCCGCTACTGCTTTCTGATTCCTAAAAATTACTCAGA
GATCACTCATGTGTTCAGTGATTCAGGTTCTGTTGAAGATACCAAAGATATTCGGTTGGTCAAAATGACG
GGCATATAAAGGCTTCTCAGGTTTCTGAGTGCAAAAGATATGAAACATCGGCTAGGTTTCCTGCTGCAAA
AATCTGATTCCTGTGAACACAATTCTTCCCACAACAAGAAGGACAAAGTGGTTATTTGCCAGAGAGTGAG
CCAAGAGGAAGTCAAGAAATGGGCTGAATCACTGGAAAACCTGATTAGTCATGAATGTGGGCTGGCAGCT
TTCAAAGCTTTCTTGAAGTCTGAATATAGTGAGGAGAATATTGACTTCTGGATCAGCTGTGAAGAGTACA
AGAAAATCAAATCACCATCTAAACTAAGTCCCAAGGCCAAAAAGATCTATAATGAATTCATCTCAGTCCA
GGCAACCAAAGAGGTGAACCTGGATTCTTGCACCAGGGAAGAGACAAGCCGGAACATGCTAGAGCCTACA
ATAACCTGCTTTGATGAGGCCCAGAAGAAGATTTTCAACCTGATGGAGAAGGATTCCTACCGCCGCTTCC
TCAAGTCTCGATTCTATCTTGATTTGGTCAACCCGTCCAGCTGTGGGGCAGAAAAGCAGAAAGGAGCCAA
GAGTTCAGCAGACTGTGCTTCCCTGGTCCCTCAGTGTGCCTAATTCTCACCTGAAGGCAGAGGGATGAAA
TGCCAAGACTCTATGCTCTGGAAAACCTGAGGCCAAATATTGATCTGTATTAAGCTCCAGTGCTTTATCC
ACATTGTAGCCTAATATTCATGCTGCCTGCCATGTGTGAGTCACTTCTACGCATAAACTAGATATAGCTT
TTGGTGTTTGAGTGTTCATCAGGGTGGGACCCCATTCCAGTCCAATTTTCCTAAGTTTCTTTGAGGGTTC
CATGGGAGCAAATATCTAAATAATGGCCTGGTAGGTCTGGATTTTCAAAGATTGTTGGCAGTTTCCTCCT
CCCAACAGTTTTACCTCGGGATGGTTGGTTAGTGCATGTCACATGACATCCACATGCACATGTATTCTGT
TGGCCAGCACGTTCTCCAGACTCTAGATGTTTAGATGAGGTTGAGCTATGATATGTGCTTGTGTGTATGT
CTATGTGTATATATTATATATACATTAGACACACATATACATTATTTCTGTATATAGATGTCTGTGTATA
CATATGTATGTGTGAGTGTATGTATACACACACACACACACACACACACACACTTTTGCAAGAGTGATGG
GAAAGACCCTAGGTGCTCATAACTAGAGTATGTGTATGTACTTACATGGGTGTTTTGATCTCTGTTCTTT
CATACTACATTTGAACAGGGCAAAATGAACTAACTGCCATGTAGGCTAAGAAAGAAATGCTAACCTGTGG
AAAGTTGGTTTTGTAAAATTCCATGGATCTTGCTGGAGAAGCATCCAAGGAACTTCATGCTTGATTTGAC
CACTGACAGCCTCCACCTTGAGCACTATTCTAAGGAGCAAATACCTTAGCTCCCTTGAGCTGGTTTTCTC
TGATGGCACTTTTGAGCTCCTAAGCTGCCAGCCTTCCCTTCTTTTCCTGGGTGCTCAGGGCATGCTTATT
AGCAGCTGGGTTGGTATGGAGTTGGCAGACAGGATGTTCAACTTAATGAAGAAATACAGCTAAGGCCTTG
CCAGCAACACCTGCCGTAAGTTACTGGCTGAGTGAGGGCATAGAAGTTAAAGGTTACTGTTTTTATCCTC
TATCCTTTTTTCCTTTCCTGATCAAGGTGCTCTTCTCATTTTTTCCTGAGAACCTTAGCCATCAGATGAG
GCTCCTTAGTTTATTGTGGTTGGTTGTTTTTTCTTTATAATGGCTCTGGGCTATATGCCTATATTTATAA
ACCAGCAGCAGGGGAAAGATTATATTTTATAAGAGGGAACAAATTTTCACAATTTGAAAAGCCCACATAA
GTTTTCTCTTTTAAGGTAGAATCTTGTTAATTTCATTCCAAACATCGGGGCTAACAGAGACTGGAGGCAT
TTCTTTTTAGGCTCTGAGACTAAATGAGAGGAAAAGAAAAGAAAAAAAAAATGATTGTCTAACCAATTGT
GAGAATTACTGTTTGAAACTTTTCAAGGCACATTGAAATACTTGAAAACTTCTCATTTATGTTATTTATG
ATGTTATTTTGTACGTGTTATTATTATTATATTGTTTTATAAATGGAGGTACAGGATATCACCTGAATTA
TTAATGAATGCCCAGGAAGTAATTTTCTTCTCATTCTTCTAAAACTACTGCCTTTCAAAGTGCACACACA
CGCGTCCACATACACTGCATTCGTTGCTCCAGTATAAATTACATGCATGAGCACCTTTCTGGCTTTTAAG
CCAATATAATGGGCTGCAAAATGAAGACACCAGAGTGTATGCATACAAATCTCACTGTATTAAAGATGCA
GGTTTTCTAATTGTACCCTTCTTGTCTCTCTGGCAATCTTGCCCTTAATATCCCTGGAGTTCCTCATCAG
TGTCATTTTCTGTTATACACAGTTCCACAATTTTGTCTCTAGTTGACTTCAAATGTGTAACTTTATTGGT
CTTGCCCTATTATAATTGTCATGACTTTCAGATTGTATCTGAACTCACAGACTGCTGTCTTACTAATAGG
TCTGGAAGGTCACGCTGAATGAGAAGTAAATTATTTTATGTAATACATTTTTGAGTGTGTTTTTCAGTTG
TATTTCCCTGTTATTTCATCACTATTTCCAATGGTGAGCTTGCCTGCTCATGCTCCCTGGACAGAATACT
CCTTCCTTTTGCATGCCTGTTTCTATCATGTGCTTGATAGGCCTCAAAGCTAATGCTTCCAGTGAAACAC
ACGCATCTTAATAATAAGGGTAAATAAACGCTCCATATGAAACTA
NM_001113381.1 Homo sapiens regulator of G protein signaling 4
(RGS4), transcript variant 4, mRNA
(SEQ ID NO: 19)
GCTGGAGAGGCAGAGGGAGACAGAGGAGCTGGTACTGCAGAGCGGTCGTCTGATTGGCTGGACGGTCGTA
GCTGGGCTATAAAAGAGACCCCTACAGGCTTAGCAGGAAGACGCTCAGAGGATTCTGACAATATCTTTAC
CGGAGAAGAGGCAAAGTACGCTCAAAGCCGAAGCCACAGCTCCTCCTGCCGCATTTCTTTCCTGCTTGCG
AATTCCAAGCTGTTAAATAAGATGTGCAAAGGGCTTGCAGGTCTGCCGGCTTCTTGCTTGAGGAGTGCAA
AAGATATGAAACATCGGCTAGGTTTCCTGCTGCAAAAATCTGATTCCTGTGAACACAATTCTTCCCACAA
CAAGAAGGACAAAGTGGTTATTTGCCAGAGAGTGAGCCAAGAGGAAGTCAAGAAATGGGCTGAATCACTG
GAAAACCTGATTAGTCATGAATGTGAACCTGGATTCTTGCACCAGGGAAGAGACAAGCCGGAACATGCTA
GAGCCTACAATAACCTGCTTTGATGAGGCCCAGAAGAAGATTTTCAACCTGATGGAGAAGGATTCCTACC
GCCGCTTCCTCAAGTCTCGATTCTATCTTGATTTGGTCAACCCGTCCAGCTGTGGGGCAGAAAAGCAGAA
AGGAGCCAAGAGTTCAGCAGACTGTGCTTCCCTGGTCCCTCAGTGTGCCTAATTCTCACCTGAAGGCAGA
GGGATGAAATGCCAAGACTCTATGCTCTGGAAAACCTGAGGCCAAATATTGATCTGTATTAAGCTCCAGT
GCTTTATCCACATTGTAGCCTAATATTCATGCTGCCTGCCATGTGTGAGTCACTTCTACGCATAAACTAG
ATATAGCTTTTGGTGTTTGAGTGTTCATCAGGGTGGGACCCCATTCCAGTCCAATTTTCCTAAGTTTCTT
TGAGGGTTCCATGGGAGCAAATATCTAAATAATGGCCTGGTAGGTCTGGATTTTCAAAGATTGTTGGCAG
TTTCCTCCTCCCAACAGTTTTACCTCGGGATGGTTGGTTAGTGCATGTCACATGACATCCACATGCACAT
GTATTCTGTTGGCCAGCACGTTCTCCAGACTCTAGATGTTTAGATGAGGTTGAGCTATGATATGTGCTTG
TGTGTATGTCTATGTGTATATATTATATATACATTAGACACACATATACATTATTTCTGTATATAGATGT
CTGTGTATACATATGTATGTGTGAGTGTATGTATACACACACACACACACACACACACACACTTTTGCAA
GAGTGATGGGAAAGACCCTAGGTGCTCATAACTAGAGTATGTGTATGTACTTACATGGGTGTTTTGATCT
CTGTTCTTTCATACTACATTTGAACAGGGCAAAATGAACTAACTGCCATGTAGGCTAAGAAAGAAATGCT
AACCTGTGGAAAGTTGGTTTTGTAAAATTCCATGGATCTTGCTGGAGAAGCATCCAAGGAACTTCATGCT
TGATTTGACCACTGACAGCCTCCACCTTGAGCACTATTCTAAGGAGCAAATACCTTAGCTCCCTTGAGCT
GGTTTTCTCTGATGGCACTTTTGAGCTCCTAAGCTGCCAGCCTTCCCTTCTTTTCCTGGGTGCTCAGGGC
ATGCTTATTAGCAGCTGGGTTGGTATGGAGTTGGCAGACAGGATGTTCAACTTAATGAAGAAATACAGCT
AAGGCCTTGCCAGCAACACCTGCCGTAAGTTACTGGCTGAGTGAGGGCATAGAAGTTAAAGGTTACTGTT
TTTATCCTCTATCCTTTTTTCCTTTCCTGATCAAGGTGCTCTTCTCATTTTTTCCTGAGAACCTTAGCCA
TCAGATGAGGCTCCTTAGTTTATTGTGGTTGGTTGTTTTTTCTTTATAATGGCTCTGGGCTATATGCCTA
TATTTATAAACCAGCAGCAGGGGAAAGATTATATTTTATAAGAGGGAACAAATTTTCACAATTTGAAAAG
CCCACATAAGTTTTCTCTTTTAAGGTAGAATCTTGTTAATTTCATTCCAAACATCGGGGCTAACAGAGAC
TGGAGGCATTTCTTTTTAGGCTCTGAGACTAAATGAGAGGAAAAGAAAAGAAAAAAAAAATGATTGTCTA
ACCAATTGTGAGAATTACTGTTTGAAACTTTTCAAGGCACATTGAAATACTTGAAAACTTCTCATTTATG
TTATTTATGATGTTATTTTGTACGTGTTATTATTATTATATTGTTTTATAAATGGAGGTACAGGATATCA
CCTGAATTATTAATGAATGCCCAGGAAGTAATTTTCTTCTCATTCTTCTAAAACTACTGCCTTTCAAAGT
GCACACACACGCGTCCACATACACTGCATTCGTTGCTCCAGTATAAATTACATGCATGAGCACCTTTCTG
GCTTTTAAGCCAATATAATGGGCTGCAAAATGAAGACACCAGAGTGTATGCATACAAATCTCACTGTATT
AAAGATGCAGGTTTTCTAATTGTACCCTTCTTGTCTCTCTGGCAATCTTGCCCTTAATATCCCTGGAGTT
CCTCATCAGTGTCATTTTCTGTTATACACAGTTCCACAATTTTGTCTCTAGTTGACTTCAAATGTGTAAC
TTTATTGGTCTTGCCCTATTATAATTGTCATGACTTTCAGATTGTATCTGAACTCACAGACTGCTGTCTT
ACTAATAGGTCTGGAAGGTCACGCTGAATGAGAAGTAAATTATTTTATGTAATACATTTTTGAGTGTGTT
TTTCAGTTGTATTTCCCTGTTATTTCATCACTATTTCCAATGGTGAGCTTGCCTGCTCATGCTCCCTGGA
CAGAATACTCCTTCCTTTTGCATGCCTGTTTCTATCATGTGCTTGATAGGCCTCAAAGCTAATGCTTCCA
GTGAAACACACGCATCTTAATAATAAGGGTAAATAAACGCTCCATATGAAACTA
NM_005613.6 Homo sapiens regulator of G protein signaling 4
(RGS4), transcript variant 2, mRNA
(SEQ ID NO: 20)
AAGACGCTCAGAGGATTCTGACAATATCTTTACCGGAGAAGAGGCAAAGTACGCTCAAAGCCGAAGCCAC
AGCTCCTCCTGCCGCATTTCTTTCCTGCTTGCGAATTCCAAGCTGTTAAATAAGATGTGCAAAGGGCTTG
CAGGTCTGCCGGCTTCTTGCTTGAGGAGTGCAAAAGATATGAAACATCGGCTAGGTTTCCTGCTGCAAAA
ATCTGATTCCTGTGAACACAATTCTTCCCACAACAAGAAGGACAAAGTGGTTATTTGCCAGAGAGTGAGC
CAAGAGGAAGTCAAGAAATGGGCTGAATCACTGGAAAACCTGATTAGTCATGAATGTGGGCTGGCAGCTT
TCAAAGCTTTCTTGAAGTCTGAATATAGTGAGGAGAATATTGACTTCTGGATCAGCTGTGAAGAGTACAA
GAAAATCAAATCACCATCTAAACTAAGTCCCAAGGCCAAAAAGATCTATAATGAATTCATCTCAGTCCAG
GCAACCAAAGAGGTGAACCTGGATTCTTGCACCAGGGAAGAGACAAGCCGGAACATGCTAGAGCCTACAA
TAACCTGCTTTGATGAGGCCCAGAAGAAGATTTTCAACCTGATGGAGAAGGATTCCTACCGCCGCTTCCT
CAAGTCTCGATTCTATCTTGATTTGGTCAACCCGTCCAGCTGTGGGGCAGAAAAGCAGAAAGGAGCCAAG
AGTTCAGCAGACTGTGCTTCCCTGGTCCCTCAGTGTGCCTAATTCTCACCTGAAGGCAGAGGGATGAAAT
GCCAAGACTCTATGCTCTGGAAAACCTGAGGCCAAATATTGATCTGTATTAAGCTCCAGTGCTTTATCCA
CATTGTAGCCTAATATTCATGCTGCCTGCCATGTGTGAGTCACTTCTACGCATAAACTAGATATAGCTTT
TGGTGTTTGAGTGTTCATCAGGGTGGGACCCCATTCCAGTCCAATTTTCCTAAGTTTCTTTGAGGGTTCC
ATGGGAGCAAATATCTAAATAATGGCCTGGTAGGTCTGGATTTTCAAAGATTGTTGGCAGTTTCCTCCTC
CCAACAGTTTTACCTCGGGATGGTTGGTTAGTGCATGTCACATGACATCCACATGCACATGTATTCTGTT
GGCCAGCACGTTCTCCAGACTCTAGATGTTTAGATGAGGTTGAGCTATGATATGTGCTTGTGTGTATGTC
TATGTGTATATATTATATATACATTAGACACACATATACATTATTTCTGTATATAGATGTCTGTGTATAC
ATATGTATGTGTGAGTGTATGTATACACACACACACACACACACACACACACTTTTGCAAGAGTGATGGG
AAAGACCCTAGGTGCTCATAACTAGAGTATGTGTATGTACTTACATGGGTGTTTTGATCTCTGTTCTTTC
ATACTACATTTGAACAGGGCAAAATGAACTAACTGCCATGTAGGCTAAGAAAGAAATGCTAACCTGTGGA
AAGTTGGTTTTGTAAAATTCCATGGATCTTGCTGGAGAAGCATCCAAGGAACTTCATGCTTGATTTGACC
ACTGACAGCCTCCACCTTGAGCACTATTCTAAGGAGCAAATACCTTAGCTCCCTTGAGCTGGTTTTCTCT
GATGGCACTTTTGAGCTCCTAAGCTGCCAGCCTTCCCTTCTTTTCCTGGGTGCTCAGGGCATGCTTATTA
GCAGCTGGGTTGGTATGGAGTTGGCAGACAGGATGTTCAACTTAATGAAGAAATACAGCTAAGGCCTTGC
CAGCAACACCTGCCGTAAGTTACTGGCTGAGTGAGGGCATAGAAGTTAAAGGTTACTGTTTTTATCCTCT
ATCCTTTTTTCCTTTCCTGATCAAGGTGCTCTTCTCATTTTTTCCTGAGAACCTTAGCCATCAGATGAGG
CTCCTTAGTTTATTGTGGTTGGTTGTTTTTTCTTTATAATGGCTCTGGGCTATATGCCTATATTTATAAA
CCAGCAGCAGGGGAAAGATTATATTTTATAAGAGGGAACAAATTTTCACAATTTGAAAAGCCCACATAAG
TTTTCTCTTTTAAGGTAGAATCTTGTTAATTTCATTCCAAACATCGGGGCTAACAGAGACTGGAGGCATT
TCTTTTTAGGCTCTGAGACTAAATGAGAGGAAAAGAAAAGAAAAAAAAAATGATTGTCTAACCAATTGTG
AGAATTACTGTTTGAAACTTTTCAAGGCACATTGAAATACTTGAAAACTTCTCATTTATGTTATTTATGA
TGTTATTTTGTACGTGTTATTATTATTATATTGTTTTATAAATGGAGGTACAGGATATCACCTGAATTAT
TAATGAATGCCCAGGAAGTAATTTTCTTCTCATTCTTCTAAAACTACTGCCTTTCAAAGTGCACACACAC
GCGTCCACATACACTGCATTCGTTGCTCCAGTATAAATTACATGCATGAGCACCTTTCTGGCTTTTAAGC
CAATATAATGGGCTGCAAAATGAAGACACCAGAGTGTATGCATACAAATCTCACTGTATTAAAGATGCAG
GTTTTCTAATTGTACCCTTCTTGTCTCTCTGGCAATCTTGCCCTTAATATCCCTGGAGTTCCTCATCAGT
GTCATTTTCTGTTATACACAGTTCCACAATTTTGTCTCTAGTTGACTTCAAATGTGTAACTTTATTGGTC
TTGCCCTATTATAATTGTCATGACTTTCAGATTGTATCTGAACTCACAGACTGCTGTCTTACTAATAGGT
CTGGAAGGTCACGCTGAATGAGAAGTAAATTATTTTATGTAATACATTTTTGAGTGTGTTTTTCAGTTGT
ATTTCCCTGTTATTTCATCACTATTTCCAATGGTGAGCTTGCCTGCTCATGCTCCCTGGACAGAATACTC
CTTCCTTTTGCATGCCTGTTTCTATCATGTGCTTGATAGGCCTCAAAGCTAATGCTTCCAGTGAAACACA
CGCATCTTAATAATAAGGGTAAATAAACGCTCCATATGAAACT

Primers or probes can be designed so that they hybridize under stringent conditions to mutant nucleotide sequences of at least one of RGS3, or RGS4, but not to the respective wild-type nucleotide sequences. Primers or probes can also be prepared that are complementary and specific for the wild-type nucleotide sequence of at least one of RGS3, or RGS4, but not to any one of the corresponding mutant nucleotide sequences. In some embodiments, the mutant nucleotide sequences of at least one of RGS3, or RGS4 may be a frameshift mutation, a missense mutation, a deletion, an insertion, a nonsense mutation, an inversion, or a translocation, that results in the loss of expression and/or activity of at least one of RGS3, or RGS4 (i.e., loss of function mutations).

In some embodiments, detection can occur through any of a variety of mobility dependent analytical techniques based on the differential rates of migration between different nucleic acid sequences. Exemplary mobility-dependent analysis techniques include electrophoresis, chromatography, mass spectroscopy, sedimentation, gradient centrifugation, field-flow fractionation, multi-stage extraction techniques, and the like. In some embodiments, mobility probes can be hybridized to amplification products, and the identity of the target nucleic acid sequence determined via a mobility dependent analysis technique of the eluted mobility probes, as described in Published PCT Applications WO04/46344 and WO01/92579. In some embodiments, detection can be achieved by various microarrays and related software such as the Applied Biosystems Array System with the Applied Biosystems 1700 Chemiluminescent Microarray Analyzer and other commercially available array systems available from Affymetrix, Agilent, Illumina, and Amersham Biosciences, among others (see also Gerry et al., J. Mol. Biol. 292:251-62, 1999; De Bellis et al., Minerva Biotec 14:247-52, 2002; and Stears et al., Nat. Med. 9:14045, including supplements, 2003).

It is also understood that detection can comprise reporter groups that are incorporated into the reaction products, either as part of labeled primers or due to the incorporation of labeled dNTPs during an amplification, or attached to reaction products, for example but not limited to, via hybridization tag complements comprising reporter groups or via linker arms that are integral or attached to reaction products. In some embodiments, unlabeled reaction products may be detected using mass spectrometry.

### NGS Platforms

In some embodiments, high throughput, massively parallel sequencing employs sequencing-by-synthesis with reversible dye terminators. In other embodiments, sequencing is performed via sequencing-by-ligation. In yet other embodiments, sequencing is single molecule sequencing. Examples of Next Generation Sequencing techniques include, but are not limited to pyrosequencing, Reversible dye-terminator sequencing, SOLID sequencing, Ion semiconductor sequencing, Helioscope single molecule sequencing etc.

The Ion Torrent™ (Life Technologies, Carlsbad, CA) amplicon sequencing system employs a flow-based approach that detects pH changes caused by the release of hydrogen ions during incorporation of unmodified nucleotides in DNA replication. For use with this system, a sequencing library is initially produced by generating DNA fragments flanked by sequencing adapters. In some embodiments, these fragments can be clonally amplified on particles by emulsion PCR. The particles with the amplified template are then placed in a silicon semiconductor sequencing chip. During replication, the chip is flooded with one nucleotide after another, and if a nucleotide complements the DNA molecule in a particular microwell of the chip, then it will be incorporated. A proton is naturally released when a nucleotide is incorporated by the polymerase in the DNA molecule, resulting in a detectable local change of pH. The pH of the solution then changes in that well and is detected by the ion sensor. If homopolymer repeats are present in the template sequence, multiple nucleotides will be incorporated in a single cycle. This leads to a corresponding number of released hydrogens and a proportionally higher electronic signal.

The 454™ GS FLX™ sequencing system (Roche, Germany), employs a light-based detection methodology in a large-scale parallel pyrosequencing system. Pyrosequencing uses DNA polymerization, adding one nucleotide species at a time and detecting and quantifying the number of nucleotides added to a given location through the light emitted by the release of attached pyrophosphates. For use with the 454™ system, adapter-ligated DNA fragments are fixed to small DNA-capture beads in a water-in-oil emulsion and amplified by PCR (emulsion PCR). Each DNA-bound bead is placed into a well on a picotiter plate and sequencing reagents are delivered across the wells of the plate. The four DNA nucleotides are added sequentially in a fixed order across the picotiter plate device during a sequencing run. During the nucleotide flow, millions of copies of DNA bound to each of the beads are sequenced in parallel. When a nucleotide complementary to the template strand is added to a well, the nucleotide is incorporated onto the existing DNA strand, generating a light signal that is recorded by a CCD camera in the instrument.

Sequencing technology based on reversible dye-terminators: DNA molecules are first attached to primers on a slide and amplified so that local clonal colonies are formed. Four types of reversible terminator bases (RT-bases) are added, and non-incorporated nucleotides are washed away. Unlike pyrosequencing, the DNA can only be extended one nucleotide at a time. A camera takes images of the fluorescently labeled nucleotides, then the dye along with the terminal 3′ blocker is chemically removed from the DNA, allowing the next cycle.

Helicos's single-molecule sequencing uses DNA fragments with added poly A tail adapters, which are attached to the flow cell surface. At each cycle, DNA polymerase and a single species of fluorescently labeled nucleotide are added, resulting in template-dependent extension of the surface-immobilized primer-template duplexes. The reads are performed by the Helioscope sequencer. After acquisition of images tiling the full array, chemical cleavage and release of the fluorescent label permits the subsequent cycle of extension and imaging.

Sequencing by synthesis (SBS), like the “old style” dye-termination electrophoretic sequencing, relies on incorporation of nucleotides by a DNA polymerase to determine the base sequence. A DNA library with affixed adapters is denatured into single strands and grafted to a flow cell, followed by bridge amplification to form a high-density array of spots onto a glass chip. Reversible terminator methods use reversible versions of dye-terminators, adding one nucleotide at a time, detecting fluorescence at each position by repeated removal of the blocking group to allow polymerization of another nucleotide. The signal of nucleotide incorporation can vary with fluorescently labeled nucleotides, phosphate-driven light reactions and hydrogen ion sensing having all been used. Examples of SBS platforms include Illumina GA and HiSeq 2000. The MiSeq® personal sequencing system (Illumina, Inc.) also employs sequencing by synthesis with reversible terminator chemistry.

In contrast to the sequencing by synthesis method, the sequencing by ligation method uses a DNA ligase to determine the target sequence. This sequencing method relies on enzymatic ligation of oligonucleotides that are adjacent through local complementarity on a template DNA strand. This technology employs a partition of all possible oligonucleotides of a fixed length, labeled according to the sequenced position. Oligonucleotides are annealed and ligated and the preferential ligation by DNA ligase for matching sequences results in a dinucleotide encoded color space signal at that position (through the release of a fluorescently labeled probe that corresponds to a known nucleotide at a known position along the oligo). This method is primarily used by Life Technologies' SOLiD™ sequencers. Before sequencing, the DNA is amplified by emulsion PCR. The resulting beads, each containing only copies of the same DNA molecule, are deposited on a solid planar substrate.

SMRT™ sequencing is based on the sequencing by synthesis approach. The DNA is synthesized in zero-mode wave-guides (ZMWs)-small well-like containers with the capturing tools located at the bottom of the well. The sequencing is performed with use of unmodified polymerase (attached to the ZMW bottom) and fluorescently labeled nucleotides flowing freely in the solution. The wells are constructed in a way that only the fluorescence occurring at the bottom of the well is detected. The fluorescent label is detached from the nucleotide at its incorporation into the DNA strand, leaving an unmodified DNA strand.

### Pharmaceutical Compositions

In one aspect, the present disclosure provides pharmaceutical compositions comprising a KRAS^{G12C} inhibitor or a downstream inhibitor of RAS signaling pathway.

The pharmaceutical compositions of the present disclosure may be prepared by any of the methods known in the pharmaceutical arts. The amount of active ingredient that can be combined with a carrier material to produce a single dosage form will vary depending upon the host being treated and the particular mode of administration. The amount of active ingredient that can be combined with a carrier material to produce a single dosage form will generally be that amount of the compound that produces a therapeutic effect. Generally, the amount of active compound will be in the range of about 0.1 to 99 percent, more typically, about 5 to 70 percent, and more typically, about 10 to 30 percent.

In some embodiments, pharmaceutical compositions of the present technology may contain one or more pharmaceutically-acceptable carriers, which as used herein, generally refers to a pharmaceutically-acceptable composition, such as a liquid or solid filler, diluent, excipient, manufacturing aid (e.g., lubricant, talc magnesium, calcium or zinc stearate, or steric acid), or solvent encapsulating material, useful for introducing the active agent into the body.

Examples of suitable aqueous and non-aqueous carriers that may be employed in the pharmaceutical compositions of the present technology include, for example, water, ethanol, polyols (such as glycerol, propylene glycol, polyethylene glycol, and the like), vegetable oils (such as olive oil), and injectable organic esters (such as ethyl oleate), and suitable mixtures thereof. Proper fluidity can be maintained, for example, by the use of coating materials, such as lecithin, by the maintenance of the required particle size in the case of dispersions, and by the use of surfactants.

In some embodiments, the formulations may include one or more of sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; alginic acid; buffering agents, such as magnesium hydroxide and aluminum hydroxide; pyrogen-free water; isotonic saline; Ringer's solution; ethyl alcohol; pH buffered solutions; polyesters, polycarbonates and/or polyanhydrides; preservatives; glidants; fillers; and other non-toxic compatible substances employed in pharmaceutical formulations.

Various auxiliary agents, such as wetting agents, emulsifiers, lubricants (e.g., sodium lauryl sulfate and magnesium stearate), coloring agents, release agents, coating agents, sweetening agents, flavoring agents, preservative agents, and antioxidants can also be included in the pharmaceutical composition of the present technology. Some examples of pharmaceutically-acceptable antioxidants include: water soluble antioxidants, such as ascorbic acid, cysteine hydrochloride, sodium bisulfate, sodium metabisulfite, sodium sulfite, and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like. In some embodiments, the pharmaceutical formulation includes an excipient selected from, for example, celluloses, liposomes, lipid nanoparticles, micelle-forming agents (e.g., bile acids), and polymeric carriers, e.g., polyesters and polyanhydrides. Suspensions, in addition to the active compounds, may contain suspending agents, such as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof. Prevention of the action of microorganisms on the active compounds may be ensured by the inclusion of various antibacterial and antifungal agents, such as, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption, such as aluminum monostearate and gelatin.

### Modes of Administration and Effective Dosages

Any method known to those in the art for contacting a cell, organ or tissue with one or more KRAS^{G12C} inhibitors or downstream inhibitors of RAS signaling pathway disclosed herein may be employed. Suitable methods include in vitro, ex vivo, or in vivo methods. In vivo methods typically include the administration of one or more KRAS^{G12C} inhibitors or downstream inhibitors of RAS signaling pathway to a mammal, suitably a human. When used in vivo for therapy, the one or more KRAS^{G12C} inhibitors or downstream inhibitors of RAS signaling pathway described herein are administered to the subject in effective amounts (i.e., amounts that have desired therapeutic effect). The dose and dosage regimen will depend upon the degree of the disease state of the subject, the characteristics of the particular KRAS^{G12C} inhibitor or downstream inhibitor of RAS signaling pathway used, e.g., its therapeutic index, and the subject's history.

The effective amount may be determined during pre-clinical trials and clinical trials by methods familiar to physicians and clinicians. An effective amount of one or more KRAS^{G12C} inhibitors or downstream inhibitors of RAS signaling pathway useful in the methods may be administered to a mammal in need thereof by any of a number of well-known methods for administering pharmaceutical compounds. The KRAS^{G12C} inhibitor or downstream inhibitor of RAS signaling pathway may be administered systemically or locally.

The one or more KRAS^{G12C} inhibitors or downstream inhibitors of RAS signaling pathway described herein can be incorporated into pharmaceutical compositions for administration, singly or in combination, to a subject for the treatment or prevention of a cancer comprising a constitutively active KRAS mutation. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., 7 days of treatment).

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The pharmaceutical compositions having one or more KRAS^{G12C} inhibitors or downstream inhibitors of RAS signaling pathway disclosed herein can include a carrier, which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressurized container or dispenser, which contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

A therapeutic agent can be formulated in a carrier system. The carrier can be a colloidal system. The colloidal system can be a liposome, a phospholipid bilayer vehicle, or a lipid nanoparticle. In one embodiment, the therapeutic agent is encapsulated in a liposome while maintaining the agent's structural integrity. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34(7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic agent can be embedded in the polymer matrix, while maintaining the agent's structural integrity. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compounds can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,”Trends Biotechnol., 13(12):527-37 (1995). Mizguchi, et al., Cancer Lett., 100:63-69 (1996), describes the use of fusogenic liposomes to deliver a protein to cells both in vivo and in vitro.

Dosage, toxicity and therapeutic efficacy of any therapeutic agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

Typically, an effective amount of the one or more KRAS^{G12C} inhibitors or downstream inhibitors of RAS signaling pathway disclosed herein sufficient for achieving a therapeutic or prophylactic effect, range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example, dosages can be 1 mg/kg body weight or 10 mg/kg body weight every day, every two days or every three days or within the range of 1-10 mg/kg every week, every two weeks or every three weeks. In one embodiment, a single dosage of the therapeutic compound ranges from 0.001-10,000 micrograms per kg body weight. In one embodiment, one or more KRAS^{G12C} inhibitor or downstream inhibitor of RAS signaling pathway concentrations in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

In some embodiments, a therapeutically effective amount of one or more KRAS^{G12C} inhibitors or downstream inhibitors of RAS signaling pathway may be defined as a concentration of inhibitor at the target tissue of 10-32 to 106 molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application).

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance with the present methods can be any mammal, including, for example, farm animals, such as sheep, pigs, cows, and horses; pet animals, such as dogs and cats; laboratory animals, such as rats, mice and rabbits. In some embodiments, the mammal is a human.

### Theranostic Methods of the Present Technology

In one aspect, the present disclosure provides a method for selecting a cancer patient harboring a constitutively active KRAS mutation for treatment with a KRAS^{G12C} inhibitor that selectively targets the inactive state of KRAS comprising (a) detecting mRNA or polypeptide expression levels and/or activity of one or more regulators of G-protein signaling in a biological sample obtained from the cancer patient; and administering to the cancer patient an effective amount of the KRAS^{G12C} inhibitor when expression levels and/or activity of the one or more regulators of G-protein signaling are comparable to a control sample obtained from a healthy subject or a predetermined threshold. In another aspect, the present disclosure provides a method for treating a cancer patient harboring a constitutively active KRAS mutation comprising administering to the cancer patient an effective amount of a KRAS^{G12C} inhibitor that selectively targets the inactive state of KRAS, wherein mRNA or polypeptide expression and/or activity levels of one or more regulators of G-protein signaling in a biological sample obtained from the cancer patient are comparable to a control sample obtained from a healthy subject or a predetermined threshold. Examples of KRAS^{G12C} inhibitors that selectively target the inactive state of KRAS comprises one or more of MRTX1257, MRTX849, AMG510, ARS-1620, ARS-3248, LY3499446, LY3537982, GDC-6036, D-1553, JDQ443, BI 1823911, CodeBreak 100, ARS-853, WW peptide, DC-032-759, PTD-RBD-VIF, AU-8653, or ADT-007.

In one aspect, the present disclosure provides a method for selecting a cancer patient harboring a constitutively active KRAS mutation for treatment with a downstream inhibitor of RAS signaling pathway comprising (a) detecting mRNA or polypeptide expression levels and/or activity of one or more regulators of G-protein signaling in a biological sample obtained from the cancer patient; and (b) administering to the cancer patient an effective amount of the downstream inhibitor of RAS signaling pathway when expression levels and/or activity of the one or more regulators of G-protein signaling are reduced relative to a control sample obtained from a healthy subject or a predetermined threshold. Also disclosed herein are methods for treating a cancer patient harboring a constitutively active KRAS mutation comprising administering to the cancer patient an effective amount of a downstream inhibitor of RAS signaling pathway, wherein mRNA or polypeptide expression and/or activity levels of one or more regulators of G-protein signaling in a biological sample obtained from the cancer patient are reduced relative to a control sample obtained from a healthy subject or a predetermined threshold. Additionally or alternatively, in some embodiments, the downstream inhibitor of RAS signaling pathway comprises one or more of BRAF inhibitors, MEK/ERK inhibitors, AURK inhibitors, and PI3K/Akt inhibitors.

Examples of BRAF inhibitors include, but are not limited to, GDC-0879, SB590885, Encorafenib, RAF265, TAK-632, PLX4720, CEP-32496, AZ628, Sorafenib Tosylate, Sorafenib, Vemurafenib (Zelboraf) and Dabrafenib (GSK2118436).

Examples of MEK/ERK inhibitors include, but are not limited to, MLN2480, Cobimetinib (GDC-0973), MEK 162, RO5126766, GDC-0623, VTX11e, Selumetinib (AZD6244), PD0325901, Trametinib (GSK1120212), U0126-EtOH, PD184352 (CI-1040), Refametinib, PD98059, BIX02189, Binimetinib, Pimasertib (AS-703026), SL327, BIX02188, AZD8330, TAK-733, PD318088, SCH772984, and FR 180204.

Examples of PI3K/Akt inhibitors include, but are not limited to, alpelisib, AMG319, apitolisib, AZD8186, BKM120, BGT226, bimiralisib, buparlisib, CH5132799, copanlisib, CUDC-907, dactolisisb, duvelisib, GDC-0941, GDC-0084, gedatolisib, GSK2292767, GSK2636771, idelalisib, IPI-549, leniolisib, LY294002, LY3023414, nemiralisib, omipalisib, PF-04691502, pictilisib, pilaralisib, PX866, RV-1729, SAR260301, SAR245408, serabelisib, SF1126, sonolisib, taselisib, umbralisib, voxtalisib, VS-5584, wortmannin, WX-037, ZSTK474, MK-2206, A-674563, A-443654, acetoxy-tirucallic acid, 3α- and 3β-acetoxy-tirucallic acids, afuresertib (GSK2110183), 4-amino-pyrido[2,3-d]pyrimidine derivative API-1, 3-aminopyrrolidine, anilinotriazole derivatives, ARQ751, ARQ 092, AT7867, AT13148, 7-azaindole, AZD5363, (−)-balanol derivatives, BAY 1125976, Boc-Phe-vinyl ketone, CCT128930, 3-chloroacetylindole, diethyl 6-methoxy-5,7-dihydroindolo [2,3-b]carbazole-2,10-dicarboxylate, diindolylmethane, 2,3-diphenylquinoxaline derivatives, DM-PIT-1, edelfosine, erucylphosphocholine, erufosine, frenolicin B, GSK-2141795, GSK690693, H-8, H-89, 4-hydroxynonenal, ilmofosine, imidazo-1,2-pyridine derivatives, indole-3-carbinol, ipatasertib, kalafungin, lactoquinomycin, medermycin, 3-methyl-xanthine, miltefosine, 1,6-naphthyridinone derivatives, NL-71-101, N-[(1-methyl-1H-pyrazol-4-yl)carbonyl]-N′-(3-bromophenyl)-thiourea, OSU-A9, perifosine, 3-oxo-tirucallic acid, PH-316, 3-phenyl-3H-imidazo[4,5-b]pyridine derivatives, 6-phenylpurine derivatives, PHT-427, PIT-1, PIT-2, 2-pyrimidyl-5-amidothiophene derivative, pyrrolo[2,3-d]pyrimidine derivatives, quinoline-4-carboxamide, 2-[4-(cyclohexa-1,3-dien-1-yl)-1H-pyrazol-3-yl]phenol, spiroindoline derivatives, triazolo[3,4-f][1,6]naphthyridin-3(2H)-one derivative, triciribine, triciribine mono-phosphate active analogue, and uprosertib.

Examples of AURK inhibitors include, but are not limited to, alisertib, tozasertib, SP-96, AT9283, danusertib (PHA-739358), AMG900, cenisertib, SNS-314, barasertib, hesperadin, AZD1152, GSK1070916, CYC116, BI 811283, AZD2811, PHA680632, reversine, CCT129202, CCT137690, quercetin, VX-680, PF-03814735, XL228, ENMD-2076, BI-847325, Ilorasertib/ABT-348, MK-5108/VX-689, and Chiauranib/CS-2164.

In any and all embodiments of the methods disclosed herein, the constitutively active KRAS mutation comprises a substitution at codon 12, 13 or 61 of KRAS. In certain embodiments, the constitutively active KRAS mutation is G12C, G12V, G12D, G12A, G12R, G12S, G12F, G12L, G13C, G13D, G13R, G13A, G13S, G13V, G13E, Q61H, Q61K, Q61L, Q61R, Q61P, or Q61E.

Additionally or alternatively, in some embodiments of the methods disclosed herein, the cancer patient suffers from a cancer selected from among pancreatic cancer, colon cancer, lung cancer, small intestine cancer, urinary tract cancer, endometrial cancer, cervical cancer, bladder cancer, liver cancer, myeloid leukemia breast cancer and biliary tract cancer. In some embodiments, the lung cancer is non-small-cell lung cancer (NSCLC) or small-cell lung cancer (SCLC).

In any and all embodiments of the methods disclosed herein, the one or more regulators of G-protein signaling comprises RGS3 and/or RGS4. In certain embodiments, RGS3 comprises p75 and/or p25 RGS3 isoforms. Additionally or alternatively, in some embodiments, the methods of the present technology comprise assaying mRNA or polypeptide expression levels encoded by a nucleic acid sequence selected from any of SEQ ID NOs: 3-20.

Additionally or alternatively, in some embodiments of the methods disclosed herein, mRNA expression levels are detected via next-generation sequencing, RNA-seq, real-time quantitative PCR (qPCR), digital PCR (dPCR), Reverse transcriptase-PCR (RT-PCR), Northern blotting, microarray, dot or slot blots, in situ hybridization, or fluorescent in situ hybridization (FISH). Additionally or alternatively, in certain embodiments of the methods disclosed herein, polypeptide expression levels are detected via Western blotting, enzyme-linked immunosorbent assays (ELISA), dot blotting, immunohistochemistry, immunofluorescence, immunoprecipitation, immunoelectrophoresis, or mass-spectrometry.

In any and all embodiments of the methods disclosed herein, the biological sample obtained from the cancer patient comprises biopsied tumor tissue, whole blood, plasma, or serum. Additionally or alternatively, in some embodiments of the methods disclosed herein, the patient is human.

Administration of any of the KRAS^{G12C} inhibitors or downstream inhibitors of RAS signaling pathway disclosed herein can be carried out by any suitable route, including but not limited to, orally, intranasally, parenterally (intravenously, intramuscularly, intraperitoneally, or subcutaneously), rectally, intrathecally, intratumorally or topically.

For therapeutic applications, a composition comprising a KRAS^{G12C} inhibitor or downstream inhibitor of RAS signaling pathway disclosed herein, is administered to the subject. In some embodiments, the KRAS^{G12C} inhibitor or downstream inhibitor of RAS signaling pathway is administered one, two, three, four, or five times per day. In some embodiments, the KRAS^{G12C} inhibitor or downstream inhibitor of RAS signaling pathway is administered more than five times per day. Additionally or alternatively, in some embodiments, the KRAS^{G12C} inhibitor or downstream inhibitor of RAS signaling pathway is administered every day, every other day, every third day, every fourth day, every fifth day, or every sixth day. In some embodiments, the KRAS^{G12C} inhibitor or downstream inhibitor of RAS signaling pathway is administered weekly, bi-weekly, tri-weekly, or monthly. In some embodiments, the KRAS^{G12C} inhibitor or downstream inhibitor of RAS signaling pathway is administered for a period of one, two, three, four, or five weeks. In some embodiments, the KRAS^{G12C} inhibitor or downstream inhibitor of RAS signaling pathway is administered for six weeks or more. In some embodiments, the KRAS^{G12C} inhibitor or downstream inhibitor of RAS signaling pathway is administered for twelve weeks or more. In some embodiments, the KRAS^{G12C} inhibitor or downstream inhibitor of RAS signaling pathway is administered for a period of less than one year. In some embodiments, the KRAS^{G12C} inhibitor or downstream inhibitor of RAS signaling pathway is administered for a period of more than one year. In some embodiments, the KRAS^{G12C} inhibitor or downstream inhibitor of RAS signaling pathway is administered throughout the subject's life.

In some embodiments of the methods of the present technology, the KRAS^{G12C} inhibitor or downstream inhibitor of RAS signaling pathway is administered daily for 1 week or more. In some embodiments of the methods of the present technology, the KRAS^{G12C} inhibitor or downstream inhibitor of RAS signaling pathway is administered daily for 2 weeks or more. In some embodiments of the methods of the present technology, the KRAS^{G12C} inhibitor or downstream inhibitor of RAS signaling pathway is administered daily for 3 weeks or more. In some embodiments of the methods of the present technology, the KRAS^{G12C} inhibitor or downstream inhibitor of RAS signaling pathway is administered daily for 4 weeks or more. In some embodiments of the methods of the present technology, the KRAS^{G12C} inhibitor or downstream inhibitor of RAS signaling pathway is administered daily for 6 weeks or more. In some embodiments of the methods of the present technology, the KRAS^{G12C} inhibitor or downstream inhibitor of RAS signaling pathway is administered daily for 12 weeks or more. In some embodiments, the KRAS^{G12C} inhibitor or downstream inhibitor of RAS signaling pathway is administered daily throughout the subject's life.

### Kits

The present disclosure also provides kits for determining whether a cancer patient harboring a constitutively active KRAS mutation will be responsive to treatment with a KRAS^{G12C} inhibitor that selectively targets the inactive state of KRAS. The kits of the present technology comprise one or more target-specific nucleic acid probes and/or primer pairs for detecting the expression levels or activity of regulators of G-protein signaling (RGS) in a biological sample and instructions for use. In certain embodiments, the constitutively active KRAS mutation is G12C, G12V, G12D, G12A, G12R, G12S, G12F, G12L, G13C, G13D, G13R, G13A, G13S, G13V, G13E, Q61H, Q61K, Q6IL, Q6IR, Q6IP, or Q61E. Additionally or alternatively, in some embodiments, the kits of the present technology further comprise one or more KRAS^{G12C} inhibitors or downstream inhibitors of RAS signaling pathway disclosed herein.

In some embodiments, the kit comprises liquid medium containing at least one target-specific nucleic acid probe or primers in a concentration of 250 nM or less. In some embodiments, the target-specific nucleic acid probes are detectably labeled.

In some embodiments, the kits further comprise buffers, enzymes having polymerase activity, enzyme cofactors such as magnesium or manganese, salts, chain extension nucleotides such as deoxynucleoside triphosphates (dNTPs) or biotinylated dNTPs, necessary to carry out an assay or reaction, such as amplification and/or detection of expression levels or activity of regulators of G-protein signaling (RGS) in a biological sample. The kit may also comprise instructions for use, software for automated analysis, containers, packages such as packaging intended for commercial sale and the like.

The kit may further comprise one or more of: wash buffers and/or reagents, hybridization buffers and/or reagents, labeling buffers and/or reagents, and detection means. The buffers and/or reagents are usually optimized for the particular amplification/detection technique for which the kit is intended. Protocols for using these buffers and reagents for performing different steps of the procedure may also be included in the kit.

The kits of the present technology may include components that are used to prepare nucleic acids from a test sample for the subsequent amplification and/or detection of expression levels or activity of regulators of G-protein signaling (RGS). Such sample preparation components can be used to produce nucleic acid extracts from any bodily fluids (such as blood, serum, plasma, etc.) or from tissue samples. The test samples used in the above-described methods will vary based on factors such as the assay format, nature of the detection method, and the specific tissues, cells or extracts used as the test sample to be assayed. Methods of extracting nucleic acids from samples are well known in the art and can be readily adapted to obtain a sample that is compatible with the system utilized. Automated sample preparation systems for extracting nucleic acids from a test sample are commercially available, e.g., Roche Molecular Systems' COBAS AmpliPrep System, Qiagen's BioRobot 9600, and Applied Biosystems' PRISM™ 6700 sample preparation system.

The kit additionally may comprise an assay definition scan card and/or instructions such as printed or electronic instructions for using the oligonucleotides in an assay. In some embodiments, a kit comprises an amplification reaction mixture or an amplification master mix. Reagents included in the kit may be contained in one or more containers, such as a vial.

Primers, probes, and/or primer-probes specific for amplification and detection of DNA internal control may be included in the amplification master mix as the target primer pairs to monitor potential PCR inhibition. Reagents necessary for amplification and detection of targets and internal control may be formulated as an all-in-one amplification master mix, which may be provided as single reaction aliquots in a kit.

### EXAMPLES

The present technology is further illustrated by the following Examples, which should not be construed as limiting in any way. The examples herein are provided to illustrate advantages of the present technology and to further assist a person of ordinary skill in the art with preparing or using the methods of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects or embodiments of the present technology.

### Example 1: Materials and Methods

### Cell Culture and Reagents

All cell lines used in this study were maintained in DMEM medium supplemented with 10% FBS, penicillin, streptomycin and 2 mM L-glutamine. Cell lines were obtained from ATCC (H358: CRL-5807; H2122: CRL-5985; H2030: CRL-5914; SW1573: CRL-2170), expanded immediately and frozen in aliquots. The cell lines tested negative for mycoplasma. All experiments were performed within 20 passages. MRTX1257 and MRTX849 were provided by Mirati Therapeutics and AMG510 was provided by Amgen. ARS1620 and ARS853 were purchased from MedChemExpress. Antibodies targeting RGS3 (sc-100762, 1:1,000) and GST (sc-138, 1:5000) were purchased from Santa Cruz Biotechnology. Antibodies targeting KRAS (WH0003845M1, 1:1,000), FLAG (F1804, 1:1,000) were obtained from Sigma. Antibodies targeting HA (C29F4 or 3724, 1:1000) or His (2365S, 1:1000) were obtained from Cell Signaling Technology. The BRAF specific antibody (sc-9002, 1:1,000) was purchased from Santa Cruz Biotechnology. The antibody dilution during immunoblotting is shown.

### Plasmids

For recombinant protein expression, DNA sequences encoding RGS3 p75 (Uniprot: P49796-1), RGS3 p25 (Uniprot: P49796-2), NF1 GAP related domain (1198-1530aa, Uniprot: P21359) and KRAS 4A (Uniprot: P01116-1) or 4B (Uniprot: P01116-2) were cloned into the pET-28a vector. In order to generate a GST-tagged KRAS, the gene was cloned into the pGEX-4T-1 vector. For mammalian expression, RGS3 was cloned into the pCDNA-3.1-c-FLAG vector, and KRAS was cloned into pCDNA-3.0-HA or pDEST27 (for GST-tagged KRAS). All indicated mutants were generated by site-directed mutagenesis. All constructs were verified by DNA sequencing.

### GTPase Assays

GTP[γ³²P] hydrolysis assay. KRAS proteins (0.5 μg) were reacted with 0.04 μM GTP[γ³²P] (6000 Ci/mmol; PerkinElmer Life Sciences) in 100 μL loading buffer (50 mM HEPES, pH 7.5, 50 mM NaCl, 1 mM DTT, 0.1 mM EGTA, 10 mM EDTA, and 1 mg/mL BSA) for 10 min at 30° C. (5, 40). Loading reactions were stopped by placing on ice and adding MgCl₂ to a final concentration of 20 mM. The GTPase assays were performed at 30° C. in 100 μL mixtures containing 20 nM of loaded KRAS and either hydrolysis buffer (25 mM HEPES pH 7.5, 50 mM NaCl, 1 mM DTT, 1 mM MgCl₂ and 1 mg/mL BSA) alone, or in combination with WCE or purified RGS3 (0.08-0.4 μg/μL). Equimolar comparisons of NF1, RASA1 and RGS3 were carried out using the continuous hydrolysis assay (see below). Reactions were stopped at the indicated time by filtering through 0.45-μm nitrocellulose membrane filters. The filters were washed 3 times with 0.2 mL of ice-cold hydrolysis buffer, air dried, and processed by autoradiography. Reaction rates were determined by fitting an exponential curve to the data, which was normalized to the baseline value. For the determination of protein stability, the hydrolysis reactions were stopped by boiling at 100° C. for 10 min in Laemmli buffer. The samples were then subjected to SDS-PAGE followed by Coomasie staining (CBB) or immunoblotting with a His-specific antibody.

GTP[α³²P] hydrolysis assay. KRAS proteins were loaded with GTP[α³²P] (3000 Ci/mmol; PerkinElmer Life Sciences) and subjected to the hydrolysis reaction as indicated above. Upon completion of the hydrolysis reaction and immobilization of KRAS-nucleotide complexes on nitrocellulose membranes, the nucleotides were eluted by incubating with elution buffer (50 mM HEPES pH 7.5, 50 mM NaCl, 1 mM DTT, 0.1 mM EGTA, 0.1% SDS, 10 mM EDTA, 1 mM GTP and 1 mM GDP) for 3 min at 85° C. The released nucleotides were resolved by thin layer chromatography (TLC) on PEI cellulose F paper (Merck) with 1 M LiCl and 1 M formic acid. The TLC papers were then dried and processed by autoradiography.

Continuous hydrolysis assay (two-step detection of inorganic phosphate). The EnzChek Phosphate Assay Kit (Thermo Fisher Scientific) was used to continuously measure phosphate release in vitro following the manufacturer's recommendation (20, 41). In brief, 100 μM KRAS protein were loaded with 2.5 mM GTP in assay buffer (10 mM EDTA, 30 mM Tris pH 7.5 and 1 mM DTT) at room temperature for 2 h. Loaded KRAS was then desalted by gel filtration in Zeba™ spin columns (Thermo Scientific). The hydrolysis reaction was preformed in 384-well microplates (Costar) containing GTP-loaded KRAS (50 μM), MESG (200 μM), PNP (5 U/mL), and MgCl₂ (40 mM) in reaction buffer (30 mM Tris pH 7.5.1 mM DTT). GAPs were added at a concentration of 50 μM or as indicated. The reaction was monitored by reading the absorbance at 360 nm every 10 to 20 s for ˜16-60 min at room temperature. The data was imported to Prism and normalized by the embedded min-max normalization strategy. Kinetic constants were obtained by fitting an exponential curve. In intrinsic hydrolysis reactions, where the maximum PO₄ release was less than observed in the paired GAP-assisted reaction (i.e., positive control), the maximum value during normalization was set as the maximum value from the paired GAP-assisted reaction.

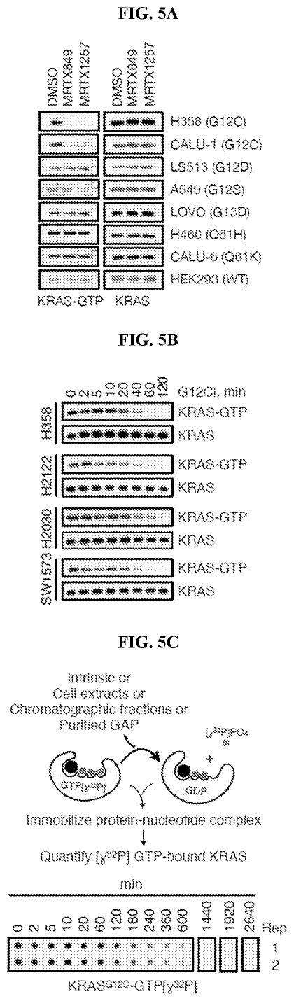

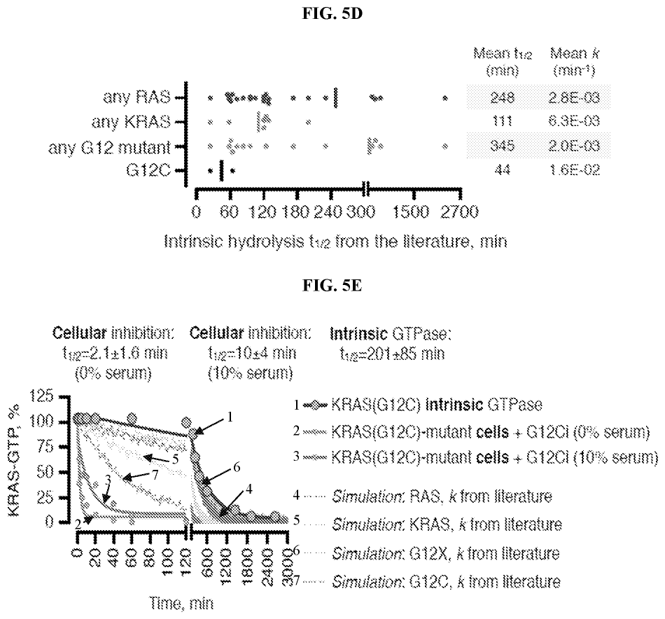

GTPase assay choice. Two-step phosphate labeling reagents are inaccurate for slow reactions. The reagent is not stable over long incubation periods, which are necessary to achieve complete hydrolysis (during intrinsic reactions) and to accurately determine the half-life. Moreover, two-step detection systems are affected by the presence of inorganic phosphate in buffers (or cellular extracts) and cannot be used to isolate cellular factors that enhance the hydrolysis rate. In order to calculate the intrinsic hydrolysis rate constant single-turnover [³²P]GTP hydrolysis assays were relied on and sufficient time was allowed for the reaction to reach a steady state maximum (i.e., undetectable levels in FIG. 5C). Validation studies were carried out using [³²P]GTP (at the α or γ position) and two-step phosphate labeling reagents. The latter were also used to determine the kinetics of the GAP-assisted reactions, since these are considerably faster than intrinsic hydrolysis.

### RAS-Binding Domain Pull-Down

This was performed as described previously (42) by using the RAS activation kit (Thermo Scientific). Briefly, 50 μg whole cell lysates were incubated with 40 μg GST-RBD and 100 μL glutathione beads for 60 min followed by three washes in NP40 wash buffer and elution of pull-down fractions with 2×SDS PAGE loading dye. The samples were then subjected to western blotting with a KRAS specific (WH0003845M1) antibody (43).

### Chromatographic Separation and Identification of KRAS^{G12C}-Directed GAP Activity.

H358 cells were chosen as a starting point during chromatography (and as the main model system for validating studies) because these are the most sensitive to G12Ci treatment. It was anticipated that extracts from these cells would be most likely to yield a successful identification of a KRAS^{G12C}-directed GAP.

The chromatographic separation and identification process comprised of two rounds of size-exclusion chromatography (SEC, steps 1 and 2), desalting (step 3), anion exchange chromatography (AEC, step 4) and mass spectrometry (step 5). All procedures were carried out at 4° C. by using the ÄKTA system.

H358 cells growing exponentially (2 g) were collected and resuspended in 5 mL hydrolysis buffer (25 mM HEPES, pH 7.5, 50 mM NaCl, 1 mM DTT, 1 mM MgCl₂). The cells were broken down by sonication and centrifuged at 13,200 rpm for 1 h to obtain the supernatant. The supernatant was passed through a 0.45 μm filter (Millipore), centrifuged at 13,200 rpm for 60 min and loaded onto a HiLoad Superdex 200PG column (GE healthcare) for the first round of SEC (step 1). The fractions (1 mL) eluted from this column were collected and a portion (5 μL) was used in a GTP[γ³²P] hydrolysis assay as described above.

Step 1 fractions that enhanced KRAS^{G12C} hydrolysis were pooled and loaded onto Superdex 200 (GE healthcare) for the second round of SEC (step 2). Again, eluted fractions (1 mL) were collected and a portion (10 μL) was subjected to the GTP[γ³²P] hydrolysis assay.

Step 2 fractions that enhanced KRAS^{G12C} hydrolysis were pooled and their buffer exchanged into one containing HEPES 20 mM pH 7.5, 20 mM NaCl, DTT 1 mM (step 3) using a HiTrap desalting column (GE healthcare, 5 mL). Desalted fractions were then loaded (step 4) onto a DEAE column (GE healthcare) and washed with a buffer containing HEPES 20 mM pH 7.5, NaCl 0.5M, DTT 1 mM using a 0-100% gradient over 5 column volumes (each 25 mL). Eluded step 4 fractions (1 mL) were collected. In order to determine their GTPase enhancing effect, 30% of each fraction was concentrated to 15 μL using a using 3 kDa molecular weight cut-off centrifugal filter and subjected to the KRAS^{G12C} GTP[γ³²P] hydrolysis assay.

AEC fractions that enhanced KRAS^{G12C} hydrolysis were evaluated by mass spectrometry (step 5) to identify proteins annotated as GAPs. Proteins from active AEC fractions were precipitated with equal volume of 20% TCA/acetone for 12 h at 4° C. The supernatant was removed by centrifugation at 13,200 rpm for 10 min at 4° C., and the pellet was washed three times with cold acetone. The protein precipitates were air-dried and submitted for trypsin digestion and mass spectrometry identification at a fee-for-service core facility. The peptides were analyzed by liquid chromatography-tandem mass spectrometry on a Q Exactive mass spectrometer (Thermo Fisher Scientific). Proteins were identified by a database search of the fragment spectra against the UniProt protein database. Only proteins previously annotated as GAPs and that were identified in all fractions/peaks with GTPase-enhancing activity were evaluated.

### KRAS^{G12C}-RGS3 Interaction

WCE (2 mg) from treated or untreated KRAS^{G12C} mutant cells were subjected to IP with either a KRAS (sc-30) or an IgG antibody agarose-conjugate followed by immunoblotting with KRAS- or RGS3-specific monoclonal antibodies. A BRAF-specific antibody were used as a control.

To determine the presence of a direct interaction purified GST-tagged RGS3 GAP domain (RGSD) and His-tagged KRAS^{G12C} were used. The latter was loaded with GTPγS (a non-hydrolyzable GTP analogue) or GDP. The proteins (0.2 μM KRAS and 1.2 μM RGSD) were mixed in binding buffer (25 mM Tris-HCl pH=7.5, 150 mM NaCl, 1% NP40, 5% glycerol and 5 mM MgCl₂) and incubated at 4ºC for 1 h. The reaction was then subject to a GST-pulldown.

### GST Pull-Down Assays

HEK293H cells (2.5×10³) were seeded in 60 mm cell culture dishes and ˜16 h later the cells were transfected with 2 μg of GST-tagged constructs and 2 μg of FLAG-tagged constructs by using Lipofectamine 2000 (Thermo Scientific) at a ratio of 1 μg: 2 μL. 24 h post-transfection, the cells were washed with 3 mL ice-cold PBS, collected and immediately centrifuged at 2000 rpm for 3 min. The cell pellets lysed in 300 μL of NP40 lysis buffer (50 mM Tris-HCl pH=7.5, 150 mM NaCl, 1% NP40, 10% glycerol and 1 mM EDTA) supplemented with protease and phosphatase inhibitors and mixed vigorously. After incubating on ice for 10 min, the samples were centrifuged at 13,200 rpm at 4° C. for 10 min. The supernatants (1 mg) were incubated and rotated with 100 μL of glutathione-sepharose beads for 60 min at 4° C. and washed three times with wash buffer (25 mM Tris-HCl PH=7.5, 150 mM NaCl, 1% NP40, 5% glycerol, 5 mM MgCl₂). GST pull-downs were resuspended in 2× loading buffer for SDS/PAGE analysis.

### Recombinant Protein Production

The cDNAs encoding for His-tagged KRAS, RGS3 and NF1 GRD were cloned into the pET28a expression vector and expressed in E. Coli BL21 (DE3) cells. Protein expression was induced by adding 0.5 mM isopropyl β-D-thiogalactopyranoside (IPTG) in Terrific Broth medium at 16° C. for 18 h. Bacterial cells were harvested and then resuspended with lysis buffer (20 mM HEPES, pH 7.5, 500 mM NaCl, 5% glycerol, 1 mM DTT, and 20 mM imidazole). The cell debris was removed by centrifugation at 16,000 rpm for 60 min at 4° C., and the soluble fraction was loaded onto nickel-sepharose (GE Healthcare) pre-equilibrated with lysis buffer. After sequential washes with lysis buffer containing 20 mM and then 40 mM imidazole, the proteins were eluted with lysis buffer containing 400 mM imidazole. The target proteins were further purified by size-exclusion chromatography in a Superdex 200 column (GE Healthcare) in buffer containing 20 mM HEPES, pH 7.5, 150 mM NaCl, and 1 mM DTT.

GST-tagged KRAS or RGS3 GAP domain were cloned in the pGEX-4T-1 expression vector and expressed in E. Coli BL21(DE3) cells. Protein expression was induced by adding 0.5 mM IPTG as above. Bacterial cells were harvested and then resuspended with phosphate-buffered saline (PBS) before being lysed. The cell debris was removed by centrifugation at 16,000 rpm for 60 min at 4° C., and the soluble fraction was loaded onto glutathione-sepharose (GE Healthcare) pre-equilibrated with PBS. After washing with PBS, proteins were eluted with 10 mM glutathione, 50 mM Tris-HCl, pH 8.0. The target proteins were further purified by size-exclusion chromatography as above.

### Molecular Modeling

Protein-protein docking was carried out in a heuristic manner using the tools in Maestro/Bioluminate (Schrodinger), as described by the manufacturer. Briefly, the model was based on several existing structures, including KRAS-GMPPNP (60B2) and RGS3 GAP domain (2OJ4). Superimposition of the structure of KRAS to that of Gial revealed a similar architecture of the key motifs responsible for GTP-hydrolysis (i.e., the P-loop, switch I and switch II regions show a high degree of overlap between the two proteins). Based on this observation, the structure of Gial-GDP·AlF4:RGS4 (1AGR) was used to define several anchoring points and refine the docking poses for KRAS-RGS3. Known contacts between RGS4 and Gial were used as docking constraints. Prior to protein-protein docking, the structure of KRAS-GMPPNP and RGS3 GAP domain were prepared by adding hydrogens, assigning bond orders, creating disulfide bonds, adding missing side chains, deleting waters beyond 5 Å, assigning H-bonds and restraining minimization. These were carried out using the protein-preparation wizard in Maestro and default settings. The poses generated were evaluated by the density of similar poses and priority was given to poses satisfying the constraints noted above. During the evaluation of initial docking poses the focus was to identify residues that could potentially mediate the GAP activity of RGS3 on KRAS. With this in mind, initial poses were evaluated manually to identify those where a residue in RGS3 would approach the catalytic core of KRAS in a manner that could enhance hydrolysis and not be impeded by G12 substitutions. This initial approach suggested a potential role for an asparagine residue in RGS3 GAP domain. Once the significance of this residue was confirmed experimentally the docking was repeated with more stringent constraints.

### RGS3 Knockout

H358 cells were transiently transfected with a plasmid (pSpCas9-2A) encoding RGS3-specific sgRNAs. Two different sgRNA were used: sgRGS3 #1: TGCGTGATCCTCTTCCTGGC (SEQ ID NO: 1); sgRGS3 #2: CTACACGCGGGAGCACACCA (SEQ ID NO: 2). After 48 h, GFP-positive cells were FACS-sorted into 96 well plates, with a single cell per well. Each cell was allowed to expand into a clone, these were then expanded and screened for RGS3 deletion by Sanger sequencing and immunobloting using a monoclonal antibody detecting RGS3.

### Live-Cell Analysis

Control or RGS3-null H358 cells were seeded at 10-20% confluence in a 24 well-plate and transfected with 5 pmol of non-targeting (NT) or KRAS^{G12C}-specific siRNA (siG12C) (18). H358 cells expressing G12C or G12C/A59G mutant KRAS were seeded at 10-20% confluence and transfected with 5 pmol of non-targeting (NT) or RGS3-specific siRNA (Horizon, L-008302-01-0010). Images of cell confluence were automatically acquired within Incucyte (Essen BioScience). The images were taken at 2 h intervals for the duration of the experiment. The % confluence was determined by using the Incucyte software (Essen BioScience).

### 3D Tumor Spheroid Assays

Cells were added at a density of 4×10³ cells/mL in a solution containing collagen I (A1048301, Gibco) at a final concentration of 2 mg/mL. The collagen-cell mixture (300 μL) was then added to collagen I pre-coated 35 mm dishes. The mixture was incubated for 30 min at 37° C., followed by addition of 2 mL culture medium and incubation for the indicated days in a 37° C., 5% CO₂ incubator. Spheroids were imaged using an Inverted Research Microscope Ti2 (Nikon) and the data were analyzed using NIS-Elements.

### Immunohistochemistry Analysis

The tissue sections from paraffin-embedded PDX tumors were stained with RGS3 antibody. The tissue sections according to the percentage of positive cells and staining intensity were quantitatively scored. IHC images were quantified in an automated manner by using the QuPath software (University of Edinburgh, Division of Pathology) (44). The score (H-score) was obtained using the formula: X3+2×X2+3×X1 giving a range of 0 to 300 (0≤[X1+X2+X3]≤100), where X3 indicates weak staining, X2 moderate staining and X1 strong staining. The H-score was correlated with pattern of G12Ci-response.

Correlation of RGS3 Expression with KRAS Mutant Transcriptional Output

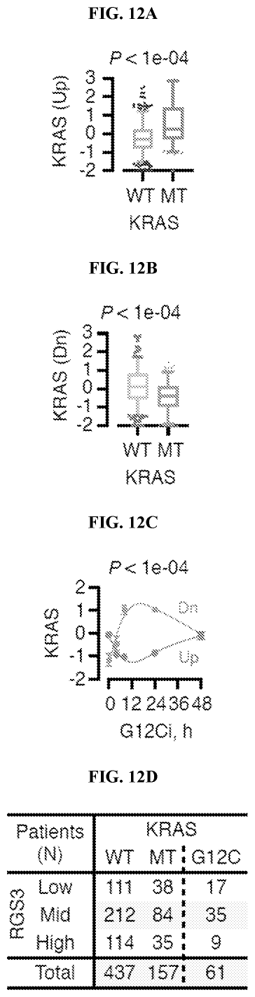

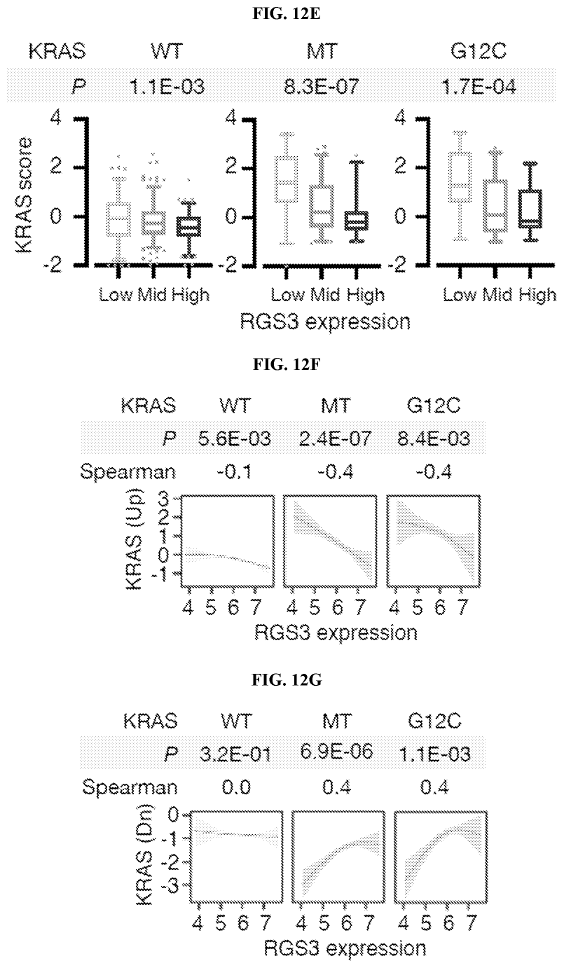

The lung adenocarcinoma TCGA RNAseq dataset was obtained from the Genomic Data Commons Data Portal as HT-Seq counts. Count data were filtered and normalized using edgeR and standard approaches. Differentially expressed genes between KRAS mutant vs. wild-type tumors were considered those with an absolute log fold change of greater than 1 and an FDR of less than 0.05. The mutant KRAS output score was defined as the mean log-transformed normalized (count per million) expression of up- or down-regulated genes. The ability of this score to determine changes in KRAS signaling was experimentally validated in H358 cells treated with a G12C inhibitor over time (0-48 h, see FIG. 12C). The trend in KRAS score was proportional to the trend in KRAS-GTP and pERK inhibition (data not shown).

The correlation between mutant KRAS output score and RGS3 expression was determined by two approaches. In the first approach, RGS3 expression in log counts per million was categorized as low (<0.25 percentile), intermediate (0.25-0.75 percentile) and high (>0.75 percentile) across the entire dataset. The distribution of KRAS-up scores along these categories, both in KRAS wild-type and in KRAS mutant lung cancers, were compared using ANOVA and Turkey tests, while correcting for multiple comparison testing. In the second approach, the correlation between RGS3 expression (log count per million, continuous variable) and mutant KRAS output score (up or down) were determined by using the Spearman coefficient.

### Animal Studies

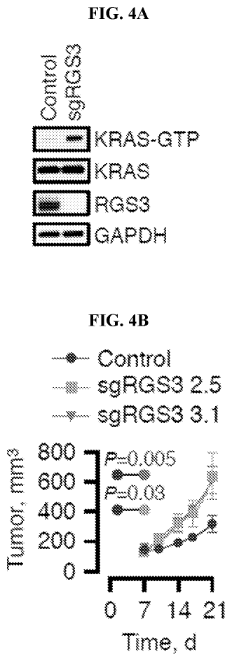

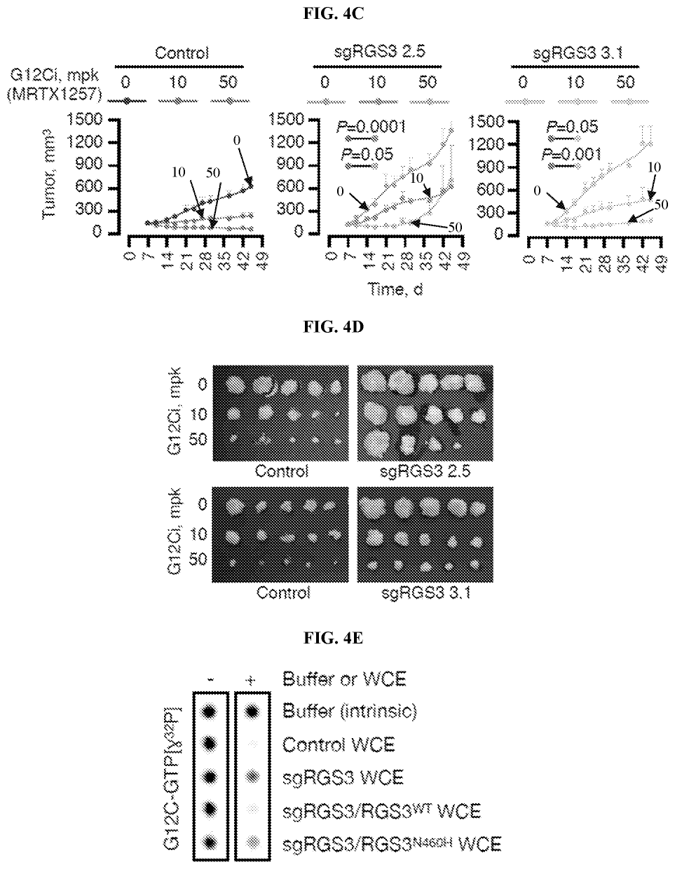

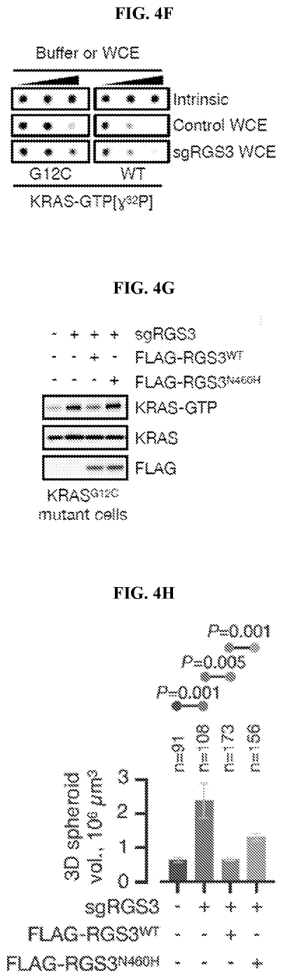

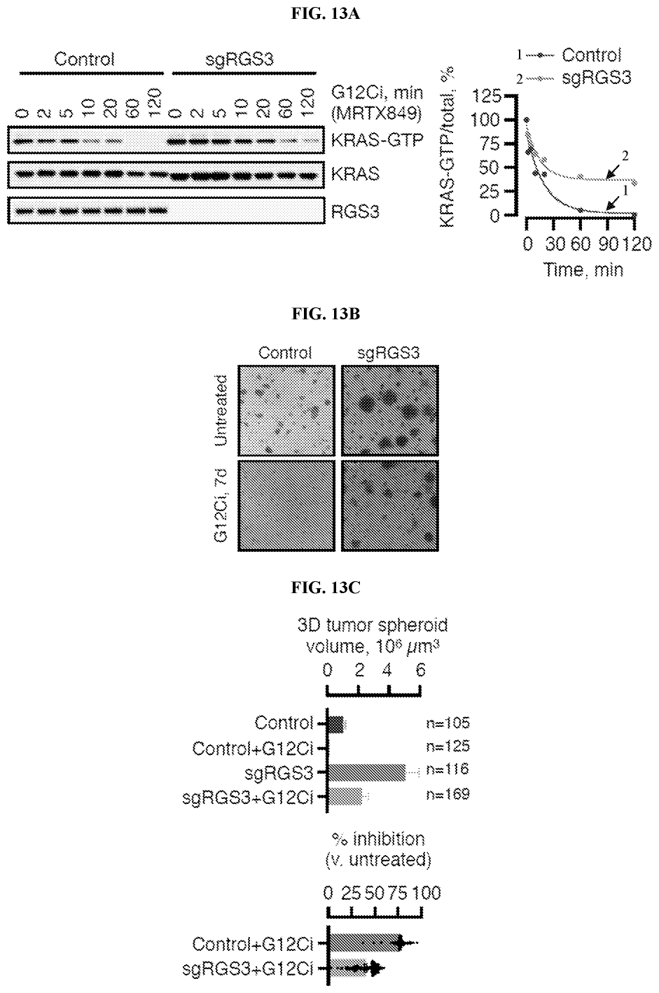

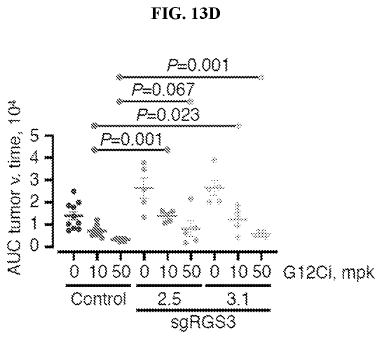

This was carried out as described previously (45). Briefly, nu/nu athymic mice were obtained from the Envigo Laboratories and maintained in compliance with IACUC guidelines under protocol 18-05-007 approved by MSKCC IACUC. The maximum tumor measurement permitted was 1.5 cm and this was not exceeded in any of the experiments disclosed herein. Animals implanted with xenografts were chosen for efficacy studies in an unbiased manner. Once tumors reached 100 mm³ volume, mice were randomized and treated with drug or the appropriate vehicle control. Treatments and tumor measurements were performed in a non-blinded manner by a research technician who was not aware of the objectives of the study. Prism (GraphPad Software Inc.) was used for data analysis. For each study arm, the tumor size was plotted over time. Statistically significant differences were determined either for endpoint tumor volumes (FIG. 4C) or for the area under the curve (AUC, FIG. 4B and FIG. 13D) by the two-tailed t-test function embedded in Prism.

### Example 2: RGS Enhances the GTPase Activity of G12/G13 KRAS Mutants

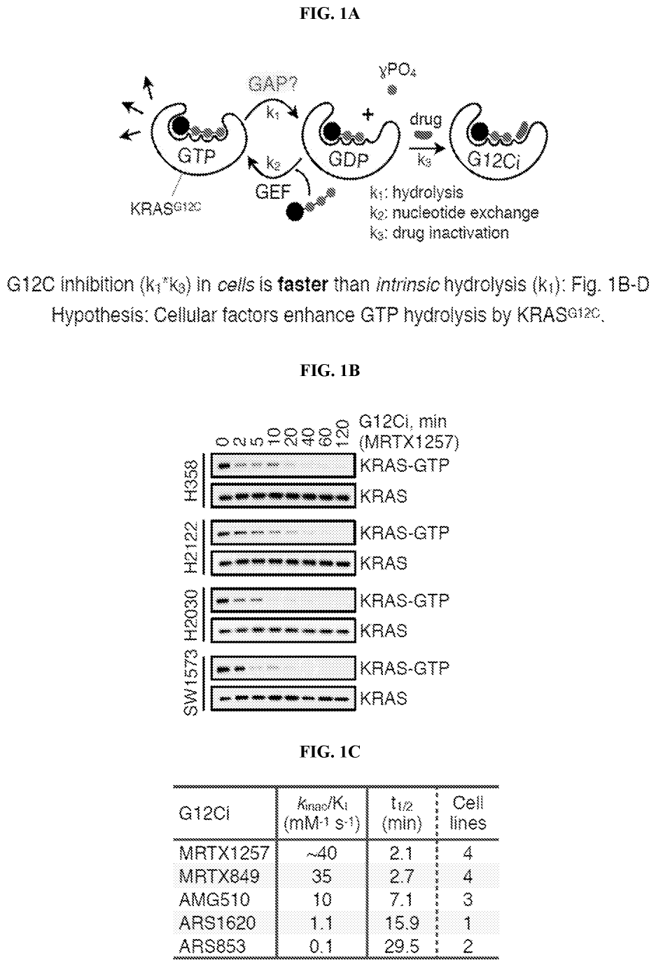

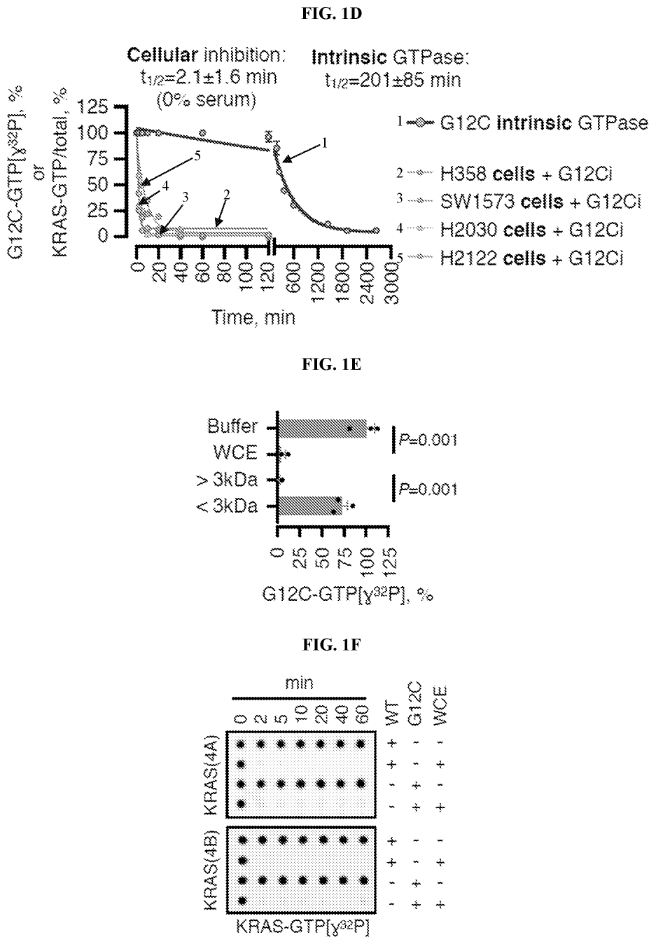

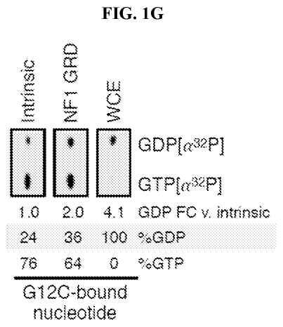

Intrinsic hydrolysis alone may be sufficient to enable inactive state-selective inhibition. To this end, the rate of inhibition in cancer cells was compared to the rate of intrinsic GTP hydrolysis by KRAS^{G12C}. The time required for maximal inhibition of KRAS in cells treated with a potent and selective G12Ci (i.e., MRTX1257, FIG. 5A) ranged from ˜20 min in serum-deprived media, a condition that suppresses nucleotide exchange (half-life ˜2 min, FIG. 1B), to ˜60 min in media with 10% serum (half-life ˜10 min, FIG. 5B). Similar cellular inhibition kinetics were observed with other potent inhibitors (k_{inac}/K₁≥10 mM⁻¹ s⁻¹), such as MRTX849 and AMG510 (FIG. 1C).

By comparison, purified KRAS^{G12C} hydrolyzed GTP[γ³²P] at a much slower rate (half-life 120-300 min, FIG. 1D and FIG. 5C). The intrinsic hydrolysis half-life for KRAS proteins varies across publications (FIG. 5D, range: 25-200 min). Regardless of this variation, the rate of cellular KRAS^{G12C} inhibition with potent inhibitors was quicker than even the fastest intrinsic hydrolysis estimate (20) reported in the literature (FIG. 5E).

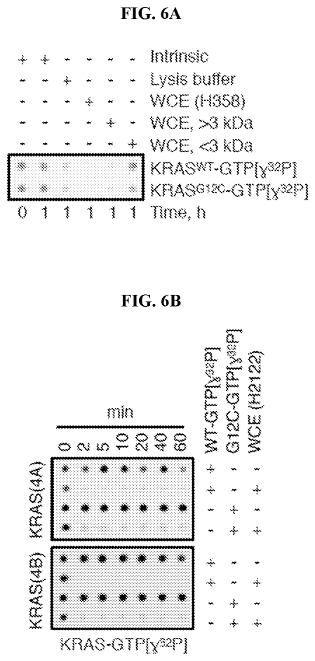

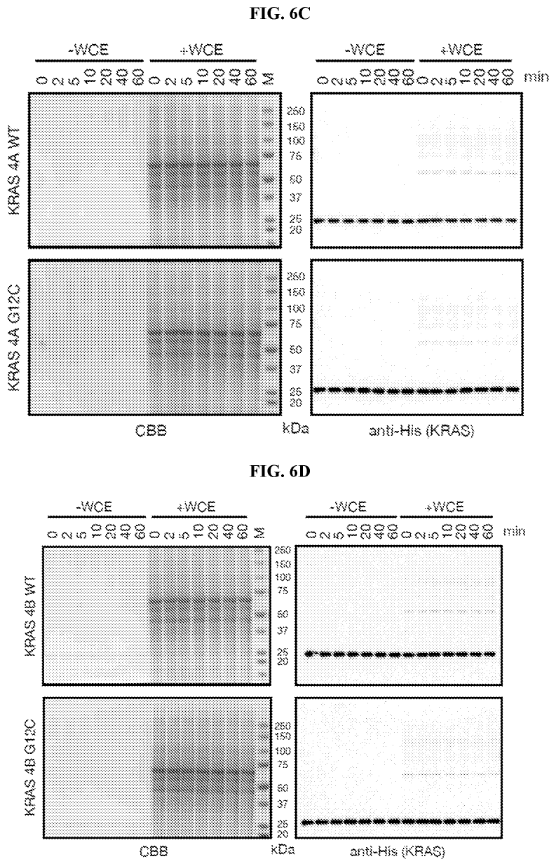

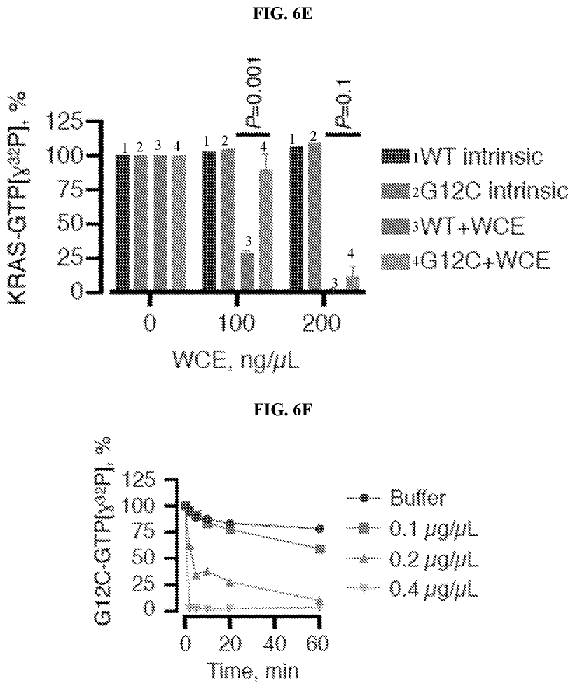

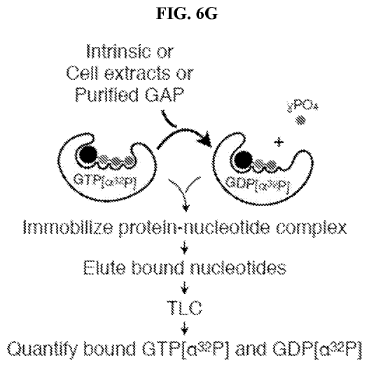

The discrepancy between the rate of cellular inactivation and the rate of the intrinsic GTPase reaction suggests the presence of cellular factors that enhance GTP-hydrolysis by KRAS^{G12C}. To test this possibility, purified KRAS was loaded with GTP[γ³²P] and incubated with whole-cell extracts (WCE) from lung cancer models. Because cytoplasmic RAS-GAPs, such as NF1, enhance the GTPase activity of wild-type (WT) KRAS, the latter was used as a positive control. The KRAS-nucleotide complex was immobilized and the remaining GTP[γ³²P] was quantified by autoradiography (hereafter referred to as the γ-phosphate assay, FIG. 5C). As shown in FIG. 1E and FIG. 6A, little intrinsic GTP[γ³²P]-hydrolysis was detected for KRAS^{G12C} at 1 h of incubation. In the presence of cellular extracts, however, there was a near complete hydrolysis of GTP[γ³²P] (FIG. 1E and FIG. 6A). Under these conditions, the reaction reached a steady-state maximum within 2 min of incubation (FIG. 1F and FIG. 6B). Cellular extracts did not affect KRAS protein stability in these experiments (FIG. 6C-6D). The activity was greatly reduced in the flow-through from a 3 kDa molecular cut-off fractionation column (FIG. 1E and FIG. 6A), suggesting that a cellular protein is responsible.

It's been reported that cellular extracts enhance hydrolysis by wild-type but not by mutant (e.g., G12V/D) NRAS (5). If the same holds true for KRAS^{G12C} has not been tested in the literature. In agreement with previous work, these results show that the GTPase activity of KRAS^{WT} was enhanced in a concentration dependent manner and that the effect of on KRAS^{WT} was more potent than KRAS^{G12C} (FIG. 6E). However, rather than being completely insensitive to cellular extracts, KRAS^{G12C} was also responsive, with activity detected at higher lysate concentrations (FIGS. 6E-6F).

It is possible that the KRAS^{G12C} effect reflects an enhanced rate of nucleotide-dissociation or the exchange of GTP for GDP. To address these possibilities, the GTPase activity of KRAS^{G12C} was assayed by loading it with GTP[α³²P] and subsequent incubation with cellular extracts (hereafter referred to as the α-phosphate assay, FIG. 6G). Following immobilization of KRAS, the bound nucleotides were eluted and subjected to separation by thin-layer chromatography (TLC). After incubation with the GAP-related domain (GRD) of NF1, the predominant [α³²P]-labeled nucleotide bound to KRAS^{G12C} was GTP (FIG. 1G). By comparison, the predominant [α³²P]-labeled nucleotide bound to KRAS^{G12C} incubated with cell extracts was GDP. If nucleotide exchange (or nucleotide dissociation) was responsible, then GTP[α³²P] would be replaced with cold nucleotide and no GDP[α³²P] signal would be detected. Because an increase in GDP[α³²P] was indeed detected (FIG. 1G), the data support the presence of a cellular protein that accelerates GTP-hydrolysis by KRAS^{G12C}.

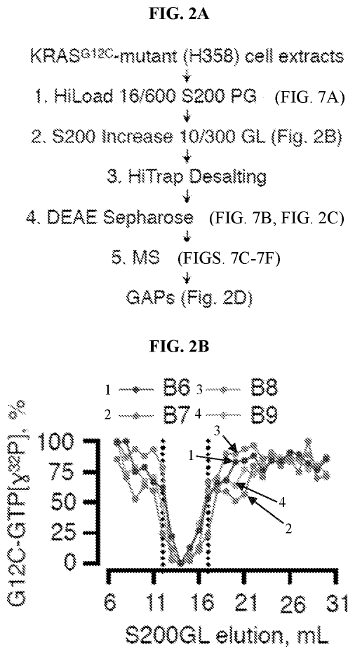

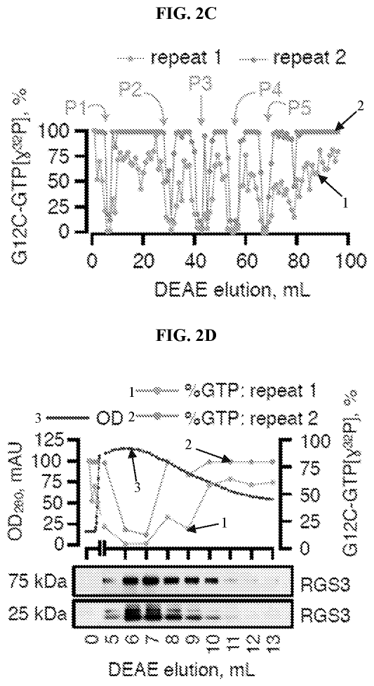

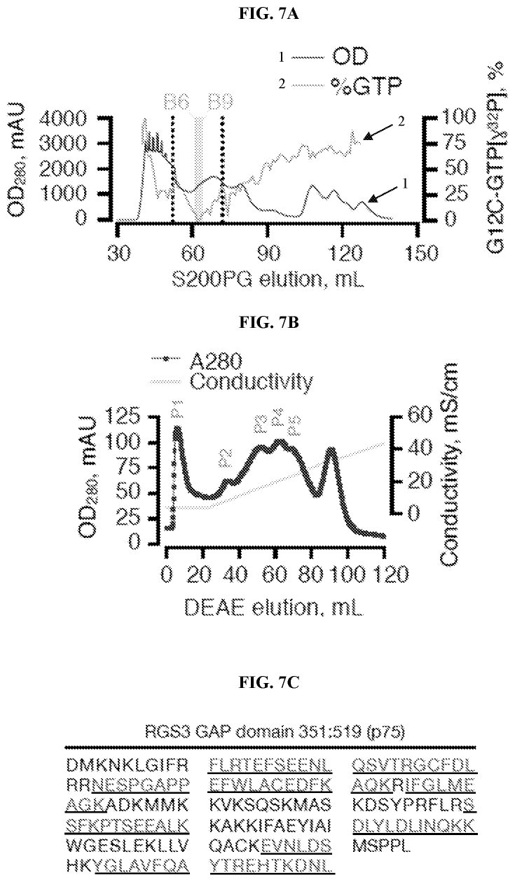

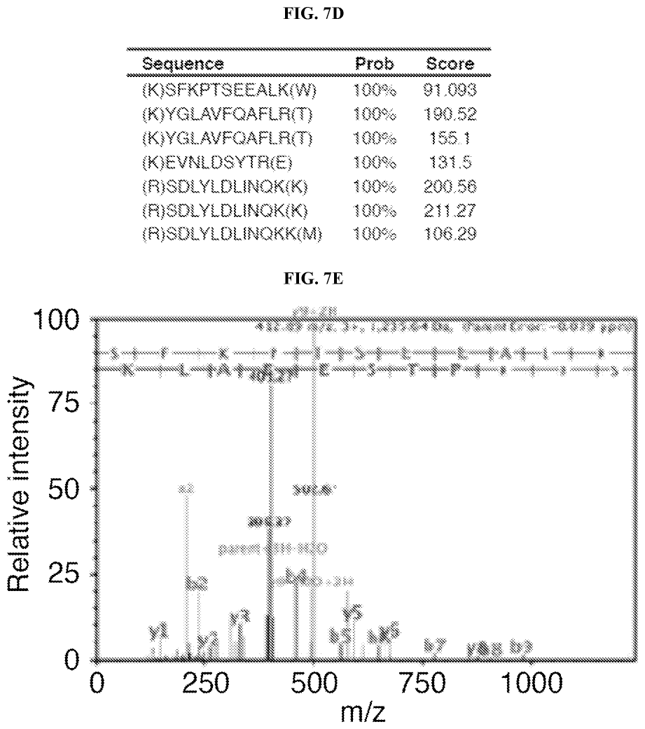

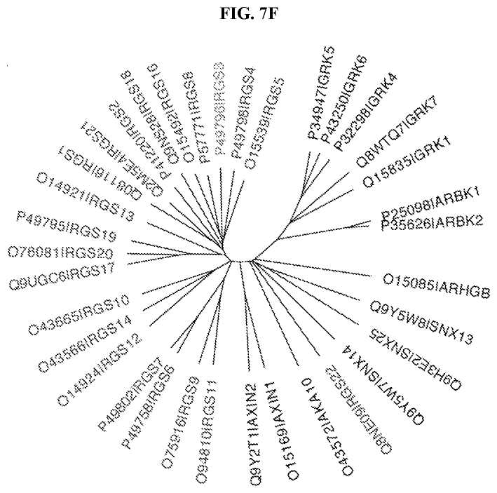

In order to isolate this protein in an unbiased manner, a purification scheme was devised wherein KRAS^{G12C}-mutant cancer cell extracts (see Materials and Methods) were subjected to two rounds of size-exclusion chromatography (SEC, steps 1 and 2), desalting (step 3), anion-exchange chromatography (AEC, step 4) and mass spectrometry (MS, step 5) to identify proteins classified as GAPs (FIG. 2A). The eluted fractions from each chromatographic step were assayed for their optical density and their ability to enhance KRAS^{G12C} hydrolysis. Fractions that retained activity were pooled and carried forward to the next step. SEC step 1 elution volumes ranging from 52 to 72 mL had KRAS^{G12C}-directed GAP activity (FIG. 7A). Four of these were independently subjected to SEC step 2, in order to confirm the activity and to identify which step 2 fractions to carry forward (FIG. 2B). SEC step 2 elution volumes ranging from 12 to 15 mL were pooled, desalted and analyzed by AEC with a linear salt-gradient (FIG. 7B). The latter identified five peaks with KRAS^{G12C}-directed GAP activity (FIG. 2C).

The active fractions eluted in step 4 were pooled, acid-precipitated and subjected to MS in order to identify proteins annotated as GAP. One of these, RGS3, is a GAP for the Gα_{i/q} subunit of heterotrimeric G-protein coupled receptors (21-23). Many of the RGS3 peptides identified by mass spectrometry localized in the RGS domain of the protein (RGSD, i.e. the GAP domain, FIGS. 7C-7E). RGS3 lacks the catalytic arginine (R)-finger present in canonical RAS-GAPs (24, 25). The hypothesis that RGS3 might not be impeded by G12 mutations was further analyzed. The broader RGS family contains 20 members (FIG. 7F), which often have alternatively-spliced variants. At least 9 variants of RGS3 have been reported. The 75 kDa (p75) and/or the 25 kDa (p25) RGS3 isoforms (both of which contain the RGS GAP domain) eluted in one or more fractions that enhanced GTP[γ³²P]-hydrolysis by KRAS^{G12C} (FIG. 2D).

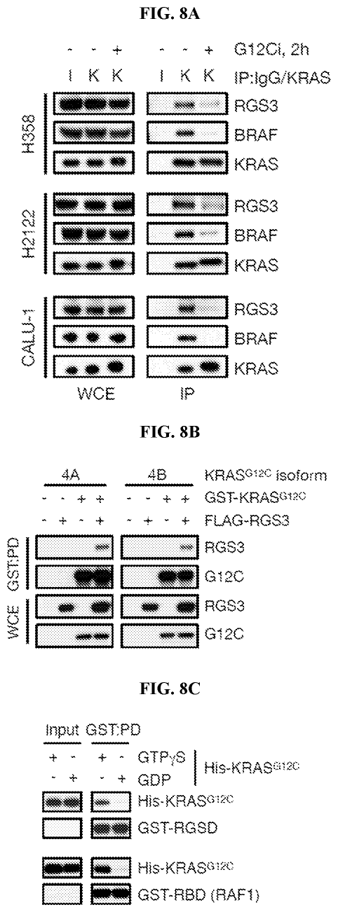

In beginning to validate the effect of RGS3, the interaction between RGS3 and KRAS^{G12C} in cells was evaluated. To this end, the ability of the G12Ci to selectively displace effector proteins from KRAS^{G12C} was relied on, while sparing those bound to wild-type KRAS, NRAS and HRAS (13, 14). Co-immunoprecipitation (IP) studies in three KRAS^{G12C}-mutant cell lines showed an interaction between endogenous KRAS^{G12C} and RGS3; an interaction that was diminished upon G12Ci-treatment (FIG. 8A). GST-pulldown experiments confirmed that both KRAS^{G12C} 4A and 4B splice variants interact with RGS3 (FIG. 8B). Direct binding assays with purified proteins suggested a preference for the active (or GTPγS-loaded) conformation of KRAS^{G12C}, as compared to the inactive (GDP-loaded) state (FIG. 8C).

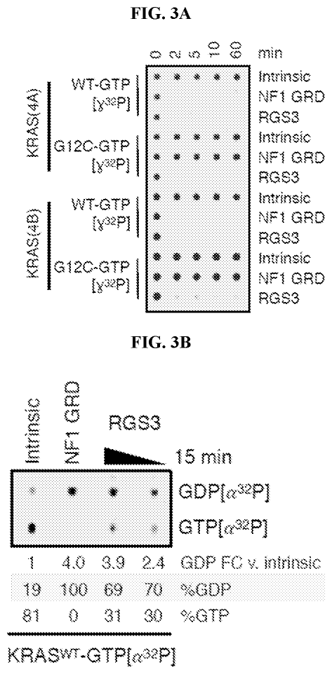

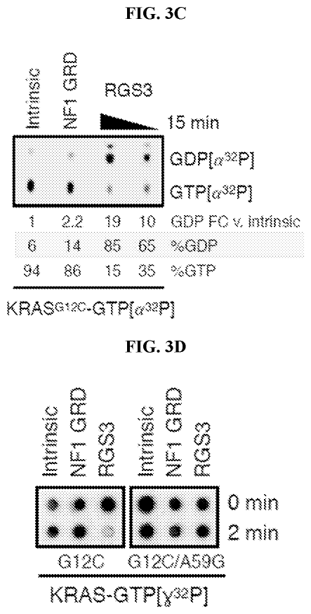

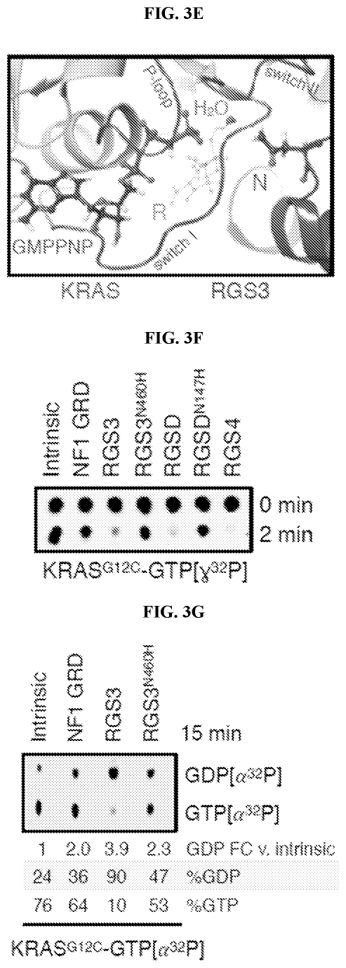

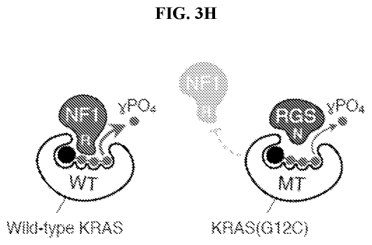

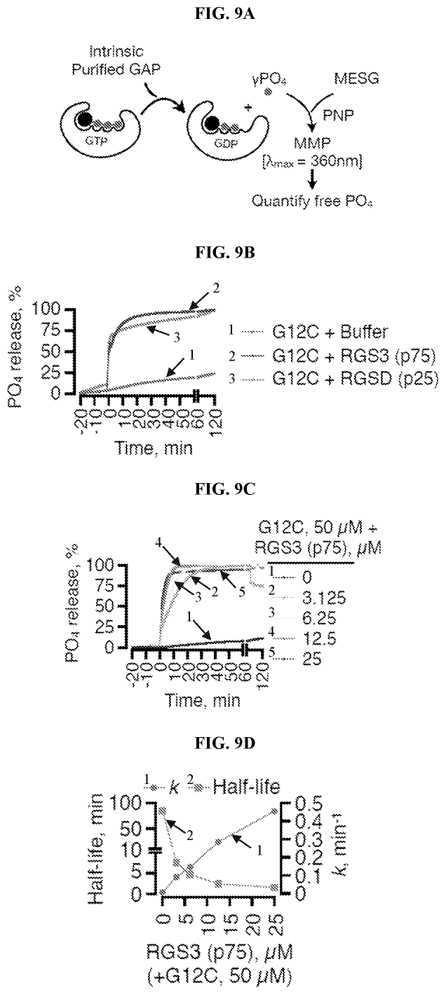

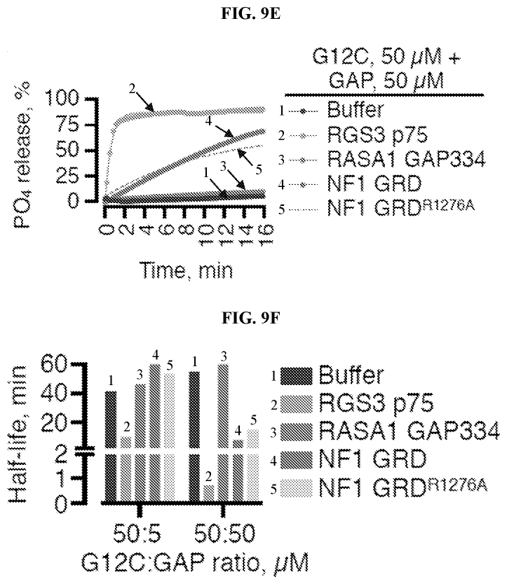

To directly test if RGS3 acts as a GAP for KRAS^{G12C}, GTP-hydrolysis assays were carried out with purified KRAS in the presence of RGS3 variants. In addition to the radioactive γ- and α-phosphate assays (FIGS. 5C and 6G), a non-radioactive hydrolysis assay that enables continuous measurement of phosphate release over time was also used (FIG. 9A). RGS3 enhanced GTP-hydrolysis by KRAS^{G12C} (FIGS. 3A-3C and FIG. 9B), leading to a concentration-dependent increase in the rate constant (FIGS. 9C-9D). RGS3 was more effective at enhancing KRAS^{G12C} hydrolysis than canonical RAS-GAPs, such as NF1 and RASA1 (FIGS. 9E-9F). Whereas no measurable effect was detected for RASA1, some activity was observed for NF1 (FIGS. 3A-3C and FIGS. 9E-9F). The latter, however, was not significantly attenuated when the catalytic arginine of NF1 was mutated to alanine (R1276A), supporting the theory that KRAS^{G12C}-directed GAP activity occurs independently of the R-finger.

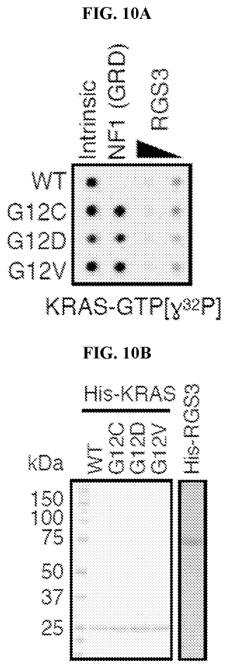

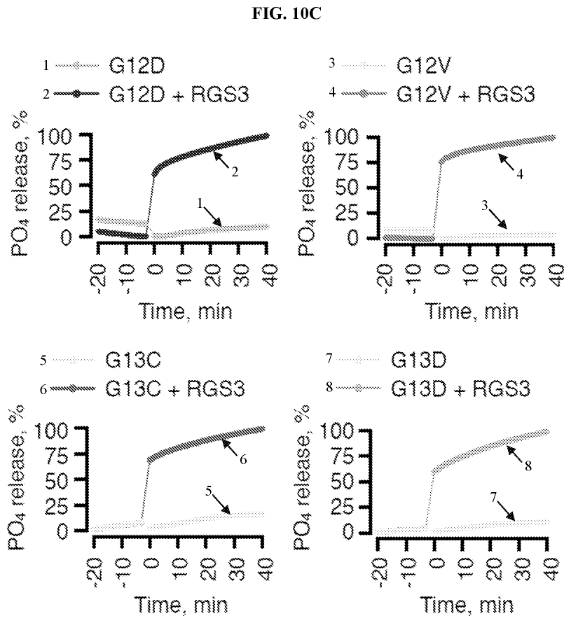

Rather than having an idiosyncratic effect on KRAS^{G12C}, RGS3 also enhanced GTP-hydrolysis by G12D/V and G13C/D mutant KRAS (FIGS. 10A-10C), which together comprise ˜95% of KRAS mutations found in cancer. RGS3 also enhanced the GTPase activity of KRAS^{WT}, but in this setting RGS3 was much less effective than NF1 or RASA1 (FIGS. 3A-3C and data not shown).

RGS3 had little effect when A59G—a transition state mutation that completely blocks GTPase activity (26)—was engineered alongside G12C (FIG. 3D). This suggests that RGS3 enhances hydrolysis by helping the reaction progress past the transition state. To better understand the mechanism, a molecular model was constructed using crystal structures of KRAS-GMPPNP (6OB2, ref. 27), RGS3 GAP domain (2OJ4, ref. 28) and Gaᵢₐ₁-GDP·AlF₄:RGS4 (IAGR, ref. 24). The model identified an asparagine (N) residue in the GAP domain of RGS3 as potentially involved in the hydrolysis reaction (FIG. 3E). This residue was predicted to orient away from the P-loop and, therefore, less likely to be impeded by G12 substitutions. Also, asparagine residues serve a catalytic function in some (non-RAS-specific) GAPs, including those enhancing GTP-hydrolysis by RAP1 and RHEB GTPases (29, 30).

The necessity of asparagine residue in RGS3 for the GTPase-activating effect on KRAS^{G12C} was tested. A histidine (H) substitution on N460 in p75 (RGS3) or on N147 in p25 (RGSD) attenuated the effect of RGS3 proteins on GTP-hydrolysis by KRAS^{G12C}, as evidenced by the γ-phosphate (FIG. 3F) and the α-phosphate (FIG. 3G) hydrolysis assays. Because RGS3 NH mutants had some residual activity against KRAS^{G12C}, it is likely that other residues also contribute to the GAP activity. This is in agreement with reports indicating that multiple RGS residues help enhance GTP hydrolysis by Gα (24). The asparagine residue is conserved between RGS isoforms, suggesting that other members of the family also enhance the KRAS GTPase activity. Indeed, this was the case for RGS4 (FIG. 3F). Together, the biochemical data suggest that RGS3 enhances the GTPase activity of KRAS in a mutant-inclusive manner (FIG. 3H).

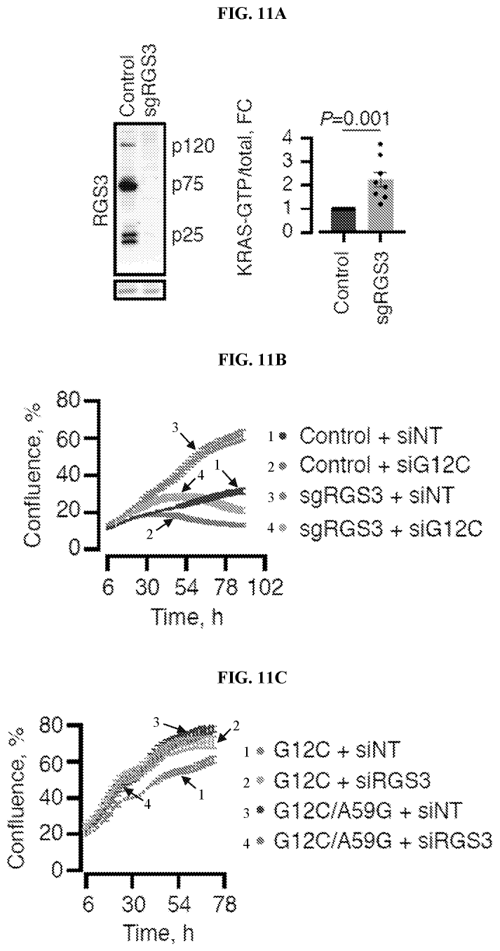

If RGS3 enhances KRAS^{G12C}-hydrolysis it ought to inactivate this oncoprotein in cells. sgRNA-mediated deletion of RGS3 in KRAS^{G12C}-mutant lung cancer cells lead to an increase in KRAS activation (FIG. 4A and FIG. 11A). Loss of RGS3 also enhanced proliferation in culture and accelerated tumor growth in xenograft studies (FIG. 4B). The proliferative advantage of RGS3^{-/-} cells was largely dependent on KRAS^{G12C}, as evidenced by a previously validated (18, 31) KRAS^{G12C}-specific siRNA (FIG. 11B). In agreement, siRNA-mediated knockdown of RGS3 increased proliferation in cells expressing KRAS^{G12C} but not significantly in cells expressing KRAS^{G12C}/A59G (FIG. 11C), which is insensitive to the GTPase-enhancing effect of RGS3 (FIG. 3D).

To determine if RGS3 expression inversely correlated with KRAS activation in lung cancer patients, a mutant KRAS-dependent transcriptional output score was established (FIGS. 12A-12B) and its ability to detect changes in KRAS^{G12C}-driven signaling following G12Ci-treatment was experimentally validated (FIG. 12C). As shown in FIGS. 12D-12E, a higher RGS3 expression was associated with lower mutant KRAS output in lung cancers harboring G12C or any other KRAS mutation. The negative correlation was observed with distinct KRAS up-regulated or down-regulated gene expression signatures (FIGS. 12F-12G). Little to no correlation was noted in KRAS^{WT} lung cancers.

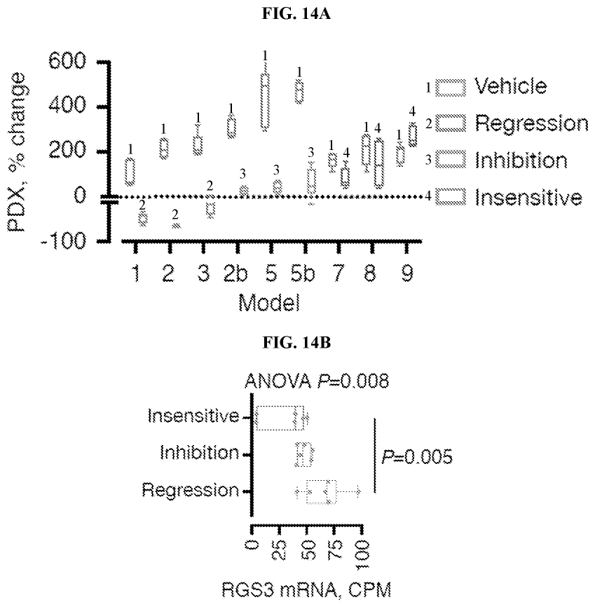

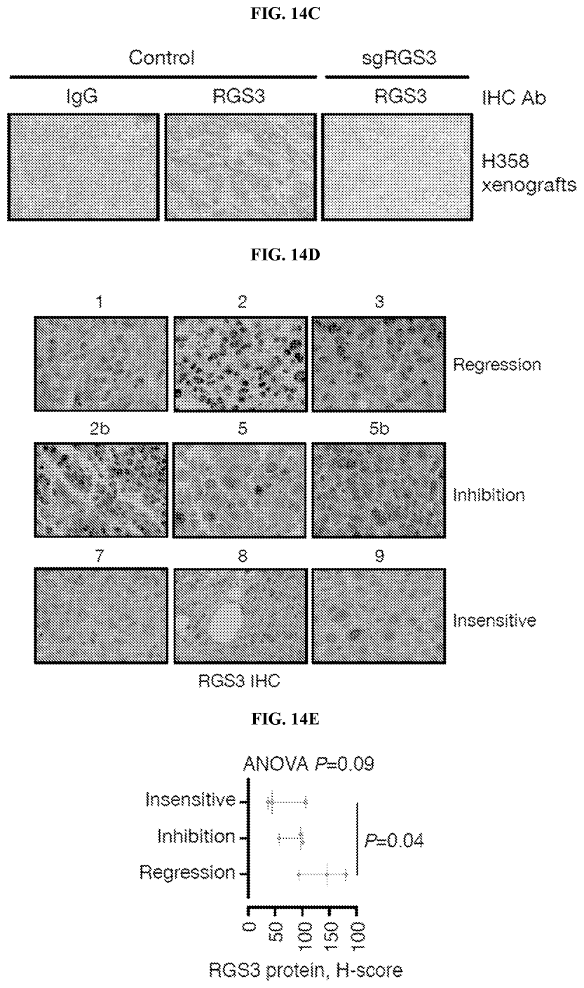

By inactivating KRAS^{G12C}, RGS3 ought to also enhance the effect of G12Ci-treatment, given that these drugs bind only to the inactive conformation of the oncoprotein. RGS3^{-/-} cells had an attenuated response to G12Ci-treatment, as compared to their isogenic RGS3 wild-type cells. This was evidenced by: 1) a diminished inhibition of KRAS-GTP levels (FIG. 13A), 2) a diminished antiproliferative effect in 3D culture (FIGS. 13B-13C), and 3) a less potent antitumor effect in vivo (FIGS. 4C-4D and FIG. 13D). Moreover, RGS3 expression correlated with susceptibility to G12Ci-treatment, in a panel of 9 lung cancer patient-derived xenograft (PDX) models. The PDX were established from 7 patients and their treatment response profile (FIG. 14A) was similar to that reported in a recent clinical study (19). RNA sequencing (FIG. 14B) and immunohistochemistry with an RGS3-specific antibody (FIGS. 14C-14E) revealed that tumors with higher RGS3 expression had a more potent treatment response. Taken together, the data suggest that RGS3 is required for a maximal inactive state-selective KRAS^{G12C} inhibition.

To provide additional evidence that RGS3 functions as a mutant KRAS-inclusive GAP, the ability of RGS3-depleted extracts to directly enhance GTP[γ³²P]-hydrolysis by KRAS was tested. As shown in FIG. 4E, almost complete KRAS^{G12C} hydrolysis was observed in the presence of control extracts (row 2 vs. row 1); an activity that was attenuated in RGS3^{-/-} extracts (row 3). By comparison, the activity was restored in extracts from RGS3^{-/-} cells expressing RGS3^{WT} (row 4) and diminished again in those expressing RGS3^{N460H} (row 5). RGS3^{-/-} extracts did not significantly affect GTP[γ³²P] hydrolysis by KRAS^{WT} (FIG. 4F). The latter agrees with the notion that RGS3^{-/-} cells still express conventional RAS-GAPs (such as NF1), which potently enhance hydrolysis by KRAS^{WT} (FIG. 3B: NF1>>RGS3), but not significantly that by KRAS^{G12C} (FIG. 3C: RGS3>NF1).

Lastly, the increase in cellular KRAS activation (FIG. 4G) and proliferation (FIG. 4H) conferred by the deletion of RGS3 were reversed by re-expression of RGS3^{WT}, but much less so by RGS3^{N460H}. Identifying that KRAS GTPases are susceptible to non-arginine dependent GAPs provides a mechanistic basis for the semiautonomous signaling capacity, which has been recently attributed to KRAS^{G12C} and several other KRAS oncoproteins (11, 13, 14, 32, 33). These results demonstrate why mutant KRAS is dependent on nucleotide exchange for activation and its vulnerability to drugs that impede this process. As evidenced above, RGS3 not only enhanced hydrolysis by KRAS^{G12C}, leading to its inactivation in cancer cells, but it was also required for maximal inactive state-selective inhibition.

RGS3 acts as a mutant KRAS-inclusive GAP to enhance GTP hydrolysis by KRAS^{G12C} in cancer cells. The effect of RGS3 was dependent on an asparagine residue—unlike the catalytic arginine-finger of canonical RAS-GAPs. This mechanism enables sufficient GTP hydrolysis in cancer cells and explains the effectiveness of drugs that trap mutant KRAS in an inactive-state (e.g., KRAS^{G12C}, SOS1 and SHP2 inhibitors). RGS3 deletion led to enhanced KRAS activation and tumor growth as well as diminished inactive state-selective KRAS^{G12C} inhibition. Although the RGS3-null phenotypes may occur via an effect on Gα, several lines of evidence suggest a dependency on KRAS^{G12C}. Selective knockdown of KRAS^{G12C} reversed the phenotype of RGS3-null cells, whereas knockdown of RGS3 enhanced the growth of KRAS^{G12C}-expressing cells but not that of KRAS^{G12C}/A59G expressing cells. Furthermore, RGS3-depleted cell extracts had a diminished GTPase-enhancing effect towards KRAS^{G12C}, as compared to RGS3 wild-type extracts. The activity was restored in RGS3-null cells expressing RGS3WT but diminished again in cells expressing RGS3^{N460H}. In a similar manner, the increase in cellular KRAS activation and proliferation caused by RGS3 deletion was rescued by RGS3 WT but not by RGS3^{N460H}. Lastly, extracts from RGS3-null cells did not significantly affect GTP[γ³²P] hydrolysis by KRAS^{WT}. If the effects were non-specific then RGS3-null lysates would have also affected KRAS^{WT} Together these lines of evidence support the conclusion that in the biological context, the consequences of RGS3 deletion are, at least in large part, due to an effect on KRAS^{G12C}

RGS3 is a weaker GAP for KRAS^{G12C} than NF1 is for KRAS^{WT}, which suggests that RGS3 is unlikely to inactivate the entire cellular pool of mutant KRAS; i.e., not to the same extent as NF1 is able to do for wild-type KRAS. Therefore, under steady-state conditions, the nucleotide cycle of mutant KRAS favors the active state, whereas that of wild-type KRAS favors the inactive state. The model disclosed herein thus provides a mechanistic basis for how KRAS mutants drive tumor growth, while at the same time remaining susceptible to GAP-assisted hydrolysis. The model also explains the susceptibility to inactive-state selective inhibition and the dependency of various KRAS oncoproteins on nucleotide-exchange for their activation.

Because KRAS^{G12C} inhibitors work by preventing nucleotide exchange, they require an intact GTPase activity to inhibit their target (13-17). If KRAS^{G12C} was completely insensitive to GAPs, these drugs would not be effective, or at least, they would require a long time to take effect (given that the time to inhibition is limited by the rate of hydrolysis). While inhibitor potency might have hindered initial observations (14), highly potent G12Ci inactivate the oncoprotein with minutes; quicker than even the fastest estimate of intrinsic hydrolysis rate in the literature (20). In addition, inhibition of nucleotide exchange by other means—as achieved by targeting SOS1 (33, 34), the adaptor phosphatase SHP2 (32, 35-37) or RTKs (38, 39)—has also been reported to suppresses mutant KRAS activation and/or tumor growth in various contexts.

A dependency on nucleotide exchange for activation would be redundant if KRAS oncoproteins were locked in a constitutively active, or persistently GTP-bound, state. RGS3-assisted hydrolysis by KRAS^{G12C} was approximately 1-2 orders of magnitude slower than NF1-stimulated hydrolysis by KRAS^{WT}, suggesting that RGS3 is unlikely to inactivate the entire cellular pool of KRAS^{G12C}; i.e., not to the same extent as NF1 is able to do for KRAS^{WT}. As a result, KRAS^{G12C} has a longer residency time in its active (GTP-bound) conformation than KRAS^{WT}, allowing the oncoprotein to drive proliferation in spite of its susceptibility to ‘atypical-for-RAS’ (i.e., non R-finger dependent) GAPs. Collectively, these experiments uncover an unexpected regulatory mechanism with fundamental implications for understanding and therapeutically-targeting KRAS oncoprotein-driven cancers.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all FIGS. and tables, to the extent they are not inconsistent with the explicit teachings of this specification.

### REFERENCES

1. M. Malumbres, M. Barbacid, RAS oncogenes: the first 30 years. Nat Rev Cancer 3, 459-465 (2003).
2. B. Papke, C. J. Der, Drugging RAS: Know the enemy. Science 355, 1158-1163 (2017).
3. D. K. Simanshu, D. V. Nissley, F. McCormick, RAS Proteins and Their Regulators in Human Disease. Cell 170, 17-33 (2017).
4. S. Li, A. Balmain, C. M. Counter, A model for RAS mutation patterns in cancers: finding the sweet spot. Nat Rev Cancer 18, 767-777 (2018).
5. M. Trahey, F. McCormick, A cytoplasmic protein stimulates normal N-ras p21 GTPase, but does not affect oncogenic mutants. Science 238, 542-545 (1987).
6. S. M. Margarit et al., Structural evidence for feedback activation by Ras. GTP of the Ras-specific nucleotide exchange factor SOS. Cell 112, 685-695 (2003).
7. J. L. Bos, H. Rehmann, A. Wittinghofer, GEFs and GAPs: critical elements in the control of small G proteins. Cell 129, 865-877 (2007).
8. Y. Pylayeva-Gupta, E. Grabocka, D. Bar-Sagi, RAS oncogenes: weaving a tumorigenic web. Nat Rev Cancer 11, 761-774 (2011).
9. G. Bollag, F. McCormick, Differential regulation of rasGAP and neurofibromatosis gene product activities. Nature 351, 576-579 (1991).
10. K. Scheffzek et al., The Ras-RasGAP complex: structural basis for GTPase activation and its loss in oncogenic Ras mutants. Science 277, 333-338 (1997).
11. T. G. Bivona, Dampening oncogenic RAS signaling. Science 363, 1280-1281 (2019).
12. J. M. Ostrem, U. Peters, M. L. Sos, J. A. Wells, K. M. Shokat, K-Ras(G12C) inhibitors allosterically control GTP affinity and effector interactions. Nature 503, 548-551 (2013).
13. P. Lito, M. Solomon, L. S. Li, R. Hansen, N. Rosen, Allele-specific inhibitors inactivate mutant KRAS G12C by a trapping mechanism. Science 351, 604-608 (2016).
14. M. P. Patricelli et al., Selective Inhibition of Oncogenic KRAS Output with Small Molecules Targeting the Inactive State. Cancer Discov 6, 316-329 (2016).
15. M. R. Janes et al., Targeting KRAS Mutant Cancers with a Covalent G12C-Specific Inhibitor. Cell 172, 578-589 e517 (2018).
16. J. Canon et al., The clinical KRAS(G12C) inhibitor AMG 510 drives anti-tumour immunity. Nature 575, 217-223 (2019).
17. J. Hallin et al., The KRAS(G12C) Inhibitor MRTX849 Provides Insight toward Therapeutic Susceptibility of KRAS-Mutant Cancers in Mouse Models and Patients. Cancer Discov 10, 54-71 (2020).
18. J. Y. Xue et al., Rapid non-uniform adaptation to conformation-specific KRAS(G12C) inhibition. Nature 577, 421-425 (2020).
19. D. S. Hong et al., KRAS(G12C) Inhibition with Sotorasib in Advanced Solid Tumors. N Engl J Med 383, 1207-1217 (2020).
20. J. C. Hunter et al., Biochemical and Structural Analysis of Common Cancer-Associated KRAS Mutations. Mol Cancer Res 13, 1325-1335 (2015).
21. N. Watson, M. E. Linder, K. M. Druey, J. H. Kehrl, K. J. Blumer, RGS family members: GTPase-activating proteins for heterotrimeric G-protein alpha-subunits. Nature 383, 172-175 (1996).
22. E. M. Ross, T. M. Wilkie, GTPase-activating proteins for heterotrimeric G proteins: regulators of G protein signaling (RGS) and RGS-like proteins. Annu Rev Biochem 69, 795-827 (2000).
23. R. R. Neubig, D. P. Siderovski, Regulators of G-protein signalling as new central nervous system drug targets. Nat Rev Drug Discov 1, 187-197 (2002).
24. J. J. Tesmer, D. M. Berman, A. G. Gilman, S. R. Sprang, Structure of RGS4 bound to AlF4-activated G(i alpha1): stabilization of the transition state for GTP hydrolysis. Cell 89, 251-261 (1997).
25. S. P. Srinivasa, N. Watson, M. C. Overton, K. J. Blumer, Mechanism of RGS4, a GTPase-activating protein for G protein alpha subunits. J Biol Chem 273, 1529-1533 (1998).
26. B. E. Hall, D. Bar-Sagi, N. Nassar, The structural basis for the transition from Ras-GTP to Ras-GDP. Proc Natl Acad Sci USA 99, 12138-12142 (2002).
27. D. Rabara et al., KRAS G13D sensitivity to neurofibromin-mediated GTP hydrolysis. Proc Natl Acad Sci USA 116, 22122-22131 (2019).
28. L. Rezabkova et al., 14-3-3 protein interacts with and affects the structure of RGS domain of regulator of G protein signaling 3 (RGS3). J Struct Biol 170, 451-461 (2010).
29. O. Daumke, M. Weyand, P. P. Chakrabarti, I. R. Vetter, A. Wittinghofer, The GTPase-activating protein Rap 1GAP uses a catalytic asparagine. Nature 429, 197-201 (2004).
30. Y. Li, K. Inoki, K. L. Guan, Biochemical and functional characterizations of small GTPase Rheb and TSC2 GAP activity. Mol Cell Biol 24, 7965-7975 (2004).
31. N. Sunaga et al., Knockdown of oncogenic KRAS in non-small cell lung cancers suppresses tumor growth and sensitizes tumor cells to targeted therapy. Mol Cancer Ther 10, 336-346 (2011).
32. R. J. Nichols et al., RAS nucleotide cycling underlies the SHP2 phosphatase dependence of mutant BRAF-, NF1- and RAS-driven cancers. Nat Cell Biol 20, 1064-1073 (2018).
33. M. H. Hofmann et al., BI-3406, a potent and selective SOS1:: KRAS interaction inhibitor, is effective in KRAS-driven cancers through combined MEK inhibition. Cancer Discov, (2020).
34. R. C. Hillig et al., Discovery of potent SOS1 inhibitors that block RAS activation via disruption of the RAS-SOS1 interaction. Proc Natl Acad Sci USA 116, 2551-2560 (2019).
35. C. Fedele et al., SHP2 Inhibition Prevents Adaptive Resistance to MEK Inhibitors in Multiple Cancer Models. Cancer Discov 8, 1237-1249 (2018).
36. S. Mainardi et al., SHP2 is required for growth of KRAS-mutant non-small-cell lung cancer in vivo. Nat Med 24, 961-967 (2018).
37. S. I. Ou et al., A12 The SHP2 Inhibitor RMC-4630 in Patients with KRAS-Mutant Non-Small Cell Lung Cancer: Preliminary Evaluation of a First-in-Man Phase 1 Clinical Trial. Journal of Thoracic Oncology 15, S15-S16 (2020).
38. C. Navas et al., EGF receptor signaling is essential for k-ras oncogene-driven pancreatic ductal adenocarcinoma. Cancer Cell 22, 318-330 (2012).
39. H. P. Moll et al., Afatinib restrains K-RAS-driven lung tumorigenesis. Sci Transl Med 10, (2018).
40. J. B. Gibbs, M. D. Schaber, W. J. Allard, I. S. Sigal, E. M. Scolnick, Purification of ras GTPase activating protein from bovine brain. Proc Natl Acad Sci USA 85, 5026-5030 (1988).
41. M. R. Webb, A continuous spectrophotometric assay for inorganic phosphate and for measuring phosphate release kinetics in biological systems. Proc Natl Acad Sci USA 89, 4884-4887 (1992).
42. P. Lito et al., Disruption of CRAF-mediated MEK activation is required for effective MEK inhibition in KRAS mutant tumors. Cancer Cell 25, 697-710 (2014).
43. A. M. Waters et al., Evaluation of the selectivity and sensitivity of isoform- and mutation-specific RAS antibodies. Sci Signal 10, (2017).
44. P. Bankhead et al., QuPath: Open source software for digital pathology image analysis. Sci Rep 7, 16878 (2017).
45. Y. Xue et al., An approach to suppress the evolution of resistance in BRAF(V600E)-mutant cancer. Nat Med 23, 929-937 (2017).

1. A method for selecting a cancer patient harboring a constitutively active KRAS mutation for treatment with a KRAS^{G12C} inhibitor that selectively targets the inactive state of KRAS comprising
a) detecting mRNA or polypeptide expression levels and/or activity of one or more regulators of G-protein signaling in a biological sample obtained from the cancer patient; and
b) administering to the cancer patient an effective amount of the KRAS^{G12C} inhibitor when expression levels and/or activity of the one or more regulators of G-protein signaling are comparable to a control sample obtained from a healthy subject or a predetermined threshold.

2. A method for treating a cancer patient harboring a constitutively active KRAS mutation comprising
(A)
administering to the cancer patient an effective amount of a KRAS^{G12C} inhibitor that selectively targets the inactive state of KRAS,
wherein mRNA or polypeptide expression and/or activity levels of one or more regulators of G-protein signaling in a biological sample obtained from the cancer patient are comparable to a control sample obtained from a healthy subject or a predetermined threshold; or
(B)
administering to the cancer patient an effective amount of a downstream inhibitor of RAS signaling pathway,
wherein mRNA or polypeptide expression and/or activity levels of one or more regulators of G-protein signaling in a biological sample obtained from the cancer patient are reduced relative to a control sample obtained from a healthy subject or a predetermined threshold,
optionally wherein the one or more regulators of G-protein signaling comprises RGS3 and/or RGS4.

3. The method of claim 1, wherein the KRAS^{G12C} inhibitor that selectively targets the inactive state of KRAS comprises one or more of MRTX1257, MRTX849, AMG510, ARS-1620, ARS-3248, LY3499446, LY3537982, GDC-6036, D-1553, JDQ443, BI 1823911, CodeBreaK100, ARS-853, WW peptide, DC-032-759, PTD-RBD-VIF, AU-8653, or ADT-007.

4. A method for selecting a cancer patient harboring a constitutively active KRAS mutation for treatment with a downstream inhibitor of RAS signaling pathway comprising
a) detecting mRNA or polypeptide expression levels and/or activity of one or more regulators of G-protein signaling in a biological sample obtained from the cancer patient; and
b) administering to the cancer patient an effective amount of the downstream inhibitor of RAS signaling pathway when expression levels and/or activity of the one or more regulators of G-protein signaling are reduced relative to a control sample obtained from a healthy subject or a predetermined threshold,
optionally wherein the one or more regulators of G-protein signaling comprises RGS3 and/or RGS4.

5. (canceled)

6. The method of claim 4, wherein the downstream inhibitor of RAS signaling pathway comprises one or more of BRAF inhibitors, MEK/ERK inhibitors, AURK inhibitors, and PI3K/Akt inhibitors.

7. The method of claim 6, wherein the BRAF inhibitors are selected from among GDC-0879, SB590885, Encorafenib, RAF265, TAK-632, PLX4720, CEP-32496, AZ628, Sorafenib Tosylate, Sorafenib, Vemurafenib (Zelboraf) and Dabrafenib (GSK2118436).

8. The method of claim 6, wherein the MEK/ERK inhibitors are selected from among MLN2480, Cobimetinib (GDC-0973), MEK 162, RO5126766, GDC-0623, VTX11e, Selumetinib (AZD6244), PD0325901, Trametinib (GSK1120212), U0126-EtOH, PD184352 (CI-1040), Refametinib, PD98059, BIX02189, Binimetinib, Pimasertib (AS-703026), SL327, BIX02188, AZD8330, TAK-733, PD318088, SCH772984, and FR 180204.

9. The method of claim 6, wherein the PI3K/Akt inhibitors are selected from among alpelisib, AMG319, apitolisib, AZD8186, BKM120, BGT226, bimiralisib, buparlisib, CH5132799, copanlisib, CUDC-907, dactolisisb, duvelisib, GDC-0941, GDC-0084, gedatolisib, GSK2292767, GSK2636771, idelalisib, IPI-549, leniolisib, LY294002, LY3023414, nemiralisib, omipalisib, PF-04691502, pictilisib, pilaralisib, PX866, RV-1729, SAR260301, SAR245408, serabelisib, SF1126, sonolisib, taselisib, umbralisib, voxtalisib, VS-5584, wortmannin, WX-037, ZSTK474, MK-2206, A-674563, A-443654, acetoxy-tirucallic acid, 3α- and 3β-acetoxy-tirucallic acids, afuresertib (GSK2110183), 4-amino-pyrido[2,3-d]pyrimidine derivative API-1, 3-aminopyrrolidine, anilinotriazole derivatives, ARQ751, ARQ 092, AT7867, AT13148, 7-azaindole, AZD5363, (−)-balanol derivatives, BAY 1125976, Boc-Phe-vinyl ketone, CCT128930, 3-chloroacetylindole, diethyl 6-methoxy-5,7-dihydroindolo [2,3-b]carbazole-2,10-dicarboxylate, diindolylmethane, 2,3-diphenylquinoxaline derivatives, DM-PIT-1, edelfosine, erucylphosphocholine, erufosine, frenolicin B, GSK-2141795, GSK690693, H-8, H-89, 4-hydroxynonenal, ilmofosine, imidazo-1,2-pyridine derivatives, indole-3-carbinol, ipatasertib, kalafungin, lactoquinomycin, medermycin, 3-methyl-xanthine, miltefosine, 1,6-naphthyridinone derivatives, NL-71-101, N-[(1-methyl-1H-pyrazol-4-yl)carbonyl]-N′-(3-bromophenyl)-thiourea, OSU-A9, perifosine, 3-oxo-tirucallic acid, PH-316, 3-phenyl-3H-imidazo[4,5-b]pyridine derivatives, 6-phenylpurine derivatives, PHT-427, PIT-1, PIT-2, 2-pyrimidyl-5-amidothiophene derivative, pyrrolo[2,3-d]pyrimidine derivatives, quinoline-4-carboxamide, 2-[4-(cyclohexa-1,3-dien-1-yl)-1H-pyrazol-3-yl]phenol, spiroindoline derivatives, triazolo[3,4-f][1,6]naphthyridin-3(2H)-one derivative, triciribine, triciribine mono-phosphate active analogue, and uprosertib.

10. The method of claim 6, wherein the AURK inhibitors are selected from among alisertib, tozasertib, SP-96, AT9283, danusertib (PHA-739358), AMG900, cenisertib, SNS-314, barasertib, hesperadin, AZD1152, GSK1070916, CYC116, BI 811283, AZD2811, PHA680632, reversine, CCT129202, CCT137690, quercetin, VX-680, PF-03814735, XL228, ENMD-2076, BI-847325, Ilorasertib/ABT-348, MK-5108/VX-689, and Chiauranib/CS-2164.

11. The method of claim 1, wherein the constitutively active KRAS mutation comprises a substitution at codon 12, 13 or 61 of KRAS.

12. The method of claim 11, wherein the constitutively active KRAS mutation is G12C, G12V, G12D, G12A, G12R, G12S, G12F, G12L, G13C, G13D, G13R, G13A, G13S, G13V, G13E, Q61H, Q61K, Q61L, Q61R, Q61P, or Q61E.

13. The method of claim 1, wherein the cancer patient suffers from a cancer selected from among pancreatic cancer, colon cancer, lung cancer, small intestine cancer, urinary tract cancer, endometrial cancer, cervical cancer, bladder cancer, liver cancer, myeloid leukemia breast cancer and biliary tract cancer.

14. The method of claim 13, wherein the lung cancer is non-small-cell lung cancer (NSCLC) or small-cell lung cancer (SCLC).

15. The method of claim 1, wherein the one or more regulators of G-protein signaling comprises RGS3 and/or RGS4.

16. The method of claim 15 wherein RGS3 comprises p75 and/or p25 RGS3 isoforms.

17. The method of claim 1, wherein mRNA expression levels are detected via next-generation sequencing, RNA-seq, real-time quantitative PCR (qPCR), digital PCR (dPCR), Reverse transcriptase-PCR (RT-PCR), Northern blotting, microarray, dot or slot blots, in situ hybridization, or fluorescent in situ hybridization (FISH).

18. The method of claim 1, wherein polypeptide expression levels are detected via Western blotting, enzyme-linked immunosorbent assays (ELISA), dot blotting, immunohistochemistry, immunofluorescence, immunoprecipitation, immunoelectrophoresis, or mass-spectrometry.

19. The method of claim 1, wherein the biological sample obtained from the cancer patient comprises biopsied tumor tissue, whole blood, plasma, or serum.

20. The method of claim 4, wherein the downstream inhibitor of RAS signaling pathway is administered orally, intranasally, systemically, intravenously, intramuscularly, intraperitoneally, intradermally, intraocularly, iontophoretically, transmucosally, subcutaneously, rectally, intrathecally, intratumorally or topically.

21. The method of claim 1, wherein the KRAS^{G12C} inhibitor is administered orally, intranasally, systemically, intravenously, intramuscularly, intraperitoneally, intradermally, intraocularly, iontophoretically, transmucosally, subcutaneously, rectally, intrathecally, intratumorally or topically.


SEQUENCE LISTING (26 sequences)

SEQ ID NO: 1 - DNA, 20 bp, Artificial Sequence
  Description of Artificial Sequence Synthetic oligonucleotide
tgcgtgatcctcttcctggc

SEQ ID NO: 2 - DNA, 20 bp, Artificial Sequence
  Description of Artificial Sequence Synthetic oligonucleotide
ctacacgcgggagcacacca

SEQ ID NO: 3 - DNA, 2653 bp, Homo sapiens
acttccctgcgcctctggctgcctcaatgtcactggggattagtgtgcatttgcggcttttggcggacactcccctcctggctgcagcctcaccctctggagcccagggatgggcttgggcctgggagccccctgaaccagggagcagccctgaccgtccgtttctctattttccctgacgtcagaaggcagagtgcttattcactttggaagcgcactcgcaggagcagaagaagagagtgtgctggtgcctgtcggagaacatcgccaagcagcaacagctggcagcatcacccccggacagcaagatgtttgagacggaggcagatgagaagagggagatggccttggaggaagggaaggggcctggtgccgaggattccccacccagcaaggagccctctcctggccaggagcttcctccaggacaagaccttccacccaacaaggactccccttctgggcaggaacccgctcccagccaagaaccactgtccagcaaagactcagctacctctgaaggatcccctccaggcccagatgctccgcccagcaaggatgtgccaccatgccaggaaccccctccagcccaagacctctcaccctgccaggacctacctgctggtcaagaacccctgcctcaccaggaccctctactcaccaaagacctccctgccatccaggaatcccccacccgggaccttccaccctgtcaagatctgcctcctagccaggtctccctgccagccaaggcccttactgaggacaccatgagctccggggacctactagcagctactggggacccacctgcggcccccaggccagccttcgtgatccctgaggtccggctggatagcacctacagccagaaggcaggggcagagcagggctgctcgggagatgaggaggatgcagaagaggccgaggaggtggaggagggggaggaaggggaggaggacgaggatgaggacaccagcgatgacaactacggagagcgcagtgaggccaagcgcagcagcatgatcgagacgggccagggggctgagggtggcctctcactgcgtgtgcagaactcgctgcggcgccggacgcacagcgagggcagcctgctgcaggagccccgagggccctgctttgcctccgacaccaccttgcactgctcagacggtgagggcgccgcctccacctggggcatgccttcgcccagcaccctcaagaaagagctgggccgcaatggtggctccatgcaccacctttccctcttcttcacaggacacaggaagatgagcggggctgacaccgttggggatgatgacgaagcctcccggaagagaaagagcaaaaacctagccaaggacatgaagaacaagctggggatcttcagacggcggaatgagtcccctggagcccctcccgcgggcaaggcagacaaaatgatgaagtcattcaagcccacctcagaggaagccctcaagtggggcgagtccttggagaagctgctggttcacaaatacgggttagcagtgttccaagccttccttcgcactgagttcagtgaggagaatctggagttctggttggcttgtgaggacttcaagaaggtcaagtcacagtccaagatggcatccaaggccaagaagatctttgctgaatacatcgcgatccaggcatgcaaggaggtcaacctggactcctacacgcgggagcacaccaaggacaacctgcagagcgtcacgcggggctgcttcgacctggcacagaagcgcatcttcgggctcatggaaaaggactcgtaccctcgctttctccgttctgacctctacctggaccttattaaccagaagaagatgagtcccccgctttaggggccactggagtcgagctcagcgttcacaccaggcgggctgggtcccctgcccacctgcctccctgccccctgtgacggagggggcaagcaagcccccagaggctgtgtctctggacagacggatagacatacggaagcgaggcctggaccaagagaggcccaggctactggaggagtagaaggatgggccccgtggggtccccactgccccggtacgagggggcccaagaccctggcaggtcaggggccctggccaagccagatctggagctgctgctccctgctgcggagaccgcggaggcttcgcgttgaccaagttccttaaagaactggctgatggggcaggaggtccaggcctgggctctcgggccctcctagagggccattggagcttgcagctcagacccccactttgagttttatttatttaaatagtagttggatgcttggcacgtcgtcctgtaataggaaacccttgcctcatcagttttcctgatttacaagtgcaatattttagccaatgccttgggagaagctgccatgcaaaggtggacaccattctccagcttcaggggatatgctcgtcccgggcaccggtggcaggcagctggccttctggactaaggcagcctggggggacactgcagtctggctacacacagagatctggcaccccctgggtggagtgtccctcgggggctttgggaaagcatggcaccctcagaccacacagtagccaagttctggagcaaataaaaggcctgtgttatttcttgttcttga

SEQ ID NO: 4 - DNA, 2631 bp, Homo sapiens
agtgcgggctcccgctgctgggggagagctgggttttcatggggcggcagccgaggcaggacccgcagccatgaaccgcttcaatgggctctgcaaggtgtgctcggagcgccgctaccgccagatcaccatcccgaggggaaaggacggctttggcttcaccatctgctgcgactctccagttcgagtccaggccgtggattccgggggtccggcggaacgggcagggctgcagcagctggacacggtgctgcagctgaatgagaggcctgtggagcactggaaatgtgtggagctggcccacgagatccggagctgccccagtgagatcatcctactcgtgtggcgcatggtcccccaggtcaagccaggaccagatggcggggtcctgcggcgggcctcctgcaagtcgacacatgacctccagtcaccccccaacaaacgggagaagaactgcacccatggggtccaggcacggcctgagcagcgccacagctgccacctggtatgtgacagctctgatgggctgctgctcggcggctgggagcgctacaccgaggtggccaagcgcgggggccagcacaccctgcctgcactgtcccgtgccactgcccccaccgaccccaactacatcatcctggccccgctgaatcctgggagccagctgctccggcctgtgtaccaggaggataccatccccgaagaatcagggagtcccagtaaagggaagtcctacacaggcctggggaagaagtcccggctgatgaagacagtgcagaccatgaagggccacgggaactaccaaaactgcccggttgtgaggccgcatgccacgcactcaagctatggcacctacgtcaccctggcccccaaagtcctggtgttccctgtctttgttcagcctctagatctctgtaatcctgcccggaccctcctgctgtcagaggagctgctgctgtatgaagggaggaacaaggctgccgaggtgacactgtttgcctattcggacctgctgctcttcaccaaggaggacgagcctggccgctgcgacgtcctgaggaaccccctctacctccagagtgtgaagctgcaggaaggttcttcagaagacctgaaattctgcgtgctctatctagcagagaaggcagagtgcttattcactttggaagcgcactcgcaggagcagaagaagagagtgtgctggtgcctgtcggagaacatcgccaagcagcaacagctggcagcatcacccccggacagcaagatgagcggggctgacaccgttggggatgatgacgaagcctcccggaagagaaagagcaaaaacctagccaaggacatgaagaacaagctggggatcttcagacggcggaatgagtcccctggagcccctcccgcgggcaaggcagacaaaatgatgaagtcattcaagcccacctcagaggaagccctcaagtggggcgagtccttggagaagctgctggttcacaaatacgggttagcagtgttccaagccttccttcgcactgagttcagtgaggagaatctggagttctggttggcttgtgaggacttcaagaaggtcaagtcacagtccaagatggcatccaaggccaagaagatctttgctgaatacatcgcgatccaggcatgcaaggaggtcaacctggactcctacacgcgggagcacaccaaggacaacctgcagagcgtcacgcggggctgcttcgacctggcacagaagcgcatcttcgggctcatggaaaaggactcgtaccctcgctttctccgttctgacctctacctggaccttattaaccagaagaagatgagtcccccgctttaggggccactggagtcgagctcagcgttcacaccaggcgggctgggtcccctgcccacctgcctccctgccccctgtgacggagggggcaagcaagcccccagaggctgtgtctctggacagacggatagacatacggaagcgaggcctggaccaagagaggcccaggctactggaggagtagaaggatgggccccgtggggtccccactgccccggtacgagggggcccaagaccctggcaggtcaggggccctggccaagccagatctggagctgctgctccctgctgcggagaccgcggaggcttcgcgttgaccaagttccttaaagaactggctgatggggcaggaggtccaggcctgggctctcgggccctcctagagggccattggagcttgcagctcagacccccactttgagttttatttatttaaatagtagttggatgcttggcacgtcgtcctgtaataggaaacccttgcctcatcagttttcctgatttacaagtgcaatattttagccaatgccttgggagaagctgccatgcaaaggtggacaccattctccagcttcaggggatatgctcgtcccgggcaccggtggcaggcagctggccttctggactaaggcagcctggggggacactgcagtctggctacacacagagatctggcaccccctgggtggagtgtccctcgggggctttgggaaagcatggcaccctcagaccacacagtagccaagttctggagcaaataaaaggcctgtgttatttcttgttcttga

SEQ ID NO: 5 - DNA, 1460 bp, Homo sapiens
agtcatcaggccaggattccagagagcgtgtgtggctgcagcctgcaccgttgctgcccgctgcccaggacgcggggtgggggacaggagccagagtggtgcctcctacagaccaatctgcggccccaaggtggggggccctacagagatgctccgagagccaaggacatgaagaacaagctggggatcttcagacggcggaatgagtcccctggagcccctcccgcgggcaaggcagacaaaatgatgaagtcattcaagcccacctcagaggaagccctcaagtggggcgagtccttggagaagctgctggttcacaaatacgggttagcagtgttccaagccttccttcgcactgagttcagtgaggagaatctggagttctggttggcttgtgaggacttcaagaaggtcaagtcacagtccaagatggcatccaaggccaagaagatctttgctgaatacatcgcgatccaggcatgcaaggaggtcaacctggactcctacacgcgggagcacaccaaggacaacctgcagagcgtcacgcggggctgcttcgacctggcacagaagcgcatcttcgggctcatggaaaaggactcgtaccctcgctttctccgttctgacctctacctggaccttattaaccagaagaagatgagtcccccgctttaggggccactggagtcgagctcagcgttcacaccaggcgggctgggtcccctgcccacctgcctccctgccccctgtgacggagggggcaagcaagcccccagaggctgtgtctctggacagacggatagacatacggaagcgaggcctggaccaagagaggcccaggctactggaggagtagaaggatgggccccgtggggtccccactgccccggtacgagggggcccaagaccctggcaggtcaggggccctggccaagccagatctggagctgctgctccctgctgcggagaccgcggaggcttcgcgttgaccaagttccttaaagaactggctgatggggcaggaggtccaggcctgggctctcgggccctcctagagggccattggagcttgcagctcagacccccactttgagttttatttatttaaatagtagttggatgcttggcacgtcgtcctgtaataggaaacccttgcctcatcagttttcctgatttacaagtgcaatattttagccaatgccttgggagaagctgccatgcaaaggtggacaccattctccagcttcaggggatatgctcgtcccgggcaccggtggcaggcagctggccttctggactaaggcagcctggggggacactgcagtctggctacacacagagatctggcaccccctgggtggagtgtccctcgggggctttgggaaagcatggcaccctcagaccacacagtagccaagttctggagcaaataaaaggcctgtgttatttcttgttcttga

SEQ ID NO: 6 - DNA, 2594 bp, Homo sapiens
actctgtgccactccgtgccaggccctgagggcacccggttgctgcttccttccgtctttccccaaggactatcagagatgccagcgtgacccctgacacgtgtgtgcagcagcctgcagctgccccaagccatggctgaacactgactcccagctgtggggcttcaccattacagactccccagggcttcaaagacttctcagcttcgagcatggcttttggctgtcagggcagctgtacaatagtggatgtttgagacggaggcagatgagaagagggagatggccttggaggaagggaaggggcctggtgccgaggattccccacccagcaaggagccctctcctggccaggagcttcctccaggacaagaccttccacccaacaaggactccccttctgggcaggaacccgctcccagccaagaaccactgtccagcaaagactcagctacctctgaaggatcccctccaggcccagatgctccgcccagcaaggatgtgccaccatgccaggaaccccctccagcccaagacctctcaccctgccaggacctacctgctggtcaagaacccctgcctcaccaggaccctctactcaccaaagacctccctgccatccaggaatcccccacccgggaccttccaccctgtcaagatctgcctcctagccaggtctccctgccagccaaggcccttactgaggacaccatgagctccggggacctactagcagctactggggacccacctgcggcccccaggccagccttcgtgatccctgaggtccggctggatagcacctacagccagaaggcaggggcagagcagggctgctcgggagatgaggaggatgcagaagaggccgaggaggtggaggagggggaggaaggggaggaggacgaggatgaggacaccagcgatgacaactacggagagcgcagtgaggccaagcgcagcagcatgatcgagacgggccagggggctgagggtggcctctcactgcgtgtgcagaactcgctgcggcgccggacgcacagcgagggcagcctgctgcaggagccccgagggccctgctttgcctccgacaccaccttgcactgctcagacggtgagggcgccgcctccacctggggcatgccttcgcccagcaccctcaagaaagagctgggccgcaatggtggctccatgcaccacctttccctcttcttcacaggacacaggaagatgagcggggctgacaccgttggggatgatgacgaagcctcccggaagagaaagagcaaaaacctagccaaggacatgaagaacaagctggggatcttcagacggcggaatgagtcccctggagcccctcccgcgggcaaggcagacaaaatgatgaagtcattcaagcccacctcagaggaagccctcaagtggggcgagtccttggagaagctgctggttcacaaatacgggttagcagtgttccaagccttccttcgcactgagttcagtgaggagaatctggagttctggttggcttgtgaggacttcaagaaggtcaagtcacagtccaagatggcatccaaggccaagaagatctttgctgaatacatcgcgatccaggcatgcaaggaggtcaacctggactcctacacgcgggagcacaccaaggacaacctgcagagcgtcacgcggggctgcttcgacctggcacagaagcgcatcttcgggctcatggaaaaggactcgtaccctcgctttctccgttctgacctctacctggaccttattaaccagaagaagatgagtcccccgctttaggggccactggagtcgagctcagcgttcacaccaggcgggctgggtcccctgcccacctgcctccctgccccctgtgacggagggggcaagcaagcccccagaggctgtgtctctggacagacggatagacatacggaagcgaggcctggaccaagagaggcccaggctactggaggagtagaaggatgggccccgtggggtccccactgccccggtacgagggggcccaagaccctggcaggtcaggggccctggccaagccagatctggagctgctgctccctgctgcggagaccgcggaggcttcgcgttgaccaagttccttaaagaactggctgatggggcaggaggtccaggcctgggctctcgggccctcctagagggccattggagcttgcagctcagacccccactttgagttttatttatttaaatagtagttggatgcttggcacgtcgtcctgtaataggaaacccttgcctcatcagttttcctgatttacaagtgcaatattttagccaatgccttgggagaagctgccatgcaaaggtggacaccattctccagcttcaggggatatgctcgtcccgggcaccggtggcaggcagctggccttctggactaaggcagcctggggggacactgcagtctggctacacacagagatctggcaccccctgggtggagtgtccctcgggggctttgggaaagcatggcaccctcagaccacacagtagccaagttctggagcaaataaaaggcctgtgttatttcttgttcttga

SEQ ID NO: 7 - DNA, 4228 bp, Homo sapiens
ggagactccggttactggggagcaacacagccgcctcgggttgcagacgctcctgtccgggtcgcagtgggacgccatggagcgctccctgcaccgcgtctccctcgggagccggcgtgcccacccggacttgtccttctacctcaccacctttggtcagctgaggctgtccattgatgcccaggaccgggttctgctgcttcacagtctctttattcgtgtggatggatatgtctatgtgtgtctctctttctcgctgtgtgtgtgtgtatgtttccattcatccaccccaatgtctgaattctcttttagttatagaaggtaaaggcctgatcagcaaacagcctggcacctgtgatccgtatgtgaagatttctttgatccctgaagatagtagactacgccaccagaagacgcagaccgttccagactgcagagacccggctttccacgagcacttcttctttcctgtccaagaggaggatgatcagaagcgtctcttggttactgtgtggaacagggccagccagtccagacagagtggactcattggctgcatgagctttggggtgaagtctctcctgactccagacaaggagatcagtggttggtactacctcctaggggagcacctgggccggaccaagcacttgaaggtggccaggcggcgactgcggccgctgagagacccgctgctgagaatgccaggaggtggggacactgagaatgggaagaaactaaagatcaccatcccgaggggaaaggacggctttggcttcaccatctgctgcgactctccagttcgagtccaggccgtggattccgggggtccggcggaacgggcagggctgcagcagctggacacggtgctgcagctgaatgagaggcctgtggagcactggaaatgtgtggagctggcccacgagatccggagctgccccagtgagatcatcctactcgtgtggcgcatggtcccccaggtcaagccaggaccagatggcggggtcctgcggcgggcctcctgcaagtcgacacatgacctccagtcaccccccaacaaacgggagaagaactgcacccatggggtccaggcacggcctgagcagcgccacagctgccacctggtatgtgacagctctgatgggctgctgctcggcggctgggagcgctacaccgaggtggccaagcgcgggggccagcacaccctgcctgcactgtcccgtgccactgcccccaccgaccccaactacatcatcctggccccgctgaatcctgggagccagctgctccggcctgtgtaccaggaggataccatccccgaagaatcagggagtcccagtaaagggaagtcctacacaggcctggggaagaagtcccggctgatgaagacagtgcagaccatgaagggccacgggaactaccaaaactgcccggttgtgaggccgcatgccacgcactcaagctatggcacctacgtcaccctggcccccaaagtcctggtgttccctgtctttgttcagcctctagatctctgtaatcctgcccggaccctcctgctgtcagaggagctgctgctgtatgaagggaggaacaaggctgccgaggtgacactgtttgcctattcggacctgctgctcttcaccaaggaggacgagcctggccgctgcgacgtcctgaggaaccccctctacctccagagtgtgaagctgcaggaaggttcttcagaagacctgaaattctgcgtgctctatctagcagagaaggcagagtgcttattcactttggaagcgcactcgcaggagcagaagaagagagtgtgctggtgcctgtcggagaacatcgccaagcagcaacagctggcagcatcacccccggacagcaagatgtttgagacggaggcagatgagaagagggagatggccttggaggaagggaaggggcctggtgccgaggattccccacccagcaaggagccctctcctggccaggagcttcctccaggacaagaccttccacccaacaaggactccccttctgggcaggaacccgctcccagccaagaaccactgtccagcaaagactcagctacctctgaaggatcccctccaggcccagatgctccgcccagcaaggatgtgccaccatgccaggaaccccctccagcccaagacctctcaccctgccaggacctacctgctggtcaagaacccctgcctcaccaggaccctctactcaccaaagacctccctgccatccaggaatcccccacccgggaccttccaccctgtcaagatctgcctcctagccaggtctccctgccagccaaggcccttactgaggacaccatgagctccggggacctactagcagctactggggacccacctgcggcccccaggccagccttcgtgatccctgaggtccggctggatagcacctacagccagaaggcaggggcagagcagggctgctcgggagatgaggaggatgcagaagaggccgaggaggtggaggagggggaggaaggggaggaggacgaggatgaggacaccagcgatgacaactacggagagcgcagtgaggccaagcgcagcagcatgatcgagacgggccagggggctgagggtggcctctcactgcgtgtgcagaactcgctgcggcgccggacgcacagcgagggcagcctgctgcaggagccccgagggccctgctttgcctccgacaccaccttgcactgctcagacggtgagggcgccgcctccacctggggcatgccttcgcccagcaccctcaagaaagagctgggccgcaatggtggctccatgcaccacctttccctcttcttcacaggacacaggaagatgagcggggctgacaccgttggggatgatgacgaagcctcccggaagagaaagagcaaaaacctagccaaggacatgaagaacaagctggggatcttcagacggcggaatgagtcccctggagcccctcccgcgggcaaggcagacaaaatgatgaagtcattcaagcccacctcagaggaagccctcaagtggggcgagtccttggagaagctgctggttcacaaatacgggttagcagtgttccaagccttccttcgcactgagttcagtgaggagaatctggagttctggttggcttgtgaggacttcaagaaggtcaagtcacagtccaagatggcatccaaggccaagaagatctttgctgaatacatcgcgatccaggcatgcaaggaggtcaacctggactcctacacgcgggagcacaccaaggacaacctgcagagcgtcacgcggggctgcttcgacctggcacagaagcgcatcttcgggctcatggaaaaggactcgtaccctcgctttctccgttctgacctctacctggaccttattaaccagaagaagatgagtcccccgctttaggggccactggagtcgagctcagcgttcacaccaggcgggctgggtcccctgcccacctgcctccctgccccctgtgacggagggggcaagcaagcccccagaggctgtgtctctggacagacggatagacatacggaagcgaggcctggaccaagagaggcccaggctactggaggagtagaaggatgggccccgtggggtccccactgccccggtacgagggggcccaagaccctggcaggtcaggggccctggccaagccagatctggagctgctgctccctgctgcggagaccgcggaggcttcgcgttgaccaagttccttaaagaactggctgatggggcaggaggtccaggcctgggctctcgggccctcctagagggccattggagcttgcagctcagacccccactttgagttttatttatttaaatagtagttggatgcttggcacgtcgtcctgtaataggaaacccttgcctcatcagttttcctgatttacaagtgcaatattttagccaatgccttgggagaagctgccatgcaaaggtggacaccattctccagcttcaggggatatgctcgtcccgggcaccggtggcaggcagctggccttctggactaaggcagcctggggggacactgcagtctggctacacacagagatctggcaccccctgggtggagtgtccctcgggggctttgggaaagcatggcaccctcagaccacacagtagccaagttctggagcaaataaaaggcctgtgttatttcttgttcttga

SEQ ID NO: 8 - DNA, 9713 bp, Homo sapiens
agtgcgggctcccgctgctgggggagagctgggttttcatggggcggcagccgaggcaggacccgcagccatgaaccgcttcaatgggctctgcaaggtgtgctcggagcgccgctaccgccagatcaccatcccgaggggaaaggacggctttggcttcaccatctgctgcgactctccagttcgagtccaggccgtggattccgggggtccggcggaacgggcagggctgcagcagctggacacggtgctgcagctgaatgagaggcctgtggagcactggaaatgtgtggagctggcccacgagatccggagctgccccagtgagatcatcctactcgtgtggcgcatggtcccccaggtcaagccaggaccagatggcggggtcctgcggcgggcctcctgcaagtcgacacatgacctccagtcaccccccaacaaacgggagaagaactgcacccatggggtccaggcacggcctgagcagcgccacagctgccacctggtatgtgacagctctgatgggctgctgctcggcggctgggagcgctacaccgaggtggccaagcgcgggggccagcacaccctgcctgcactgtcccgtgccactgcccccaccgaccccaactacatcatcctggccccgctgaatcctgggagccagctgctccggcctgtgtaccaggaggataccatccccgaagaatcagggagtcccagtaaagggaagtcctacacaggcctggggaagaagtcccggctgatgaagacagtgcagaccatgaagggccacgggaactaccaaaactgcccggttgtgaggccgcatgccacgcactcaagctatggcacctacgtcaccctggcccccaaagtcctggtgttccctgtctttgttcagcctctagatctctgtaatcctgcccggaccctcctgctgtcagaggagctgctgctgtatgaagggaggaacaaggctgccgaggtgacactgtttgcctattcggacctgctgctcttcaccaaggaggacgagcctggccgctgcgacgtcctgaggaaccccctctacctccagagtgtgaagctgcaggaaggttcttcagaagacctgaaattctgcgtgctctatctagcagagaaggcagagtgcttattcactttggaagcgcactcgcaggagcagaagaagagagtgtgctggtgcctgtcggagaacatcgccaagcagcaacagctggcagcatcacccccggacagcaagaaactccaccctttcggctctctccagcaggagatggggccggtcaactcaaccaatgccacccaggatagaagctttacctcaccaggacagactctgattggctgagcaaacccaagggcgggactctgcttctggcaacttaaccctttcttgggcatcccctaccacacagtaacctcacctcaactggacctgaaacagaggaccaagatggtgggtctagggtcatctgaagggagtggtcccaagggtgcatgtggatgtcaacagaaggaccacaatggtgtgagaggccttgtgggcatgcagagactggcagcagaaagtcccaagaggagaggactcagaggcttcttccttccaaggaggaggaggaggaggacattccacatctgggcttccagtggtggctcccggacctgccttgggggcagcaggagaggttgggagaggtcaccctgatgaagaacaatgccatgatctgctgctgccagaggacatctccgcccatccactcattttccacctgtacacattggctcattcattcgatggatatttcgggtgcatctactttatgccaggctctatgctaggagctggggatactgtggagaaaaagtcggatatggtctcgctctcatcaagatttcagtctagctgggagtgagcgttaaataagtcaataccaacacacacacgtgcacacacgtgatggcaagctgtgatcatctatgaaagaaaaaacacagatttctttaagagggaattgtacttagagtgctgtgtgtgggggtggggatggggaaggacataacttttaaaccgagacttgtgggctggagaggaaggagccagtcctggacttggggaagagtgttcgcggtggatggaatagcacgtggagagtccctgagatgggaaggagctcagtgggctggaggaatggcgagcaggccagtgtggctggagcttagtgaggtgcaagacacagtgggtgaggtagttgatgtcttgttgatttcattctagatacactggagagccattgaaggcttttgaggaagggagttgcaagtacatttttacaagatccgtttacacataggaggggcccaagtgggtgtgagatcagttagggggctcgttcagttctctaggtgaggactgtcctggtggcttgtcctggatggtggctggggaagtggatgcctgaatgccaggctctggagaagagatgactccctgccccatccccctgcctaccctcctcctcaatggcccctctctcagtggatgtgagtgaggattaggaccttccccacagagccaccttttggccaggcctcgtgggggatagtgggggcagtgctgagttctaggagtgttcctggaagttgggggcagtgagtgctcagcctggcccaggtgggagagttcactgctgcccaggccttagcaggggctgctgtgctgtctttctgtcccatgctgagcattgtgataggaagctgccacacagtgccctggaaactgacttcgcatgctttccatggggcttccgagggccagggactgggctgggggtggcctagcagctctagtggagactgtgctgccgcagggctgaaggaagaccaaccaaggggacagccgtttgagcccagcccactcactgcctgctgcacctccagctggagcccagggttgggcagctctcatactcttatctaactgccagacctagagggagggaagcccaaggctgcaggtctgagggacacggctcatgggcaaaagggtatcaagaggattagagtcacttgcataggaaaagagggtcagagaggctgagggctggctgaggccagagaggttccctgaccttggacattctttgtgttgtgtaagacctcggcatctttagagaaaggttcttgtgcccctgtgagcttgctcagctgctatggccacttgaaccccaaaaatggtggcctagaagagaatacaccccttacttcccacttatcctcctctcaacctaggaaagatcagacatattgactcaacagtctaacaggggatgggctggatctgcccgtgaccagaccaatgccccccacccagcaagggagtaaacgctgcctgaagtgggaccagtgatacacagcatctctgtctgccagggctcagcccgcgggggggatttgtgagaggctgagcagctgggtctgcacagacagcaagctgcactttgatttcatttgattcccttacaaaatgacattaaggggaatgtgaggcacaggagactcgcctgtctgggaaggcagcaagctcatctctcctggacctggtgacctacctctgtctaccagacaggtgcagaggacaggagctctggccacaaagaaatggacagtctgaatgcagcacacagccctggggacaggacttttcacccagcctgtcctgacaagctgctgagtcgagcagatgctcacatcagggtcccaggtccggctgttagggaagtggacatcagttcctggtgggccctgggcagtgactgtggtgagccacagcaccattgctgctctgcctctcaaatcgatcgaacaattaatttggctccacaggctcgagaagatgaaacatgacctgatggccactgctgtccagcatcccagtccatcctagctgtccaggatgatgaacaagctatggaaattgcacttgagagctataatcacgggaagcattgccgagcccctccctgctacataaatcatgctaagaagcacacaatggcctctttcctgatgggctaattgtcttggtttggtgtctcaggcccaggagctgggaagatgggagcacaaccatttattgttggaaagaccatctgtgcaacttgaatacaaagtgattcttttagttccccacacacccccgaccctctgatcccattttcatgttttttttttttttttctttttatgtctcaggctctggttcttttaaaactcttcatctatcctgtaagccatcaggcctggatctctgctggcctgggtcttttggctatttgggggttgggacattgcagaccttttggaaataatgacatttatttatatatgtcttatatttttattctttatttttccccataaattctattaccaaagtcctcatttgtcacacatggcaccaaggcaccaagagggtttttgcaaagtgaaaatgcaggcaattttgcaacgtggaagggctccccacagcgaaagtcccacagtgagagcctaaactttggagatggcaactggggtccccaggcatgatctccccccagcctcccagcagccttgttttatggatgaaagacaccaaggctctgagaagttaagtgttttatcctaggtcacacagctggtaagggccaagcttgccctcaagccaaggcctttgaacctaatctaccactcatttctgataaaaaggtttgtcttttagggatagaagtaagagaaatcaataatctccaggaagaggcaagagagggatatccggcatgtcagaagctgtggcccatcaagctgtagagggaatgcttttaggataaataaaatgagatgccttattttccacccagtggagaataaacttccagaatgcattttcctgagggtggcacaggctgaaaagatcaatagcttcccacaagatttgaaaaaagcctaaaatgagagatcagtttgagctccttaagacaggttctcagggagctctggaatggttaggttacaccccttccccttttgaatttggcatcaaaaggatcagcagacagagtcatggagtgcttggcagtgatggaatattagccaaaggcagagaggtatagcttcaggctcaggctggcttgtgcccagcgtaagtactggaaggaggcctttgacctgagaccagctgagggcacagagagggcctgcccatgtggaacctgccaggttttgtgcgcagattttgctaattgactgatttagtatctcagccctgaataaaggggaatcaaatggatctgatttcagccatctcccctaagagatgcagactcttctccctaaaaactgtttccaggcagccaccagctctcctccccattccttgctgctgtcctatcagtggacagactgccttcttcgctgtcttgctcttggtctcagccatcttgctattgttttttttagcccctgaagaagacagtgcaaccttaccctctatcctgtcttcgccttctcttcctcgaagactttctctgaatagggccatccatcctccaaactgttcccagatccttggttttaaaggtagaatgataacataaggtaacctggtgggcacttacaagatgagccaggcacagggtcaagctttttacctaccctgtctcatctaatcctcatgcagacttacaaggtgtgttacccctgttatacatttactctcattatgcccaatttagatatgaagacgctgagtctcagagaagttaagaaacgtgcccaaggtcacacagctagtgggtagctaggattgaaacttggacagcccagctccagggtacctgcttttgattattttgctatatggctttcctgtaactgcagaccatccaagctggaagggccagggagactggactgtcacactcctgcttaaagaccattgtgggaggccactgtcttcagagtaaagcttggctttccaggctctccttggtagcactacactcctgctttgcacctggagcccggccacaccataaattttccccttctctaaactcactgtgcacattcacagttagggtgtttgatctgcctgacgttgactgccctccttggctgtccctgctggatgcaatcctcaaccttcaaggcttagtggctcctctgagaacacctttctcaggcatgcatcaggagtcctgacttcccccttcgtggttcccagagtactttttcccccttagcacaattcatccttagcatctggatgactgtgagcttcttcggagcaagtagatgttatattttatttgtctttgattctattgttgtccggtccgtagtaggtgctcagtagaggttgtaggattgaattaggctgtccagttcaactctcctatattctagaatttagacaaggaaacaggtgcaaaaagagaagtgacctggccagggtcatgggaaaagttagtgtcagagccaggactagagctcaggtctcctgattctttggattcagagctttttctgttgcacctcagctttgtggagaccagagcatggatctgcacttggtttatgaaggtctgaccaggttcacttcagggtcatggataccactgtgttcccatgctgacactggggaattcccagagggaaaggaagcccagctggagctgctgctgtaggttgcggggctcacctctcaccgagccccttgcttacagagaaaaccagcgagtgattctgaaaatcttgcctcgagtcctcattttgctggtattcaccgtgtgaccttgagcaaattgcttcacatctctgatcttctgttcctttaccaagaaaaatgagggagttgaattcagatggtctccaggagtcatctgattccagaatatcaggatataacaataaagatgaggataaagtagctactcccctcagaaaaggtggagatccagatctagggggagaattggctaggttcttcatcacagtcactgtcttgagacacataggtggtccatggaaacatttccatgagggatttcagaggggagaatgggcagactgcaaagcctcggtactctaataccaggcaaaaagaagaaagggaacaggcattttagttttcacattgaaacagagtgtatgtgtatggccctatgcaaatgttgttcctggtgaatattcatcaatgtactcatcacacataggtatatttagacagaaaggggtcctcatcacagacacccaaattatgagcacattactgcctcctctcccctagaggtgcttcttccagcctcccccattggaggccagcacaagacgggctctccaacccaatatcttccccttccctccttcctttctgctcaggtggctgagtatacacattgaactcttttgtttcaacttcacgtgtatataggttggtgcaaaagtaactgtggtgttttccattaaaagcaatgtcaagaaccgcaattactttggcaccaacctaatatgtatgcaagtacattctttaacctttccaactcccagtccaagccatcaggctaatttccgcgtggaatttgtggtcactggtttgcagtcctttcatttctggtgtggtcagaagtctccttcagactcttcccgataagttgccattggctttttcatcacctttagtacctacccacttttgctgggataacactacccaagataaattcaaaaaaattgtcctaagtgttagtgtgtttctgttttggttgaagtctattattgctatatggttttctactgtttctacttgatggaagactattaagggttaagtaggctggcctaggaagctgaccaccatttataacatacttttgagtgggaaaatgcattcagaggcctgtaatcaatacttaggtggactttgggaatatagtatttattttatgagtgactagaatattggtagaaccatagaaactttgaatgtggagctggaaagcctcctccttcccccactgagatcaattgattaacaagctaggttgtagaaaccaagtgaaagaggcaaaaacttggccaagggcatgtagtcggggacttctgaagattctgctgggctgggggcaggaaaggggtcacagacggtcttagggttgtctcactcagcaactcactgtgttggccttgggaaatgtaagtcactgtgttgggctgtaatggaaatattgagaaagcagctcagctagtggcgtggggagaggttgtctagtcctctggcttatctgtaaattcctggcatgtgcttgttggagcagaatgaatggggctgctgtgttttacctgcagctaggagcatggctggagtggtggagacactccctgtggtctgttctaaaaatagcagtgggaacagagctgaggggaagaggagggggctccttcgggagctgggtggggaggcctcacccccttcctcttcctgccaggcccgatgtgaggaagtcccatggagtcacataattccatctgggagagtcctggagccatcagccctcacaccccctcctcatacaggcgaggaggccctggaggcccggagagcagaaagcactggctggtgtcaagcaagcccagagagaagggcccagttggcaggctgtttttccctggctgtttcagcacagtggctgcaggccttgtgctgaggtttgctgtcactgcctttgggcgcacagagactgggaactcctgggaccggatgtggtcaggcttcctgagagtcgcctggcaggcgaggagtccccagctggcccaccctgagccctgacctcagccagccggctgtccagtgccacacctcctcttgctggtgtcccctcccttgctgacttgtcctcctttctttggactgtctgcagggagggaggacaagggccagcctagatctggcctgcaggacagaggcctcagtggctcaggacctttccctgcccctccccaggaacaagcagaggcagctgaggtagtagcagcctcctgcaggtttagagacagacaggccagggttcaaatcctagctcttcctctcactagccatgggattactgatgggtcacagagcatctgtaaaatgagacaagaacgctgatttcactggggtcttgtgaagatcaaaggggatctgttgtaatgcctggcatgcagtaggtacttaatagtagctcttgttgcaaaaccaagcagtgaacatgtatctgactttctataacccccttccttggggcctccactcatgaggcatgggaaggtgttttttttctttgtttgtttgtttgtttgaatgcagttgactttttttttgttgtattgttgcaaaagtaatccatgtgattgatgtttattatgaagaaaacatttaatacagatgaaccaggagaagaaaatgaagttcatccttaatcccagcacccagagataaaccactgttaacattttggaatgtgtcctgtcaggcattcgtttatacacacatatatcttatttgcaaattggggttactggtgcaatttttctaacctgctttttctcccccatttaacagcgtagtgtatccatgtttccatttcaataaatattcttctacaacatca

SEQ ID NO: 9 - DNA, 2649 bp, Homo sapiens
agtgccagggggtgccgttgtgagggatggacgcctctcccccggagcagcactttggggccagcttgggccctggggatgagccacaggggacccacagcctatgcgtgtgagcatgtaacccgggagccagctgggcccctcgcggggtgggcagaaggacgggctggcccaggaccctcttctttgagacatcccggactccatctcggatgaaagtgctttgagaaaccacagagttttctgtttaatggaagaaagaaatccagcctctctccagagtggcggtggccggctagacaggatgtttgagacggaggcagatgagaagagggagatggccttggaggaagggaaggggcctggtgccgaggattccccacccagcaaggagccctctcctggccaggagcttcctccaggacaagaccttccacccaacaaggactccccttctgggcaggaacccgctcccagccaagaaccactgtccagcaaagactcagctacctctgaaggatcccctccaggcccagatgctccgcccagcaaggatgtgccaccatgccaggaaccccctccagcccaagacctctcaccctgccaggacctacctgctggtcaagaacccctgcctcaccaggaccctctactcaccaaagacctccctgccatccaggaatcccccacccgggaccttccaccctgtcaagatctgcctcctagccaggtctccctgccagccaaggcccttactgaggacaccatgagctccggggacctactagcagctactggggacccacctgcggcccccaggccagccttcgtgatccctgaggtccggctggatagcacctacagccagaaggcaggggcagagcagggctgctcgggagatgaggaggatgcagaagaggccgaggaggtggaggagggggaggaaggggaggaggacgaggatgaggacaccagcgatgacaactacggagagcgcagtgaggccaagcgcagcagcatgatcgagacgggccagggggctgagggtggcctctcactgcgtgtgcagaactcgctgcggcgccggacgcacagcgagggcagcctgctgcaggagccccgagggccctgctttgcctccgacaccaccttgcactgctcagacggtgagggcgccgcctccacctggggcatgccttcgcccagcaccctcaagaaagagctgggccgcaatggtggctccatgcaccacctttccctcttcttcacaggacacaggaagatgagcggggctgacaccgttggggatgatgacgaagcctcccggaagagaaagagcaaaaacctagccaaggacatgaagaacaagctggggatcttcagacggcggaatgagtcccctggagcccctcccgcgggcaaggcagacaaaatgatgaagtcattcaagcccacctcagaggaagccctcaagtggggcgagtccttggagaagctgctggttcacaaatacgggttagcagtgttccaagccttccttcgcactgagttcagtgaggagaatctggagttctggttggcttgtgaggacttcaagaaggtcaagtcacagtccaagatggcatccaaggccaagaagatctttgctgaatacatcgcgatccaggcatgcaaggaggtcaacctggactcctacacgcgggagcacaccaaggacaacctgcagagcgtcacgcggggctgcttcgacctggcacagaagcgcatcttcgggctcatggaaaaggactcgtaccctcgctttctccgttctgacctctacctggaccttattaaccagaagaagatgagtcccccgctttaggggccactggagtcgagctcagcgttcacaccaggcgggctgggtcccctgcccacctgcctccctgccccctgtgacggagggggcaagcaagcccccagaggctgtgtctctggacagacggatagacatacggaagcgaggcctggaccaagagaggcccaggctactggaggagtagaaggatgggccccgtggggtccccactgccccggtacgagggggcccaagaccctggcaggtcaggggccctggccaagccagatctggagctgctgctccctgctgcggagaccgcggaggcttcgcgttgaccaagttccttaaagaactggctgatggggcaggaggtccaggcctgggctctcgggccctcctagagggccattggagcttgcagctcagacccccactttgagttttatttatttaaatagtagttggatgcttggcacgtcgtcctgtaataggaaacccttgcctcatcagttttcctgatttacaagtgcaatattttagccaatgccttgggagaagctgccatgcaaaggtggacaccattctccagcttcaggggatatgctcgtcccgggcaccggtggcaggcagctggccttctggactaaggcagcctggggggacactgcagtctggctacacacagagatctggcaccccctgggtggagtgtccctcgggggctttgggaaagcatggcaccctcagaccacacagtagccaagttctggagcaaataaaaggcctgtgttatttcttgttcttga

SEQ ID NO: 10 - DNA, 3033 bp, Homo sapiens
agtgccagggggtgccgttgtgagggatggacgcctctcccccggagcagcactttggggccagcttgggccctggggatgagccacaggggacccacagcctatgcgtgtgagcatgtaacccgggagccagctgggcccctcgcggggtgggcagaaggacgggctggcccaggaccctcttctttgagacatcccggactccatctcggatgaaagtgctttgagaaaccacagagttttctgtttaatggaagaaagaaatccagcctctctccagagtggcggtggccggctagacagggtatcgctcccctggggaacttgggtaagtccataaatagctctgctgacacaaaggagctctgtctggggaaggcagccgcttgacacggccgccaggagctgccacagccacggccgcccgcctgcgggaggagccgggtggaaacctgggcgggcgagctggcaggagcggcagccaggagtcgcctgggtccctgctggagtatgctggatgccagcgtgacccctgacacgtgtgtgcagcagcctgcagctgccccaagccatggctgaacactgactcccagctgtggggcttcaccattacagactccccagggcttcaaagacttctcagcttcgagcatggcttttggctgtcagggcagctgtacaatagtggatgtttgagacggaggcagatgagaagagggagatggccttggaggaagggaaggggcctggtgccgaggattccccacccagcaaggagccctctcctggccaggagcttcctccaggacaagaccttccacccaacaaggactccccttctgggcaggaacccgctcccagccaagaaccactgtccagcaaagactcagctacctctgaaggatcccctccaggcccagatgctccgcccagcaaggatgtgccaccatgccaggaaccccctccagcccaagacctctcaccctgccaggacctacctgctggtcaagaacccctgcctcaccaggaccctctactcaccaaagacctccctgccatccaggaatcccccacccgggaccttccaccctgtcaagatctgcctcctagccaggtctccctgccagccaaggcccttactgaggacaccatgagctccggggacctactagcagctactggggacccacctgcggcccccaggccagccttcgtgatccctgaggtccggctggatagcacctacagccagaaggcaggggcagagcagggctgctcgggagatgaggaggatgcagaagaggccgaggaggtggaggagggggaggaaggggaggaggacgaggatgaggacaccagcgatgacaactacggagagcgcagtgaggccaagcgcagcagcatgatcgagacgggccagggggctgagggtggcctctcactgcgtgtgcagaactcgctgcggcgccggacgcacagcgagggcagcctgctgcaggagccccgagggccctgctttgcctccgacaccaccttgcactgctcagacggtgagggcgccgcctccacctggggcatgccttcgcccagcaccctcaagaaagagctgggccgcaatggtggctccatgcaccacctttccctcttcttcacaggacacaggaagatgagcggggctgacaccgttggggatgatgacgaagcctcccggaagagaaagagcaaaaacctagccaaggacatgaagaacaagctggggatcttcagacggcggaatgagtcccctggagcccctcccgcgggcaaggcagacaaaatgatgaagtcattcaagcccacctcagaggaagccctcaagtggggcgagtccttggagaagctgctggttcacaaatacgggttagcagtgttccaagccttccttcgcactgagttcagtgaggagaatctggagttctggttggcttgtgaggacttcaagaaggtcaagtcacagtccaagatggcatccaaggccaagaagatctttgctgaatacatcgcgatccaggcatgcaaggaggtcaacctggactcctacacgcgggagcacaccaaggacaacctgcagagcgtcacgcggggctgcttcgacctggcacagaagcgcatcttcgggctcatggaaaaggactcgtaccctcgctttctccgttctgacctctacctggaccttattaaccagaagaagatgagtcccccgctttaggggccactggagtcgagctcagcgttcacaccaggcgggctgggtcccctgcccacctgcctccctgccccctgtgacggagggggcaagcaagcccccagaggctgtgtctctggacagacggatagacatacggaagcgaggcctggaccaagagaggcccaggctactggaggagtagaaggatgggccccgtggggtccccactgccccggtacgagggggcccaagaccctggcaggtcaggggccctggccaagccagatctggagctgctgctccctgctgcggagaccgcggaggcttcgcgttgaccaagttccttaaagaactggctgatggggcaggaggtccaggcctgggctctcgggccctcctagagggccattggagcttgcagctcagacccccactttgagttttatttatttaaatagtagttggatgcttggcacgtcgtcctgtaataggaaacccttgcctcatcagttttcctgatttacaagtgcaatattttagccaatgccttgggagaagctgccatgcaaaggtggacaccattctccagcttcaggggatatgctcgtcccgggcaccggtggcaggcagctggccttctggactaaggcagcctggggggacactgcagtctggctacacacagagatctggcaccccctgggtggagtgtccctcgggggctttgggaaagcatggcaccctcagaccacacagtagccaagttctggagcaaataaaaggcctgtgttatttcttgttcttga

SEQ ID NO: 11 - DNA, 4122 bp, Homo sapiens
ggagactccggttactggggagcaacacagccgcctcgggttgcagacgctcctgtccgggtcgcagtgggacgccatggagcgctccctgcaccgcgtctccctcgggagccggcgtgcccacccggacttgtccttctacctcaccacctttggtcagctgaggctgtccattgatgcccaggaccgggttctgctgcttcacattatagaaggtaaaggcctgatcagcaaacagcctggcacctgtgatccgtatgtgaagatttctttgatccctgaagatagtagactacgccaccagaagacgcagaccgttccagactgcagagacccggctttccacgagcacttcttctttcctgtccaagaggaggatgatcagaagcgtctcttggttactgtgtggaacagggccagccagtccagacagagtggactcattggctgcatgagctttggggtgaagtctctcctgactccagacaaggagatcagtggttggtactacctcctaggggagcacctgggccggaccaagcacttgaaggtggccaggcggcgactgcggccgctgagagacccgctgctgagaatgccaggaggtggggacactgagaatgggaagaaactaaagatcaccatcccgaggggaaaggacggctttggcttcaccatctgctgcgactctccagttcgagtccaggccgtggattccgggggtccggcggaacgggcagggctgcagcagctggacacggtgctgcagctgaatgagaggcctgtggagcactggaaatgtgtggagctggcccacgagatccggagctgccccagtgagatcatcctactcgtgtggcgcatggtcccccaggtcaagccaggaccagatggcggggtcctgcggcgggcctcctgcaagtcgacacatgacctccagtcaccccccaacaaacgggagaagaactgcacccatggggtccaggcacggcctgagcagcgccacagctgccacctggtatgtgacagctctgatgggctgctgctcggcggctgggagcgctacaccgaggtggccaagcgcgggggccagcacaccctgcctgcactgtcccgtgccactgcccccaccgaccccaactacatcatcctggccccgctgaatcctgggagccagctgctccggcctgtgtaccaggaggataccatccccgaagaatcagggagtcccagtaaagggaagtcctacacaggcctggggaagaagtcccggctgatgaagacagtgcagaccatgaagggccacgggaactaccaaaactgcccggttgtgaggccgcatgccacgcactcaagctatggcacctacgtcaccctggcccccaaagtcctggtgttccctgtctttgttcagcctctagatctctgtaatcctgcccggaccctcctgctgtcagaggagctgctgctgtatgaagggaggaacaaggctgccgaggtgacactgtttgcctattcggacctgctgctcttcaccaaggaggacgagcctggccgctgcgacgtcctgaggaaccccctctacctccagagtgtgaagctgcaggaaggttcttcagaagacctgaaattctgcgtgctctatctagcagagaaggcagagtgcttattcactttggaagcgcactcgcaggagcagaagaagagagtgtgctggtgcctgtcggagaacatcgccaagcagcaacagctggcagcatcacccccggacagcaagatgtttgagacggaggcagatgagaagagggagatggccttggaggaagggaaggggcctggtgccgaggattccccacccagcaaggagccctctcctggccaggagcttcctccaggacaagaccttccacccaacaaggactccccttctgggcaggaacccgctcccagccaagaaccactgtccagcaaagactcagctacctctgaaggatcccctccaggcccagatgctccgcccagcaaggatgtgccaccatgccaggaaccccctccagcccaagacctctcaccctgccaggacctacctgctggtcaagaacccctgcctcaccaggaccctctactcaccaaagacctccctgccatccaggaatcccccacccgggaccttccaccctgtcaagatctgcctcctagccaggtctccctgccagccaaggcccttactgaggacaccatgagctccggggacctactagcagctactggggacccacctgcggcccccaggccagccttcgtgatccctgaggtccggctggatagcacctacagccagaaggcaggggcagagcagggctgctcgggagatgaggaggatgcagaagaggccgaggaggtggaggagggggaggaaggggaggaggacgaggatgaggacaccagcgatgacaactacggagagcgcagtgaggccaagcgcagcagcatgatcgagacgggccagggggctgagggtggcctctcactgcgtgtgcagaactcgctgcggcgccggacgcacagcgagggcagcctgctgcaggagccccgagggccctgctttgcctccgacaccaccttgcactgctcagacggtgagggcgccgcctccacctggggcatgccttcgcccagcaccctcaagaaagagctgggccgcaatggtggctccatgcaccacctttccctcttcttcacaggacacaggaagatgagcggggctgacaccgttggggatgatgacgaagcctcccggaagagaaagagcaaaaacctagccaaggacatgaagaacaagctggggatcttcagacggcggaatgagtcccctggagcccctcccgcgggcaaggcagacaaaatgatgaagtcattcaagcccacctcagaggaagccctcaagtggggcgagtccttggagaagctgctggttcacaaatacgggttagcagtgttccaagccttccttcgcactgagttcagtgaggagaatctggagttctggttggcttgtgaggacttcaagaaggtcaagtcacagtccaagatggcatccaaggccaagaagatctttgctgaatacatcgcgatccaggcatgcaaggaggtcaacctggactcctacacgcgggagcacaccaaggacaacctgcagagcgtcacgcggggctgcttcgacctggcacagaagcgcatcttcgggctcatggaaaaggactcgtaccctcgctttctccgttctgacctctacctggaccttattaaccagaagaagatgagtcccccgctttaggggccactggagtcgagctcagcgttcacaccaggcgggctgggtcccctgcccacctgcctccctgccccctgtgacggagggggcaagcaagcccccagaggctgtgtctctggacagacggatagacatacggaagcgaggcctggaccaagagaggcccaggctactggaggagtagaaggatgggccccgtggggtccccactgccccggtacgagggggcccaagaccctggcaggtcaggggccctggccaagccagatctggagctgctgctccctgctgcggagaccgcggaggcttcgcgttgaccaagttccttaaagaactggctgatggggcaggaggtccaggcctgggctctcgggccctcctagagggccattggagcttgcagctcagacccccactttgagttttatttatttaaatagtagttggatgcttggcacgtcgtcctgtaataggaaacccttgcctcatcagttttcctgatttacaagtgcaatattttagccaatgccttgggagaagctgccatgcaaaggtggacaccattctccagcttcaggggatatgctcgtcccgggcaccggtggcaggcagctggccttctggactaaggcagcctggggggacactgcagtctggctacacacagagatctggcaccccctgggtggagtgtccctcgggggctttgggaaagcatggcaccctcagaccacacagtagccaagttctggagcaaataaaaggcctgtgttatttcttgttcttga

SEQ ID NO: 12 - DNA, 10226 bp, Homo sapiens
ggagactccggttactggggagcaacacagccgcctcgggttgcagacgctcctgtccgggtcgcagtgggacgccatggagcgctccctgcaccgcgtctccctcgggagccggcgtgcccacccggacttgtccttctacctcaccacctttggtcagctgaggctgtccattgatgcccaggaccgggttctgctgcttcacattatagaaggtaaaggcctgatcagcaaacagcctggcacctgtgatccgtatgtgaagatttctttgatccctgaagatagtagactacgccaccagaagacgcagaccgttccagactgcagagacccggctttccacgagcacttcttctttcctgtccaagaggaggatgatcagaagcgtctcttggttactgtgtggaacagggccagccagtccagacagagtggactcattggctgcatgagctttggggtgaagtctctcctgactccagacaaggagatcagtggttggtactacctcctaggggagcacctgggccggaccaagcacttgaaggtggccaggcggcgactgcggccgctgagagacccgctgctgagaatgccaggaggtggggacactgagaatgggaagaaactaaagatcaccatcccgaggggaaaggacggctttggcttcaccatctgctgcgactctccagttcgagtccaggccgtggattccgggggtccggcggaacgggcagggctgcagcagctggacacggtgctgcagctgaatgagaggcctgtggagcactggaaatgtgtggagctggcccacgagatccggagctgccccagtgagatcatcctactcgtgtggcgcatggtcccccaggtcaagccaggaccagatggcggggtcctgcggcgggcctcctgcaagtcgacacatgacctccagtcaccccccaacaaacgggagaagaactgcacccatggggtccaggcacggcctgagcagcgccacagctgccacctggtatgtgacagctctgatgggctgctgctcggcggctgggagcgctacaccgaggtggccaagcgcgggggccagcacaccctgcctgcactgtcccgtgccactgcccccaccgaccccaactacatcatcctggccccgctgaatcctgggagccagctgctccggcctgtgtaccaggaggataccatccccgaagaatcagggagtcccagtaaagggaagtcctacacaggcctggggaagaagtcccggctgatgaagacagtgcagaccatgaagggccacgggaactaccaaaactgcccggttgtgaggccgcatgccacgcactcaagctatggcacctacgtcaccctggcccccaaagtcctggtgttccctgtctttgttcagcctctagatctctgtaatcctgcccggaccctcctgctgtcagaggagctgctgctgtatgaagggaggaacaaggctgccgaggtgacactgtttgcctattcggacctgctgctcttcaccaaggaggacgagcctggccgctgcgacgtcctgaggaaccccctctacctccagagtgtgaagctgcaggaaggttcttcagaagacctgaaattctgcgtgctctatctagcagagaaggcagagtgcttattcactttggaagcgcactcgcaggagcagaagaagagagtgtgctggtgcctgtcggagaacatcgccaagcagcaacagctggcagcatcacccccggacagcaagaaactccaccctttcggctctctccagcaggagatggggccggtcaactcaaccaatgccacccaggatagaagctttacctcaccaggacagactctgattggctgagcaaacccaagggcgggactctgcttctggcaacttaaccctttcttgggcatcccctaccacacagtaacctcacctcaactggacctgaaacagaggaccaagatggtgggtctagggtcatctgaagggagtggtcccaagggtgcatgtggatgtcaacagaaggaccacaatggtgtgagaggccttgtgggcatgcagagactggcagcagaaagtcccaagaggagaggactcagaggcttcttccttccaaggaggaggaggaggaggacattccacatctgggcttccagtggtggctcccggacctgccttgggggcagcaggagaggttgggagaggtcaccctgatgaagaacaatgccatgatctgctgctgccagaggacatctccgcccatccactcattttccacctgtacacattggctcattcattcgatggatatttcgggtgcatctactttatgccaggctctatgctaggagctggggatactgtggagaaaaagtcggatatggtctcgctctcatcaagatttcagtctagctgggagtgagcgttaaataagtcaataccaacacacacacgtgcacacacgtgatggcaagctgtgatcatctatgaaagaaaaaacacagatttctttaagagggaattgtacttagagtgctgtgtgtgggggtggggatggggaaggacataacttttaaaccgagacttgtgggctggagaggaaggagccagtcctggacttggggaagagtgttcgcggtggatggaatagcacgtggagagtccctgagatgggaaggagctcagtgggctggaggaatggcgagcaggccagtgtggctggagcttagtgaggtgcaagacacagtgggtgaggtagttgatgtcttgttgatttcattctagatacactggagagccattgaaggcttttgaggaagggagttgcaagtacatttttacaagatccgtttacacataggaggggcccaagtgggtgtgagatcagttagggggctcgttcagttctctaggtgaggactgtcctggtggcttgtcctggatggtggctggggaagtggatgcctgaatgccaggctctggagaagagatgactccctgccccatccccctgcctaccctcctcctcaatggcccctctctcagtggatgtgagtgaggattaggaccttccccacagagccaccttttggccaggcctcgtgggggatagtgggggcagtgctgagttctaggagtgttcctggaagttgggggcagtgagtgctcagcctggcccaggtgggagagttcactgctgcccaggccttagcaggggctgctgtgctgtctttctgtcccatgctgagcattgtgataggaagctgccacacagtgccctggaaactgacttcgcatgctttccatggggcttccgagggccagggactgggctgggggtggcctagcagctctagtggagactgtgctgccgcagggctgaaggaagaccaaccaaggggacagccgtttgagcccagcccactcactgcctgctgcacctccagctggagcccagggttgggcagctctcatactcttatctaactgccagacctagagggagggaagcccaaggctgcaggtctgagggacacggctcatgggcaaaagggtatcaagaggattagagtcacttgcataggaaaagagggtcagagaggctgagggctggctgaggccagagaggttccctgaccttggacattctttgtgttgtgtaagacctcggcatctttagagaaaggttcttgtgcccctgtgagcttgctcagctgctatggccacttgaaccccaaaaatggtggcctagaagagaatacaccccttacttcccacttatcctcctctcaacctaggaaagatcagacatattgactcaacagtctaacaggggatgggctggatctgcccgtgaccagaccaatgccccccacccagcaagggagtaaacgctgcctgaagtgggaccagtgatacacagcatctctgtctgccagggctcagcccgcgggggggatttgtgagaggctgagcagctgggtctgcacagacagcaagctgcactttgatttcatttgattcccttacaaaatgacattaaggggaatgtgaggcacaggagactcgcctgtctgggaaggcagcaagctcatctctcctggacctggtgacctacctctgtctaccagacaggtgcagaggacaggagctctggccacaaagaaatggacagtctgaatgcagcacacagccctggggacaggacttttcacccagcctgtcctgacaagctgctgagtcgagcagatgctcacatcagggtcccaggtccggctgttagggaagtggacatcagttcctggtgggccctgggcagtgactgtggtgagccacagcaccattgctgctctgcctctcaaatcgatcgaacaattaatttggctccacaggctcgagaagatgaaacatgacctgatggccactgctgtccagcatcccagtccatcctagctgtccaggatgatgaacaagctatggaaattgcacttgagagctataatcacgggaagcattgccgagcccctccctgctacataaatcatgctaagaagcacacaatggcctctttcctgatgggctaattgtcttggtttggtgtctcaggcccaggagctgggaagatgggagcacaaccatttattgttggaaagaccatctgtgcaacttgaatacaaagtgattcttttagttccccacacacccccgaccctctgatcccattttcatgttttttttttttttttctttttatgtctcaggctctggttcttttaaaactcttcatctatcctgtaagccatcaggcctggatctctgctggcctgggtcttttggctatttgggggttgggacattgcagaccttttggaaataatgacatttatttatatatgtcttatatttttattctttatttttccccataaattctattaccaaagtcctcatttgtcacacatggcaccaaggcaccaagagggtttttgcaaagtgaaaatgcaggcaattttgcaacgtggaagggctccccacagcgaaagtcccacagtgagagcctaaactttggagatggcaactggggtccccaggcatgatctccccccagcctcccagcagccttgttttatggatgaaagacaccaaggctctgagaagttaagtgttttatcctaggtcacacagctggtaagggccaagcttgccctcaagccaaggcctttgaacctaatctaccactcatttctgataaaaaggtttgtcttttagggatagaagtaagagaaatcaataatctccaggaagaggcaagagagggatatccggcatgtcagaagctgtggcccatcaagctgtagagggaatgcttttaggataaataaaatgagatgccttattttccacccagtggagaataaacttccagaatgcattttcctgagggtggcacaggctgaaaagatcaatagcttcccacaagatttgaaaaaagcctaaaatgagagatcagtttgagctccttaagacaggttctcagggagctctggaatggttaggttacaccccttccccttttgaatttggcatcaaaaggatcagcagacagagtcatggagtgcttggcagtgatggaatattagccaaaggcagagaggtatagcttcaggctcaggctggcttgtgcccagcgtaagtactggaaggaggcctttgacctgagaccagctgagggcacagagagggcctgcccatgtggaacctgccaggttttgtgcgcagattttgctaattgactgatttagtatctcagccctgaataaaggggaatcaaatggatctgatttcagccatctcccctaagagatgcagactcttctccctaaaaactgtttccaggcagccaccagctctcctccccattccttgctgctgtcctatcagtggacagactgccttcttcgctgtcttgctcttggtctcagccatcttgctattgttttttttagcccctgaagaagacagtgcaaccttaccctctatcctgtcttcgccttctcttcctcgaagactttctctgaatagggccatccatcctccaaactgttcccagatccttggttttaaaggtagaatgataacataaggtaacctggtgggcacttacaagatgagccaggcacagggtcaagctttttacctaccctgtctcatctaatcctcatgcagacttacaaggtgtgttacccctgttatacatttactctcattatgcccaatttagatatgaagacgctgagtctcagagaagttaagaaacgtgcccaaggtcacacagctagtgggtagctaggattgaaacttggacagcccagctccagggtacctgcttttgattattttgctatatggctttcctgtaactgcagaccatccaagctggaagggccagggagactggactgtcacactcctgcttaaagaccattgtgggaggccactgtcttcagagtaaagcttggctttccaggctctccttggtagcactacactcctgctttgcacctggagcccggccacaccataaattttccccttctctaaactcactgtgcacattcacagttagggtgtttgatctgcctgacgttgactgccctccttggctgtccctgctggatgcaatcctcaaccttcaaggcttagtggctcctctgagaacacctttctcaggcatgcatcaggagtcctgacttcccccttcgtggttcccagagtactttttcccccttagcacaattcatccttagcatctggatgactgtgagcttcttcggagcaagtagatgttatattttatttgtctttgattctattgttgtccggtccgtagtaggtgctcagtagaggttgtaggattgaattaggctgtccagttcaactctcctatattctagaatttagacaaggaaacaggtgcaaaaagagaagtgacctggccagggtcatgggaaaagttagtgtcagagccaggactagagctcaggtctcctgattctttggattcagagctttttctgttgcacctcagctttgtggagaccagagcatggatctgcacttggtttatgaaggtctgaccaggttcacttcagggtcatggataccactgtgttcccatgctgacactggggaattcccagagggaaaggaagcccagctggagctgctgctgtaggttgcggggctcacctctcaccgagccccttgcttacagagaaaaccagcgagtgattctgaaaatcttgcctcgagtcctcattttgctggtattcaccgtgtgaccttgagcaaattgcttcacatctctgatcttctgttcctttaccaagaaaaatgagggagttgaattcagatggtctccaggagtcatctgattccagaatatcaggatataacaataaagatgaggataaagtagctactcccctcagaaaaggtggagatccagatctagggggagaattggctaggttcttcatcacagtcactgtcttgagacacataggtggtccatggaaacatttccatgagggatttcagaggggagaatgggcagactgcaaagcctcggtactctaataccaggcaaaaagaagaaagggaacaggcattttagttttcacattgaaacagagtgtatgtgtatggccctatgcaaatgttgttcctggtgaatattcatcaatgtactcatcacacataggtatatttagacagaaaggggtcctcatcacagacacccaaattatgagcacattactgcctcctctcccctagaggtgcttcttccagcctcccccattggaggccagcacaagacgggctctccaacccaatatcttccccttccctccttcctttctgctcaggtggctgagtatacacattgaactcttttgtttcaacttcacgtgtatataggttggtgcaaaagtaactgtggtgttttccattaaaagcaatgtcaagaaccgcaattactttggcaccaacctaatatgtatgcaagtacattctttaacctttccaactcccagtccaagccatcaggctaatttccgcgtggaatttgtggtcactggtttgcagtcctttcatttctggtgtggtcagaagtctccttcagactcttcccgataagttgccattggctttttcatcacctttagtacctacccacttttgctgggataacactacccaagataaattcaaaaaaattgtcctaagtgttagtgtgtttctgttttggttgaagtctattattgctatatggttttctactgtttctacttgatggaagactattaagggttaagtaggctggcctaggaagctgaccaccatttataacatacttttgagtgggaaaatgcattcagaggcctgtaatcaatacttaggtggactttgggaatatagtatttattttatgagtgactagaatattggtagaaccatagaaactttgaatgtggagctggaaagcctcctccttcccccactgagatcaattgattaacaagctaggttgtagaaaccaagtgaaagaggcaaaaacttggccaagggcatgtagtcggggacttctgaagattctgctgggctgggggcaggaaaggggtcacagacggtcttagggttgtctcactcagcaactcactgtgttggccttgggaaatgtaagtcactgtgttgggctgtaatggaaatattgagaaagcagctcagctagtggcgtggggagaggttgtctagtcctctggcttatctgtaaattcctggcatgtgcttgttggagcagaatgaatggggctgctgtgttttacctgcagctaggagcatggctggagtggtggagacactccctgtggtctgttctaaaaatagcagtgggaacagagctgaggggaagaggagggggctccttcgggagctgggtggggaggcctcacccccttcctcttcctgccaggcccgatgtgaggaagtcccatggagtcacataattccatctgggagagtcctggagccatcagccctcacaccccctcctcatacaggcgaggaggccctggaggcccggagagcagaaagcactggctggtgtcaagcaagcccagagagaagggcccagttggcaggctgtttttccctggctgtttcagcacagtggctgcaggccttgtgctgaggtttgctgtcactgcctttgggcgcacagagactgggaactcctgggaccggatgtggtcaggcttcctgagagtcgcctggcaggcgaggagtccccagctggcccaccctgagccctgacctcagccagccggctgtccagtgccacacctcctcttgctggtgtcccctcccttgctgacttgtcctcctttctttggactgtctgcagggagggaggacaagggccagcctagatctggcctgcaggacagaggcctcagtggctcaggacctttccctgcccctccccaggaacaagcagaggcagctgaggtagtagcagcctcctgcaggtttagagacagacaggccagggttcaaatcctagctcttcctctcactagccatgggattactgatgggtcacagagcatctgtaaaatgagacaagaacgctgatttcactggggtcttgtgaagatcaaaggggatctgttgtaatgcctggcatgcagtaggtacttaatagtagctcttgttgcaaaaccaagcagtgaacatgtatctgactttctataacccccttccttggggcctccactcatgaggcatgggaaggtgttttttttctttgtttgtttgtttgtttgaatgcagttgactttttttttgttgtattgttgcaaaagtaatccatgtgattgatgtttattatgaagaaaacatttaatacagatgaaccaggagaagaaaatgaagttcatccttaatcccagcacccagagataaaccactgttaacattttggaatgtgtcctgtcaggcattcgtttatacacacatatatcttatttgcaaattggggttactggtgcaatttttctaacctgctttttctcccccatttaacagcgtagtgtatccatgtttccatttcaataaatattcttctacaacatca

SEQ ID NO: 13 - DNA, 3609 bp, Homo sapiens
agtgcgggctcccgctgctgggggagagctgggttttcatggggcggcagccgaggcaggacccgcagccatgaaccgcttcaatgggctctgcaaggtgtgctcggagcgccgctaccgccagatcaccatcccgaggggaaaggacggctttggcttcaccatctgctgcgactctccagttcgagtccaggccgtggattccgggggtccggcggaacgggcagggctgcagcagctggacacggtgctgcagctgaatgagaggcctgtggagcactggaaatgtgtggagctggcccacgagatccggagctgccccagtgagatcatcctactcgtgtggcgcatggtcccccaggtcaagccaggaccagatggcggggtcctgcggcgggcctcctgcaagtcgacacatgacctccagtcaccccccaacaaacgggagaagaactgcacccatggggtccaggcacggcctgagcagcgccacagctgccacctggtatgtgacagctctgatgggctgctgctcggcggctgggagcgctacaccgaggtggccaagcgcgggggccagcacaccctgcctgcactgtcccgtgccactgcccccaccgaccccaactacatcatcctggccccgctgaatcctgggagccagctgctccggcctgtgtaccaggaggataccatccccgaagaatcagggagtcccagtaaagggaagtcctacacaggcctggggaagaagtcccggctgatgaagacagtgcagaccatgaagggccacgggaactaccaaaactgcccggttgtgaggccgcatgccacgcactcaagctatggcacctacgtcaccctggcccccaaagtcctggtgttccctgtctttgttcagcctctagatctctgtaatcctgcccggaccctcctgctgtcagaggagctgctgctgtatgaagggaggaacaaggctgccgaggtgacactgtttgcctattcggacctgctgctcttcaccaaggaggacgagcctggccgctgcgacgtcctgaggaaccccctctacctccagagtgtgaagctgcaggaaggttcttcagaagacctgaaattctgcgtgctctatctagcagagaaggcagagtgcttattcactttggaagcgcactcgcaggagcagaagaagagagtgtgctggtgcctgtcggagaacatcgccaagcagcaacagctggcagcatcacccccggacagcaagatgtttgagacggaggcagatgagaagagggagatggccttggaggaagggaaggggcctggtgccgaggattccccacccagcaaggagccctctcctggccaggagcttcctccaggacaagaccttccacccaacaaggactccccttctgggcaggaacccgctcccagccaagaaccactgtccagcaaagactcagctacctctgaaggatcccctccaggcccagatgctccgcccagcaaggatgtgccaccatgccaggaaccccctccagcccaagacctctcaccctgccaggacctacctgctggtcaagaacccctgcctcaccaggaccctctactcaccaaagacctccctgccatccaggaatcccccacccgggaccttccaccctgtcaagatctgcctcctagccaggtctccctgccagccaaggcccttactgaggacaccatgagctccggggacctactagcagctactggggacccacctgcggcccccaggccagccttcgtgatccctgaggtccggctggatagcacctacagccagaaggcaggggcagagcagggctgctcgggagatgaggaggatgcagaagaggccgaggaggtggaggagggggaggaaggggaggaggacgaggatgaggacaccagcgatgacaactacggagagcgcagtgaggccaagcgcagcagcatgatcgagacgggccagggggctgagggtggcctctcactgcgtgtgcagaactcgctgcggcgccggacgcacagcgagggcagcctgctgcaggagccccgagggccctgctttgcctccgacaccaccttgcactgctcagacggtgagggcgccgcctccacctggggcatgccttcgcccagcaccctcaagaaagagctgggccgcaatggtggctccatgcaccacctttccctcttcttcacaggacacaggaagatgagcggggctgacaccgttggggatgatgacgaagcctcccggaagagaaagagcaaaaacctagccaaggacatgaagaacaagctggggatcttcagacggcggaatgagtcccctggagcccctcccgcgggcaaggcagacaaaatgatgaagtcattcaagcccacctcagaggaagccctcaagtggggcgagtccttggagaagctgctggttcacaaatacgggttagcagtgttccaagccttccttcgcactgagttcagtgaggagaatctggagttctggttggcttgtgaggacttcaagaaggtcaagtcacagtccaagatggcatccaaggccaagaagatctttgctgaatacatcgcgatccaggcatgcaaggaggtcaacctggactcctacacgcgggagcacaccaaggacaacctgcagagcgtcacgcggggctgcttcgacctggcacagaagcgcatcttcgggctcatggaaaaggactcgtaccctcgctttctccgttctgacctctacctggaccttattaaccagaagaagatgagtcccccgctttaggggccactggagtcgagctcagcgttcacaccaggcgggctgggtcccctgcccacctgcctccctgccccctgtgacggagggggcaagcaagcccccagaggctgtgtctctggacagacggatagacatacggaagcgaggcctggaccaagagaggcccaggctactggaggagtagaaggatgggccccgtggggtccccactgccccggtacgagggggcccaagaccctggcaggtcaggggccctggccaagccagatctggagctgctgctccctgctgcggagaccgcggaggcttcgcgttgaccaagttccttaaagaactggctgatggggcaggaggtccaggcctgggctctcgggccctcctagagggccattggagcttgcagctcagacccccactttgagttttatttatttaaatagtagttggatgcttggcacgtcgtcctgtaataggaaacccttgcctcatcagttttcctgatttacaagtgcaatattttagccaatgccttgggagaagctgccatgcaaaggtggacaccattctccagcttcaggggatatgctcgtcccgggcaccggtggcaggcagctggccttctggactaaggcagcctggggggacactgcagtctggctacacacagagatctggcaccccctgggtggagtgtccctcgggggctttgggaaagcatggcaccctcagaccacacagtagccaagttctggagcaaataaaaggcctgtgttatttcttgttcttga

SEQ ID NO: 14 - DNA, 1606 bp, Homo sapiens
agtgccagggggtgccgttgtgagggatggacgcctctcccccggagcagcactttggggccagcttgggccctggggatgagccacaggggacccacagcctatgcgtgtgagcatgtaacccgggagccagctgggcccctcgcggggtgggcagaaggacgggctggcccaggaccctcttctttgagacatcccggactccatctcggatgaaagtgctttgagaaaccacagagttttctgtttaatggaagaaagaaatccagcctctctccagagtggcggtggccggctagacaggagccaaggacatgaagaacaagctggggatcttcagacggcggaatgagtcccctggagcccctcccgcgggcaaggcagacaaaatgatgaagtcattcaagcccacctcagaggaagccctcaagtggggcgagtccttggagaagctgctggttcacaaatacgggttagcagtgttccaagccttccttcgcactgagttcagtgaggagaatctggagttctggttggcttgtgaggacttcaagaaggtcaagtcacagtccaagatggcatccaaggccaagaagatctttgctgaatacatcgcgatccaggcatgcaaggaggtcaacctggactcctacacgcgggagcacaccaaggacaacctgcagagcgtcacgcggggctgcttcgacctggcacagaagcgcatcttcgggctcatggaaaaggactcgtaccctcgctttctccgttctgacctctacctggaccttattaaccagaagaagatgagtcccccgctttaggggccactggagtcgagctcagcgttcacaccaggcgggctgggtcccctgcccacctgcctccctgccccctgtgacggagggggcaagcaagcccccagaggctgtgtctctggacagacggatagacatacggaagcgaggcctggaccaagagaggcccaggctactggaggagtagaaggatgggccccgtggggtccccactgccccggtacgagggggcccaagaccctggcaggtcaggggccctggccaagccagatctggagctgctgctccctgctgcggagaccgcggaggcttcgcgttgaccaagttccttaaagaactggctgatggggcaggaggtccaggcctgggctctcgggccctcctagagggccattggagcttgcagctcagacccccactttgagttttatttatttaaatagtagttggatgcttggcacgtcgtcctgtaataggaaacccttgcctcatcagttttcctgatttacaagtgcaatattttagccaatgccttgggagaagctgccatgcaaaggtggacaccattctccagcttcaggggatatgctcgtcccgggcaccggtggcaggcagctggccttctggactaaggcagcctggggggacactgcagtctggctacacacagagatctggcaccccctgggtggagtgtccctcgggggctttgggaaagcatggcaccctcagaccacacagtagccaagttctggagcaaataaaaggcctgtgttatttcttgttcttga

SEQ ID NO: 15 - DNA, 4592 bp, Homo sapiens
gaactttggctcagcctgtcttcatccactgtatcctgacacctctcctactccttctctttaatgtacagctgagactgggatacccttagactgaaaggaaggacacggctatgtagaaaagaattgactgaaggggaggagtactcgtgaaccccttgcttcagcctcccaatgttctgagagaggaggagccaggcgcagtggctcatgcctgtaatcccagcactttgggaggttgagatgggcagatcgcaaggtcaggagatcgagaccatcctggctaacaggtcacattcagacagcacacctttgcccaattttctttctggatctcaccgtcctgagtgttgtacctgcaggttgctcacagcctctggagcccaagatagtctcccctttgggaggaggctctacagtggtccctggcgaagttgtgaagaggtctgccacgtctctgtgctcagtgtcctctctacatcctgtggcttgagcctgagcttgcccatattccctggctggatggagtggctaagccctgatatcgctctgcccagaagagatgagtggactcaaacttctccagccaggaagaggatcacgcatgccaaagtccagggtgcaggtcagctgaggctgtccattgatgcccaggaccgggttctgctgcttcacattatagaaggtaaaggcctgatcagcaaacagcctggcacctgtgatccgtatgtgaagatttctttgatccctgaagatagtagactacgccaccagaagacgcagaccgttccagactgcagagacccggctttccacgagcacttcttctttcctgtccaagaggaggatgatcagaagcgtctcttggttactgtgtggaacagggccagccagtccagacagagtggactcattggctgcatgagctttggggtgaagtctctcctgactccagacaaggagatcagtggttggtactacctcctaggggagcacctgggccggaccaagcacttgaaggtggccaggcggcgactgcggccgctgagagacccgctgctgagaatgccaggaggtggggacactgagaatgggaagaaactaaagatcaccatcccgaggggaaaggacggctttggcttcaccatctgctgcgactctccagttcgagtccaggccgtggattccgggggtccggcggaacgggcagggctgcagcagctggacacggtgctgcagctgaatgagaggcctgtggagcactggaaatgtgtggagctggcccacgagatccggagctgccccagtgagatcatcctactcgtgtggcgcatggtcccccaggtcaagccaggaccagatggcggggtcctgcggcgggcctcctgcaagtcgacacatgacctccagtcaccccccaacaaacgggagaagaactgcacccatggggtccaggcacggcctgagcagcgccacagctgccacctggtatgtgacagctctgatgggctgctgctcggcggctgggagcgctacaccgaggtggccaagcgcgggggccagcacaccctgcctgcactgtcccgtgccactgcccccaccgaccccaactacatcatcctggccccgctgaatcctgggagccagctgctccggcctgtgtaccaggaggataccatccccgaagaatcagggagtcccagtaaagggaagtcctacacaggcctggggaagaagtcccggctgatgaagacagtgcagaccatgaagggccacgggaactaccaaaactgcccggttgtgaggccgcatgccacgcactcaagctatggcacctacgtcaccctggcccccaaagtcctggtgttccctgtctttgttcagcctctagatctctgtaatcctgcccggaccctcctgctgtcagaggagctgctgctgtatgaagggaggaacaaggctgccgaggtgacactgtttgcctattcggacctgctgctcttcaccaaggaggacgagcctggccgctgcgacgtcctgaggaaccccctctacctccagagtgtgaagctgcaggaaggttcttcagaagacctgaaattctgcgtgctctatctagcagagaaggcagagtgcttattcactttggaagcgcactcgcaggagcagaagaagagagtgtgctggtgcctgtcggagaacatcgccaagcagcaacagctggcagcatcacccccggacagcaagatgtttgagacggaggcagatgagaagagggagatggccttggaggaagggaaggggcctggtgccgaggattccccacccagcaaggagccctctcctggccaggagcttcctccaggacaagaccttccacccaacaaggactccccttctgggcaggaacccgctcccagccaagaaccactgtccagcaaagactcagctacctctgaaggatcccctccaggcccagatgctccgcccagcaaggatgtgccaccatgccaggaaccccctccagcccaagacctctcaccctgccaggacctacctgctggtcaagaacccctgcctcaccaggaccctctactcaccaaagacctccctgccatccaggaatcccccacccgggaccttccaccctgtcaagatctgcctcctagccaggtctccctgccagccaaggcccttactgaggacaccatgagctccggggacctactagcagctactggggacccacctgcggcccccaggccagccttcgtgatccctgaggtccggctggatagcacctacagccagaaggcaggggcagagcagggctgctcgggagatgaggaggatgcagaagaggccgaggaggtggaggagggggaggaaggggaggaggacgaggatgaggacaccagcgatgacaactacggagagcgcagtgaggccaagcgcagcagcatgatcgagacgggccagggggctgagggtggcctctcactgcgtgtgcagaactcgctgcggcgccggacgcacagcgagggcagcctgctgcaggagccccgagggccctgctttgcctccgacaccaccttgcactgctcagacggtgagggcgccgcctccacctggggcatgccttcgcccagcaccctcaagaaagagctgggccgcaatggtggctccatgcaccacctttccctcttcttcacaggacacaggaagatgagcggggctgacaccgttggggatgatgacgaagcctcccggaagagaaagagcaaaaacctagccaaggacatgaagaacaagctggggatcttcagacggcggaatgagtcccctggagcccctcccgcgggcaaggcagacaaaatgatgaagtcattcaagcccacctcagaggaagccctcaagtggggcgagtccttggagaagctgctggttcacaaatacgggttagcagtgttccaagccttccttcgcactgagttcagtgaggagaatctggagttctggttggcttgtgaggacttcaagaaggtcaagtcacagtccaagatggcatccaaggccaagaagatctttgctgaatacatcgcgatccaggcatgcaaggaggtcaacctggactcctacacgcgggagcacaccaaggacaacctgcagagcgtcacgcggggctgcttcgacctggcacagaagcgcatcttcgggctcatggaaaaggactcgtaccctcgctttctccgttctgacctctacctggaccttattaaccagaagaagatgagtcccccgctttaggggccactggagtcgagctcagcgttcacaccaggcgggctgggtcccctgcccacctgcctccctgccccctgtgacggagggggcaagcaagcccccagaggctgtgtctctggacagacggatagacatacggaagcgaggcctggaccaagagaggcccaggctactggaggagtagaaggatgggccccgtggggtccccactgccccggtacgagggggcccaagaccctggcaggtcaggggccctggccaagccagatctggagctgctgctccctgctgcggagaccgcggaggcttcgcgttgaccaagttccttaaagaactggctgatggggcaggaggtccaggcctgggctctcgggccctcctagagggccattggagcttgcagctcagacccccactttgagttttatttatttaaatagtagttggatgcttggcacgtcgtcctgtaataggaaacccttgcctcatcagttttcctgatttacaagtgcaatattttagccaatgccttgggagaagctgccatgcaaaggtggacaccattctccagcttcaggggatatgctcgtcccgggcaccggtggcaggcagctggccttctggactaaggcagcctggggggacactgcagtctggctacacacagagatctggcaccccctgggtggagtgtccctcgggggctttgggaaagcatggcaccctcagaccacacagtagccaagttctggagcaaataaaaggcctgtgttatttcttgttcttga

SEQ ID NO: 16 - DNA, 1512 bp, Homo sapiens
agtcatcaggccaggattccagagagcgtgtgtggctgcagcctgcaccgttgctgcccgctgcccaggacgcggggtgggggacaggagccagagtggtgcctcctacagaccaatctgcggccccaaggtggggggccctacagagatgctccgaggcatgtacctcactcgcaacgggaacctgcagaggcgacacacgatgaaggaagccaaggacatgaagaacaagctggggatcttcagacggcggaatgagtcccctggagcccctcccgcgggcaaggcagacaaaatgatgaagtcattcaagcccacctcagaggaagccctcaagtggggcgagtccttggagaagctgctggttcacaaatacgggttagcagtgttccaagccttccttcgcactgagttcagtgaggagaatctggagttctggttggcttgtgaggacttcaagaaggtcaagtcacagtccaagatggcatccaaggccaagaagatctttgctgaatacatcgcgatccaggcatgcaaggaggtcaacctggactcctacacgcgggagcacaccaaggacaacctgcagagcgtcacgcggggctgcttcgacctggcacagaagcgcatcttcgggctcatggaaaaggactcgtaccctcgctttctccgttctgacctctacctggaccttattaaccagaagaagatgagtcccccgctttaggggccactggagtcgagctcagcgttcacaccaggcgggctgggtcccctgcccacctgcctccctgccccctgtgacggagggggcaagcaagcccccagaggctgtgtctctggacagacggatagacatacggaagcgaggcctggaccaagagaggcccaggctactggaggagtagaaggatgggccccgtggggtccccactgccccggtacgagggggcccaagaccctggcaggtcaggggccctggccaagccagatctggagctgctgctccctgctgcggagaccgcggaggcttcgcgttgaccaagttccttaaagaactggctgatggggcaggaggtccaggcctgggctctcgggccctcctagagggccattggagcttgcagctcagacccccactttgagttttatttatttaaatagtagttggatgcttggcacgtcgtcctgtaataggaaacccttgcctcatcagttttcctgatttacaagtgcaatattttagccaatgccttgggagaagctgccatgcaaaggtggacaccattctccagcttcaggggatatgctcgtcccgggcaccggtggcaggcagctggccttctggactaaggcagcctggggggacactgcagtctggctacacacagagatctggcaccccctgggtggagtgtccctcgggggctttgggaaagcatggcaccctcagaccacacagtagccaagttctggagcaaataaaaggcctgtgttatttcttgttcttga

SEQ ID NO: 17 - DNA, 3215 bp, Homo sapiens
actgcgtggagacgatgatcctgccagctcccttttggaaatcgtgaggatcagatcttggaccatgtataatatgatgcttctaatccaaaagaggaaaggcattgggagtcagctcctaagggctggagaggcagagggagacagaggagctggtactgcagagcggtcgtctgattggctggacggtcgtagctgggctataaaagagacccctacaggcttagcaggaagacgctcagaggattctgacaatatctttaccggagaagaggcaaagtacgctcaaagccgaagccacagctcctcctgccgcatttctttcctgcttgcgaattccaagctgttaaataagatgtgcaaagggcttgcaggtctgccggcttcttgcttgaggagtgcaaaagatatgaaacatcggctaggtttcctgctgcaaaaatctgattcctgtgaacacaattcttcccacaacaagaaggacaaagtggttatttgccagagagtgagccaagaggaagtcaagaaatgggctgaatcactggaaaacctgattagtcatgaatgtgggctggcagctttcaaagctttcttgaagtctgaatatagtgaggagaatattgacttctggatcagctgtgaagagtacaagaaaatcaaatcaccatctaaactaagtcccaaggccaaaaagatctataatgaattcatctcagtccaggcaaccaaagaggtgaacctggattcttgcaccagggaagagacaagccggaacatgctagagcctacaataacctgctttgatgaggcccagaagaagattttcaacctgatggagaaggattcctaccgccgcttcctcaagtctcgattctatcttgatttggtcaacccgtccagctgtggggcagaaaagcagaaaggagccaagagttcagcagactgtgcttccctggtccctcagtgtgcctaattctcacctgaaggcagagggatgaaatgccaagactctatgctctggaaaacctgaggccaaatattgatctgtattaagctccagtgctttatccacattgtagcctaatattcatgctgcctgccatgtgtgagtcacttctacgcataaactagatatagcttttggtgtttgagtgttcatcagggtgggaccccattccagtccaattttcctaagtttctttgagggttccatgggagcaaatatctaaataatggcctggtaggtctggattttcaaagattgttggcagtttcctcctcccaacagttttacctcgggatggttggttagtgcatgtcacatgacatccacatgcacatgtattctgttggccagcacgttctccagactctagatgtttagatgaggttgagctatgatatgtgcttgtgtgtatgtctatgtgtatatattatatatacattagacacacatatacattatttctgtatatagatgtctgtgtatacatatgtatgtgtgagtgtatgtatacacacacacacacacacacacacacacttttgcaagagtgatgggaaagaccctaggtgctcataactagagtatgtgtatgtacttacatgggtgttttgatctctgttctttcatactacatttgaacagggcaaaatgaactaactgccatgtaggctaagaaagaaatgctaacctgtggaaagttggttttgtaaaattccatggatcttgctggagaagcatccaaggaacttcatgcttgatttgaccactgacagcctccaccttgagcactattctaaggagcaaataccttagctcccttgagctggttttctctgatggcacttttgagctcctaagctgccagccttcccttcttttcctgggtgctcagggcatgcttattagcagctgggttggtatggagttggcagacaggatgttcaacttaatgaagaaatacagctaaggccttgccagcaacacctgccgtaagttactggctgagtgagggcatagaagttaaaggttactgtttttatcctctatccttttttcctttcctgatcaaggtgctcttctcattttttcctgagaaccttagccatcagatgaggctccttagtttattgtggttggttgttttttctttataatggctctgggctatatgcctatatttataaaccagcagcaggggaaagattatattttataagagggaacaaattttcacaatttgaaaagcccacataagttttctcttttaaggtagaatcttgttaatttcattccaaacatcggggctaacagagactggaggcatttctttttaggctctgagactaaatgagaggaaaagaaaagaaaaaaaaaatgattgtctaaccaattgtgagaattactgtttgaaacttttcaaggcacattgaaatacttgaaaacttctcatttatgttatttatgatgttattttgtacgtgttattattattatattgttttataaatggaggtacaggatatcacctgaattattaatgaatgcccaggaagtaattttcttctcattcttctaaaactactgcctttcaaagtgcacacacacgcgtccacatacactgcattcgttgctccagtataaattacatgcatgagcacctttctggcttttaagccaatataatgggctgcaaaatgaagacaccagagtgtatgcatacaaatctcactgtattaaagatgcaggttttctaattgtacccttcttgtctctctggcaatcttgcccttaatatccctggagttcctcatcagtgtcattttctgttatacacagttccacaattttgtctctagttgacttcaaatgtgtaactttattggtcttgccctattataattgtcatgactttcagattgtatctgaactcacagactgctgtcttactaataggtctggaaggtcacgctgaatgagaagtaaattattttatgtaatacatttttgagtgtgtttttcagttgtatttccctgttatttcatcactatttccaatggtgagcttgcctgctcatgctccctggacagaatactccttccttttgcatgcctgtttctatcatgtgcttgataggcctcaaagctaatgcttccagtgaaacacacgcatcttaataataagggtaaataaacgctccatatgaaacta

SEQ ID NO: 18 - DNA, 3055 bp, Homo sapiens
actttcccgaggtgcttctacagttccctctgccagcaggggaacagatggaaatagcaatcacctgccagaaggtggcgtgcagcaaggatgtgcatcttttgccgctactgctttctgattcctaaaaattactcagagatcactcatgtgttcagtgattcaggttctgttgaagataccaaagatattcggttggtcaaaatgacgggcatataaaggcttctcaggtttctgagtgcaaaagatatgaaacatcggctaggtttcctgctgcaaaaatctgattcctgtgaacacaattcttcccacaacaagaaggacaaagtggttatttgccagagagtgagccaagaggaagtcaagaaatgggctgaatcactggaaaacctgattagtcatgaatgtgggctggcagctttcaaagctttcttgaagtctgaatatagtgaggagaatattgacttctggatcagctgtgaagagtacaagaaaatcaaatcaccatctaaactaagtcccaaggccaaaaagatctataatgaattcatctcagtccaggcaaccaaagaggtgaacctggattcttgcaccagggaagagacaagccggaacatgctagagcctacaataacctgctttgatgaggcccagaagaagattttcaacctgatggagaaggattcctaccgccgcttcctcaagtctcgattctatcttgatttggtcaacccgtccagctgtggggcagaaaagcagaaaggagccaagagttcagcagactgtgcttccctggtccctcagtgtgcctaattctcacctgaaggcagagggatgaaatgccaagactctatgctctggaaaacctgaggccaaatattgatctgtattaagctccagtgctttatccacattgtagcctaatattcatgctgcctgccatgtgtgagtcacttctacgcataaactagatatagcttttggtgtttgagtgttcatcagggtgggaccccattccagtccaattttcctaagtttctttgagggttccatgggagcaaatatctaaataatggcctggtaggtctggattttcaaagattgttggcagtttcctcctcccaacagttttacctcgggatggttggttagtgcatgtcacatgacatccacatgcacatgtattctgttggccagcacgttctccagactctagatgtttagatgaggttgagctatgatatgtgcttgtgtgtatgtctatgtgtatatattatatatacattagacacacatatacattatttctgtatatagatgtctgtgtatacatatgtatgtgtgagtgtatgtatacacacacacacacacacacacacacacttttgcaagagtgatgggaaagaccctaggtgctcataactagagtatgtgtatgtacttacatgggtgttttgatctctgttctttcatactacatttgaacagggcaaaatgaactaactgccatgtaggctaagaaagaaatgctaacctgtggaaagttggttttgtaaaattccatggatcttgctggagaagcatccaaggaacttcatgcttgatttgaccactgacagcctccaccttgagcactattctaaggagcaaataccttagctcccttgagctggttttctctgatggcacttttgagctcctaagctgccagccttcccttcttttcctgggtgctcagggcatgcttattagcagctgggttggtatggagttggcagacaggatgttcaacttaatgaagaaatacagctaaggccttgccagcaacacctgccgtaagttactggctgagtgagggcatagaagttaaaggttactgtttttatcctctatccttttttcctttcctgatcaaggtgctcttctcattttttcctgagaaccttagccatcagatgaggctccttagtttattgtggttggttgttttttctttataatggctctgggctatatgcctatatttataaaccagcagcaggggaaagattatattttataagagggaacaaattttcacaatttgaaaagcccacataagttttctcttttaaggtagaatcttgttaatttcattccaaacatcggggctaacagagactggaggcatttctttttaggctctgagactaaatgagaggaaaagaaaagaaaaaaaaaatgattgtctaaccaattgtgagaattactgtttgaaacttttcaaggcacattgaaatacttgaaaacttctcatttatgttatttatgatgttattttgtacgtgttattattattatattgttttataaatggaggtacaggatatcacctgaattattaatgaatgcccaggaagtaattttcttctcattcttctaaaactactgcctttcaaagtgcacacacacgcgtccacatacactgcattcgttgctccagtataaattacatgcatgagcacctttctggcttttaagccaatataatgggctgcaaaatgaagacaccagagtgtatgcatacaaatctcactgtattaaagatgcaggttttctaattgtacccttcttgtctctctggcaatcttgcccttaatatccctggagttcctcatcagtgtcattttctgttatacacagttccacaattttgtctctagttgacttcaaatgtgtaactttattggtcttgccctattataattgtcatgactttcagattgtatctgaactcacagactgctgtcttactaataggtctggaaggtcacgctgaatgagaagtaaattattttatgtaatacatttttgagtgtgtttttcagttgtatttccctgttatttcatcactatttccaatggtgagcttgcctgctcatgctccctggacagaatactccttccttttgcatgcctgtttctatcatgtgcttgataggcctcaaagctaatgcttccagtgaaacacacgcatcttaataataagggtaaataaacgctccatatgaaacta

SEQ ID NO: 19 - DNA, 2924 bp, Homo sapiens
gctggagaggcagagggagacagaggagctggtactgcagagcggtcgtctgattggctggacggtcgtagctgggctataaaagagacccctacaggcttagcaggaagacgctcagaggattctgacaatatctttaccggagaagaggcaaagtacgctcaaagccgaagccacagctcctcctgccgcatttctttcctgcttgcgaattccaagctgttaaataagatgtgcaaagggcttgcaggtctgccggcttcttgcttgaggagtgcaaaagatatgaaacatcggctaggtttcctgctgcaaaaatctgattcctgtgaacacaattcttcccacaacaagaaggacaaagtggttatttgccagagagtgagccaagaggaagtcaagaaatgggctgaatcactggaaaacctgattagtcatgaatgtgaacctggattcttgcaccagggaagagacaagccggaacatgctagagcctacaataacctgctttgatgaggcccagaagaagattttcaacctgatggagaaggattcctaccgccgcttcctcaagtctcgattctatcttgatttggtcaacccgtccagctgtggggcagaaaagcagaaaggagccaagagttcagcagactgtgcttccctggtccctcagtgtgcctaattctcacctgaaggcagagggatgaaatgccaagactctatgctctggaaaacctgaggccaaatattgatctgtattaagctccagtgctttatccacattgtagcctaatattcatgctgcctgccatgtgtgagtcacttctacgcataaactagatatagcttttggtgtttgagtgttcatcagggtgggaccccattccagtccaattttcctaagtttctttgagggttccatgggagcaaatatctaaataatggcctggtaggtctggattttcaaagattgttggcagtttcctcctcccaacagttttacctcgggatggttggttagtgcatgtcacatgacatccacatgcacatgtattctgttggccagcacgttctccagactctagatgtttagatgaggttgagctatgatatgtgcttgtgtgtatgtctatgtgtatatattatatatacattagacacacatatacattatttctgtatatagatgtctgtgtatacatatgtatgtgtgagtgtatgtatacacacacacacacacacacacacacacttttgcaagagtgatgggaaagaccctaggtgctcataactagagtatgtgtatgtacttacatgggtgttttgatctctgttctttcatactacatttgaacagggcaaaatgaactaactgccatgtaggctaagaaagaaatgctaacctgtggaaagttggttttgtaaaattccatggatcttgctggagaagcatccaaggaacttcatgcttgatttgaccactgacagcctccaccttgagcactattctaaggagcaaataccttagctcccttgagctggttttctctgatggcacttttgagctcctaagctgccagccttcccttcttttcctgggtgctcagggcatgcttattagcagctgggttggtatggagttggcagacaggatgttcaacttaatgaagaaatacagctaaggccttgccagcaacacctgccgtaagttactggctgagtgagggcatagaagttaaaggttactgtttttatcctctatccttttttcctttcctgatcaaggtgctcttctcattttttcctgagaaccttagccatcagatgaggctccttagtttattgtggttggttgttttttctttataatggctctgggctatatgcctatatttataaaccagcagcaggggaaagattatattttataagagggaacaaattttcacaatttgaaaagcccacataagttttctcttttaaggtagaatcttgttaatttcattccaaacatcggggctaacagagactggaggcatttctttttaggctctgagactaaatgagaggaaaagaaaagaaaaaaaaaatgattgtctaaccaattgtgagaattactgtttgaaacttttcaaggcacattgaaatacttgaaaacttctcatttatgttatttatgatgttattttgtacgtgttattattattatattgttttataaatggaggtacaggatatcacctgaattattaatgaatgcccaggaagtaattttcttctcattcttctaaaactactgcctttcaaagtgcacacacacgcgtccacatacactgcattcgttgctccagtataaattacatgcatgagcacctttctggcttttaagccaatataatgggctgcaaaatgaagacaccagagtgtatgcatacaaatctcactgtattaaagatgcaggttttctaattgtacccttcttgtctctctggcaatcttgcccttaatatccctggagttcctcatcagtgtcattttctgttatacacagttccacaattttgtctctagttgacttcaaatgtgtaactttattggtcttgccctattataattgtcatgactttcagattgtatctgaactcacagactgctgtcttactaataggtctggaaggtcacgctgaatgagaagtaaattattttatgtaatacatttttgagtgtgtttttcagttgtatttccctgttatttcatcactatttccaatggtgagcttgcctgctcatgctccctggacagaatactccttccttttgcatgcctgtttctatcatgtgcttgataggcctcaaagctaatgcttccagtgaaacacacgcatcttaataataagggtaaataaacgctccatatgaaacta

SEQ ID NO: 20 - DNA, 2983 bp, Homo sapiens
aagacgctcagaggattctgacaatatctttaccggagaagaggcaaagtacgctcaaagccgaagccacagctcctcctgccgcatttctttcctgcttgcgaattccaagctgttaaataagatgtgcaaagggcttgcaggtctgccggcttcttgcttgaggagtgcaaaagatatgaaacatcggctaggtttcctgctgcaaaaatctgattcctgtgaacacaattcttcccacaacaagaaggacaaagtggttatttgccagagagtgagccaagaggaagtcaagaaatgggctgaatcactggaaaacctgattagtcatgaatgtgggctggcagctttcaaagctttcttgaagtctgaatatagtgaggagaatattgacttctggatcagctgtgaagagtacaagaaaatcaaatcaccatctaaactaagtcccaaggccaaaaagatctataatgaattcatctcagtccaggcaaccaaagaggtgaacctggattcttgcaccagggaagagacaagccggaacatgctagagcctacaataacctgctttgatgaggcccagaagaagattttcaacctgatggagaaggattcctaccgccgcttcctcaagtctcgattctatcttgatttggtcaacccgtccagctgtggggcagaaaagcagaaaggagccaagagttcagcagactgtgcttccctggtccctcagtgtgcctaattctcacctgaaggcagagggatgaaatgccaagactctatgctctggaaaacctgaggccaaatattgatctgtattaagctccagtgctttatccacattgtagcctaatattcatgctgcctgccatgtgtgagtcacttctacgcataaactagatatagcttttggtgtttgagtgttcatcagggtgggaccccattccagtccaattttcctaagtttctttgagggttccatgggagcaaatatctaaataatggcctggtaggtctggattttcaaagattgttggcagtttcctcctcccaacagttttacctcgggatggttggttagtgcatgtcacatgacatccacatgcacatgtattctgttggccagcacgttctccagactctagatgtttagatgaggttgagctatgatatgtgcttgtgtgtatgtctatgtgtatatattatatatacattagacacacatatacattatttctgtatatagatgtctgtgtatacatatgtatgtgtgagtgtatgtatacacacacacacacacacacacacacacttttgcaagagtgatgggaaagaccctaggtgctcataactagagtatgtgtatgtacttacatgggtgttttgatctctgttctttcatactacatttgaacagggcaaaatgaactaactgccatgtaggctaagaaagaaatgctaacctgtggaaagttggttttgtaaaattccatggatcttgctggagaagcatccaaggaacttcatgcttgatttgaccactgacagcctccaccttgagcactattctaaggagcaaataccttagctcccttgagctggttttctctgatggcacttttgagctcctaagctgccagccttcccttcttttcctgggtgctcagggcatgcttattagcagctgggttggtatggagttggcagacaggatgttcaacttaatgaagaaatacagctaaggccttgccagcaacacctgccgtaagttactggctgagtgagggcatagaagttaaaggttactgtttttatcctctatccttttttcctttcctgatcaaggtgctcttctcattttttcctgagaaccttagccatcagatgaggctccttagtttattgtggttggttgttttttctttataatggctctgggctatatgcctatatttataaaccagcagcaggggaaagattatattttataagagggaacaaattttcacaatttgaaaagcccacataagttttctcttttaaggtagaatcttgttaatttcattccaaacatcggggctaacagagactggaggcatttctttttaggctctgagactaaatgagaggaaaagaaaagaaaaaaaaaatgattgtctaaccaattgtgagaattactgtttgaaacttttcaaggcacattgaaatacttgaaaacttctcatttatgttatttatgatgttattttgtacgtgttattattattatattgttttataaatggaggtacaggatatcacctgaattattaatgaatgcccaggaagtaattttcttctcattcttctaaaactactgcctttcaaagtgcacacacacgcgtccacatacactgcattcgttgctccagtataaattacatgcatgagcacctttctggcttttaagccaatataatgggctgcaaaatgaagacaccagagtgtatgcatacaaatctcactgtattaaagatgcaggttttctaattgtacccttcttgtctctctggcaatcttgcccttaatatccctggagttcctcatcagtgtcattttctgttatacacagttccacaattttgtctctagttgacttcaaatgtgtaactttattggtcttgccctattataattgtcatgactttcagattgtatctgaactcacagactgctgtcttactaataggtctggaaggtcacgctgaatgagaagtaaattattttatgtaatacatttttgagtgtgtttttcagttgtatttccctgttatttcatcactatttccaatggtgagcttgcctgctcatgctccctggacagaatactccttccttttgcatgcctgtttctatcatgtgcttgataggcctcaaagctaatgcttccagtgaaacacacgcatcttaataataagggtaaataaacgctccatatgaaact

SEQ ID NO: 21 - protein, 169 aa, Homo sapiens
DMKNKLGIFRFLRTEFSEENLQSVTRGCFDLRRNESPGAPPEFWLACEDFKAQKRIFGLMEAGKADKMMKKVKSQSKMASKDSYPRFLRSSFKPTSEEALKKAKKIFAEYIAIDLYLDLINQKKWGESLEKLLVQACKEVNLDSMSPPLHKYGLAVFQAYTREHTKDNL

SEQ ID NO: 22 - protein, 13 aa, Artificial Sequence
  Description of Artificial Sequence Synthetic peptide
KSFKPTSEEALKW

SEQ ID NO: 23 - protein, 13 aa, Artificial Sequence
  Description of Artificial Sequence Synthetic peptide
KYGLAVFQAFLRT

SEQ ID NO: 24 - protein, 11 aa, Artificial Sequence
  Description of Artificial Sequence Synthetic peptide
KEVNLDSYTRE

SEQ ID NO: 25 - protein, 13 aa, Artificial Sequence
  Description of Artificial Sequence Synthetic peptide
RSDLYLDLINQKK

SEQ ID NO: 26 - protein, 14 aa, Artificial Sequence
  Description of Artificial Sequence Synthetic peptide
RSDLYLDLINQKKM